(19) United States
(12) Patent Application Publication
(10) Pub. No.: US 2003/0064379 A1
(43) Pub. Date: Apr. 3, 2003

(54) Novel polynucleotides and method of use thereof

(75) Inventors: Kevin P. Baker, Darnestown, MD (US); Audrey Goddard, San Francisco, CA (US); William I. Wood, Hillsborough, CA (US)

Correspondence Address: Mark T. Kresnak, Ph.D., Genentech, Inc., 1 DNA Way, MS 49, South San Francisco, CA, 94080, US

(73) Assignee: GENENTECH, INC.

(21) Appl. No.: 10/052,283

(22) Filed: Jan. 15, 2002

Related U.S. Application Data
(60) Provisional application No. 60/145,701, filed on Jul. 26, 1999.

(30) Foreign Application Priority Data
Jul. 21, 2000 (US) PCT/US00/20006

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

The present invention is directed to novel polynucleotides and to polypeptides encoded thereby. Also provided herein are vectors and host cells comprising those nucleic acid sequences, chimeric polypeptide molecules comprising the polypeptides of the present invention fused to heterologous polypeptide sequences, antibodies which bind to the polypeptides of the present invention and to methods for producing the polypeptides of the present invention.

### FIELD OF THE INVENTION

The present invention relates generally to the identification and isolation of novel nucleic acid molecules which constitute at least a portion of full-length cDNA molecules that encode human polypeptides.

### BACKGROUND OF THE INVENTION

Extracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Secreted proteins have various industrial applications, including as pharmaceuticals, diagnostics, biosensors and bioreactors. Most protein drugs available at present, such as thrombolytic agents, interferons, interleukins, erythropoietins, colony stimulating factors, and various other cytokines, are secretory proteins. Their receptors, which are membrane proteins, also have potential as therapeutic or diagnostic agents. Efforts are being undertaken by both industry and academia to identify new, native secreted proteins. Many efforts are focused on the screening of mammalian recombinant DNA libraries to identify the coding sequences for novel secreted proteins. Examples of screening methods and techniques are described in the literature [see, for example, Klein et al., Proc. Natl. Acad. Sci., 93:7108-7113 (1996); U.S. Pat. No. 5,536,637)].

Membrane-bound proteins and receptors can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, cytokine receptors, receptor kinases, receptor phosphatases, receptors involved in cell-cell interactions, and cellular adhesin molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Membrane-bound proteins and receptor molecules have various industrial applications, including as pharmaceutical and diagnostic agents. Receptor immunoadhesins, for instance, can be employed as therapeutic agents to block receptor-ligand interactions. The membrane-bound proteins can also be employed for screening of potential peptide or small molecule inhibitors of the relevant receptor/ligand interaction. Efforts are being undertaken by both industry and academia to identify new, native receptor or membrane-bound proteins. Many efforts are focused on the screening of mammalian recombinant DNA libraries to identify the coding sequences for novel receptor or membrane-bound proteins.

Recently, significant progress has been made in identifying and isolating unique nucleic acid moelcules which encode all or a portion of many mammalian proteins. We herein describe the identification and characterization of novel polynucleotides which constitute at least partial cDNA molecules that encode various human polypeptides.

### SUMMARY OF THE INVENTION

Novel polynucleotides have been identified and isolated which constitute at least partial cDNA molecules that encode human polypeptides.

In one embodiment, the invention provides an isolated nucleic acid molecule comprising any one of the nucleic acid sequences shown in the accompanying figures, or the complement thereof, or polynucleotide variants of those nucleic acid sequences as defined below.

In another embodiment, the invention provides an isolated nucleic acid molecule consisting essentially of any one of the nucleic acid sequences shown in the accompanying figures, or the complement thereof, or polynucleotide variants of those nucleic acid sequences as defined below.

In another embodiment, the invention provides an isolated nucleic acid molecule consisting of any one of the nucleic acid sequences shown in the accompanying figures, or the complement thereof, or polynucleotide variants of those nucleic acid sequences as defined below.

In yet another embodiment, the invention provides an isolated nucleic acid molecule that comprises a nucleotide sequence having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity and yet more preferably at least about 99% sequence identity to (a) the DNA molecule of any one of FIG. 1 to 562, or (b) the complement of the DNA molecule of (a).

In another aspect, the isolated nucleic acid molecule consists essentially of a nucleotide sequence having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity and yet more preferably at least about 99% sequence identity to (a) the DNA molecule of any one of FIG. 1 to 562, or (b) the complement of the DNA molecule of (a).

In yet another aspect, the isolated nucleic acid molecule consists of a nucleotide sequence having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity and yet more preferably at least about 99% sequence identity to (a) the DNA molecule of any one of FIG. 1 to 562, or (b) the complement of the DNA molecule of (a).

In another embodiment, the invention concerns an isolated nucleic acid molecule which comprises a nucleotide sequence that hybridizes to (a) the DNA molecule of any one of FIG. 1 to 562, or (b) the complement of the DNA molecule of (a). Preferably, hybridization occurs under stringent hybridization and wash conditions. Also, it is preferred that the isolated nucleic acid molecule is fully complementary to (a) the DNA molecule of any one of FIG. 1 to 562, or (b) the complement of the DNA molecule of (a).

In yet another embodiment, the present invention provides an isolated nucleic acid molecule which comprises at least about 10 consecutive nucleotides contained within (a) the DNA molecule of any one of FIG. 1 to 562, or (b) the complement of the DNA molecule of (a) which may find use as, for example, hybridizing oligonucleotide probes or for encoding polypeptide fragments that may optionally comprise a binding site for an antibody. In particular aspects, the isolated nucleic acid molecule is from about 10 to about 1000, about 10 to about 900, about 10 to about 800, about 10 to about 700, about 10 to about 600, about 10 to about 500, about 10 to about 400, about 10 to about 300, about 10 to about 200, about 10 to about 100, about 10 to about 90, about 10 to about 80, about 10 to about 70, about 10 to about 60, about 10 to about 50, about 10 to about 40, about 10 to about 30 or about 10 to about 20 nucleotides in length, where the term “about” means the referenced nucleotide sequence length plus or minus 10% of that referenced length. In yet other aspects, the isolated nucleic acid molecule comprises at least about 15, 20, 25, 30, 35, 40, 45, 50, 55, 60, 65, 70, 75, 80, 85, 90, 95 or 100 consecutive nucleotides contained within (a) the DNA molecule of any one of FIG. 1 to 562, or (b) the complement of the DNA molecule of (a).

The present invention is also directed to a method of using an oligonucleotide probe having a nucleotide sequence derived from a nucleic acid molecule described herein for detecting the presence of and/or obtaining a full-length mammalian cDNA molecule from a mammalian cDNA library which encodes a mammalian polypeptide. Preferably, the mammal is human. The methods comprise the step of screening a mammalian cDNA library with one or more of the herein described oligonucleotides to detect the presence of a full-length cDNA and, optionally, obtaining the full-length cDNA from that library.

In another embodiment, the invention provides a vector comprising any of the isolated nucleic acid molecules described herein or their variants.

A host cell comprising such a vector is also provided. By way of example, the host cells may be CHO cells, E. coli, or yeast. A process for producing polypeptides is further provided and comprises culturing the host cells under conditions suitable for expression of a polypeptide and recovering the polypeptide from the cell culture.

In another embodiment, the invention provides isolated polypeptides encoded by any of the isolated nucleic acids described herein, wherein thise polypeptides are herein designated as SRT polypeptides.

In yet another embodiment, the invention provides antibodies which specifically bind to a polypeptide encoded by a nucleic acid molecule described herein. Preferably, the antibodies are monoclonal antibodies.

### BRIEF DESCRIPTION OF THE DRAWINGS

FIG. 1 shows a nucleotide sequence (SEQ ID NO:1) designated herein as DNA8284.

FIG. 2 shows a nucleotide sequence (SEQ ID NO:2) designated herein as DNA8328.

FIG. 3 shows a nucleotide sequence (SEQ ID NO:3) designated herein as DNA8350.

FIG. 4 shows a nucleotide sequence (SEQ ID NO:4) designated herein as DNA8369.

FIG. 5 shows a nucleotide sequence (SEQ ID NO:5) designated herein as DNA8377.

FIG. 6 shows a nucleotide sequence (SEQ ID NO:6) designated herein as DNA8456.

FIG. 7 shows a nucleotide sequence (SEQ ID NO:7) designated herein as DNA8555.

FIG. 8 shows a nucleotide sequence (SEQ ID NO:8) designated herein as DNA8576.

FIG. 9 shows a nucleotide sequence (SEQ ID NO:9) designated herein as DNA9383.

FIG. 10 shows a nucleotide sequence (SEQ ID NO:10) designated herein as DNA9840.

FIG. 11 shows a nucleotide sequence (SEQ ID NO:11) designated herein as DNA10028.

FIG. 12 shows a nucleotide sequence (SEQ ID NO:12) designated herein as DNA10072.

FIG. 13 shows a nucleotide sequence (SEQ ID NO:13) designated herein as DNA10242.

FIG. 14 shows a nucleotide sequence (SEQ ID NO:14) designated herein as DNA10281.

FIG. 15 shows a nucleotide sequence (SEQ ID NO:15) designated herein as DNA12628.

FIG. 16 shows a nucleotide sequence (SEQ ID NO:16) designated herein as DNA12646.

FIG. 17 shows a nucleotide sequence (SEQ ID NO:17) designated herein as DNA12655.

FIG. 18 shows a nucleotide sequence (SEQ ID NO:18) designated herein as DNA12660.

FIG. 19 shows a nucleotide sequence (SEQ ID NO:19) designated herein as DNA12668.

FIG. 20 shows a nucleotide sequence (SEQ ID NO:20) designated herein as DNA12726.

FIG. 21 shows a nucleotide sequence (SEQ ID NO:21) designated herein as DNA12728.

FIG. 22 shows a nucleotide sequence (SEQ ID NO:22) designated herein as DNA12729.

FIG. 23 shows a nucleotide sequence (SEQ ID NO:23) designated herein as DNA12732.

FIG. 24 shows a nucleotide sequence (SEQ ID NO:24) designated herein as DNA12733.

FIG. 25 shows a nucleotide sequence (SEQ ID NO:25) designated herein as DNA12741.

FIG. 26 shows a nucleotide sequence (SEQ ID NO:26) designated herein as DNA12742.

FIG. 27 shows a nucleotide sequence (SEQ ID NO:27) designated herein as DNA 12747.

FIG. 28 shows a nucleotide sequence (SEQ ID NO:28) designated herein as DNA12752.

FIG. 29 shows a nucleotide sequence (SEQ ID NO:29) designated herein as DNA 12797.

FIG. 30 shows a nucleotide sequence (SEQ ID NO:30) designated herein as DNA12801.

FIG. 31 shows a nucleotide sequence (SEQ ID NO:31) designated herein as DNA12802.

FIG. 32 shows a nucleotide sequence (SEQ ID NO:32) designated herein as DNA12817.

FIG. 33 shows a nucleotide sequence (SEQ ID NO:33) designated herein as DNA12819.

FIG. 34 shows a nucleotide sequence (SEQ ID NO:34) designated herein as DNA12829.

FIG. 35 shows a nucleotide sequence (SEQ ID NO:35) designated herein as DNA12830.

FIG. 36 shows a nucleotide sequence (SEQ ID NO:36) designated herein as DNA12834.

FIG. 37 shows a nucleotide sequence (SEQ ID NO:37) designated herein as DNA12837.

FIG. 38 shows a nucleotide sequence (SEQ ID NO:38) designated herein as DNA12840.

FIG. 39 shows a nucleotide sequence (SEQ ID NO:39) designated herein as DNA12841.

FIG. 40 shows a nucleotide sequence (SEQ ID NO:40) designated herein as DNA 12844.

FIG. 41 shows a nucleotide sequence (SEQ ID NO:41) designated herein as DNA 12846.

FIG. 42 shows a nucleotide sequence (SEQ ID NO:42) designated herein as DNA12850.

FIG. 43 shows a nucleotide sequence (SEQ ID NO:43) designated herein as DNA 12865.

FIG. 44 shows a nucleotide sequence (SEQ ID NO:44) designated herein as DNA12867.

FIG. 45 shows a nucleotide sequence (SEQ ID NO:45) designated herein as DNA12884.

FIG. 46 shows a nucleotide sequence (SEQ ID NO:46) designated herein as DNA 12889.

FIG. 47 shows a nucleotide sequence (SEQ ID NO:47) designated herein as DNA12891.

FIG. 48 shows a nucleotide sequence (SEQ ID NO:48) designated herein as DNA12900.

FIG. 49 shows a nucleotide sequence (SEQ ID NO:49) designated herein as DNA12922.

FIG. 50 shows a nucleotide sequence (SEQ ID NO:50) designated herein as DNA12946.

FIG. 51 shows a nucleotide sequence (SEQ ID NO:51) designated herein as DNA12967.

FIG. 52 shows a nucleotide sequence (SEQ ID NO:52) designated herein as DNA12974.

FIG. 53 shows a nucleotide sequence (SEQ ID NO:53) designated herein as DNA12982.

FIG. 54 shows a nucleotide sequence (SEQ ID NO:54) designated herein as DNA12983.

FIG. 55 shows a nucleotide sequence (SEQ ID NO:55) designated herein as DNA12991.

FIG. 56 shows a nucleotide sequence (SEQ ID NO:56) designated herein as DNA12998.

FIG. 57 shows a nucleotide sequence (SEQ ID NO:57) designated herein as DNA12999.

FIG. 58 shows a nucleotide sequence (SEQ ID NO:58) designated herein as DNA13101.

FIG. 59 shows a nucleotide sequence (SEQ ID NO:59) designated herein as DNA13104.

FIG. 60 shows a nucleotide sequence (SEQ ID NO:60) designated herein as DNA13110.

FIG. 61 shows a nucleotide sequence (SEQ ID NO:61) designated herein as DNA13114.

FIG. 62 shows a nucleotide sequence (SEQ ID NO:62) designated herein as DNA13115.

FIG. 63 shows a nucleotide sequence (SEQ ID NO:63) designated herein as DNA13116.

FIG. 64 shows a nucleotide sequence (SEQ ID NO:64) designated herein as DNA13118.

FIG. 65 shows a nucleotide sequence (SEQ ID NO:65) designated herein as DNA13124.

FIG. 66 shows a nucleotide sequence (SEQ ID NO:66) designated herein as DNA13132.

FIG. 67 shows a nucleotide sequence (SEQ ID NO:67) designated herein as DNA13133.

FIG. 68 shows a nucleotide sequence (SEQ ID NO:68) designated herein as DNA13146.

FIG. 69 shows a nucleotide sequence (SEQ ID NO:69) designated herein as DNA13152.

FIG. 70 shows a nucleotide sequence (SEQ ID NO:70) designated herein as DNA13156.

FIG. 71 shows a nucleotide sequence (SEQ ID NO:71) designated herein as DNA13163.

FIG. 72 shows a nucleotide sequence (SEQ ID NO:72) designated herein as DNA13185.

FIG. 73 shows a nucleotide sequence (SEQ ID NO:73) designated herein as DNA 13992.

FIG. 74 shows a nucleotide sequence (SEQ ID NO:74) designated herein as DNA 14523.

FIG. 75 shows a nucleotide sequence (SEQ ID NO:75) designated herein as DNA 14656.

FIG. 76 shows a nucleotide sequence (SEQ ID NO:76) designated herein as DNA14938.

FIG. 77 shows a nucleotide sequence (SEQ ID NO:77) designated herein as DNA15172.

FIG. 78 shows a nucleotide sequence (SEQ ID NO:78) designated herein as DNA15618.

FIG. 79 shows a nucleotide sequence (SEQ ID NO:79) designated herein as DNA16546.

FIG. 80 shows a nucleotide sequence (SEQ ID NO:80) designated herein as DNA16669.

FIG. 81 shows a nucleotide sequence (SEQ ID NO:81) designated herein as DNA 17244.

FIG. 82 shows a nucleotide sequence (SEQ ID NO:82) designated herein as DNA18382.

FIG. 83 shows a nucleotide sequence (SEQ ID NO:83) designated herein as DNA18444.

FIG. 84 shows a nucleotide sequence (SEQ ID NO:84) designated herein as DNA18649.

FIG. 85 shows a nucleotide sequence (SEQ ID NO:85) designated herein as DNA19597.

FIG. 86 shows a nucleotide sequence (SEQ ID NO:86) designated herein as DNA19601.

FIG. 87 shows a nucleotide sequence (SEQ ID NO:87) designated herein as DNA21386.

FIG. 88 shows a nucleotide sequence (SEQ ID NO:88) designated herein as DNA22868.

FIG. 89 shows a nucleotide sequence (SEQ ID NO:89) designated herein as DNA23694.

FIG. 90 shows a nucleotide sequence (SEQ ID NO:90) designated herein as DNA24050.

FIG. 91 shows a nucleotide sequence (SEQ ID NO:91) designated herein as DNA24074.

FIG. 92 shows a nucleotide sequence (SEQ ID NO:92) designated herein as DNA24787.

FIG. 93 shows a nucleotide sequence (SEQ ID NO:93) designated herein as DNA28242.

FIG. 94 shows a nucleotide sequence (SEQ ID NO:94) designated herein as DNA28254.

FIG. 95 shows a nucleotide sequence (SEQ ID NO:95) designated herein as DNA31751.

FIG. 96 shows a nucleotide sequence (SEQ ID NO:96) designated herein as DNA32922.

FIG. 97 shows a nucleotide sequence (SEQ ID NO:97) designated herein as DNA33439.

FIG. 98 shows a nucleotide sequence (SEQ ID NO:98) designated herein as DNA34508.

FIG. 99 shows a nucleotide sequence (SEQ ID NO:99) designated herein as DNA34807.

FIG. 100 shows a nucleotide sequence (SEQ ID NO:100) designated herein as DNA34832.

FIG. 101 shows a nucleotide sequence (SEQ ID NO:101) designated herein as DNA36223.

FIG. 102 shows a nucleotide sequence (SEQ ID NO:102) designated herein as DNA36240.

FIG. 103 shows a nucleotide sequence (SEQ ID NO:103) designated herein as DNA36490.

FIG. 104 shows a nucleotide sequence (SEQ ID NO:104) designated herein as DNA36516.

FIG. 105 shows a nucleotide sequence (SEQ ID NO:105) designated herein as DNA36533.

FIG. 106 shows a nucleotide sequence (SEQ ID NO:106) designated herein as DNA36538.

FIG. 107 shows a nucleotide sequence (SEQ ID NO:107) designated herein as DNA36788.

FIG. 108 shows a nucleotide sequence (SEQ ID NO:108) designated herein as DNA36818.

FIG. 109 shows a nucleotide sequence (SEQ ID NO:109) designated herein as DNA36868.

FIG. 110 shows a nucleotide sequence (SEQ ID NO:110) designated herein as DNA37393.

FIG. 111 shows a nucleotide sequence (SEQ ID NO:111) designated herein as DNA27588.

FIG. 112 shows a nucleotide sequence (SEQ ID NO:112) designated herein as DNA37602.

FIG. 113 shows a nucleotide sequence (SEQ ID NO:113) designated herein as DNA37642.

FIG. 114 shows a nucleotide sequence (SEQ ID NO:114) designated herein as DNA37676.

FIG. 115 shows a nucleotide sequence (SEQ ID NO:115) designated herein as DNA37721.

FIG. 116 shows a nucleotide sequence (SEQ ID NO:116) designated herein as DNA37759.

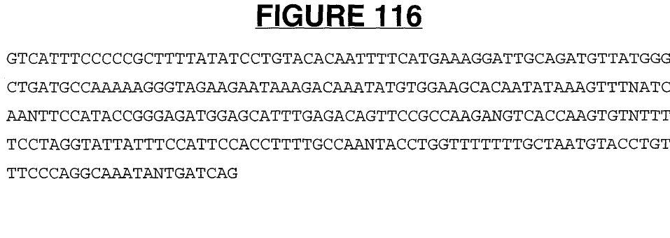

FIG. 117 shows a nucleotide sequence (SEQ ID NO:117) designated herein as DNA37857.

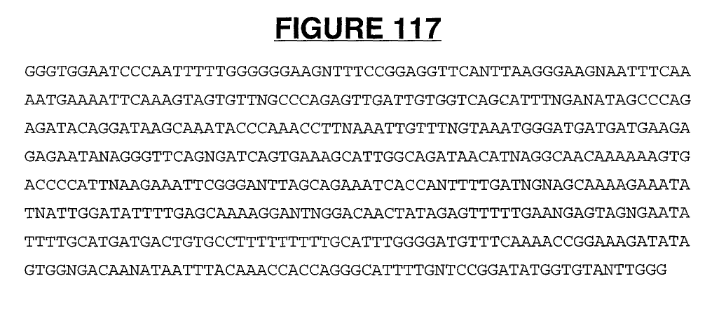

FIG. 118 shows a nucleotide sequence (SEQ ID NO:118) designated herein as DNA37937.

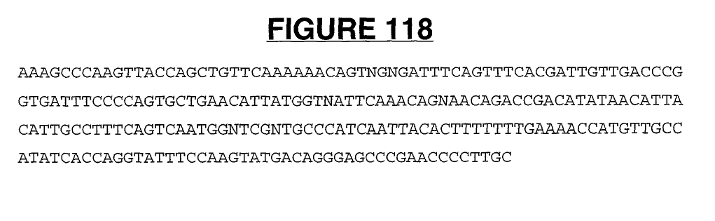

FIG. 119 shows a nucleotide sequence (SEQ ID NO:119) designated herein as DNA38037.

FIG. 120 shows a nucleotide sequence (SEQ ID NO:120) designated herein as DNA38050.

FIG. 121 shows a nucleotide sequence (SEQ ID NO:121) designated herein as DNA38053.

FIG. 122 shows a nucleotide sequence (SEQ ID NO:122) designated herein as DNA38312.

FIG. 123 shows a nucleotide sequence (SEQ ID NO:123) designated herein as DNA38360.

FIG. 124 shows a nucleotide sequence (SEQ ID NO:124) designated herein as DNA38600.

FIG. 125 shows a nucleotide sequence (SEQ ID NO:125) designated herein as DNA38720.

FIG. 126 shows a nucleotide sequence (SEQ ID NO:126) designated herein as DNA38727.

FIG. 127 shows a nucleotide sequence (SEQ ID NO:127) designated herein as DNA38731.

FIG. 128 shows a nucleotide sequence (SEQ ID NO:128) designated herein as DNA38810.

FIG. 129 shows a nucleotide sequence (SEQ ID NO:129) designated herein as DNA38814.

FIG. 130 shows a nucleotide sequence (SEQ ID NO:130) designated herein as DNA39378.

FIG. 131 shows a nucleotide sequence (SEQ ID NO:131) designated herein as DNA40050.

FIG. 132 shows a nucleotide sequence (SEQ ID NO:132) designated herein as DNA40375.

FIG. 133 shows a nucleotide sequence (SEQ ID NO:133) designated herein as DNA40382.

FIG. 134 shows a nucleotide sequence (SEQ ID NO:134) designated herein as DNA40394.

FIG. 135 shows a nucleotide sequence (SEQ ID NO:135) designated herein as DNA40461.

FIG. 136 shows a nucleotide sequence (SEQ ID NO:136) designated herein as DNA40735.

FIG. 137 shows a nucleotide sequence (SEQ ID NO:137) designated herein as DNA40736.

FIG. 138 shows a nucleotide sequence (SEQ ID NO:138) designated herein as DNA40738.

FIG. 139 shows a nucleotide sequence (SEQ ID NO:139) designated herein as DNA40739.

FIG. 140 shows a nucleotide sequence (SEQ ID NO:140) designated herein as DNA41144.

FIG. 141 shows a nucleotide sequence (SEQ ID NO:141) designated herein as DNA41161.

FIG. 142 shows a nucleotide sequence (SEQ ID NO:142) designated herein as DNA41186.

FIG. 143 shows a nucleotide sequence (SEQ ID NO:143) designated herein as DNA41250.

FIG. 144 shows a nucleotide sequence (SEQ ID NO:144) designated herein as DNA41284.

FIG. 145 shows a nucleotide sequence (SEQ ID NO:145) designated herein as DNA41303.

FIG. 146 shows a nucleotide sequence (SEQ ID NO:146) designated herein as DNA41326.

FIG. 147 shows a nucleotide sequence (SEQ ID NO:147) designated herein as DNA41444.

FIG. 148 shows a nucleotide sequence (SEQ ID NO:148) designated herein as DNA41445.

FIG. 149 shows a nucleotide sequence (SEQ ID NO:149) designated herein as DNA41452.

FIG. 150 shows a nucleotide sequence (SEQ ID NO:150) designated herein as DNA41456.

FIG. 151 shows a nucleotide sequence (SEQ ID NO:151) designated herein as DNA41458.

FIG. 152 shows a nucleotide sequence (SEQ ID NO:152) designated herein as DNA41462.

FIG. 153 shows a nucleotide sequence (SEQ ID NO:153) designated herein as DNA41465.

FIG. 154 shows a nucleotide sequence (SEQ ID NO:154) designated herein as DNA41475.

FIG. 155 shows a nucleotide sequence (SEQ ID NO:155) designated herein as DNA41514.

FIG. 156 shows a nucleotide sequence (SEQ ID NO:156) designated herein as DNA41565.

FIG. 157 shows a nucleotide sequence (SEQ ID NO:157) designated herein as DNA41566.

FIG. 158 shows a nucleotide sequence (SEQ ID NO:158) designated herein as DNA41626.

FIG. 159 shows a nucleotide sequence (SEQ ID NO:159) designated herein as DNA41709.

FIG. 160 shows a nucleotide sequence (SEQ ID NO:160) designated herein as DNA41775.

FIG. 161 shows a nucleotide sequence (SEQ ID NO:161) designated herein as DNA41784.

FIG. 162 shows a nucleotide sequence (SEQ ID NO:162) designated herein as DNA42194.

FIG. 163 shows a nucleotide sequence (SEQ ID NO:163) designated herein as DNA42279.

FIG. 164 shows a nucleotide sequence (SEQ ID NO:164) designated herein as DNA42314.

FIG. 165 shows a nucleotide sequence (SEQ ID NO:165) designated herein as DNA42331.

FIG. 166 shows a nucleotide sequence (SEQ ID NO:166) designated herein as DNA42358.

FIG. 167 shows a nucleotide sequence (SEQ ID NO:167) designated herein as DNA42858.

FIG. 168 shows a nucleotide sequence (SEQ ID NO:168) designated herein as DNA42870.

FIG. 169 shows a nucleotide sequence (SEQ ID NO:169) designated herein as DNA42875.

FIG. 170 shows a nucleotide sequence (SEQ ID NO:170) designated herein as DNA43197.

FIG. 171 shows a nucleotide sequence (SEQ ID NO:171) designated herein as DNA43203.

FIG. 172 shows a nucleotide sequence (SEQ ID NO:172) designated herein as DNA43295.

FIG. 173 shows a nucleotide sequence (SEQ ID NO:173) designated herein as DNA43301.

FIG. 174 shows a nucleotide sequence (SEQ ID NO:174) designated herein as DNA43363.

FIG. 175 shows a nucleotide sequence (SEQ ID NO:175) designated herein as DNA43420.

FIG. 176 shows a nucleotide sequence (SEQ ID NO:176) designated herein as DNA443479.

FIG. 177 shows a nucleotide sequence (SEQ ID NO:177) designated herein as DNA43489.

FIG. 178 shows a nucleotide sequence (SEQ ID NO:178) designated herein as DNA43498.

FIG. 179 shows a nucleotide sequence (SEQ ID NO:179) designated herein as DNA43509.

FIG. 180 shows a nucleotide sequence (SEQ ID NO:180) designated herein as DNA43512.

FIG. 181 shows a nucleotide sequence (SEQ ID NO:181) designated herein as DNA43531.

FIG. 182 shows a nucleotide sequence (SEQ ID NO:182) designated herein as DNA43546.

FIG. 183 shows a nucleotide sequence (SEQ ID NO:183) designated herein as DNA43586.

FIG. 184 shows a nucleotide sequence (SEQ ID NO:184) designated herein as DNA43862.

FIG. 185 shows a nucleotide sequence (SEQ ID NO:185) designated herein as DNA43887.

FIG. 186 shows a nucleotide sequence (SEQ ID NO:186) designated herein as DNA43936.

FIG. 187 shows a nucleotide sequence (SEQ ID NO:187) designated herein as DNA43961.

FIG. 188 shows a nucleotide sequence (SEQ ID NO:188) designated herein as DNA43971.

FIG. 189 shows a nucleotide sequence (SEQ ID NO:189) designated herein as DNA44048.

FIG. 190 shows a nucleotide sequence (SEQ ID NO:190) designated herein as DNA44920.

FIG. 191 shows a nucleotide sequence (SEQ ID NO:191) designated herein as DNA44922.

FIG. 192 shows a nucleotide sequence (SEQ ID NO:192) designated herein as DNA44934.

FIG. 193 shows a nucleotide sequence (SEQ ID NO:193) designated herein as DNA44987.

FIG. 194 shows a nucleotide sequence (SEQ ID NO:194) designated herein as DNA45014.

FIG. 195 shows a nucleotide sequence (SEQ ID NO:195) designated herein as DNA45030.

FIG. 196 shows a nucleotide sequence (SEQ ID NO:196) designated herein as DNA45051.

FIG. 197 shows a nucleotide sequence (SEQ ID NO:197) designated herein as DNA45064.

FIG. 198 shows a nucleotide sequence (SEQ ID NO:198) designated herein as DNA45282.

FIG. 199 shows a nucleotide sequence (SEQ ID NO:199) designated herein as DNA45288.

FIG. 200 shows a nucleotide sequence (SEQ ID NO:200) designated herein as DNA45300.

FIG. 201 shows a nucleotide sequence (SEQ ID NO:201) designated herein as DNA45740.

FIG. 202 shows a nucleotide sequence (SEQ ID NO:202) designated herein as DNA45759.

FIG. 203 shows a nucleotide sequence (SEQ ID NO:203) designated herein as DNA45784.

FIG. 204 shows a nucleotide sequence (SEQ ID NO:204) designated herein as DNA45789.

FIG. 205 shows a nucleotide sequence (SEQ ID NO:205) designated herein as DNA45816.

FIG. 206 shows a nucleotide sequence (SEQ ID NO:206) designated herein as DNA45944.

FIG. 207 shows a nucleotide sequence (SEQ ID NO:207) designated herein as DNA45954.

FIG. 208 shows a nucleotide sequence (SEQ ID NO:208) designated herein as DNA45964.

FIG. 209 shows a nucleotide sequence (SEQ ID NO:209) designated herein as DNA45993.

FIG. 210 shows a nucleotide sequence (SEQ ID NO:210) designated herein as DNA46092.

FIG. 211 shows a nucleotide sequence (SEQ ID NO:211) designated herein as DNA46213.

FIG. 212 shows a nucleotide sequence (SEQ ID NO:212) designated herein as DNA46215.

FIG. 213 shows a nucleotide sequence (SEQ ID NO:213) designated herein as DNA46226.

FIG. 214 shows a nucleotide sequence (SEQ ID NO:214) designated herein as DNA46328.

FIG. 215 shows a nucleotide sequence (SEQ ID NO:215) designated herein as DNA47580.

FIG. 216 shows a nucleotide sequence (SEQ ID NO:216) designated herein as DNA47691.

FIG. 217 shows a nucleotide sequence (SEQ ID NO:217) designated herein as DNA47751.

FIG. 218 shows a nucleotide sequence (SEQ ID NO:218) designated herein as DNA47835.

FIG. 219 shows a nucleotide sequence (SEQ ID NO:219) designated herein as DNA47858.

FIG. 220 shows a nucleotide sequence (SEQ ID NO:220) designated herein as DNA47890.

FIG. 221 shows a nucleotide sequence (SEQ ID NO:221) designated herein as DNA47930.

FIG. 222 shows a nucleotide sequence (SEQ ID NO:222) designated herein as DNA47990.

FIG. 223 shows a nucleotide sequence (SEQ ID NO:223) designated herein as DNA48054.

FIG. 224 shows a nucleotide sequence (SEQ ID NO:224) designated herein as DNA48124.

FIG. 225 shows a nucleotide sequence (SEQ ID NO:225) designated herein as DNA48131.

FIG. 226 shows a nucleotide sequence (SEQ ID NO:226) designated herein as DNA48162.

FIG. 227 shows a nucleotide sequence (SEQ ID NO:227) designated herein as DNA48209.

FIG. 228 shows a nucleotide sequence (SEQ ID NO:228) designated herein as DNA48389.

FIG. 229 shows a nucleotide sequence (SEQ ID NO:229) designated herein as DNA48446.

FIG. 230 shows a nucleotide sequence (SEQ ID NO:230) designated herein as DNA48466.

FIG. 231 shows a nucleotide sequence (SEQ ID NO:231) designated herein as DNA48576.

FIG. 232 shows a nucleotide sequence (SEQ ID NO:232) designated herein as DNA48598.

FIG. 233 shows a nucleotide sequence (SEQ ID NO:233) designated herein as DNA48666.

FIG. 234 shows a nucleotide sequence (SEQ ID NO:234) designated herein as DNA48748.

FIG. 235 shows a nucleotide sequence (SEQ ID NO:235) designated herein as DNA48777.

FIG. 236 shows a nucleotide sequence (SEQ ID NO:236) designated herein as DNA48830.

FIG. 237 shows a nucleotide sequence (SEQ ID NO:237) designated herein as DNA49352.

FIG. 238 shows a nucleotide sequence (SEQ ID NO:238) designated herein as DNA49407.

FIG. 239 shows a nucleotide sequence (SEQ ID NO:239) designated herein as DNA49448.

FIG. 240 shows a nucleotide sequence (SEQ ID NO:240) designated herein as DNA49528.

FIG. 241 shows a nucleotide sequence (SEQ ID NO:241) designated herein as DNA49529.

FIG. 242 shows a nucleotide sequence (SEQ ID NO:242) designated herein as DNA49948.

FIG. 243 shows a nucleotide sequence (SEQ ID NO:243) designated herein as DNA49956.

FIG. 244 shows a nucleotide sequence (SEQ ID NO:244) designated herein as DNA49992.

FIG. 245 shows a nucleotide sequence (SEQ ID NO:245) designated herein as DNA50307.

FIG. 246 shows a nucleotide sequence (SEQ ID NO:246) designated herein as DNA50319.

FIG. 247 shows a nucleotide sequence (SEQ ID NO:247) designated herein as DNA50346.

FIG. 248 shows a nucleotide sequence (SEQ ID NO:248) designated herein as DNA50354.

FIG. 249 shows a nucleotide sequence (SEQ ID NO:249) designated herein as DNA50356.

FIG. 250 shows a nucleotide sequence (SEQ ID NO:250) designated herein as DNA50405.

FIG. 251 shows a nucleotide sequence (SEQ ID NO:251) designated herein as DNA50421.

FIG. 252 shows a nucleotide sequence (SEQ ID NO:252) designated herein as DNA50423.

FIG. 253 shows a nucleotide sequence (SEQ ID NO:253) designated herein as DNA50527.

FIG. 254 shows a nucleotide sequence (SEQ ID NO:254) designated herein as DNA50584.

FIG. 255 shows a nucleotide sequence (SEQ ID NO:255) designated herein as DNA50626.

FIG. 256 shows a nucleotide sequence (SEQ ID NO:256) designated herein as DNA50637.

FIG. 257 shows a nucleotide sequence (SEQ ID NO:257) designated herein as DNA50650.

FIG. 258 shows a nucleotide sequence (SEQ ID NO:258) designated herein as DNA50674.

FIG. 259 shows a nucleotide sequence (SEQ ID NO:259) designated herein as DNA50675.

FIG. 260 shows a nucleotide sequence (SEQ ID NO:260) designated herein as DNA50698.

FIG. 261 shows a nucleotide sequence (SEQ ID NO:261) designated herein as DNA50730.

FIG. 262 shows a nucleotide sequence (SEQ ID NO:262) designated herein as DNA50737.

FIG. 263 shows a nucleotide sequence (SEQ ID NO:263) designated herein as DNA51003.

FIG. 264 shows a nucleotide sequence (SEQ ID NO:264) designated herein as DNA51010.

FIG. 265 shows a nucleotide sequence (SEQ ID NO:265) designated herein as DNA51059.

FIG. 266 shows a nucleotide sequence (SEQ ID NO:266) designated herein as DNA51413.

FIG. 267 shows a nucleotide sequence (SEQ ID NO:267) designated herein as DNA51712.

FIG. 268 shows a nucleotide sequence (SEQ ID NO:268) designated herein as DNA51795.

FIG. 269 shows a nucleotide sequence (SEQ ID NO:269) designated herein as DNA52199.

FIG. 270 shows a nucleotide sequence (SEQ ID NO:270) designated herein as DNA52218.

FIG. 271 shows a nucleotide sequence (SEQ ID NO:271) designated herein as DNA52352.

FIG. 272 shows a nucleotide sequence (SEQ ID NO:272) designated herein as DNA54446.

FIG. 273 shows a nucleotide sequence (SEQ ID NO:273) designated herein as DNA54552.

FIG. 274 shows a nucleotide sequence (SEQ ID NO:274) designated herein as DNA54580.

FIG. 275 shows a nucleotide sequence (SEQ ID NO:275) designated herein as DNA54623.

FIG. 276 shows a nucleotide sequence (SEQ ID NO:276) designated herein as DNA54672.

FIG. 277 shows a nucleotide sequence (SEQ ID NO:277) designated herein as DNA54840.

FIG. 278 shows a nucleotide sequence (SEQ ID NO:278) designated herein as DNA54856.

FIG. 279 shows a nucleotide sequence (SEQ ID NO:279) designated herein as DNA54882.

FIG. 280 shows a nucleotide sequence (SEQ ID NO:280) designated herein as DNA54943.

FIG. 281 shows a nucleotide sequence (SEQ ID NO:281) designated herein as DNA54970.

FIG. 282 shows a nucleotide sequence (SEQ I) NO:282) designated herein as DNA55134.

FIG. 283 shows a nucleotide sequence (SEQ ID NO:283) designated herein as DNA55198.

FIG. 284 shows a nucleotide sequence (SEQ ID NO:284) designated herein as DNA55199.

FIG. 285 shows a nucleotide sequence (SEQ ID NO:285) designated herein as DNA55292.

FIG. 286 shows a nucleotide sequence (SEQ ID NO:286) designated herein as DNA55646.

FIG. 287 shows a nucleotide sequence (SEQ ID NO:287) designated herein as DNA56553.

FIG. 288 shows a nucleotide sequence (SEQ I) NO:288) designated herein as DNA56554.

FIG. 289 shows a nucleotide sequence (SEQ ID NO:289) designated herein as DNA56556.

FIG. 290 shows a nucleotide sequence (SEQ ID NO:290) designated herein as DNA56587.

FIG. 291 shows a nucleotide sequence (SEQ ID NO:291) designated herein as DNA56590.

FIG. 292 shows a nucleotide sequence (SEQ ID NO:292) designated herein as DNA56600.

FIG. 293 shows a nucleotide sequence (SEQ ID NO:293) designated herein as DNA56648.

FIG. 294 shows a nucleotide sequence (SEQ ID NO:294) designated herein as DNA56650.

FIG. 295 shows a nucleotide sequence (SEQ ID NO:295) designated herein as DNA56707.

FIG. 296 shows a nucleotide sequence (SEQ ID NO:296) designated herein as DNA56717.

FIG. 297 shows a nucleotide sequence (SEQ ID NO:297) designated herein as DNA58387.

FIG. 298 shows a nucleotide sequence (SEQ ID NO:298) designated herein as DNA58414.

FIG. 299 shows a nucleotide sequence (SEQ ID NO:299) designated herein as DNA58529.

FIG. 300 shows a nucleotide sequence (SEQ ID NO:300) designated herein as DNA59385.

FIG. 301 shows a nucleotide sequence (SEQ ID NO:301) designated herein as DNA59789.

FIG. 302 shows a nucleotide sequence (SEQ ID NO:302) designated herein as DNA60321.

FIG. 303 shows a nucleotide sequence (SEQ ID NO:303) designated herein as DNA60370.

FIG. 304 shows a nucleotide sequence (SEQ ID NO:304) designated herein as DNA60406.

FIG. 305 shows a nucleotide sequence (SEQ ID NO:305) designated herein as DNA60438.

FIG. 306 shows a nucleotide sequence (SEQ ID NO:306) designated herein as DNA60460.

FIG. 307 shows a nucleotide sequence (SEQ ID NO:307) designated herein as DNA60466.

FIG. 308 shows a nucleotide sequence (SEQ ID NO:308) designated herein as DNA60508.

FIG. 309 shows a nucleotide sequence (SEQ ID NO:309) designated herein as DNA60542.

FIG. 310 shows a nucleotide sequence (SEQ ID NO:310) designated herein as DNA60590.

FIG. 311 shows a nucleotide sequence (SEQ ID NO:311) designated herein as DNA61350.

FIG. 312 shows a nucleotide sequence (SEQ ID NO:312) designated herein as DNA61356.

FIG. 313 shows a nucleotide sequence (SEQ ID NO:313) designated herein as DNA61478.

FIG. 314 shows a nucleotide sequence (SEQ ID NO:314) designated herein as DNA61513.

FIG. 315 shows a nucleotide sequence (SEQ ID NO:315) designated herein as DNA61561.

FIG. 316 shows a nucleotide sequence (SEQ ID NO:316) designated herein as DNA61895.

FIG. 317 shows a nucleotide sequence (SEQ ID NO:317) designated herein as DNA61930.

FIG. 318 shows a nucleotide sequence (SEQ ID NO:318) designated herein as DNA61953.

FIG. 319 shows a nucleotide sequence (SEQ ID NO:319) designated herein as DNA62011.

FIG. 320 shows a nucleotide sequence (SEQ ID NO:320) designated herein as DNA62080.

FIG. 321 shows a nucleotide sequence (SEQ ID NO:321) designated herein as DNA62126.

FIG. 322 shows a nucleotide sequence (SEQ ID NO:322) designated herein as DNA62154.

FIG. 323 shows a nucleotide sequence (SEQ ID NO:323) designated herein as DNA62170.

FIG. 324 shows a nucleotide sequence (SEQ ID NO:324) designated herein as DNA62193.

FIG. 325 shows a nucleotide sequence (SEQ ID NO:325) designated herein as DNA62261.

FIG. 326 shows a nucleotide sequence (SEQ ID NO:326) designated herein as DNA62291.

FIG. 327 shows a nucleotide sequence (SEQ ID NO:327) designated herein as DNA62422.

FIG. 328 shows a nucleotide sequence (SEQ ID NO:328) designated herein as DNA62436.

FIG. 329 shows a nucleotide sequence (SEQ ID NO:329) designated herein as DNA62524.

FIG. 330 shows a nucleotide sequence (SEQ ID NO:330) designated herein as DNA62589.

FIG. 331 shows a nucleotide sequence (SEQ ID NO:331) designated herein as DNA63878.

FIG. 332 shows a nucleotide sequence (SEQ ID NO:332) designated herein as DNA64017.

FIG. 333 shows a nucleotide sequence (SEQ ID NO:333) designated herein as DNA64045.

FIG. 334 shows a nucleotide sequence (SEQ ID NO:334) designated herein as DNA64101.

FIG. 335 shows a nucleotide sequence (SEQ ID NO:335) designated herein as DNA64183.

FIG. 336 shows a nucleotide sequence (SEQ ID NO:336) designated herein as DNA64193.

FIG. 337 shows a nucleotide sequence (SEQ ID NO:337) designated herein as DNA64199.

FIG. 338 shows a nucleotide sequence (SEQ ID NO:338) designated herein as DNA64268.

FIG. 339 shows a nucleotide sequence (SEQ ID NO:339) designated herein as DNA64304.

FIG. 340 shows a nucleotide sequence (SEQ ID NO:340) designated herein as DNA64453.

FIG. 341 shows a nucleotide sequence (SEQ ID NO:341) designated herein as DNA64458.

FIG. 342 shows a nucleotide sequence (SEQ ID NO:342) designated herein as DNA64512.

FIG. 343 shows a nucleotide sequence (SEQ ID NO:343) designated herein as DNA64540.

FIG. 344 shows a nucleotide sequence (SEQ ID NO:344) designated herein as DNA64552.

FIG. 345 shows a nucleotide sequence (SEQ ID NO:345) designated herein as DNA64557.

FIG. 346 shows a nucleotide sequence (SEQ ID NO:346) designated herein as DNA64569.

FIG. 347 shows a nucleotide sequence (SEQ ID NO:347) designated herein as DNA64627.

FIG. 348 shows a nucleotide sequence (SEQ ID NO:348) designated herein as DNA64745.

FIG. 349 shows a nucleotide sequence (SEQ ID NO:349) designated herein as DNA64784.

FIG. 350 shows a nucleotide sequence (SEQ ID NO:350) designated herein as DNA65609.

FIG. 351 shows a nucleotide sequence (SEQ ID NO:351) designated herein as DNA65644.

FIG. 352 shows a nucleotide sequence (SEQ ID NO:352) designated herein as DNA65720.

FIG. 353 shows a nucleotide sequence (SEQ ID NO:353) designated herein as DNA65752.

FIG. 354 shows a nucleotide sequence (SEQ ID NO:354) designated herein as DNA65771.

FIG. 355 shows a nucleotide sequence (SEQ ID NO:355) designated herein as DNA65833.

FIG. 356 shows a nucleotide sequence (SEQ ID NO:356) designated herein as DNA65836.

FIG. 357 shows a nucleotide sequence (SEQ ID NO:357) designated herein as DNA65864.

FIG. 358 shows a nucleotide sequence (SEQ ID NO:358) designated herein as DNA65869.

FIG. 359 shows a nucleotide sequence (SEQ ID NO:359) designated herein as DNA65928.

FIG. 360 shows a nucleotide sequence (SEQ ID NO:360) designated herein as DNA66065.

FIG. 361 shows a nucleotide sequence (SEQ ID NO:361) designated herein as DNA66095.

FIG. 362 shows a nucleotide sequence (SEQ ID NO:362) designated herein as DNA66197.

FIG. 363 shows a nucleotide sequence (SEQ ID NO:363) designated herein as DNA66217.

FIG. 364 shows a nucleotide sequence (SEQ ID NO:364) designated herein as DNA66231.

FIG. 365 shows a nucleotide sequence (SEQ ID NO:365) designated herein as DNA66404.

FIG. 366 shows a nucleotide sequence (SEQ ID NO:366) designated herein as DNA66432.

FIG. 367 shows a nucleotide sequence (SEQ ID NO:367) designated herein as DNA67076.

FIG. 368 shows a nucleotide sequence (SEQ ID NO:368) designated herein as DNA68013.

FIG. 369 shows a nucleotide sequence (SEQ ID NO:369) designated herein as DNA68018.

FIG. 370 shows a nucleotide sequence (SEQ ID NO:370) designated herein as DNA68034.

FIG. 371 shows a nucleotide sequence (SEQ ID NO:371) designated herein as DNA68119.

FIG. 372 shows a nucleotide sequence (SEQ ID NO:372) designated herein as DNA68248.

FIG. 373 shows a nucleotide sequence (SEQ ID NO:373) designated herein as DNA68383.

FIG. 374 shows a nucleotide sequence (SEQ ID NO:374) designated herein as DNA68423.

FIG. 375 shows a nucleotide sequence (SEQ ID NO:375) designated herein as DNA68441.

FIG. 376 shows a nucleotide sequence (SEQ ID NO:376) designated herein as DNA68459.

FIG. 377 shows a nucleotide sequence (SEQ ID NO:377) designated herein as DNA68509.

FIG. 378 shows a nucleotide sequence (SEQ ID NO:378) designated herein as DNA68514.

FIG. 379 shows a nucleotide sequence (SEQ ID NO:379) designated herein as DNA68521.

FIG. 380 shows a nucleotide sequence (SEQ ID NO:380) designated herein as DNA68532.

FIG. 381 shows a nucleotide sequence (SEQ ID NO:381) designated herein as DNA68540.

FIG. 382 shows a nucleotide sequence (SEQ ID NO:382) designated herein as DNA68561.

FIG. 383 shows a nucleotide sequence (SEQ ID NO:383) designated herein as DNA68585.

FIG. 384 shows a nucleotide sequence (SEQ ID NO:384) designated herein as DNA69491.

FIG. 385 shows a nucleotide sequence (SEQ ID NO:385) designated herein as DNA70222.

FIG. 386 shows a nucleotide sequence (SEQ ID NO:386) designated herein as DNA70239.

FIG. 387 shows a nucleotide sequence (SEQ ID NO:387) designated herein as DNA70244.

FIG. 388 shows a nucleotide sequence (SEQ ID NO:388) designated herein as DNA70349.

FIG. 389 shows a nucleotide sequence (SEQ ID NO:389) designated herein as DNA70400.

FIG. 390 shows a nucleotide sequence (SEQ ID NO:390) designated herein as DNA70413.

FIG. 391 shows a nucleotide sequence (SEQ ID NO:391) designated herein as DNA70526.

FIG. 392 shows a nucleotide sequence (SEQ ID NO:392) designated herein as DNA70685.

FIG. 393 shows a nucleotide sequence (SEQ ID NO:393) designated herein as DNA70732.

FIG. 394 shows a nucleotide sequence (SEQ ID NO:394) designated herein as DNA72634.

FIG. 395 shows a nucleotide sequence (SEQ ID NO:395) designated herein as DNA72683.

FIG. 396 shows a nucleotide sequence (SEQ ID NO:396) designated herein as DNA72695.

FIG. 397 shows a nucleotide sequence (SEQ ID NO:397) designated herein as DNA72864.

FIG. 398 shows a nucleotide sequence (SEQ ID NO:398) designated herein as DNA73156.

FIG. 399 shows a nucleotide sequence (SEQ ID NO:399) designated herein as DNA73275.

FIG. 400 shows a nucleotide sequence (SEQ ID NO:400) designated herein as DNA74052.

FIG. 401 shows a nucleotide sequence (SEQ ID NO:401) designated herein as DNA74063.

FIG. 402 shows a nucleotide sequence (SEQ ID NO:402) designated herein as DNA74072.

FIG. 403 shows a nucleotide sequence (SEQ ID NO:403) designated herein as DNA74140.

FIG. 404 shows a nucleotide sequence (SEQ ID NO:404) designated herein as DNA74216.

FIG. 405 shows a nucleotide sequence (SEQ ID NO:405) designated herein as DNA74218.

FIG. 406 shows a nucleotide sequence (SEQ ID NO:406) designated herein as DNA74228.

FIG. 407 shows a nucleotide sequence (SEQ ID NO:407) designated herein as DNA74256.

FIG. 408 shows a nucleotide sequence (SEQ ID NO:408) designated herein as DNA75062.

FIG. 409 shows a nucleotide sequence (SEQ ID NO:409) designated herein as DNA76137.

FIG. 410 shows a nucleotide sequence (SEQ ID NO:410) designated herein as DNA76158.

FIG. 411 shows a nucleotide sequence (SEQ ID NO:411) designated herein as DNA77098.

FIG. 412 shows a nucleotide sequence (SEQ ID NO:412) designated herein as DNA77791.

FIG. 413 shows a nucleotide sequence (SEQ ID NO:413) designated herein as DNA77968.

FIG. 414 shows a nucleotide sequence (SEQ ID NO:414) designated herein as DNA77976.

FIG. 415 shows a nucleotide sequence (SEQ ID NO:415) designated herein as DNA78017.

FIG. 416 shows a nucleotide sequence (SEQ ID NO:416) designated herein as DNA78095.

FIG. 417 shows a nucleotide sequence (SEQ ID NO:417) designated herein as DNA78103.

FIG. 418 shows a nucleotide sequence (SEQ ID NO:418) designated herein as DNA78113.

FIG. 419 shows a nucleotide sequence (SEQ ID NO:419) designated herein as DNA78746.

FIG. 420 shows a nucleotide sequence (SEQ ID NO:420) designated herein as DNA78759.

FIG. 421 shows a nucleotide sequence (SEQ ID NO:421) designated herein as DNA78796.

FIG. 422 shows a nucleotide sequence (SEQ ID NO:422) designated herein as DNA79561.

FIG. 423 shows a nucleotide sequence (SEQ ID NO:423) designated herein as DNA79602.

FIG. 424 shows a nucleotide sequence (SEQ ID NO:424) designated herein as DNA79617.

FIG. 425 shows a nucleotide sequence (SEQ ID NO:425) designated herein as DNA79628.

FIG. 426 shows a nucleotide sequence (SEQ ID NO:426) designated herein as DNA79640.

FIG. 427 shows a nucleotide sequence (SEQ ID NO:427) designated herein as DNA79661.

FIG. 428 shows a nucleotide sequence (SEQ ID NO:428) designated herein as DNA79684.

FIG. 429 shows a nucleotide sequence (SEQ ID NO:429) designated herein as DNA79717.

FIG. 430 shows a nucleotide sequence (SEQ ID NO:430) designated herein as DNA79733.

FIG. 431 shows a nucleotide sequence (SEQ ID NO:431) designated herein as DNA79970.

FIG. 432 shows a nucleotide sequence (SEQ ID NO:432) designated herein as DNA80050.

FIG. 433 shows a nucleotide sequence (SEQ ID NO:433) designated herein as DNA80247.

FIG. 434 shows a nucleotide sequence (SEQ ID NO:434) designated herein as DNA80265.

FIG. 435 shows a nucleotide sequence (SEQ ID NO:435) designated herein as DNA80615.

FIG. 436 shows a nucleotide sequence (SEQ ID NO:436) designated herein as DNA80623.

FIG. 437 shows a nucleotide sequence (SEQ ID NO:437) designated herein as DNA80627.

FIG. 438 shows a nucleotide sequence (SEQ ID NO:438) designated herein as DNA81896.

FIG. 439 shows a nucleotide sequence (SEQ ID NO:439) designated herein as DNA81918.

FIG. 440 shows a nucleotide sequence (SEQ ID NO:440) designated herein as DNA81976.

FIG. 441 shows a nucleotide sequence (SEQ ID NO:441) designated herein as DNA82017.

FIG. 442 shows a nucleotide sequence (SEQ ID NO:442) designated herein as DNA82024.

FIG. 443 shows a nucleotide sequence (SEQ ID NO:443) designated herein as DNA82027.

FIG. 444 shows a nucleotide sequence (SEQ ID NO:444) designated herein as DNA82115.

FIG. 445 shows a nucleotide sequence (SEQ ID NO:445) designated herein as DNA82154.

FIG. 446 shows a nucleotide sequence (SEQ ID NO:446) designated herein as DNA82157.

FIG. 447 shows a nucleotide sequence (SEQ ID NO:447) designated herein as DNA82166.

FIG. 448 shows a nucleotide sequence (SEQ ID NO:448) designated herein as DNA82182.

FIG. 449 shows a nucleotide sequence (SEQ ID NO:449) designated herein as DNA82212.

FIG. 450 shows a nucleotide sequence (SEQ ID NO:450) designated herein as DNA82498.

FIG. 451 shows a nucleotide sequence (SEQ ID NO:451) designated herein as DNA82499.

FIG. 452 shows a nucleotide sequence (SEQ ID NO:452) designated herein as DNA82504.

FIG. 453 shows a nucleotide sequence (SEQ ID NO:453) designated herein as DNA82531.

FIG. 454 shows a nucleotide sequence (SEQ ID NO:454) designated herein as DNA82693.

FIG. 455 shows a nucleotide sequence (SEQ ID NO:455) designated herein as DNA82702.

FIG. 456 shows a nucleotide sequence (SEQ ID NO:456) designated herein as DNA82786.

FIG. 457 shows a nucleotide sequence (SEQ ID NO:457) designated herein as DNA82851.

FIG. 458 shows a nucleotide sequence (SEQ ID NO:458) designated herein as DNA82898.

FIG. 459 shows a nucleotide sequence (SEQ ID NO:459) designated herein as DNA82935.

FIG. 460 shows a nucleotide sequence (SEQ ID NO:460) designated herein as DNA82977.

FIG. 461 shows a nucleotide sequence (SEQ ID NO:461) designated herein as DNA82989.

FIG. 462 shows a nucleotide sequence (SEQ ID NO:462) designated herein as DNA83628.

FIG. 463 shows a nucleotide sequence (SEQ ID NO:463) designated herein as DNA83630.

FIG. 464 shows a nucleotide sequence (SEQ ID NO:464) designated herein as DNA83749.

FIG. 465 shows a nucleotide sequence (SEQ ID NO:465) designated herein as DNA83772.

FIG. 466 shows a nucleotide sequence (SEQ ID NO:466) designated herein as DNA83800.

FIG. 467 shows a nucleotide sequence (SEQ ID NO:467) designated herein as DNA83950.

FIG. 468 shows a nucleotide sequence (SEQ ID NO:468) designated herein as DNA84027.

FIG. 469 shows a nucleotide sequence (SEQ ID NO:469) designated herein as DNA84076.

FIG. 470 shows a nucleotide sequence (SEQ ID NO:470) designated herein as DNA84109.

FIG. 471 shows a nucleotide sequence (SEQ ID NO:471) designated herein as DNA85072.

FIG. 472 shows a nucleotide sequence (SEQ ID NO:472) designated herein as DNA85154.

FIG. 473 shows a nucleotide sequence (SEQ ID NO:473) designated herein as DNA85193.

FIG. 474 shows a nucleotide sequence (SEQ ID NO:474) designated herein as DNA85224.

FIG. 475 shows a nucleotide sequence (SEQ ID NO:475) designated herein as DNA85237.

FIG. 476 shows a nucleotide sequence (SEQ ID NO:476) designated herein as DNA85289.

FIG. 477 shows a nucleotide sequence (SEQ ID NO:477) designated herein as DNA85357.

FIG. 478 shows a nucleotide sequence (SEQ ID NO:478) designated herein as DNA85361.

FIG. 479 shows a nucleotide sequence (SEQ ID NO:479) designated herein as DNA85371.

FIG. 480 shows a nucleotide sequence (SEQ ID NO:480) designated herein as DNA86875.

FIG. 481 shows a nucleotide sequence (SEQ ID NO:481) designated herein as DNA86876.

FIG. 482 shows a nucleotide sequence (SEQ ID NO:482) designated herein as DNA86905.

FIG. 483 shows a nucleotide sequence (SEQ ID NO:483) designated herein as DNA86945.

FIG. 484 shows a nucleotide sequence (SEQ ID NO:484) designated herein as DNA86969.

FIG. 485 shows a nucleotide sequence (SEQ ID NO:485) designated herein as DNA87050.

FIG. 486 shows a nucleotide sequence (SEQ ID NO:486) designated herein as DNA87094.

FIG. 487 shows a nucleotide sequence (SEQ ID NO:487) designated herein as DNA87126.

FIG. 488 shows a nucleotide sequence (SEQ ID NO:488) designated herein as DNA87493.

FIG. 489 shows a nucleotide sequence (SEQ ID NO:489) designated herein as DNA87494.

FIG. 490 shows a nucleotide sequence (SEQ ID NO:490) designated herein as DNA87505.

FIG. 491 shows a nucleotide sequence (SEQ ID NO:491) designated herein as DNA87566.

FIG. 492 shows a nucleotide sequence (SEQ ID NO:492) designated herein as DNA87586.

FIG. 493 shows a nucleotide sequence (SEQ ID NO:493) designated herein as DNA87649.

FIG. 494 shows a nucleotide sequence (SEQ ID NO:494) designated herein as DNA89340.

FIG. 495 shows a nucleotide sequence (SEQ ID NO:495) designated herein as DNA89355.

FIG. 496 shows a nucleotide sequence (SEQ ID NO:496) designated herein as DNA89365.

FIG. 497 shows a nucleotide sequence (SEQ ID NO:497) designated herein as DNA89419.

FIG. 498 shows a nucleotide sequence (SEQ ID NO:498) designated herein as DNA89470.

FIG. 499 shows a nucleotide sequence (SEQ ID NO:499) designated herein as DNA89480.

FIG. 500 shows a nucleotide sequence (SEQ ID NO:500) designated herein as DNA89549.

FIG. 501 shows a nucleotide sequence (SEQ ID NO:501) designated herein as DNA89606.

FIG. 502 shows a nucleotide sequence (SEQ ID NO:502) designated herein as DNA89615.

FIG. 503 shows a nucleotide sequence (SEQ ID NO:503) designated herein as DNA89669.

FIG. 504 shows a nucleotide sequence (SEQ ID NO:504) designated herein as DNA89760.

FIG. 505 shows a nucleotide sequence (SEQ ID NO:505) designated herein as DNA89766.

FIG. 506 shows a nucleotide sequence (SEQ ID NO:506) designated herein as DNA89772.

FIG. 507 shows a nucleotide sequence (SEQ ID NO:507) designated herein as DNA89773.

FIG. 508 shows a nucleotide sequence (SEQ ID NO:508) designated herein as DNA89774.

FIG. 509 shows a nucleotide sequence (SEQ ID NO:509) designated herein as DNA89872.

FIG. 510 shows a nucleotide sequence (SEQ ID NO:510) designated herein as DNA89918.

FIG. 511 shows a nucleotide sequence (SEQ ID NO:511) designated herein as DNA89928.

FIG. 512 shows a nucleotide sequence (SEQ ID NO:512) designated herein as DNA89930.

FIG. 513 shows a nucleotide sequence (SEQ ID NO:513) designated herein as DNA91463.

FIG. 514 shows a nucleotide sequence (SEQ ID NO:514) designated herein as DNA91507.

FIG. 515 shows a nucleotide sequence (SEQ ID NO:515) designated herein as DNA93615.

FIG. 516 shows a nucleotide sequence (SEQ ID NO:516) designated herein as DNA94011.

FIG. 517 shows a nucleotide sequence (SEQ ID NO:517) designated herein as DNA94043.

FIG. 518 shows a nucleotide sequence (SEQ ID NO:518) designated herein as DNA94050.

FIG. 519 shows a nucleotide sequence (SEQ ID NO:519) designated herein as DNA94097.

FIG. 520 shows a nucleotide sequence (SEQ ID NO:520) designated herein as DNA94098.

FIG. 521 shows a nucleotide sequence (SEQ ID NO:521) designated herein as DNA94100.

FIG. 522 shows a nucleotide sequence (SEQ ID NO:522) designated herein as DNA94126.

FIG. 523 shows a nucleotide sequence (SEQ ID NO:523) designated herein as DNA94136.

FIG. 524 shows a nucleotide sequence (SEQ ID NO:524) designated herein as DNA94156.

FIG. 525 shows a nucleotide sequence (SEQ ID NO:525) designated herein as DNA94219.

FIG. 526 shows a nucleotide sequence (SEQ ID NO:526) designated herein as DNA94254.

FIG. 527 shows a nucleotide sequence (SEQ ID NO:527) designated herein as DNA94274.

FIG. 528 shows a nucleotide sequence (SEQ ID NO:528) designated herein as DNA94292.

FIG. 529 shows a nucleotide sequence (SEQ ID NO:529) designated herein as DNA94360.

FIG. 530 shows a nucleotide sequence (SEQ ID NO:530) designated herein as DNA94377.

FIG. 531 shows a nucleotide sequence (SEQ ID NO:531) designated herein as DNA94477.

FIG. 532 shows a nucleotide sequence (SEQ ID NO:532) designated herein as DNA94518.

FIG. 533 shows a nucleotide sequence (SEQ ID NO:533) designated herein as DNA94533.

FIG. 534 shows a nucleotide sequence (SEQ ID NO:534) designated herein as DNA95370.

FIG. 535 shows a nucleotide sequence (SEQ ID NO:535) designated herein as DNA97358.

FIG. 536 shows a nucleotide sequence (SEQ ID NO:536) designated herein as DNA97374.

FIG. 537 shows a nucleotide sequence (SEQ ID NO:537) designated herein as DNA97470.

FIG. 538 shows a nucleotide sequence (SEQ ID NO:538) designated herein as DNA97581.

FIG. 539 shows a nucleotide sequence (SEQ ID NO:539) designated herein as DNA97767.

FIG. 540 shows a nucleotide sequence (SEQ ID NO:540) designated herein as DNA97842.

FIG. 541 shows a nucleotide sequence (SEQ ID NO:541) designated herein as DNA97949.

FIG. 542 shows a nucleotide sequence (SEQ ID NO:542) designated herein as DNA97987.

FIG. 543 shows a nucleotide sequence (SEQ ID NO:543) designated herein as DNA97995.

FIG. 544 shows a nucleotide sequence (SEQ ID NO:544) designated herein as DNA98293.

FIG. 545 shows a nucleotide sequence (SEQ ID NO:545) designated herein as DNA98294.

FIG. 546 shows a nucleotide sequence (SEQ ID NO:546) designated herein as DNA98346.

FIG. 547 shows a nucleotide sequence (SEQ ID NO:547) designated herein as DNA98360.

FIG. 548 shows a nucleotide sequence (SEQ ID NO:548) designated herein as DNA98829.

FIG. 549 shows a nucleotide sequence (SEQ ID NO:549) designated herein as DNA101514.

FIG. 550 shows a nucleotide sequence (SEQ ID NO:550) designated herein as DNA101572.

FIG. 551 shows a nucleotide sequence (SEQ ID NO:551) designated herein as DNA101580.

FIG. 552 shows a nucleotide sequence (SEQ ID NO:552) designated herein as DNA101595.

FIG. 553 shows a nucleotide sequence (SEQ ID NO:553) designated herein as DNA101633.

FIG. 554 shows a nucleotide sequence (SEQ ID NO:554) designated herein as DNA101717.

FIG. 555 shows a nucleotide sequence (SEQ ID NO:555) designated herein as DNA101768.

FIG. 556 shows a nucleotide sequence (SEQ ID NO:556) designated herein as DNA107332.

FIG. 557 shows a nucleotide sequence (SEQ ID NO:557) designated herein as DNA43499.

FIG. 558 shows a nucleotide sequence (SEQ ID NO:558) designated herein as DNA45713.

FIG. 559 shows a nucleotide sequence (SEQ ID NO:559) designated herein as DNA46089.

FIG. 560 shows a nucleotide sequence (SEQ ID NO:560) designated herein as DNA68256.

FIG. 561 shows a nucleotide sequence (SEQ ID NO:561) designated herein as DNA70305.

FIG. 562 shows a nucleotide sequence (SEQ ID NO:562) designated herein as DNA82953.

### DETAILED DESCRIPTION OF THE PREFERRED EMBODIMENTS

I. Definitions

The term “SRT polypeptide” when used herein encompasses “native sequence SRT polypeptides” and “SRT polypeptide variants” (which are further defined herein). “SRT” is a designation given to those polypeptides which are encoded by the nucleic acid molecules shown in the accompanying figures and variants thereof, nucleic acid molecules comprising the sequence shown in the accompanying figures and variants thereof as well as fragments of the above. The SRT polypeptides of the invention may be isolated from a variety of sources, such as from human tissue types or from another source, or prepared by recombinant and/or synthetic methods.

A “native sequence” SRT polypeptide comprises a polypeptide having the same amino acid sequence as the corresponding SRT polypeptide derived from nature. Such native sequence SRT polypeptides can be isolated from nature or can be produced by recombinant and/or synthetic means. The term “native sequence SRT polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptide.

An SRT polypeptide “extracellular domain” or “ECD” refers to a form of the SRT polypeptide which is essentially free of the transmembrane and cytoplasmic domains. Ordinarily, an SRT polypeptide ECD will have less than about 1% of such transmembrane and/or cytoplasmic domains and preferably, will have less than about 0.5% of such domains. It will be understood that any transmembrane domain(s) identified for the SRT polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified.

“Variant SRT polypeptide” means an active SRT polypeptide as defined below having at least about 80% amino acid sequence identity with the amino acid sequence of a specifically derived fragment of any other polypeptide which will be specifically recited. Such variant SRT polypeptides include, for instance, SRT polypeptides wherein one or more amino acid residues are added, or deleted, at the N- and/or C-terminus, as well as within one or more internal domains, of the full-length amino acid sequence. Ordinarily, a variant SRT polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with an SRT polypeptide encoded by a nucleic acid molecule shown in one of the accompanying figures or a specified fragment thereof. SRT variant polypeptides do not encompass the native SRT polypeptide sequence. Ordinarily, SRT variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

“Percent (%) amino acid sequence identity” with respect to the SRT polypeptide sequences identified herein is defined as the percentage of amino acid residues in a candidate sequence that are identical with the amino acid residues in a SRT sequence, after aligning the sequences and introducing gaps, if necessary, to achieve the maximum percent sequence identity, and not considering any conservative substitutions as part of the sequence identity. Alignment for purpose s of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Tables 2 and 3 demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“SRT variant polynucleotide” or “SRT variant nucleic acid sequence” means a nucleic acid molecule which has at least about 80% nucleic acid sequence identity with any of the nucleic acid sequences shown in the accompanying figures or a specified fragment thereof. Ordinarily, a SRT variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with any of the nucleic acid sequences shown in the accompanying figures or a specified fragment thereof. SRT polynucleotide variants do not encompass the native SRT nucleotide sequence.

Ordinarily, SRT variant polynucleotides are at least about 10 nucleotides in length, often at least about 15 nucleotides in length, often at least about 20 nucleotides in length, often at least about 25 nucleotides in length, often at least about 30 nucleotides in length, often at least about 35 nucleotides in length, often at least about 40 nucleotides in length, often at least about 45 nucleotides in length, often at least about 50 nucleotides in length, often at least about 55 nucleotides in length, often at least about 60 nucleotides in length, often at least about 65 nucleotides in length, often at least about 65 nucleotides in length, often at least about 70 nucleotides in length, often at least about 75 nucleotides in length, often at least about 80 nucleotides in length, often at least about 85 nucleotides in length, often at least about 90 nucleotides in length, often at least about 95 nucleotides in length, often at least about 100 nucleotides in length, or more.

“Percent (%) nucleic acid sequence identity” with respect to the SRT polypeptide-encoding nucleic acid sequences identified herein is defined as the percentage of nucleotides in a candidate sequence that are identical with the nucleotides in a SRT polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 1. The ALIGN-2 sequence comparison computer program was authored by Genentech, Inc. and the source code shown in Table 1 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5 demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, SRT variant polynucleotides are nucleic acid molecules that encode an active SRT polypeptide and which are capable of hybridizing, preferably under stringent hybridization conditions, to any of the nucleotide sequences shown in the accompanying figures or their complements. SRT variant polypeptides may be those that are encoded by a SRT variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 6 below) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows:

100 times the fraction X/Y

where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the SRT natural environment will not be present. Ordinarily, however, isolated polypeptide will be prepared by at least one purification step.

An “isolated” nucleic acid molecule encoding a SRT polypeptide is a nucleic acid molecule that is identified and separated from at least one contaminant nucleic acid molecule with which it is ordinarily associated in the natural source of the SRT-encoding nucleic acid. Preferably, the isolated nucleic is free of association with all components with which it is naturally associated. An isolated SRT-encoding nucleic acid molecule is other than in the form or setting in which it is found in nature. Isolated nucleic acid molecules therefore are distinguished from the SRT-encoding nucleic acid molecule as it exists in natural cells. However, an isolated nucleic acid molecule encoding a SRT polypeptide includes SRT-encoding nucleic acid molecules contained in cells that ordinarily express SRT where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-SRT monoclonal antibodies (including agonist, antagonist, and neutralizing antibodies), anti-SRT antibody compositions with polyepitopic specificity, single chain anti-SRT antibodies, and fragments of anti-SRT antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a SRT polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of SRT which retain a biological and/or an immunological activity of native or naturally-occurring SRT, wherein “biological” activity refers to a biological function (either inhibitory or stimulatory) caused by a native or naturally-occurring SRT other than the ability to induce the production of an antibody against an antigenic epitope possessed by a native or naturally-occurring SRT and an “immunological” activity refers to the ability to induce the production of an antibody against an antigenic epitope possessed by a native or naturally-occurring SRT.

The term “antagonist” is used in the broadest sense, and includes any molecule that partially or fully blocks, inhibits, or neutralizes a biological activity of a native SRT polypeptide disclosed herein. In a similar manner, the term “agonist” is used in the broadest sense and includes any molecule that mimics a biological activity of a native SRT polypeptide disclosed herein. Suitable agonist or antagonist molecules specifically include agonist or antagonist antibodies or antibody fragments, fragments or amino acid sequence variants of native SRT polypeptides, peptides, small organic molecules, etc. Methods for identifying agonists or antagonists of a SRT polypeptide may comprise contacting a SRT polypeptide with a candidate agonist or antagonist molecule and measuring a detectable change in one or more biological activities normally associated with the SRT polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a SRT polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

An “oligonucleotide” or “oligomer” is a stretch of nucleotide residues which has a sufficient number of bases to be used in a polymerase chain reaction (PCR). These sequences are based on (or designed from) genomic or cDNA sequences and may be used to amplify, confirm, or reveal the presence of an identical, similar or complementary DNA or RNA in a particular cell or tissue. Oligonucleotides or oligomers comprise portions of a DNA sequence having at least about 10 nucleotides as described above. Oligonucleotides may be chemically synthesized and may be used as probes.

“Probes” are nucleic acid sequences of variable length, preferably between about 10 and as many as about 6000 nucleotides, depending upon use. They are used in the detection of identical, similar or complementary nucleic acid sequences. Longer length probes are usually obtained from a natural or recombinant source, are highly specific and are often much slower to hybridize to a target nucleic acid than are oligomers. Probes may be single- or double-stranded and may be carefully designaed to have specificity in PCR, hybridization membrane-based, or ELISA-like technologies.

“Detectably labeled” with regard to a nucleic acid molecule of the present invention means that the molecule has attached thereto, either covalently or non-covalently, a compound which is detectable such as, for example, radionuclides, enzymes, fluorescent, chemi-luminescent, or chromogenic agents. Detectable labels associate with, establish the presence of, and may allow quantification of a particular nucleic or amino acid sequence.

A “portion” or “fragment” of a polynucleotide or nucleic acid molecule comprises all or any part of the nucleotide sequence having fewer nucleotides than about 6 kb, preferably fewer than about 1 kb which can be used as a probe. Such probes may be labelled with detectable labels using nick translation, Klenow fill-in reaction, PCR or other methods well known in the art. After pretesting to optimize reaction conditions and to eliminate false positives, nucleic acid probes may be used in Southern, Northern or in situ hybridizations to determine whether DNA or RNA encoding the protein is present in a biological sample, cell type, tissue, organ or organism.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity =
(the number of identically matching amino acid residues between the two
polypeptide sequences as determined by ALIGN-2) divided by (the total
number of amino acid residues of the PRO polypeptide) =
5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity =
(the number of identically matching amino acid residues between the two
polypeptide sequences as determined by ALIGN-2) divided by (the total
number of amino acid residues of the PRO polypeptide) =
5 divided by 10 = 50%

TABLE 4

| | | |
|---|---|---|
| PRO-DNA | NNNNNNNNNNNNNN | (Length = 14 nucleotides) |
| Comparison DNA | NNNNNNLLLLLLLLLL | (Length = 16 nucleotides) |

% nucleic acid sequence identity =
(the number of identically matching nucleotides between the two nucleic
acid sequences as determined by ALIGN-2) divided by (the total number
of nucleotides of the PRO-DNA nucleic acid sequence) =
6 divided by 14 = 42.9%

TABLE 5

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity =
(the number of identically matching nucleotides between the two nucleic
acid sequences as determined by ALIGN-2) divided by (the total number
of nucleotides of the PRO-DNA nucleic acid sequence) =
4 divided by 12 = 33.3%

II. Compositions and Methods of the Invention

A. Full-length SRT Polypeptides

The present invention provides newly identified and isolated polynucleotide sequences encoding at least a portion of full-length human polypeptides referred to in the present application as SRT polypeptides. In particular, cDNAs encoding at least a portion of SRT polypeptides have been identified and isolated, as disclosed in further detail in the Examples below. For sake of simplicity, in the present specification the polypeptides encoded by nucleic acid molecules disclosed herein as well as all further native homologues and variants included in the foregoing definition of SRT, will be referred to as “SRT”, regardless of their origin or mode of preparation.

B. SRT Polypeptide Variants

In addition to the native sequence SRT polypeptides described herein, it is contemplated that SRT variants can be prepared. SRT variants can be prepared by introducing appropriate nucleotide changes into the SRT DNA, and/or by synthesis of the desired SRT polypeptide. Those skilled in the art will appreciate that amino acid changes may alter post-translational processes of the SRT, such as changing the number or position of glycosylation sites or altering the membrane anchoring characteristics.

Variations in the native sequence SRT or in various domains of the SRT described herein, can be made, for example, using any of the techniques and guidelines for conservative and non-conservative mutations set forth, for instance, in U.S. Pat. No. 5,364,934. Variations may be a substitution, deletion or insertion of one or more codons encoding the SRT that results in a change in the amino acid sequence of the SRT as compared with the native sequence SRT. Optionally the variation is by substitution of at least one amino acid with any other amino acid in one or more of the domains of the SRT. Guidance in determining which amino acid residue may be inserted, substituted or deleted without adversely affecting the desired activity may be found by comparing the sequence of the SRT with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

SRT polypeptide fragments are provided herein. Such fragments may be truncated at the N-terminus or C-terminus, or may lack internal residues, for example, when compared with a full-length native protein. Certain fragments lack amino acid residues that are not essential for a desired biological activity of the SRT polypeptide.

SRT fragments may be prepared by any of a number of conventional techniques. Desired peptide fragments may be chemically synthesized. An alternative approach involves generating SRT fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, SRT polypeptide fragments share at least one biological and/or immunological activity with the corresponding native SRT polypeptide.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | |
| | ala; norleucine | leu |

Substantial modifications in function or immunological identity of the SRT polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the SRT variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

C. Modifications of SRT Polypeptides

Covalent modifications of SRT polypeptides are included within the scope of this invention. One type of covalent modification includes reacting targeted amino acid residues of a SRT polypeptide with an organic derivatizing agent that is capable of reacting with selected side chains or the N- or C- terminal residues of the SRT. Derivatization with bifunctional agents is useful, for instance, for crosslinking SRT to a water-insoluble support matrix or surface for use in the method for purifying anti-SRT antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the SRT polypeptide included within the scope of this invention comprises altering the native glycosylation pattern of the polypeptide. “Altering the native glycosylation pattern” is intended for purposes herein to mean deleting one or more carbohydrate moieties found in native sequence SRT (either by removing the underlying glycosylation site or by deleting the glycosylation by chemical and/or enzymatic means), and/or adding one or more glycosylation sites that are not present in the native sequence SRT. In addition, the phrase includes qualitative changes in the glycosylation of the native proteins, involving a change in the nature and proportions of the various carbohydrate moieties present.

Addition of glycosylation sites to the SRT polypeptide may be accomplished by altering the amino acid sequence. The alteration may be made, for example, by the addition of, or substitution by, one or more serine or threonine residues to the native sequence SRT (for O-linked glycosylation sites). The SRT amino acid sequence may optionally be altered through changes at the DNA level, particularly by mutating the DNA encoding the SRT polypeptide at preselected bases such that codons are generated that will translate into the desired amino acids.

Another means of increasing the number of carbohydrate moieties on the SRT polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the SRT polypeptide maybe accomplished chemically or enzymatically or by mutational substitution of codons encoding for amino acid residues that serve as targets for glycosylation. Chemical deglycosylation techniques are known in the art and described, for instance, by Hakimuddin et al., Arch. Biochem. Biophys., 259:52 (1987) and by Edge et al., Anal. Biochem. 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of SRT comprises linking the SRT polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The SRT polypeptides of the present invention may also be modified in a way to form chimeric molecules comprising SRT fused to another, heterologous polypeptide or amino acid sequence.

In one embodiment, such a chimeric molecule comprises a fusion of the SRT with a tag polypeptide which provides an epitope to which an anti-tag antibody can selectively bind. The epitope tag is generally placed at the amino- or carboxyl- terminus of the SRT. The presence of such epitope-tagged forms of the SRT can be detected using an antibody against the tag polypeptide. Also, provision of the epitope tag enables the SRT to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the SRT with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a SRT polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of SRT Polypeptides

The description below relates primarily to production of SRT by culturing cells transformed or transfected with a vector containing SRT nucleic acid. It is, of course, contemplated that alternative methods, which are well known in the art, may be employed to prepare SRT. For instance, the SRT sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the SRT may be chemically synthesized separately and combined using chemical or enzymatic methods to produce the full-length SRT.

1. Isolation of DNA Encoding SRT

DNA encoding SRT may be obtained from a cDNA library prepared from tissue believed to possess the SRT mRNA and to express it at a detectable level. Accordingly, human SRT DNA can be conveniently obtained from a cDNA library prepared from human tissue, such as described in the Examples. The SRT-encoding gene may also be obtained from a genomic library or by known synthetic procedures (e.g., automated nucleic acid synthesis).

Libraries can be screened with probes (such as antibodies to the SRT or oligonucleotides of at least about 20-80 bases) designed to identify the gene of interest or the protein encoded by it, wherein those probes may be based upon the polynucleotide sequences shown in the accompanying figures. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding SRT is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for SRT production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 199 1) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published Jun. 29, 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued Aug. 7, 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for SRT-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated SRT are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (WI38, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding SRT may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The SRT may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the SRT-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published Apr. 4, 1990), or the signal described in WO 90/13646 published Nov. 15, 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the SRT-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10: 157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the SRT-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding SRT.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

SRT transcription from vectors in mammalian host cells is controlled, for example, by promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the SRT by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the SRT coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding SRT.

Still other methods, vectors, and host cells suitable for adaptation to the synthesis of SRT in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies maybe employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence SRT polypeptide or against a synthetic peptide based on the DNA sequences provided herein or against exogenous sequence fused to SRT DNA and encoding a specific antibody epitope.

5. Purification of Polypeptide

Forms of SRT may be recovered from culture medium or from host cell lysates. If membrane-bound, it can be released from the membrane using a suitable detergent solution (e.g. Triton-X 100) or by enzymatic cleavage. Cells employed in expression of SRT can be disrupted by various physical or chemical means, such as freeze-thaw cycling, sonication, mechanical disruption, or cell lysing agents.

It may be desired to purify SRT from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the SRT. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular SRT produced.

E. Uses for SRT Polynucleotides and Polypeptides

SRT nucleotide sequences (and/or their complements) disclosed herein have various applications in the art of molecular biology, including for example uses as hybridization probes, in chromosome and gene mapping, in tissue typing, disease tissue detection, in PCR technologies, in screening for new therapeutic molecules and in the generation of anti-sense RNA and DNA. SRT nucleic acid will also be useful for the preparation of SRT polypeptides by the recombinant techniques described herein.

The SRT polynucleotides disclosed herein, or portions thereof, may be used as hybridization probes for a cDNA library to isolate the full-length SRT cDNA or to isolate still other cDNAs (for instance, those encoding naturally-occurring variants of SRT or SRT from other species) which have a desired sequence identity to the SRT sequence of interest. Optionally, the length of the probes will be about 20 to about 50 bases. The hybridization probes may be derived from at least partially novel regions of the nucleotide sequences disclosed herein wherein those regions may be determined without undue experimentation or from genomic sequences including promoters, enhancer elements and introns of native sequence SRT. By way of example, a screening method will comprise isolating the coding region of the SRT gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the SRT gene of the present invention can be used to screen libraries of human cDNA, genomic DNA or mRNA to determine which members of such libraries the probe hybridizes to. Hybridization techniques are described in further detail in the Examples below.

PCR as described in U.S. Pat. Nos. 4,683,195; 4,800,195; and 4,965,188 provides additional uses for oligonucleotides based upon the polynucleotide sequences disclosed in the accompanying figures. Such oligomers are generally chemically synthesized, but they may be of recombinant origin or a mixture of both. Oligomers generally comprise two nucleotide sequences, one with sense orientation (5′ to 3′) and one with antisense (3′ to 5′) employed under optimized conditions for identification of a specific gene or diagnostic use. The same two oligomers, nested sets of oligomers, or even a degenerate pool of oligomers may be employed under less stringent conditions for identification and/or quantitation of closely related DNA or RNA sequences.

Full length genes may be cloned utilizing partial nucleotide sequence and various methods known in the art. Gobinda et al. PCR Methods Applic. 2:318-322 (1993) disclose “restriction-site PCR” as a direct method which uses universal primers to retrieve unknown sequence adjacent to a known locus. First, genomic DNA is amplified in the presence of primer to linker and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase. Gobinda et al present data concerning Factor IX for which they identified a conserved stretch of 20 nucleotides in the 3′ noncoding region of the gene.

Inverse PCR is the first method to report successful acquisition of unknown sequences starting with primers based on a known region (Triglia et al., Nucleic Acids Res. 16:8186 (1988). The method uses several restriction enzymes to generate a suitable fragment in the known region of a gene. The fragment is then circularized by intramolecular ligation and used as a PCR template. Divergent primers are designed from the known region. The multiple rounds of restriction enzyme digestions and ligations that are necessary prior to PCR make the procedure slow and expensive (Gobinda et al, supra).

Capture PCR (Lagerstrom et al., PCR Methods Applic. 1:111-119 (1991) is a method for PCR amplification of DNA fragments adjacent to a known sequence in human and YAC DNA. As noted by Gobinda et al. (supra), capture PCR also requires multiple restriction enzyme digestions and ligations to place an engineered double-stranded sequence into an unknown portion of the DNA molecule before PCR. Although the restriction and ligation reactions are carried out simultaneously, the requirements for extension, immobilization and two rounds of PCR and purification prior to sequencing render the method cumbersome and time consuming.

Parker et al., Nucleic Acids Res. 19:3055-3060 (1991) teach walking PCR, a method for targeted gene walking which permits retrieval of unknown sequence. PromoterFinder™ is a new kit available from Clontech (Palo Alto, Calif.) which uses PCR and primers derived from p53 to walk in genomic DNA. Nested primers and special PromoterFinder libraries are used to detect upstream sequences such as promoters and regulatory elements. This process avoids the need to screen libraries and is useful in finding intron/exon junctions.

Another new PCR method, “Improved Method for Obtaining Full Length cDNA Sequences” (see U.S. Pat. No. 5,817,479, issued Oct. 6, 1998), employs XL-PCR (Perkin-Elmer, Foster City, Calif.) to amplify and extend partial nucleotide sequence into longer pieces of DNA. This method was developed to allow a single researcher to process multiple genes (up to 20 or more) at one time and to obtain an extended (possibly full-length) sequence within 6-10 days. This new method replaces methods which use labelled probes to screen plasmid libraries and allow one researcher to process only about 3-5 genes in 14-40 days.

In the first step, which can be performed in about two days, any two of a plurality of primers are designed and synthesized based on a known partial sequence. In step 2, which takes about six to eight hours, the sequence is extended by PCR amplification of a selected library. Steps 3 and 4, which take about one day, are purification of the amplified cDNA and its ligation into an appropriate vector. Step 5, which takes about one day, involves transforming and growing up host bacteria. In step 6, which takes approximately five hours, PCR is used to screen bacterial clones for extended sequence. The final steps, which take about one day, involve the preparation and sequencing of selected clones.

If the full length cDNA has not been obtained, the entire procedure is repeated using either the original library or some other preferred library. The preferred library may be one that has been size-selected to include only larger cDNAs or may consist of single or combined commercially available libraries, eg. lung, liver, heart and brain from Gibco/BRL (Gaithersburg, Md.). The cDNA library may have been prepared with oligo (dT) or random priming. Random primed libraries are preferred in that they will contain more sequences which contain 5′ ends of genes. A randomly primed library may be particularly useful if an oligo (dT) library does not yield a complete gene.

The nucleotide sequence for any particular polynucleotide shown in the accompanying figures can also be used to generate probes for mapping the native genomic sequence. The sequence may be mapped to a particular chromosome or to a specific region of the chromosome using well known techniques. These include in situ hybridization to chromosomal spreads (Verma et al., “Human Chromosomes: A Manual of Basic Techniques”, Pergamon Press, New York City, 1988), flow-sorted chromosomal preparations, or artificial chromosome constructions such as yeast artificial chromosomes (YACs), bacterial artificial chromosomes (BACs), bacterial P1 constructions or single chromosome cDNA libraries.

In situ hybridization of chromosomal preparations and physical mapping techniques such as linkage analysis using established chromosomal markers are invaluable in extending genetic maps. Examples of genetic maps can be found in the 1994 Genome Issue of Science (265:1981 f). Often the placement of a gene on the chromosome of another mammalian species may reveal associated markers even if the number or arm of a particular human chromosome is not known. New partial nucleotide sequences can be assigned to chromosomal arms, or parts thereof, by physical mapping. This provides valuable information to investigators searching for disease genes using positional cloning or other gene discovery techniques. Once a disease or syndrome, such as ataxia telangiectasia (AT), has been crudely localized by genetic linkage to a particular genomic region, for example, AT to 11q22-23 (Gatti et al., Nature 336:577-580 (1988), any sequences mapping to that area may represent genes for further investigation. The nucleotide sequences of the subject invention may also be used to detect differences in the chromosomal location of nucleotide sequences due to translocation, inversion, etc., between normal and carrier or affected individuals.

The partial nucleotide sequence encoding a particular SRT polypeptide may be used to produce an amino acid sequence using well known methods of recombinant DNA technology. The amino acid or peptide may be expressed in a variety of host cells, either prokaryotic or eukaryotic. Host cells may be from the same species from which the nucleotide sequence was derived or from a different species. Advantages of producing an amino acid sequence or peptide by recombinant DNA technology include obtaining adequate amounts for purification and the availability of simplified purification procedures.

Cells transformed with an SRT nucleotide sequence may be cultured under conditions suitable for the expression and recovery of peptide from cell culture as described above. The peptide produced by a recombinant cell may be secreted or may be contained intracellularly depending on the sequence itself and/or the vector used. In general, it is more convenient to prepare recombinant proteins in secreted form, and this is accomplished by ligating SRT to a recombinant nucleotide sequence which directs its movement through a particular prokaryotic or eukaryotic cell membrane. Other recombinant constructions may join SRT to nucleotide sequence encoding a polypeptide domain which will facilitate protein purification (Kroll et al., DNA Cell Biol. 12:441-53 (1993).

Other useful fragments of the SRT nucleic acids include antisense or sense oligonucleotides comprising a singe-stranded nucleic acid sequence (either RNA or DNA) capable of binding to target SRT mRNA (sense) or SRT DNA (antisense) sequences. Antisense or sense oligonucleotides, according to the present invention, comprise a fragment of the coding region of SRT DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of SRT proteins. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

The probes may also be employed in PCR techniques to generate a pool of sequences for identification of closely related SRT coding sequences.

Nucleotide sequences encoding an SRT can also be used to construct hybridization probes for mapping the gene which encodes that SRT and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for SRT encode a protein which binds to another protein (example, where the SRT is a receptor), the SRT can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor SRT can be used to isolate correlative ligand(s). Screening assays can be designed to find lead compounds that mimic the biological activity of a native SRT or a receptor for SRT. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode SRT or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding SRT can be used to clone genomic DNA encoding SRT in accordance with established techniques and the genomic sequences used to generate transgenic animals that contain cells which express DNA encoding SRT. Methods for generating transgenic animals, particularly animals such as mice or rats, have become conventional in the art and are described, for example, in U.S. Pat. Nos. 4,736,866 and 4,870,009. Typically, particular cells would be targeted for SRT transgene incorporation with tissue-specific enhancers. Transgenic animals that include a copy of a transgene encoding SRT introduced into the germ line of the animal at an embryonic stage can be used to examine the effect of increased expression of DNA encoding SRT. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of SRT can be used to construct a SRT “knock out” animal which has a defective or altered gene encoding SRT as a result of homologous recombination between the endogenous gene encoding SRT and altered genomic DNA encoding SRT introduced into an embryonic stem cell of the animal. For example, cDNA encoding SRT can be used to clone genomic DNA encoding SRT in accordance with established techniques. A portion of the genomic DNA encoding SRT can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the SRT polypeptide.

Nucleic acid encoding the SRT polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The SRT polypeptides described herein may also be employed as molecular weight markers for protein electrophoresis purposes.

The nucleic acid molecules encoding the SRT polypeptides or fragments thereof described herein are useful for chromosome identification. In this regard, there exists an ongoing need to identify new chromosome markers, since relatively few chromosome marking reagents, based upon actual sequence data are presently available. Each SRT nucleic acid molecule of the present invention can be used as a chromosome marker.

The SRT polypeptides and nucleic acid molecules of the present invention may also be used for tissue typing, wherein the SRT polypeptides of the present invention may be differentially expressed in one tissue as compared to another, for example in a diseased tissue versus a normal tissue. SRT nucleic acid molecules will find use for generating probes for PCR, Northern analysis, Southern analysis and Western analysis.

The SRT polypeptides described herein and antibodies thereagainst may also be employed as therapeutic agents. The SRT polypeptides of the present invention can be formulated according to known methods to prepare pharmaceutically useful compositions, whereby the SRT product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a SRT polypeptide or agonist or antagonist thereof is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of a SRT polypeptide is desired in a formulation with release characteristics suitable for the treatment of any disease or disorder requiring administration of the SRT polypeptide, microencapsulation of the SRT polypeptide is contemplated. Microencapsulation of recombinant proteins for sustained release has been successfully performed with human growth hormone (rhGH), interferon-(rhIFN- ), interleukin-2, and MN rgp120. Johnson et al., Nat. Med., 2:795-799 (1996); Yasuda, Biomed. Ther., 27:1221-1223 (1993); Hora et al., Bio/Technology 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp.439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp.1-41.

This invention encompasses methods of screening compounds to identify those that mimic the SRT polypeptide (agonists) or prevent the effect of the SRT polypeptide (antagonists). Screening assays for antagonist drug candidates are designed to identify compounds that bind or complex with the SRT polypeptides encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a SRT polypeptide encoded by a nucleic acid identified herein under conditions and for a time sufficient to allow these two components to interact.

In binding assays, the interaction is binding and the complex formed can be isolated or detected in the reaction mixture. In a particular embodiment, the SRT polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the SRT polypeptide and drying. Alternatively, an immobilized antibody, e.g., a monoclonal antibody, specific for the SRT polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular SRT polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GALA activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a SRT polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the SRT polypeptide may be added to a cell along with the compound to be screened for a particular activity and the ability of the compound to inhibit the activity of interest in the presence of the SRT polypeptide indicates that the compound is an antagonist to the SRT polypeptide. Alternatively, antagonists may be detected by combining the SRT polypeptide and a potential antagonist with membrane-bound SRT polypeptide receptors or recombinant receptors under appropriate conditions for a competitive inhibition assay. The SRT polypeptide can be labeled, such as by radioactivity, such that the number of SRT polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the SRT polypeptide and a cDNA library created from this RNA is divided into pools and used to transfect COS cells or other cells that are not responsive to the SRT polypeptide. Transfected cells that are grown on glass slides are exposed to labeled SRT polypeptide. The SRT polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled SRT polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled SRT polypeptide in the presence of the candidate compound. The ability of the compound to enhance or block this interaction could then be measured.

More specific examples of potential antagonists include an oligonucleotide that binds to the fusions of immunoglobulin with SRT polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the SRT polypeptide that recognizes the receptor but imparts no effect, thereby competitively inhibiting the action of the SRT polypeptide.

Another potential SRT polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature SRT polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the SRT polypeptide. The antisense RNA oligonucleotide hybridizes to the mRNA in vivo and blocks translation of the mRNA molecule into the SRT polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the SRT polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the SRT polypeptide, thereby blocking the normal biological activity of the SRT polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

F. Anti-SRT Polypeptide Antibodies

The present invention further provides anti-SRT antibodies. Exemplary antibodies include polyclonal, monoclonal, humanized, bispecific, and heteroconjugate antibodies.

1. Polyclonal Antibodies

The anti-SRT antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the SRT polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-SRT antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the SRT polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against SRT. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-SRT antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

4. Bispecific Antibodies

Bispecific antibodies are monoclonal, preferably human or humanized, antibodies that have binding specificities for at least two different antigens. In the present case, one of the binding specificities is for the SRT, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al, Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given SRT polypeptide herein. Alternatively, an anti-SRT polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD 16) so as to focus cellular defense mechanisms to the cell expressing the particular SRT polypeptide. Bispecific antibodies may also be used to localize cytotoxic agents to cells which express a particular SRT polypeptide. These antibodies possess a SRT-binding arm and an arm which binds a cytotoxic agent or a radionuclide chelator, such as EOTUBE, DPTA, DOTA, or TETA. Another bispecific antibody of interest binds the SRT polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al, Anti-Cancer Drug Design, 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See W094/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

9. Pharmaceutical Compositions of Antibodies

Antibodies specifically binding a SRT polypeptide identified herein, as well as other molecules identified by the screening assays disclosed hereinbefore, can be administered for the treatment of various disorders in the form of pharmaceutical compositions.

If the SRT polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

G. Uses for anti-SRT Antibodies

The anti-SRT antibodies of the invention have various utilities. For example, anti-SRT antibodies may be used in diagnostic assays for SRT, e.g., detecting its expression in specific cells, tissues, or serum. Various diagnostic assay techniques known in the art may be used, such as competitive binding assays, direct or indirect sandwich assays and immunoprecipitation assays conducted in either heterogeneous or homogeneous phases [Zola, Monoclonal Antibodies: A Manual of Techniques, CRC Press, Inc. (1987) pp. 147-158]. The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-SRT antibodies also are useful for the affinity purification of SRT from recombinant cell culture or natural sources. In this process, the antibodies against SRT are immobilized on a suitable support, such a Sephadex resin or filter paper, using methods well known in the art. The immobilized antibody then is contacted with a sample containing the SRT to be purified, and thereafter the support is washed with a suitable solvent that will remove substantially all the material in the sample except the SRT, which is bound to the immobilized antibody. Finally, the support is washed with another suitable solvent that will release the SRT from the antibody.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### EXAMPLE 1

### Isolation of SRT cDNAs

1. Preparation of oligo dT primed cDNA library

mRNA was isolated from human tissue using reagents and protocols from Invitrogen, San Diego, Calif. (Fast Track 2). This RNA was used to generate an oligo dT primed cDNA library in the vector pRK5D using reagents and protocols from Life Technologies, Gaithersburg, Md. (Super Script Plasmid System). In this procedure, the double stranded cDNA was sized to greater than 1000 bp and the SalI/NotI linkered cDNA was cloned into Xhol/NotI cleaved vector. pRK5D is a cloning vector that has an sp6 transcription initiation site followed by an SfiI restriction enzyme site preceding the XhoI/NotI cDNA cloning sites.

2. Preparation of random primed cDNA library

A secondary cDNA library was generated in order to preferentially represent the 5′ ends of the primary cDNA clones. Sp6 RNA was generated from the primary library (described above), and this RNA was used to generate a random primed cDNA library in the vector pSST-AMY.0 using reagents and protocols from Life Technologies (Super Script Plasmid System, referenced above). In this procedure the double stranded cDNA was sized to 500-1000 bp, linkered with blunt to NotI adaptors, cleaved with SfiI, and cloned into SfiI/NotI cleaved vector. pSST-AMY.0 is a cloning vector that has a yeast alcohol dehydrogenase promoter preceding the cDNA cloning sites and the mouse amylase sequence (the mature sequence without the secretion signal) followed by the yeast alcohol dehydrogenase terminator, after the cloning sites. Thus, cDNAs cloned into this vector that are fused in frame with the amylase sequence will lead to the secretion of amylase from appropriately transfected yeast colonies.

3. Transformation and Detection

DNA from the library described in paragraph 2 above was chilled on ice to which was added electrocompetent DH10B bacteria (Life Technologies, 20 ml). The bacteria and vector mixture was then electroporated as recommended by the manufacturer. Subsequently, SOC media (Life Technologies, 1 ml) was added and the mixture was incubated at 37° C. for 30 minutes. The transformants were then plated onto 20 standard 150 mm LB plates containing ampicillin and incubated for 16 hours (37° C.). Positive colonies were scraped off the plates and the DNA was isolated from the bacterial pellet using standard protocols, e.g. CsCl-gradient. The purified DNA was then carried on to the yeast protocols below.

The yeast methods were divided into three categories: (1) Transformation of yeast with the plasmid/cDNA combined vector; (2) Detection and isolation of yeast clones secreting amylase; and (3) PCR amplification of the insert directly from the yeast colony and purification of the DNA for sequencing and further analysis.

The yeast strain used was HD56-5A (ATCC-90785). This strain has the following genotype: MAT alpha, ura3-52, leu2-3, leu2-112, his3-11, his3-15, MAL⁺, SUC⁺, GAL⁺. Preferably, yeast mutants can be employed that have deficient post-translational pathways. Such mutants may have translocation deficient alleles in sec71, sec72, sec62, with truncated sec71 being most preferred. Alternatively, antagonists (including antisense nucleotides and/or ligands) which interfere with the normal operation of these genes, other proteins implicated in this post translation pathway (e.g., SEC61p, SEC72p, SEC62p, SEC63p, TDJ1p or SSA1p-4p) or the complex formation of these proteins may also be preferably employed in combination with the amylase-expressing yeast.

Transformation was performed based on the protocol outlined by Gietz et al., Nucl. Acid. Res., 20:1425 (1992). Transformed cells were then inoculated from agar into YEPD complex media broth (100 ml) and grown overnight at 30° C. The YEPD broth was prepared as described in Kaiser et al., Methods in Yeast Genetics, Cold Spring Harbor Press, Cold Spring Harbor, N.Y., p. 207 (1994). The overnight culture was then diluted to about 2×10⁶ cells/ml (approx. OD₆₀₀=0.1) into fresh YEPD broth (500 ml) and regrown to 1×10⁷ cells/ml (approx. OD₆₀₀=0.4-0.5).

The cells were then harvested and prepared for transformation by transfer into GS3 rotor bottles in a Sorval GS3 rotor at 5,000 rpm for 5 minutes, the supernatant discarded, and then resuspended into sterile water, and centrifuged again in 50 ml falcon tubes at 3,500 rpm in a Beckman GS-6KR centrifuge. The supernatant was discarded and the cells were subsequently washed with LiAc/TE (10 ml, 10 mM Tris-HCl, 1 mM EDTA pH 7.5, 100 mM Li₂OOCCH₃), and resuspended into LiAc/TE (2.5 ml).

Transformation took place by mixing the prepared cells (100 μl) with freshly denatured single stranded salmon testes DNA (Lofstrand Labs, Gaithersburg, Md.) and transforming DNA (1 μg, vol.<10 μl) in microfuge tubes. The mixture was mixed briefly by vortexing, then 40% PEG/TE (600 μl, 40% polyethylene glycol-4000, 10 mM Tris-HCl, 1 mM EDTA, 100 mM Li₂OOCCH₃, pH 7.5) was added. This mixture was gently mixed and incubated at 30° C. while agitating for 30 minutes. The cells were then heat shocked at 42° C. for 15 minutes, and the reaction vessel centrifuged in a microfuge at 12,000 rpm for 5-10 seconds, decanted and resuspended into TE (500 μl, 10 mM Tris-HCl, 1 mM EDTA pH 7.5) followed by recentrifugation. The cells were then diluted into TE (1 ml) and aliquots (200 μl) were spread onto the selective media previously prepared in 150 mm growth plates (VWR).

Alternatively, instead of multiple small reactions, the transformation was performed using a single, large scale reaction, wherein reagent amounts were scaled up accordingly.

The selective media used was a synthetic complete dextrose agar lacking uracil (SCD-Ura) prepared as described in Kaiser et al., Methods in Yeast Genetics, Cold Spring Harbor Press, Cold Spring Harbor, N.Y., p. 208-210 (1994). Transformants were grown at 30° C. for 2-3 days.

The detection of colonies secreting amylase was performed by including red starch in the selective growth media. Starch was coupled to the red dye (Reactive Red-120, Sigma) as per the procedure described by Biely et al., Anal. Biochem., 172: 176-179 (1988). The coupled starch was incorporated into the SCD-Ura agar plates at a final concentration of 0.15% (w/v), and was buffered with potassium phosphate to a pH of 7.0 (50-100 mM final concentration).

The positive colonies were picked and streaked across fresh selective media (onto 150 mm plates) in order to obtain well isolated and identifiable single colonies. Well isolated single colonies positive for amylase secretion were detected by direct incorporation of red starch into buffered SCD-Ura agar. Positive colonies were determined by their ability to break down starch resulting in a clear halo around the positive colony visualized directly.

4. Isolation of DNA by PCR Amplification

When a positive colony was isolated, a portion of it was picked by a toothpick and diluted into sterile water (30 μl) in a 96 well plate. At this time, the positive colonies were either frozen and stored for subsequent analysis or immediately amplified. An aliquot of cells (5 μl) was used as a template for the PCR reaction in a 25 μl volume containing: 0.5 μl Klentaq (Clontech, Palo Alto, Calif.); 4.0 μl 10 mM dNTP's (Perkin Elmer-Cetus); 2.5 μl Klentaq buffer (Clontech); 0.25 μl forward oligo 1; 0.25 μl reverse oligo 2; 12.5 μl distilled water. The sequence of the forward oligonucleotide 1 was:

5′-TGTAAAACGACGGCCAGTTAAATAGACCTGCAATTATTAATCT-3′ (SEQ ID NO:563)

The sequence of reverse oligonucleotide 2 was:

5′-CAGGAAACAGCTATGACCACCTGCACACCTGCAAATCCATT-3′ (SEQ ID NO:564)

PCR was then performed as follows:

| | | | |
|---|---|---|---|
| a. | | Denature | 92° C., 5 minutes |
| b. | 3 cycles of: | Denature | 92° C., 30 seconds |
| | | Anneal | 59° C., 30 seconds |
| | | Extend | 72° C., 60 seconds |
| c. | 3 cycles of: | Denature | 92° C., 30 seconds |
| | | Anneal | 57° C., 30 seconds |
| | | Extend | 72° C., 60 seconds |
| d. | 25 cycles of: | Denature | 92° C., 30 seconds |
| | | Anneal | 55° C., 30 seconds |
| | | Extend | 72° C., 60 seconds |
| e. | | Hold | 4° C. |

The underlined regions of the oligonucleotides disclosed above annealed to the ADH promoter region and the amylase region, respectively, and amplified a 307 bp region from vector pSST-AMY.0 when no insert was present. Typically, the first 18 nucleotides of the 5′ end of these oligonucleotides contained annealing sites for the sequencing primers. Thus, the total product of the PCR reaction from an empty vector was 343 bp. However, signal sequence-fused cDNA resulted in considerably longer nucleotide sequences.

Following the PCR, an aliquot of the reaction (5 μl) was examined by agarose gel electrophoresis in a 1% agarose gel using a Tris-Borate-EDTA (TBE) buffering system as described by Sambrook et al., supra. Clones resulting in a single strong PCR product larger than 400 bp were further analyzed by DNA sequencing after purification with a 96 Qiaquick PCR clean-up column (Qiagen Inc., Chatsworth, Calif.).

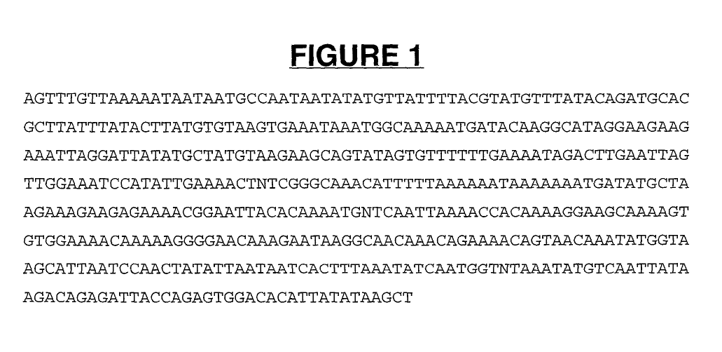

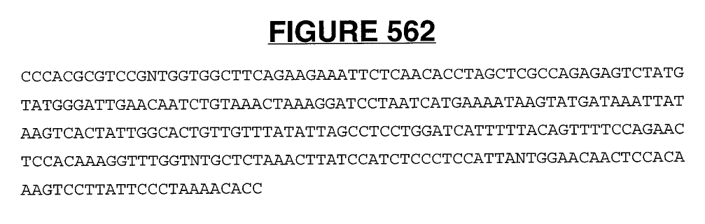

cDNA molecules isolated from this amylase screen are shown in FIGS. 1-562 (SEQ ID NOS: 1-562, respectively), wherein the nucleotides “N” and “X” represent any nucleotide. The cDNA libraries from which these cDNA molecules were obtained are as follows:

(a) Human liver tissue

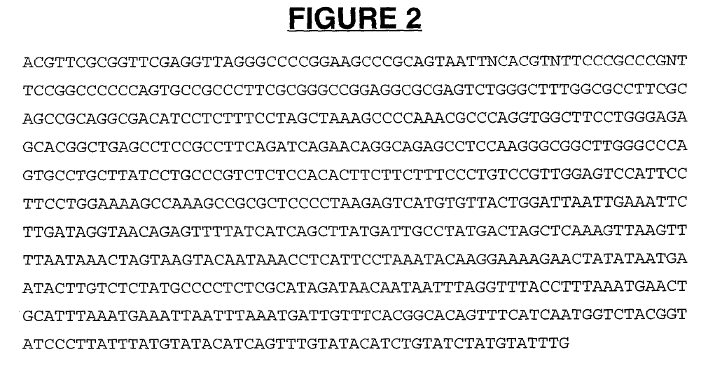

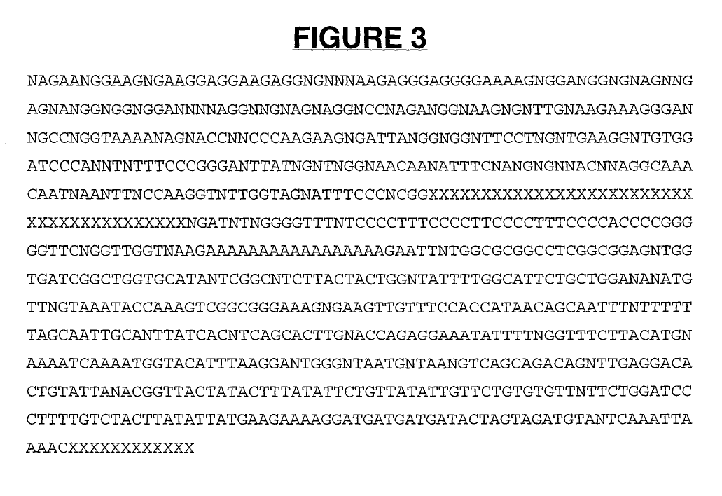

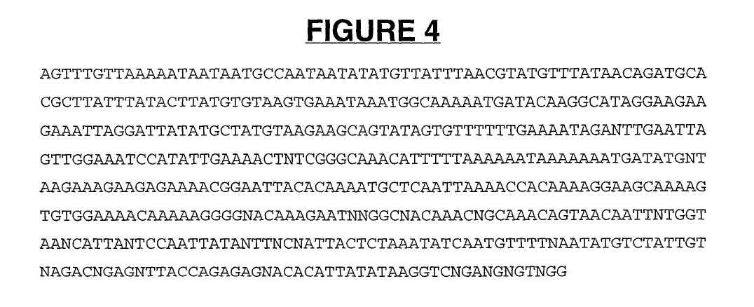

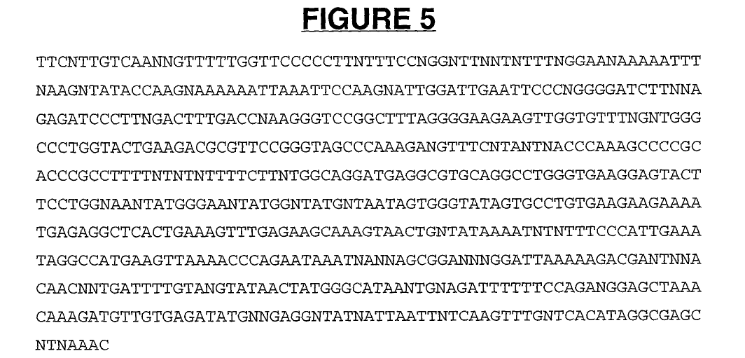

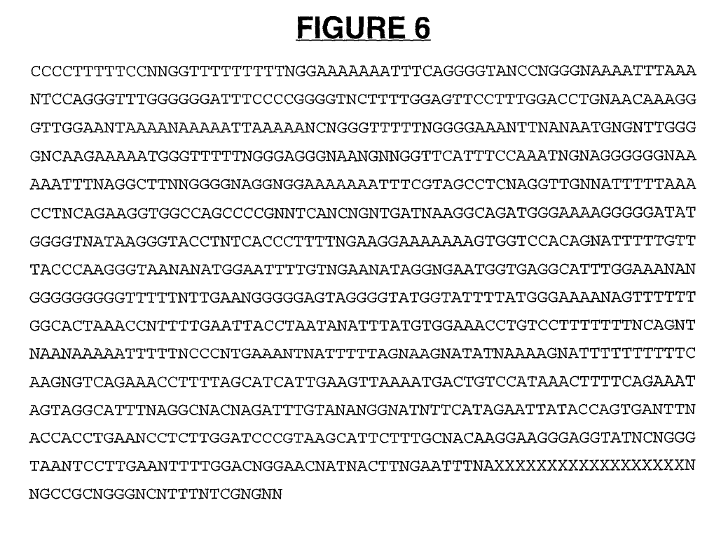

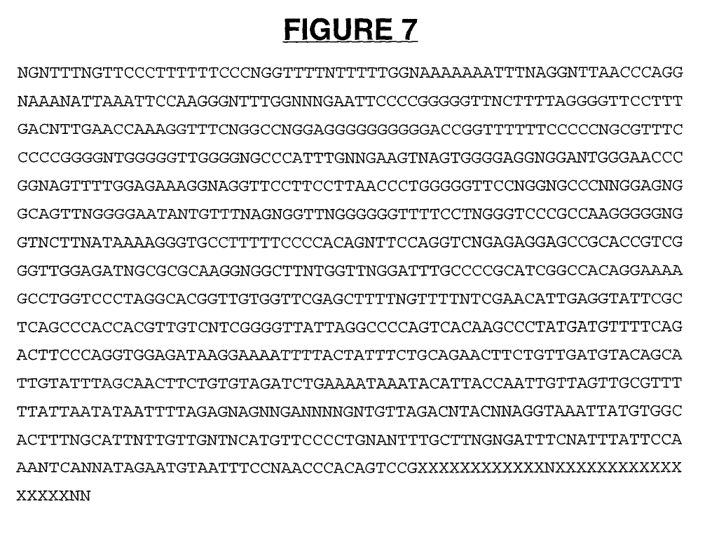

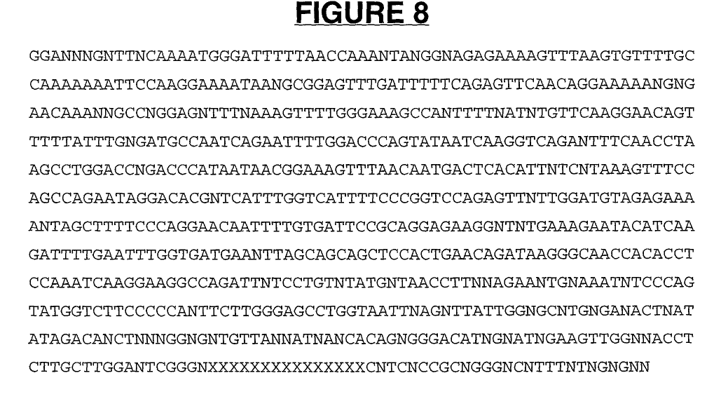

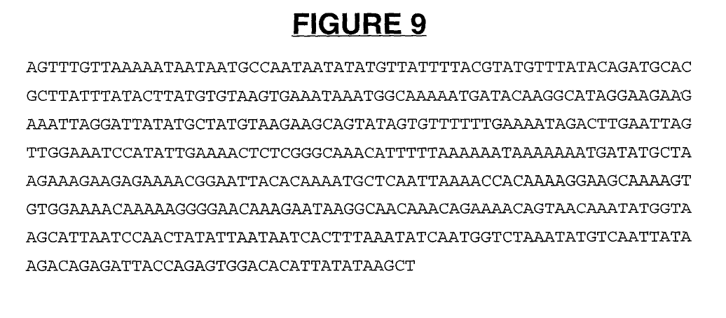

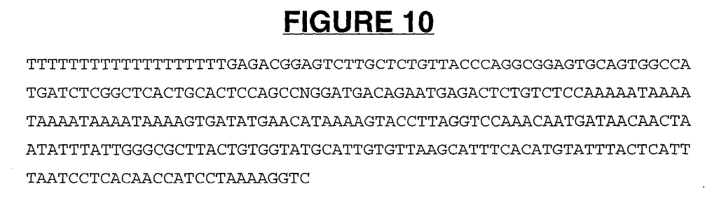

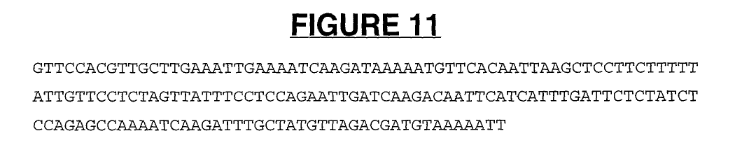

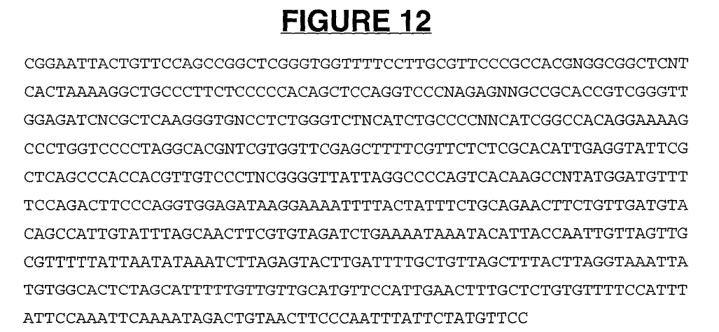

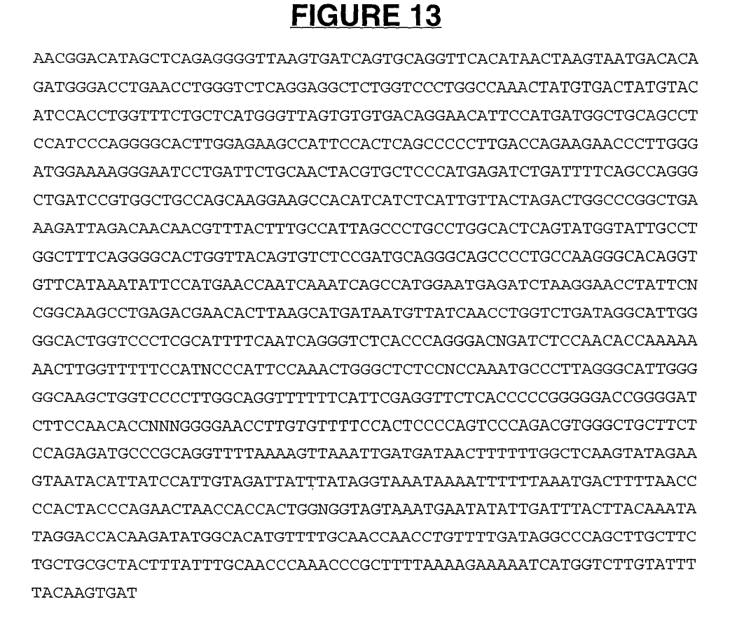

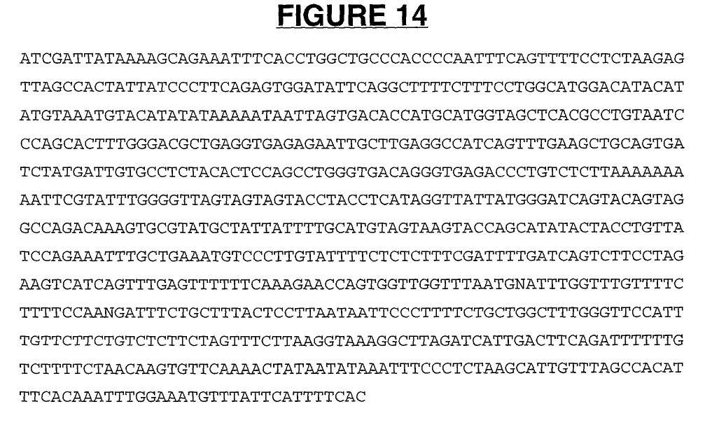

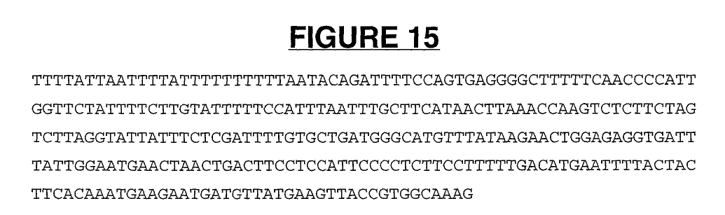

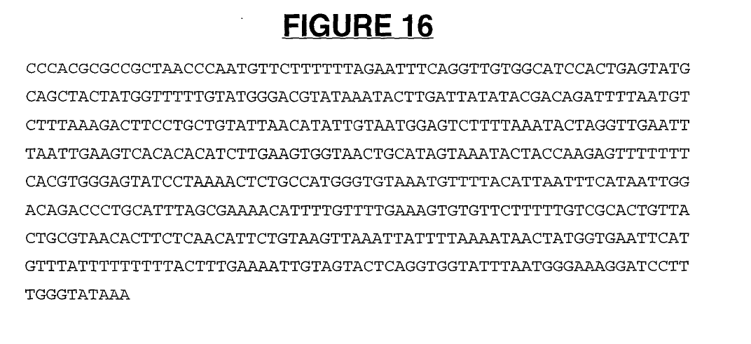

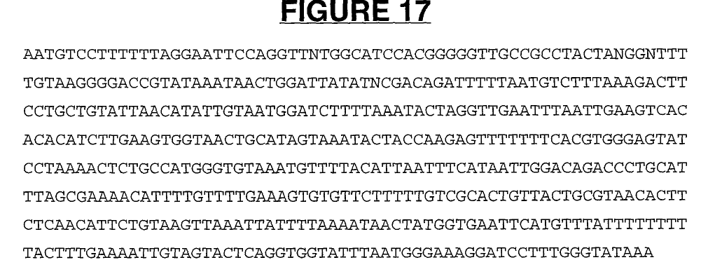

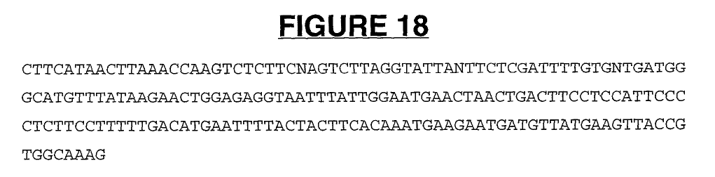

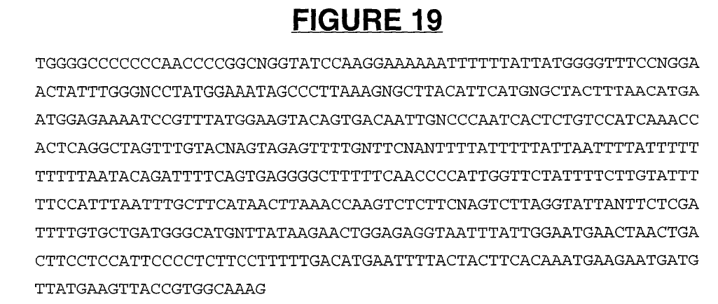

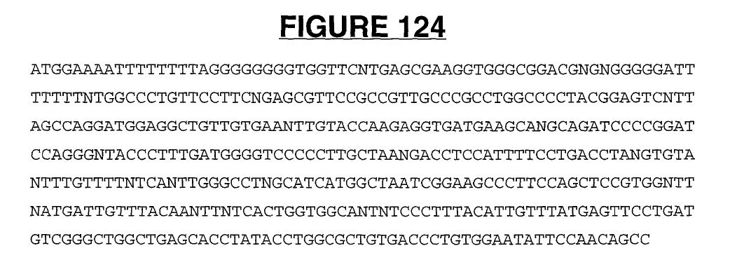

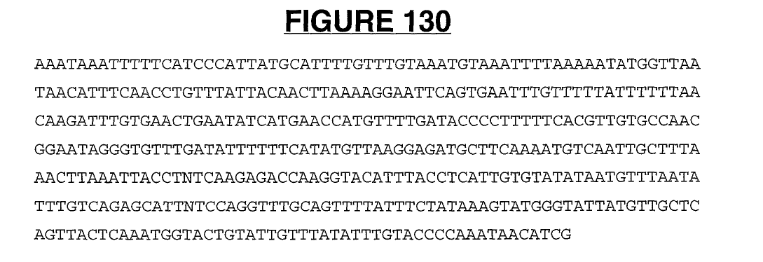

FIGS. 1-19, 124 and 130.

(b) Human placenta tissue

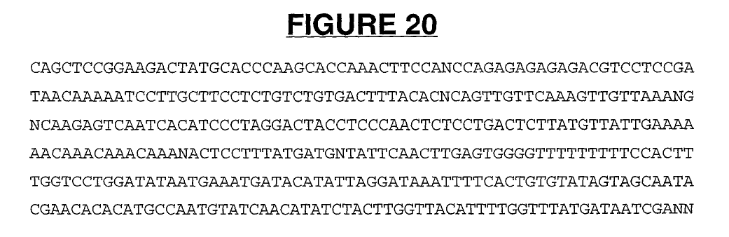

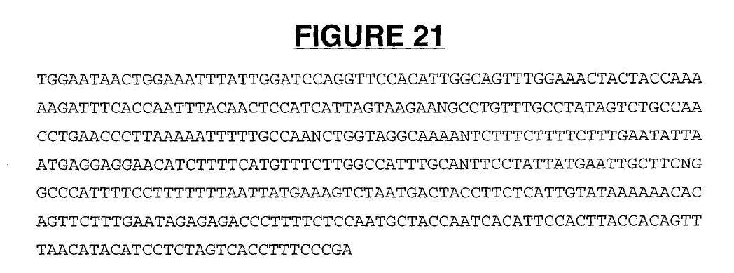

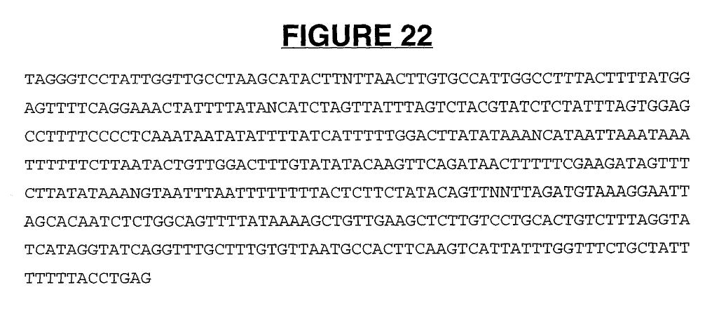

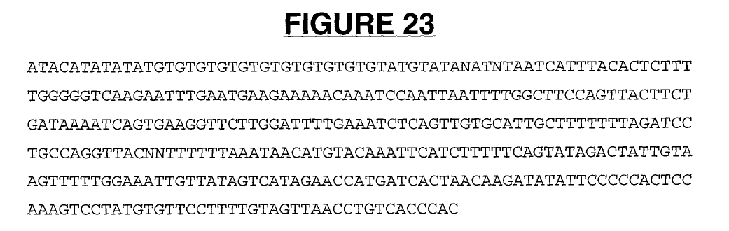

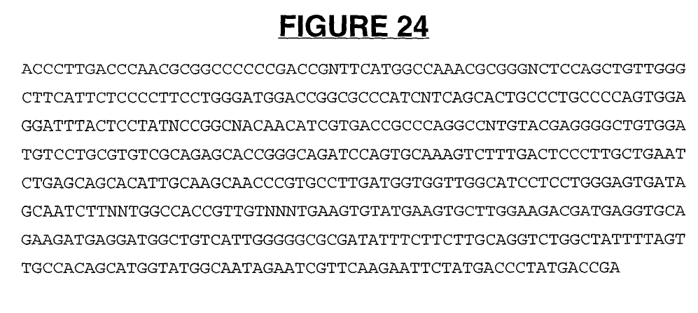

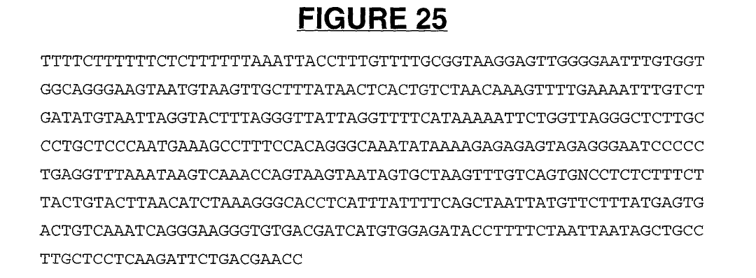

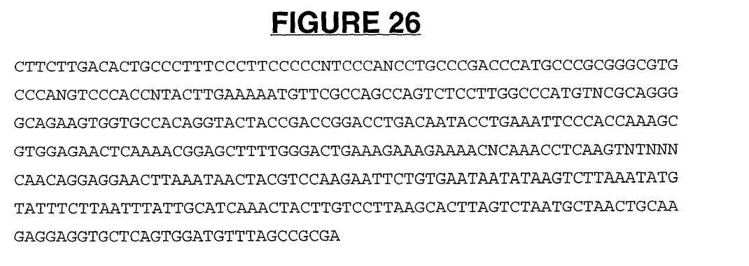

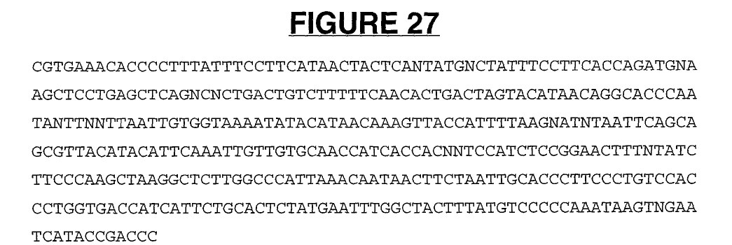

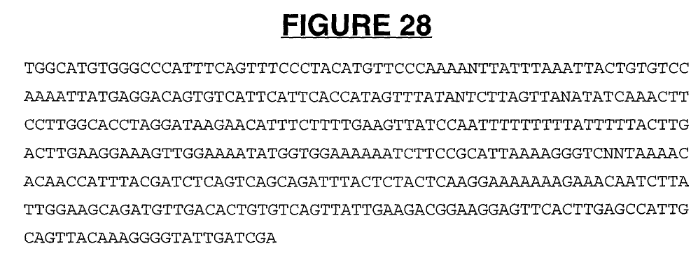

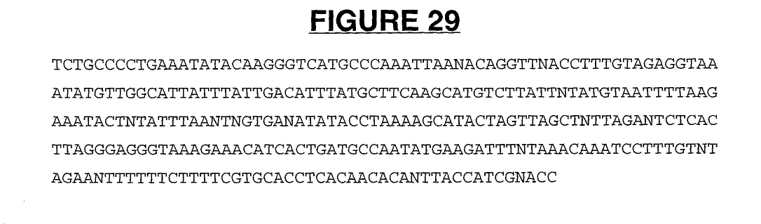

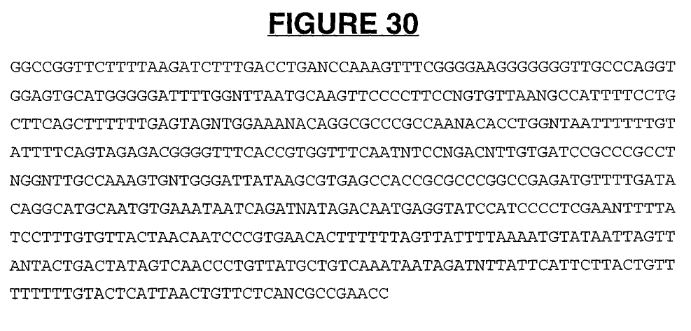

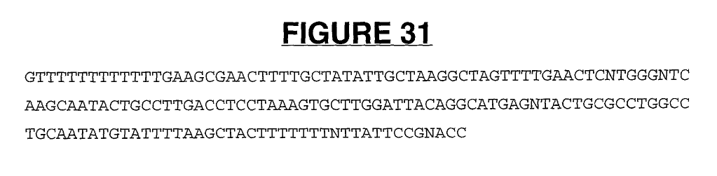

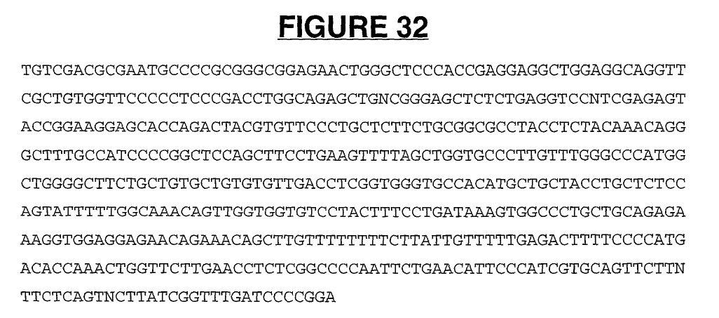

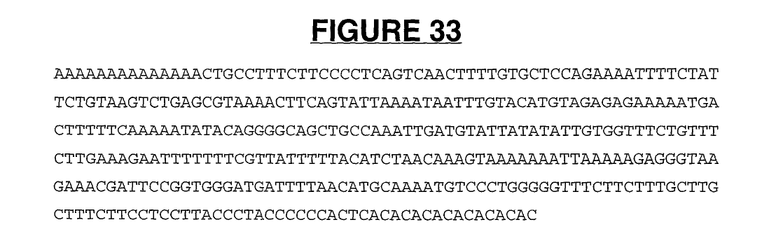

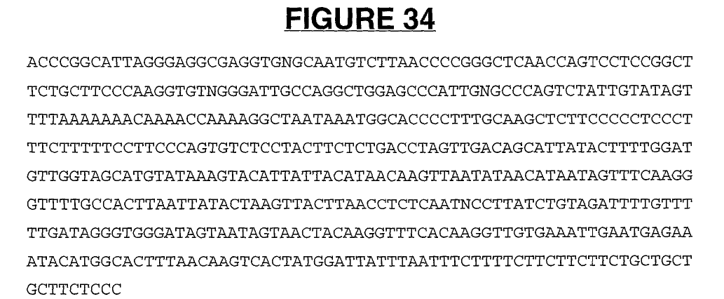

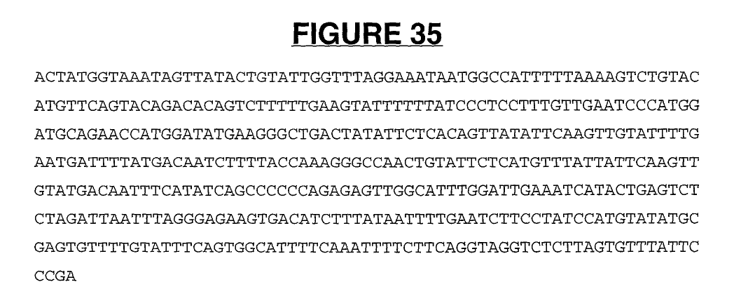

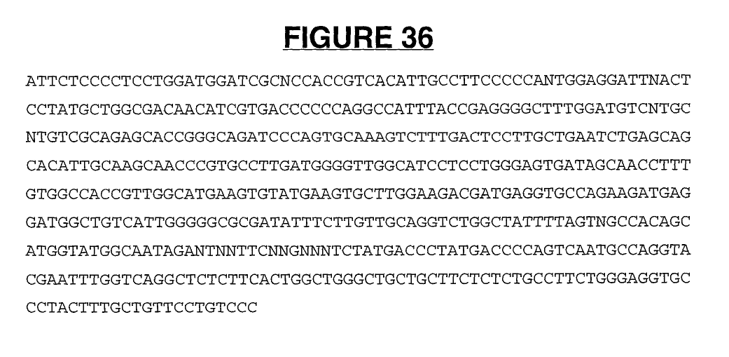

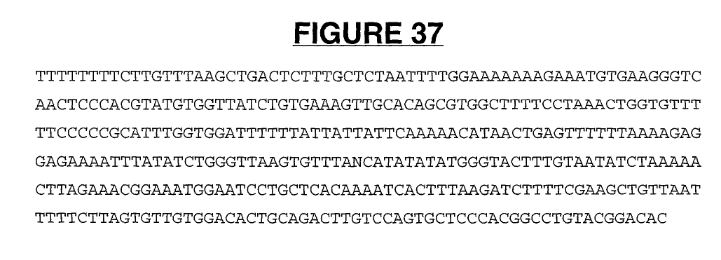

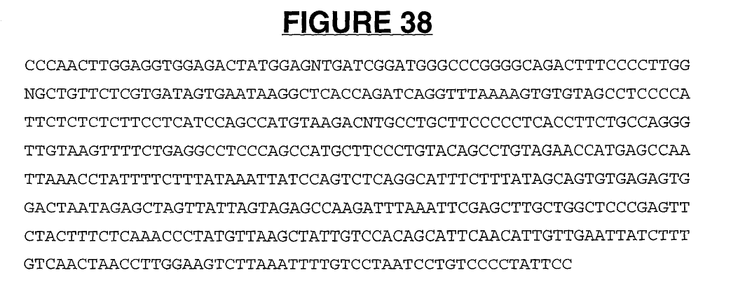

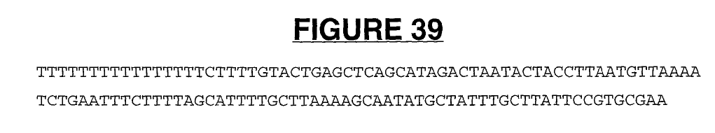

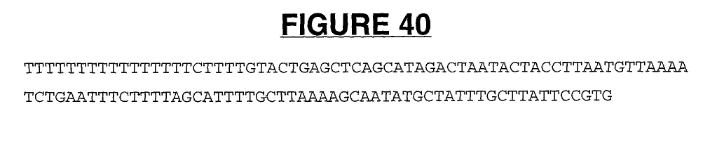

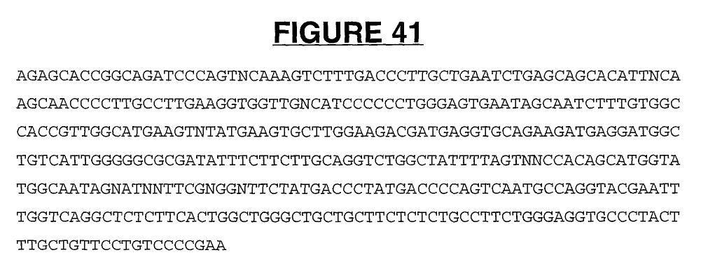

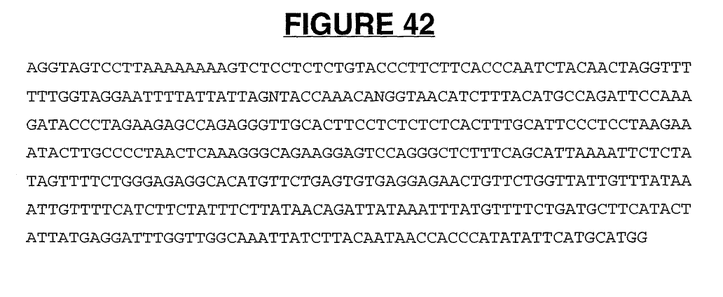

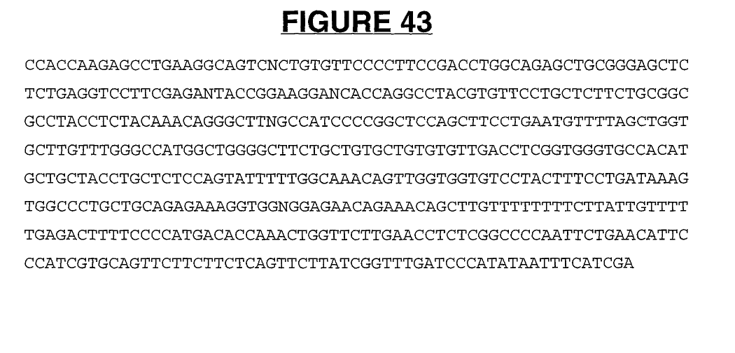

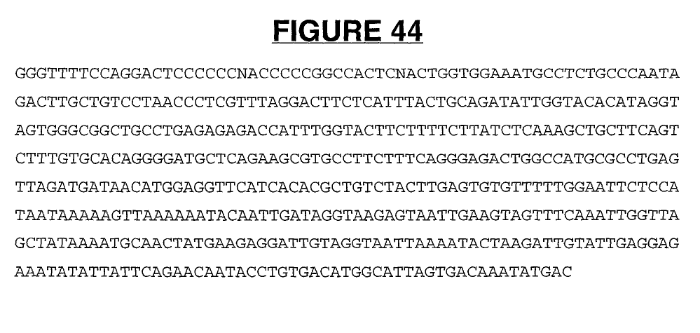

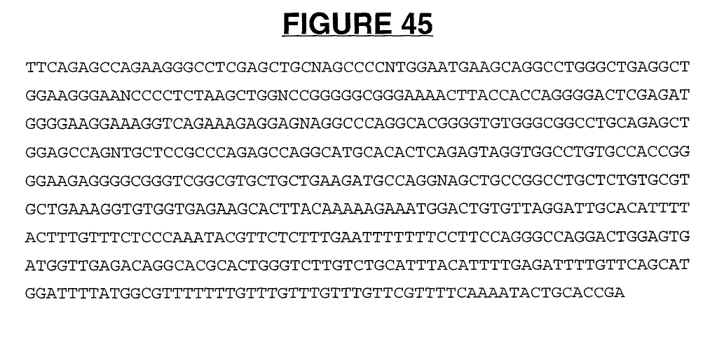

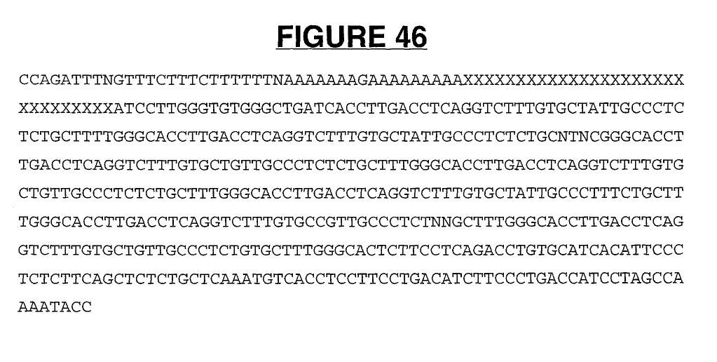

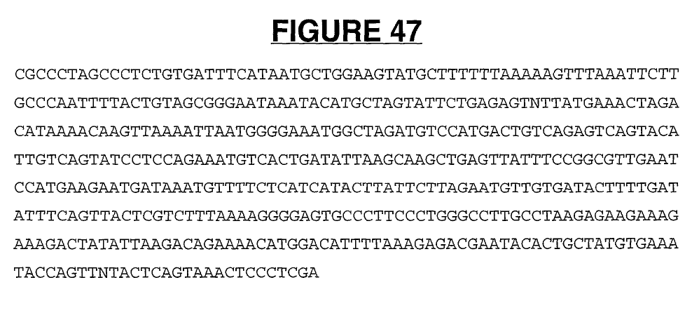

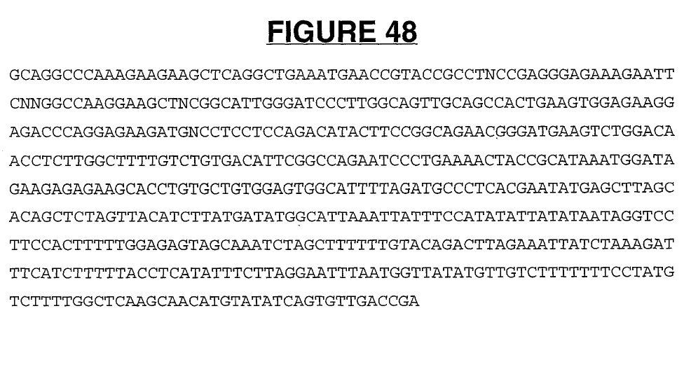

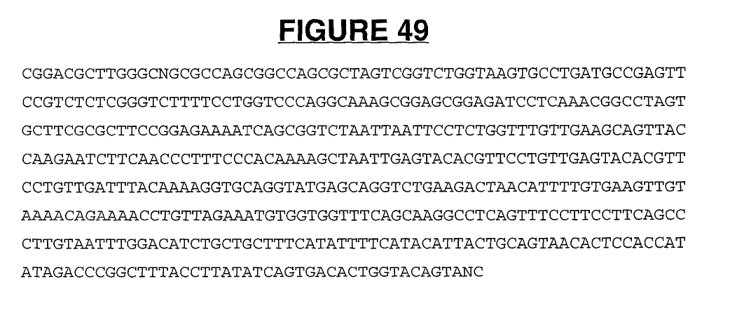

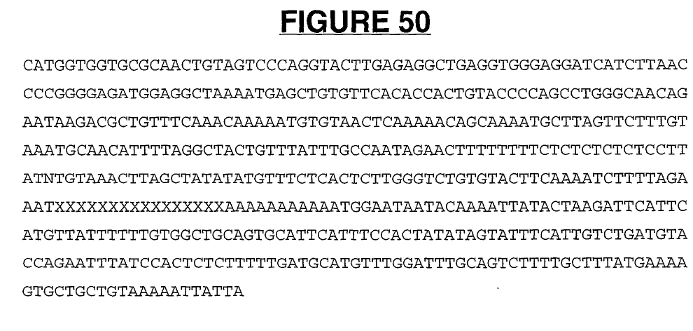

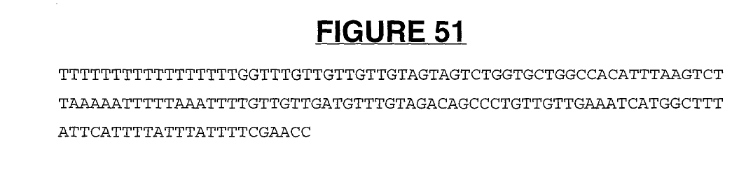

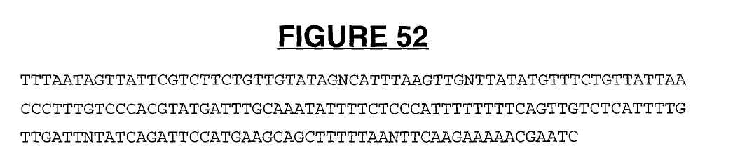

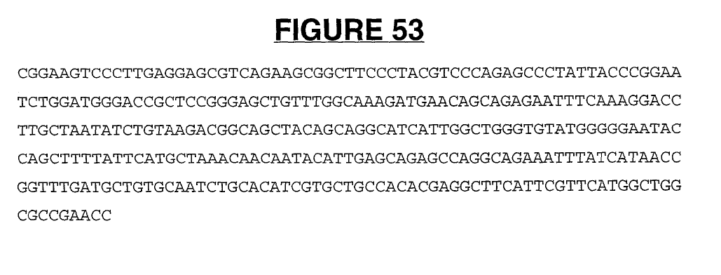

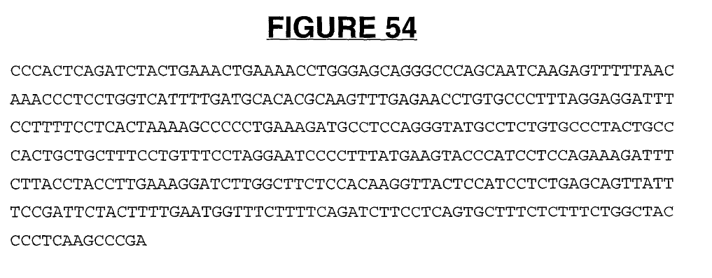

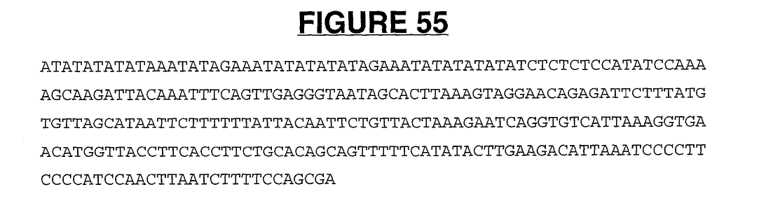

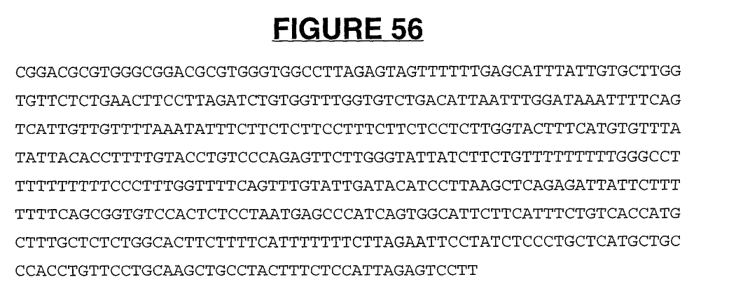

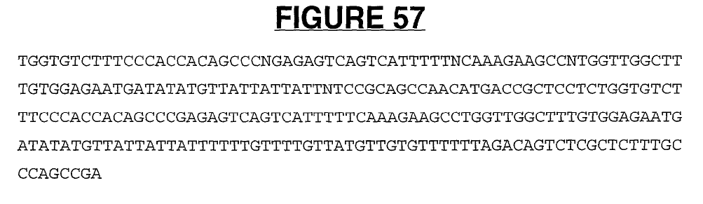

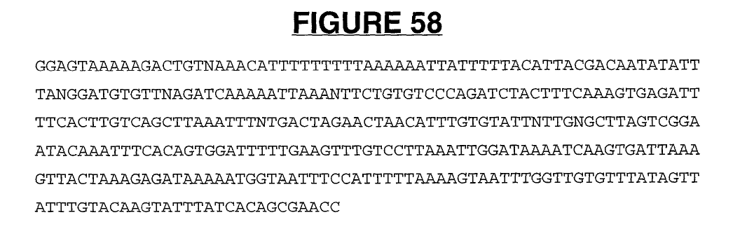

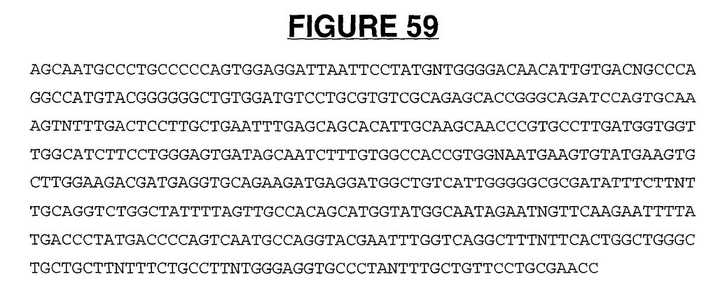

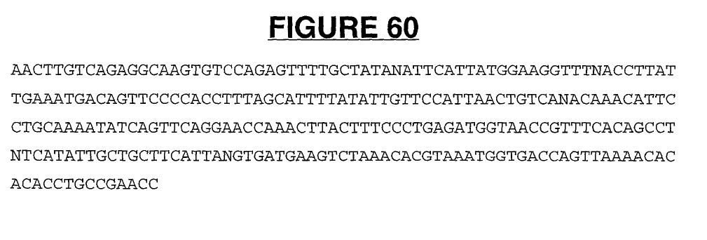

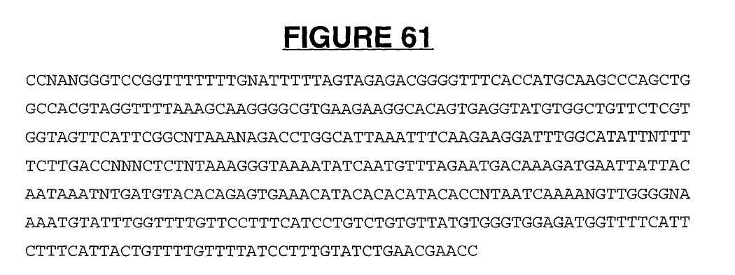

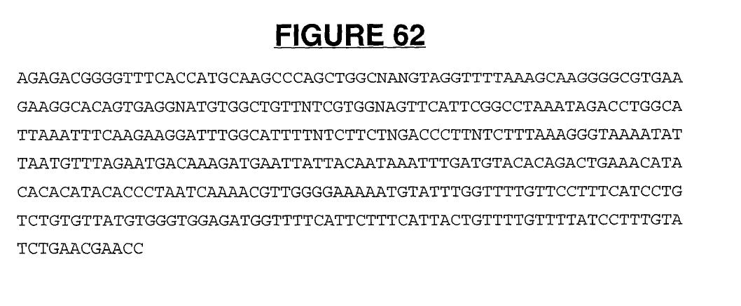

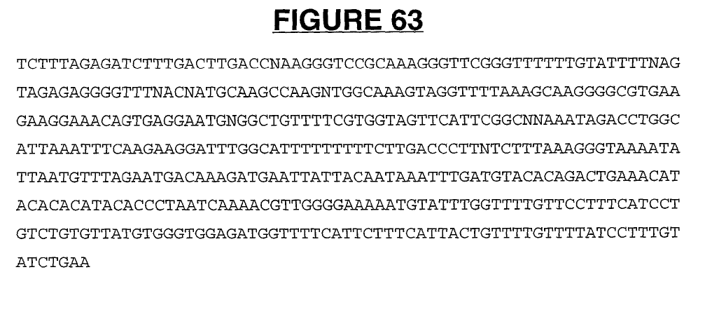

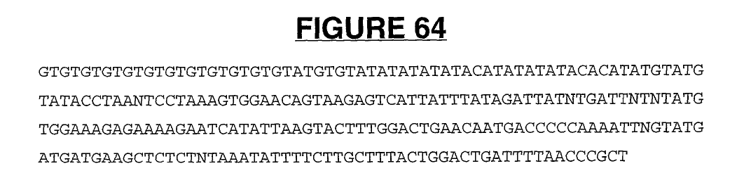

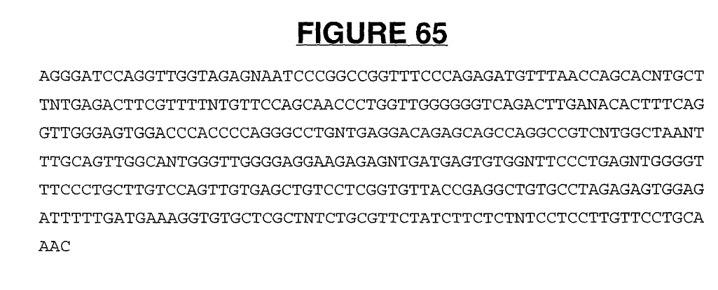

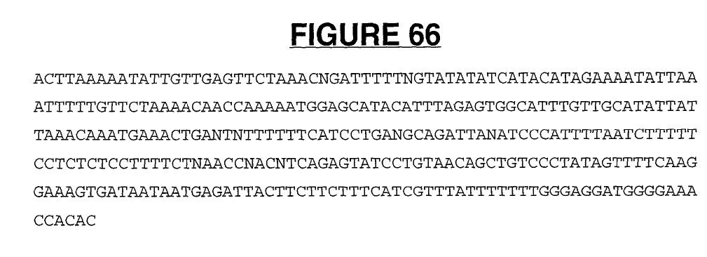

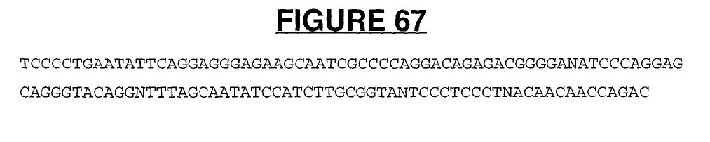

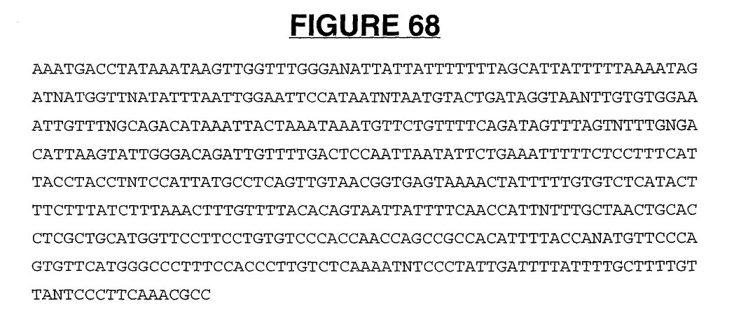

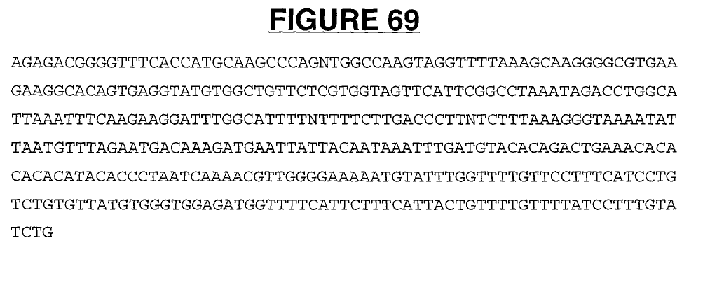

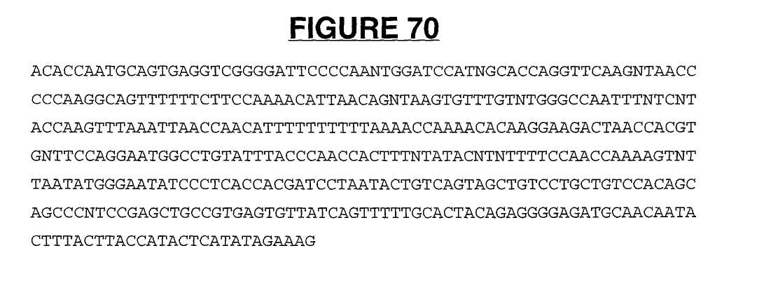

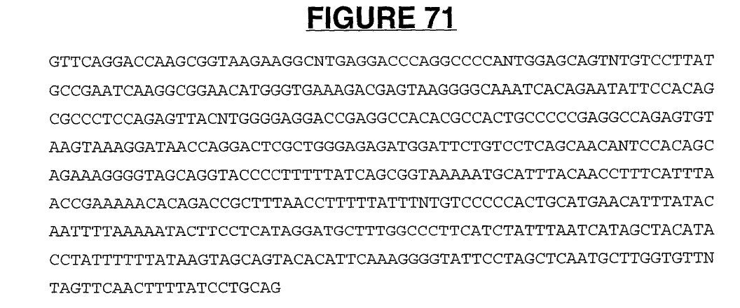

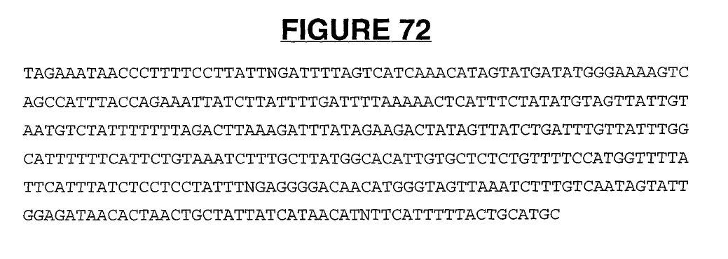

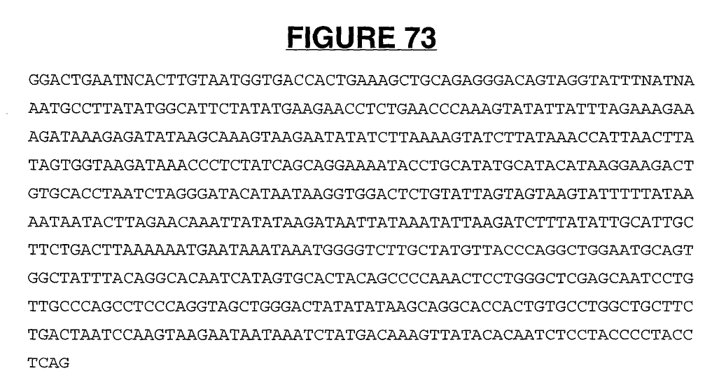

FIGS. 20-73.

(c) Human retina tissue

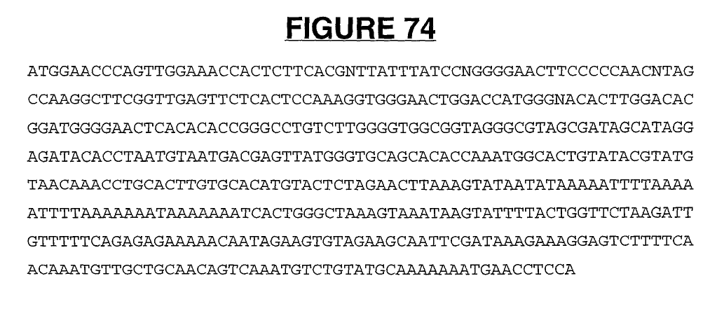

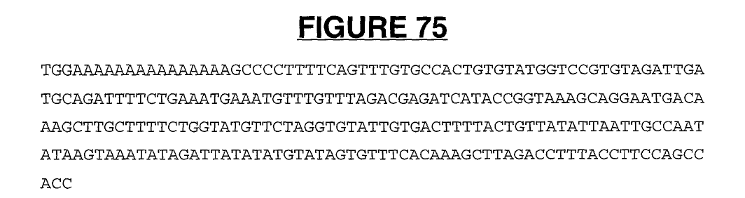

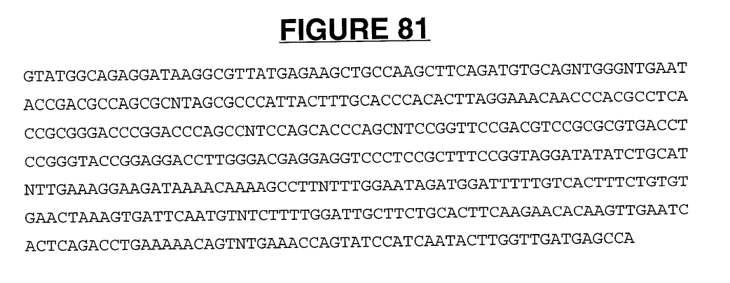

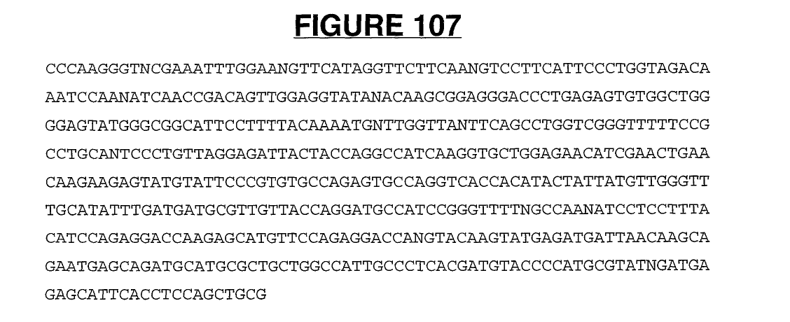

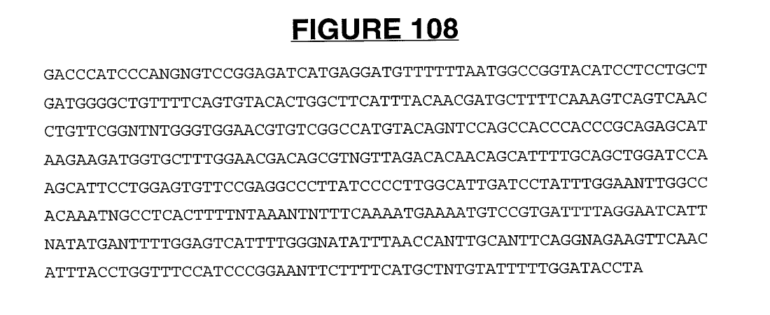

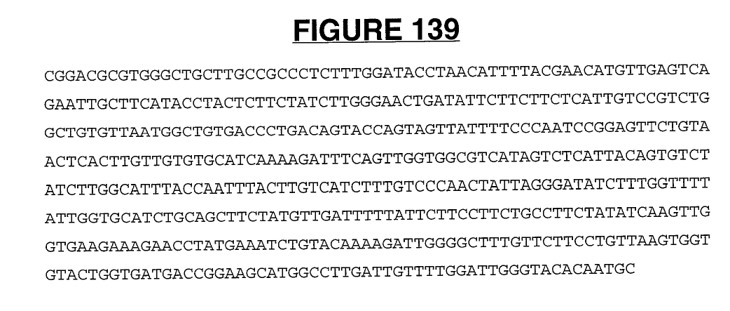

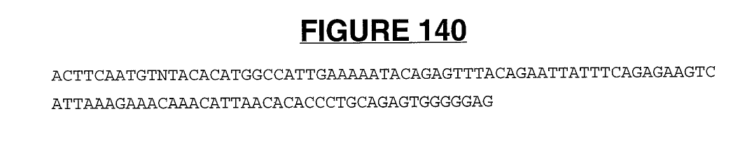

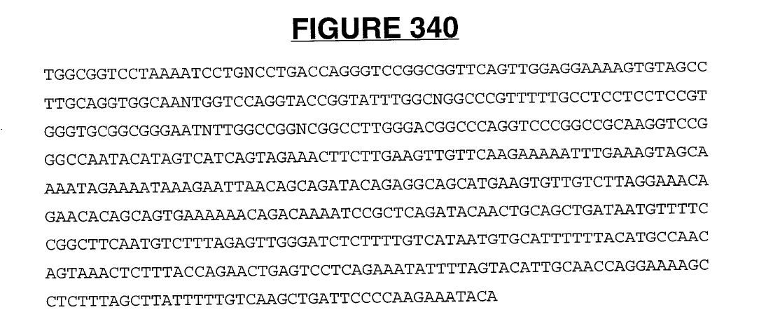

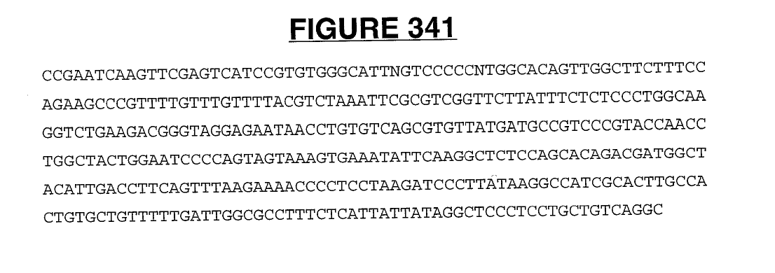

FIGS. 74-75, 81, 107-108, 139-140 and 340-341.

(d) Human salivary gland tissue

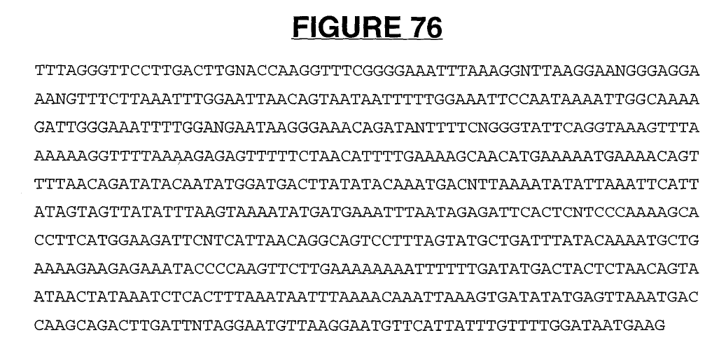

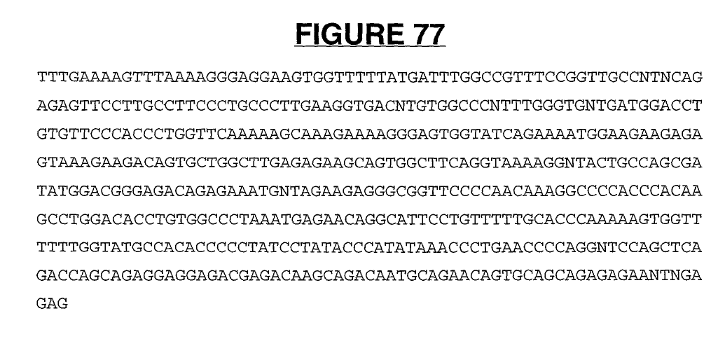

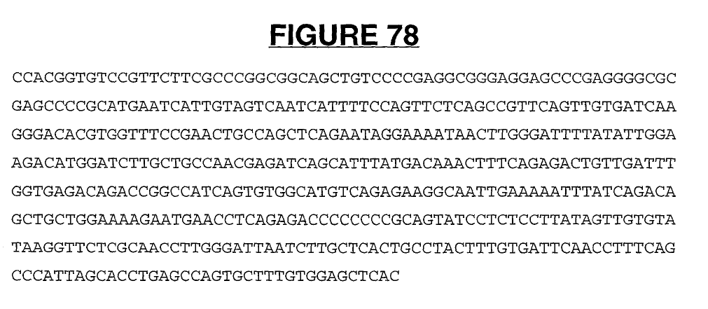

FIGS. 76-78.

(e) Human umbilical vein endothelial cells

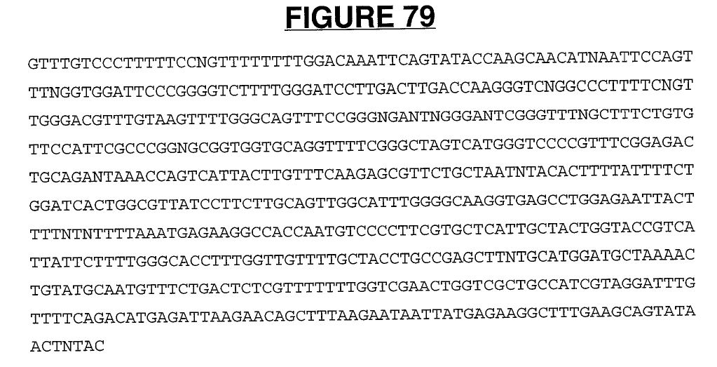

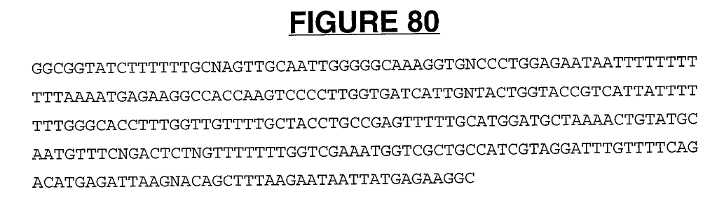

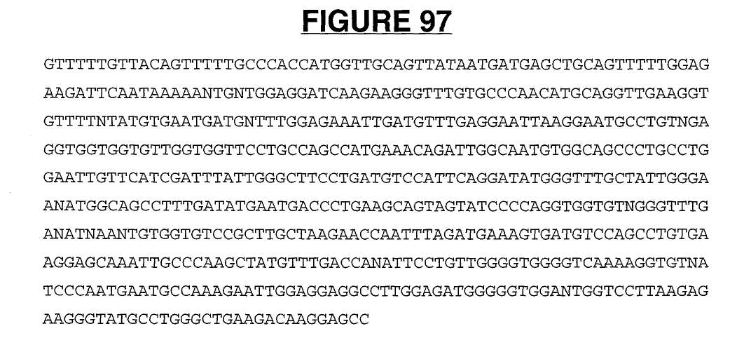

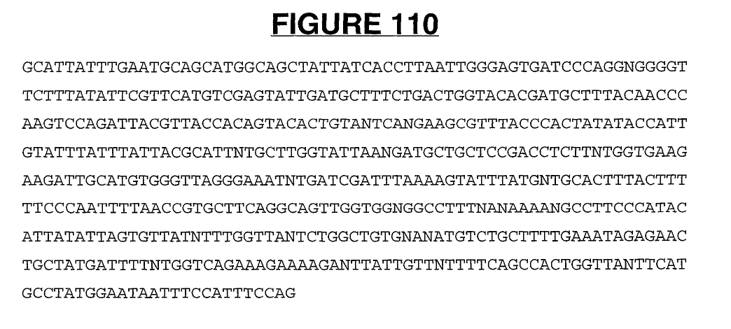

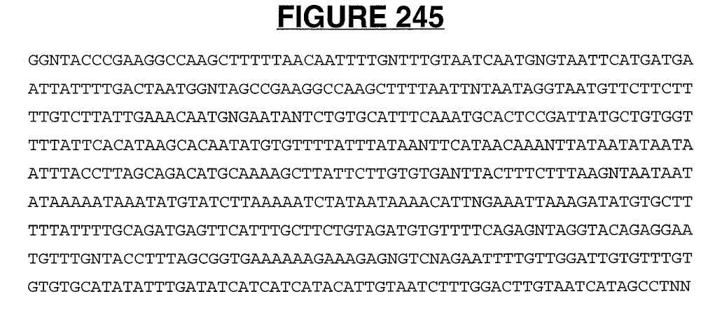

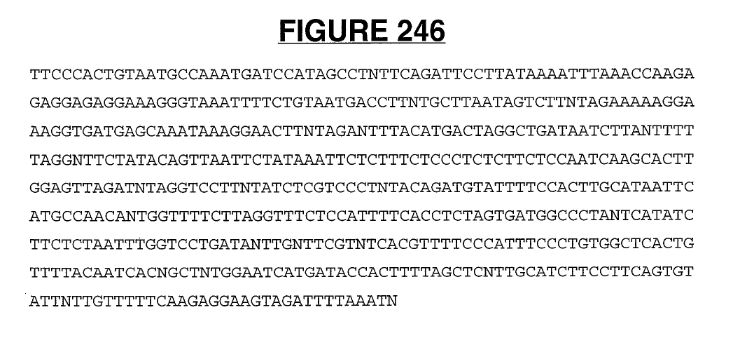

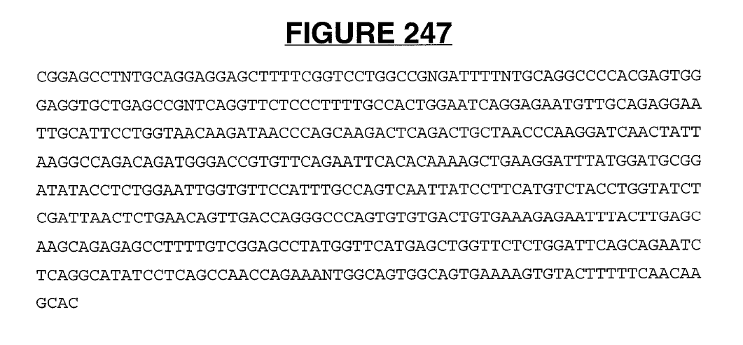

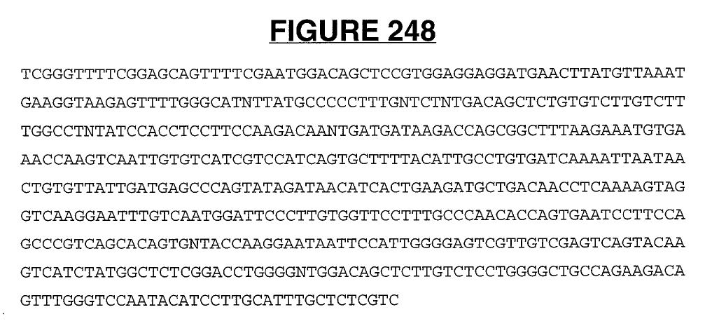

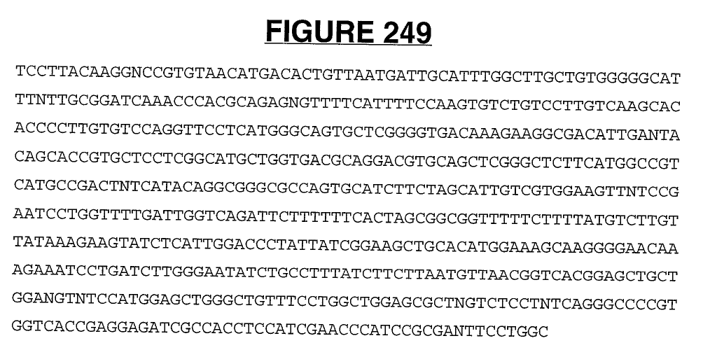

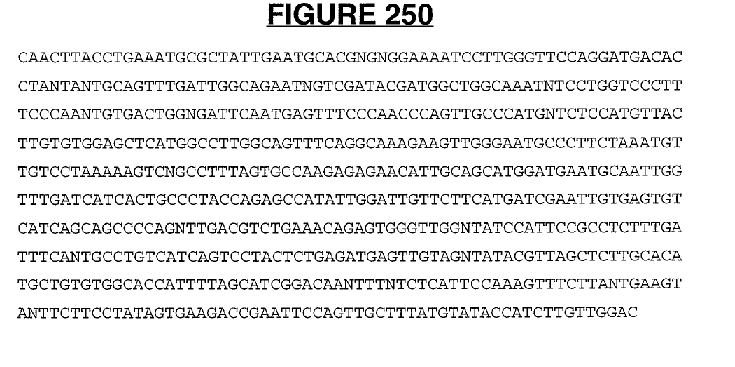

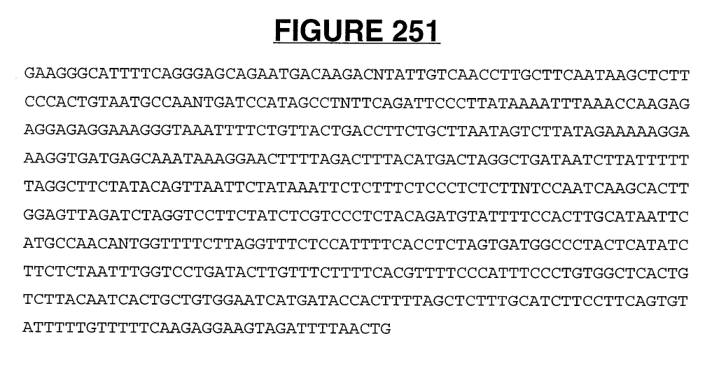

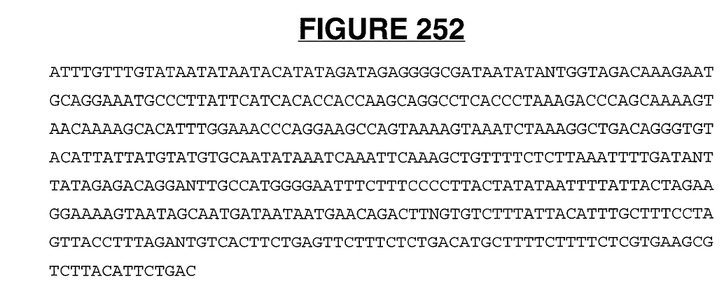

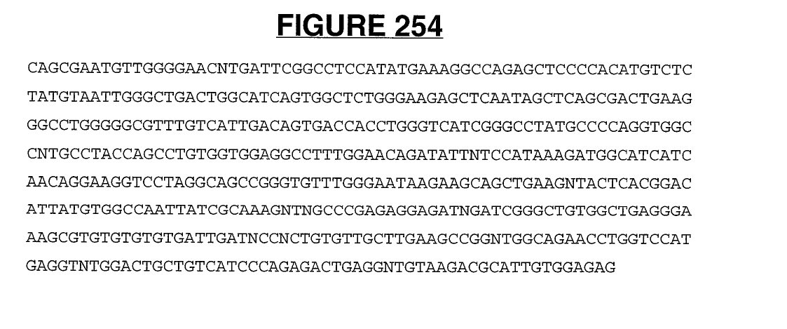

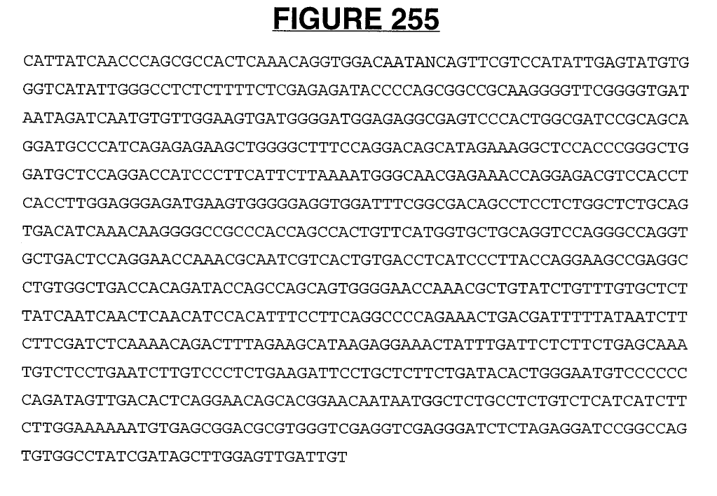

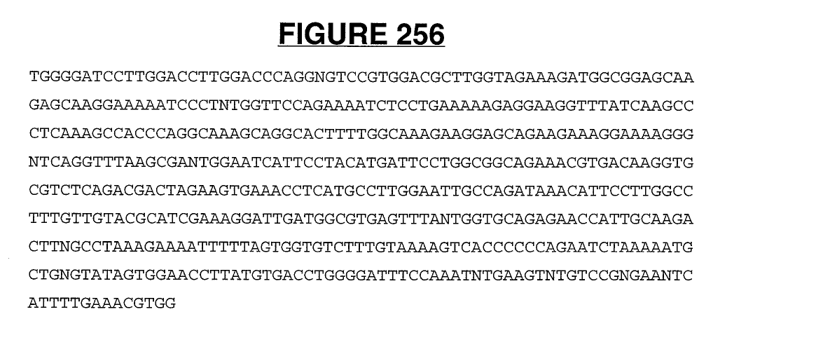

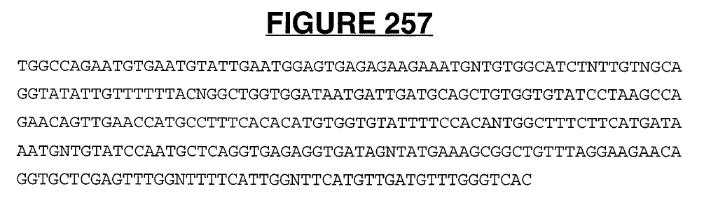

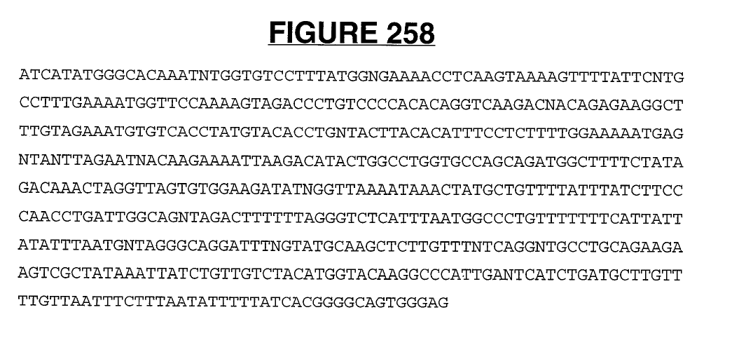

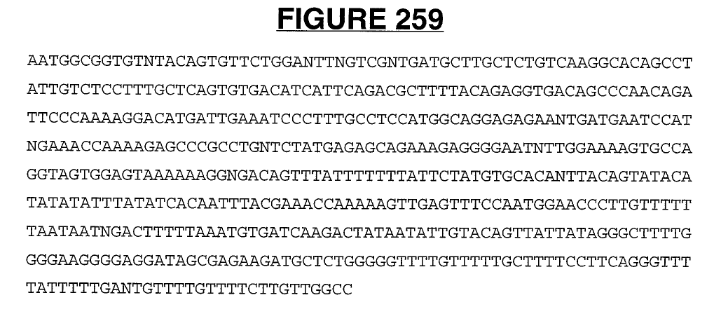

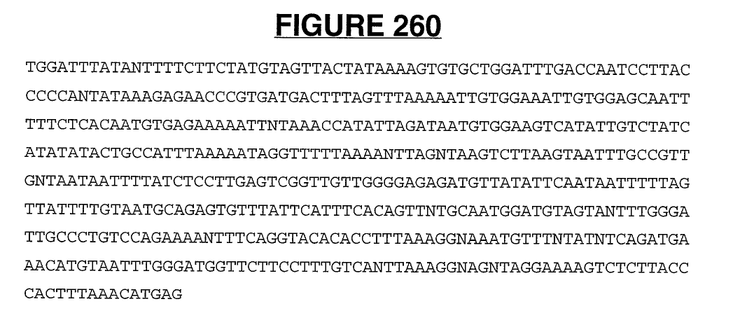

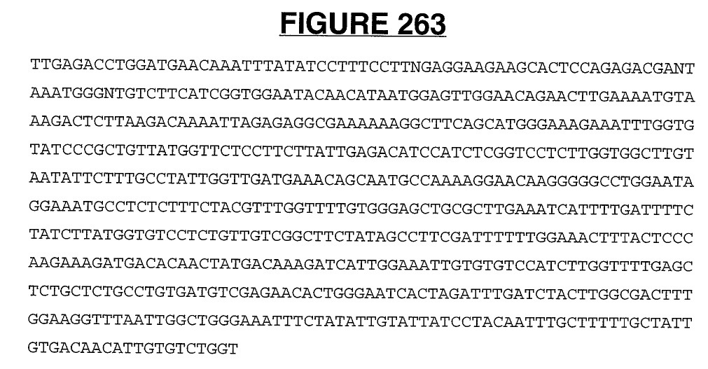

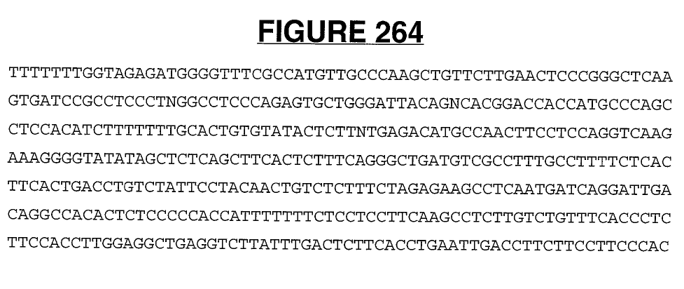

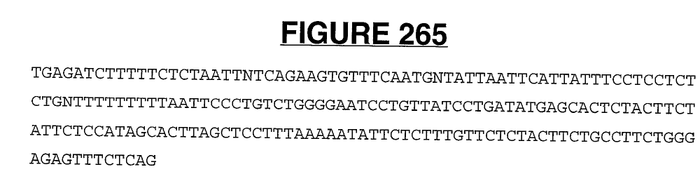

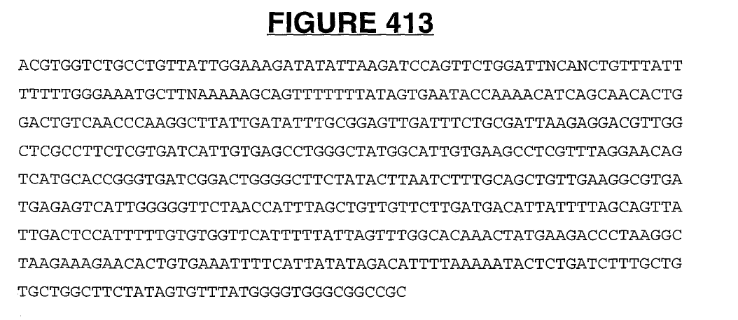

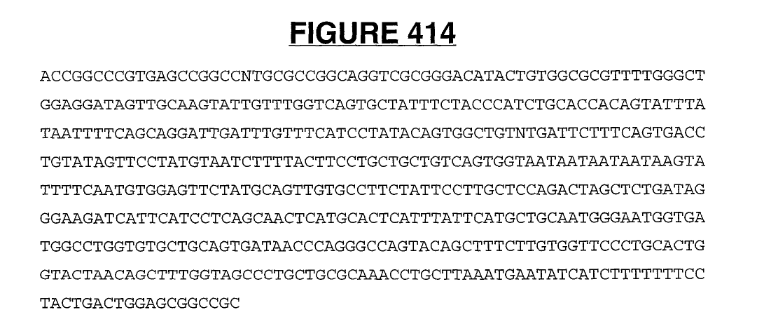

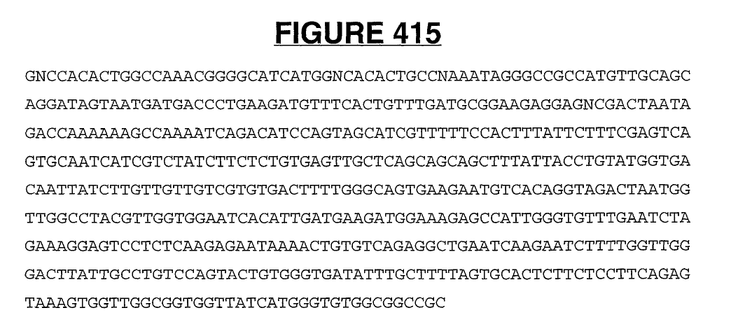

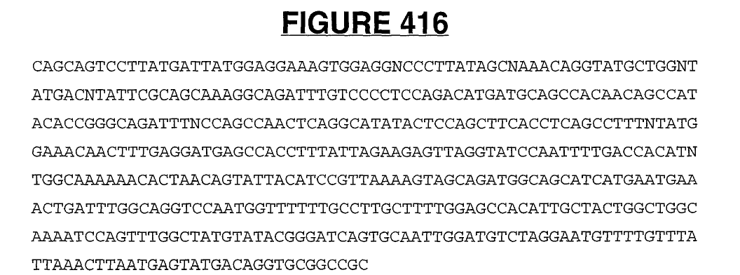

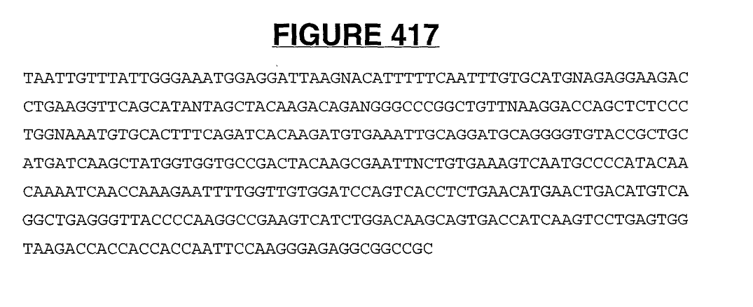

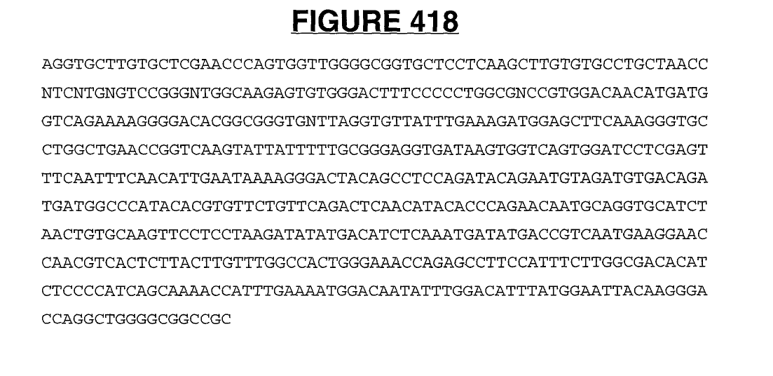

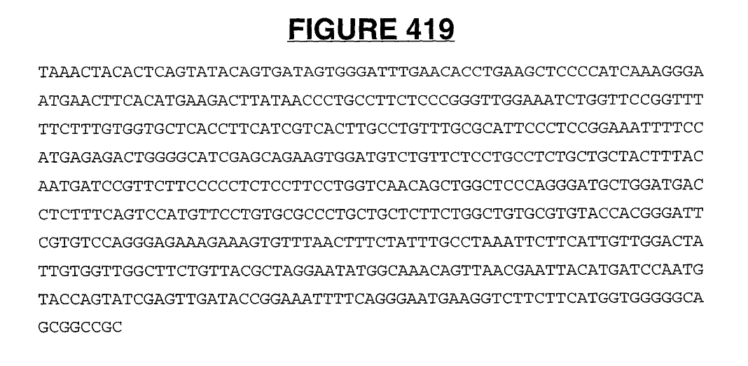

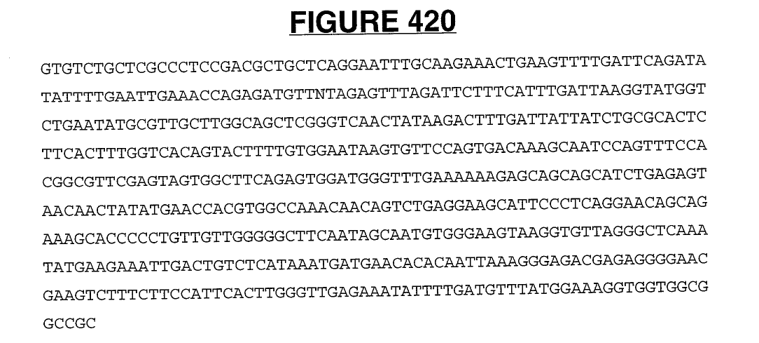

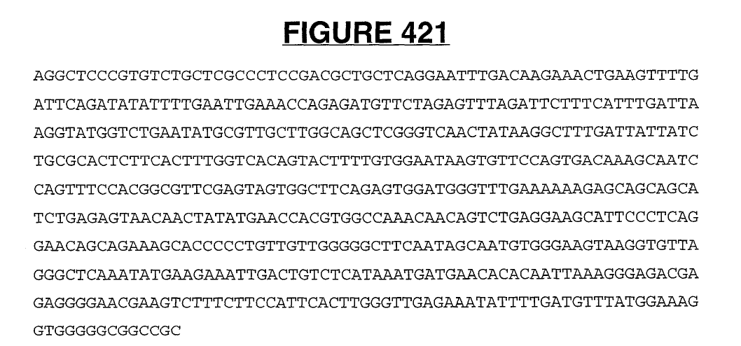

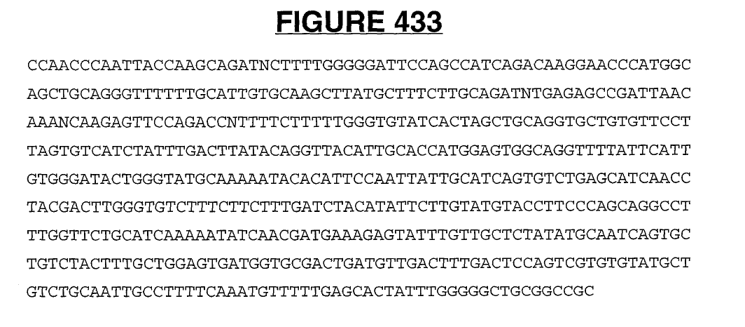

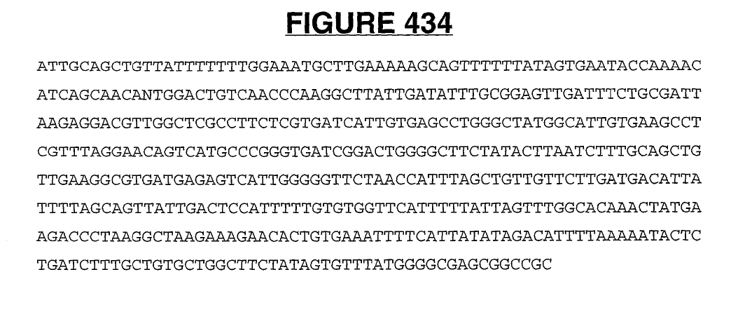

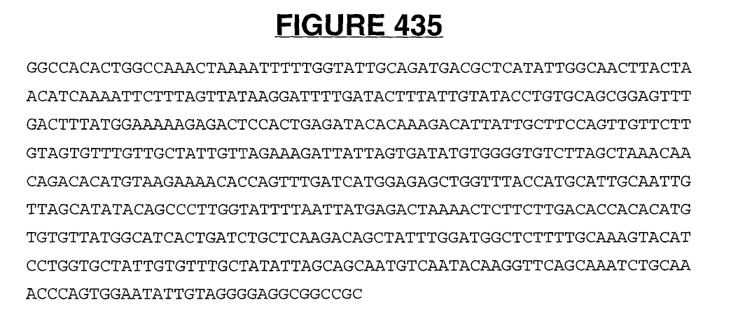

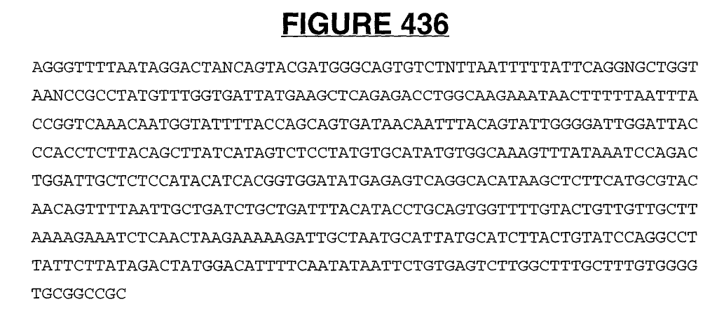

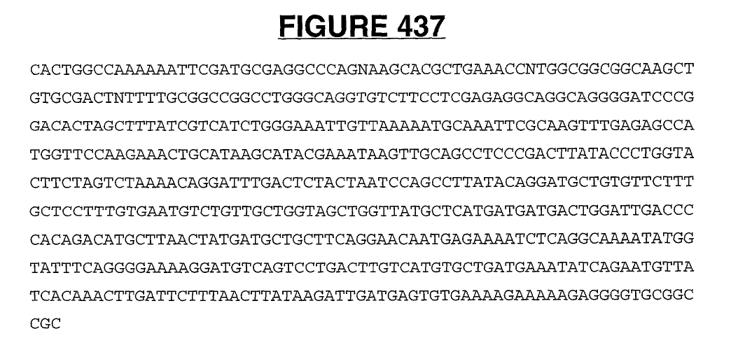

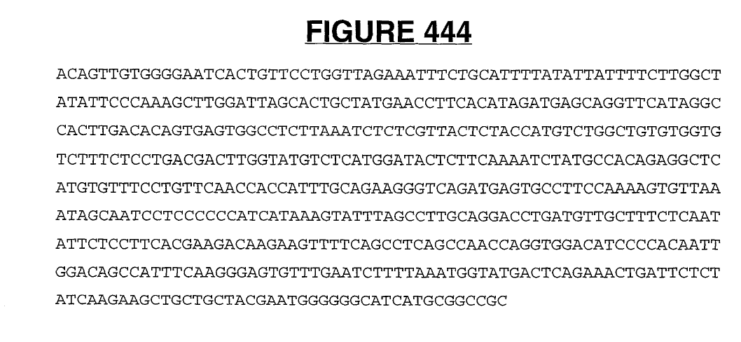

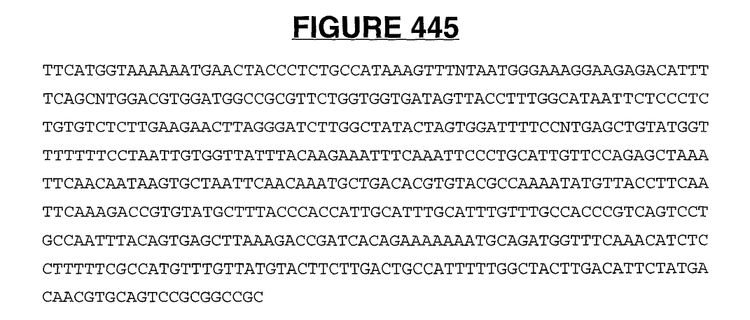

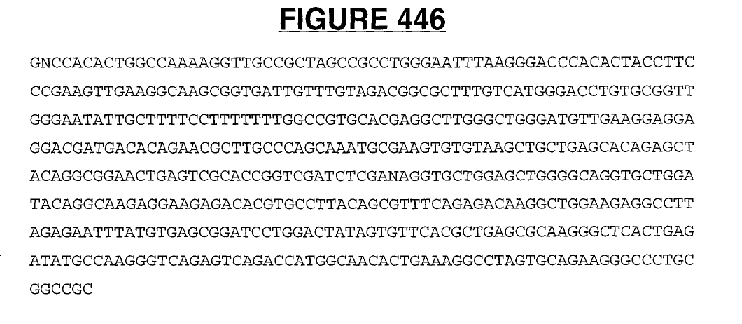

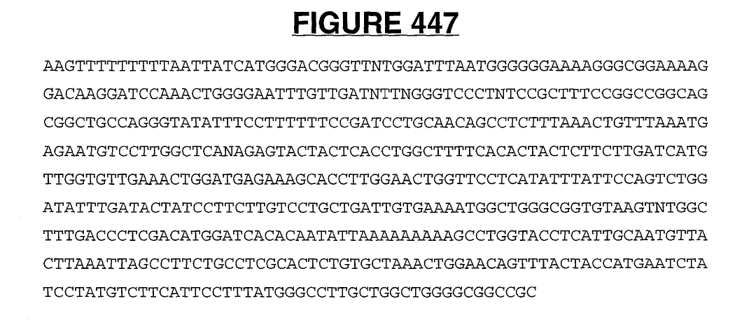

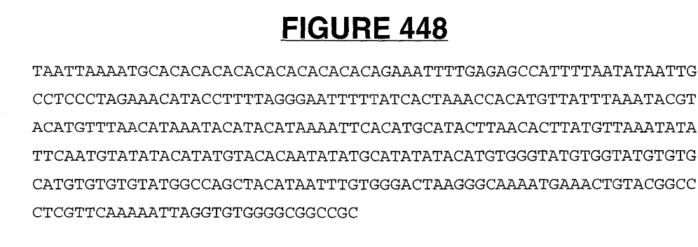

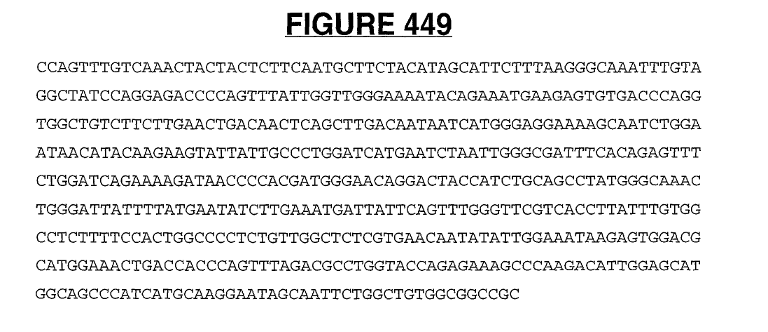

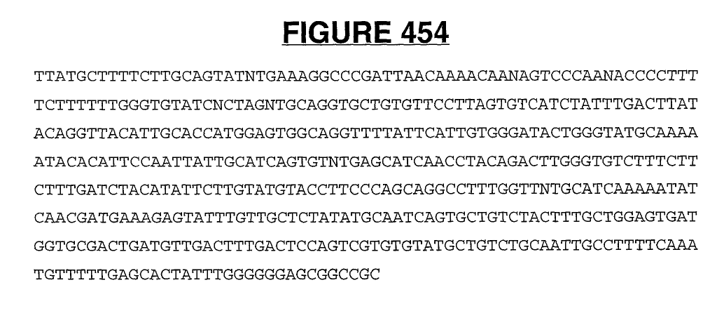

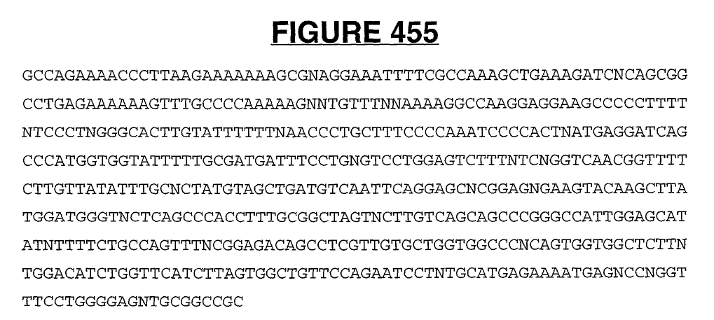

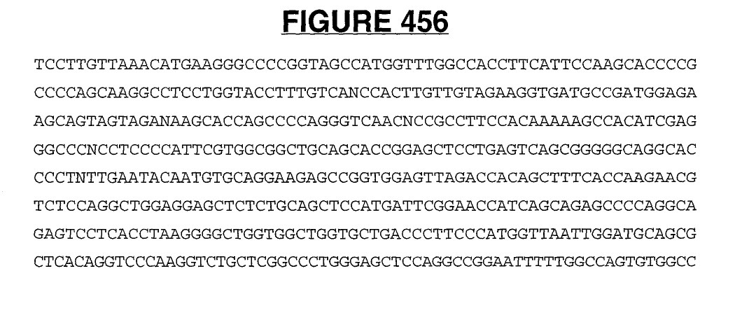

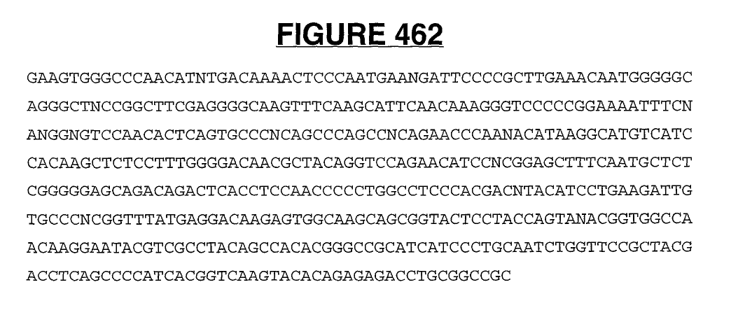

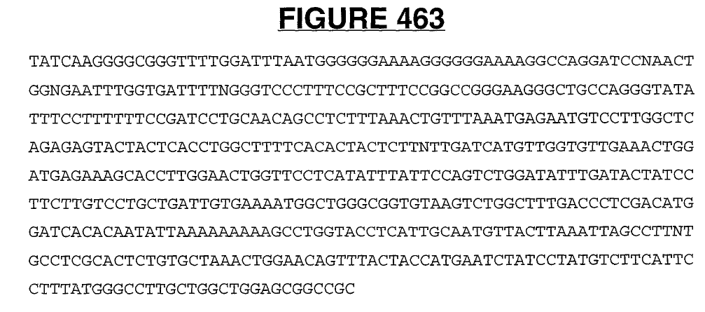

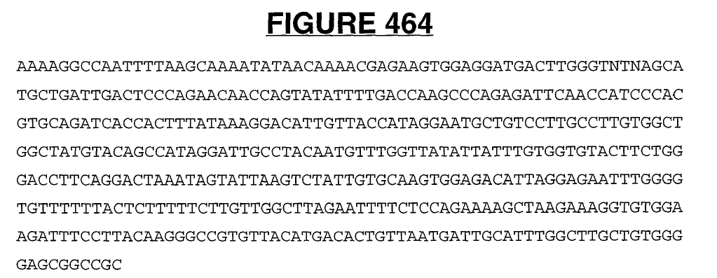

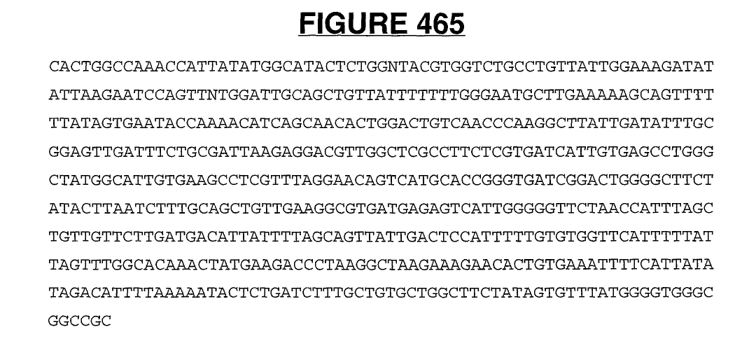

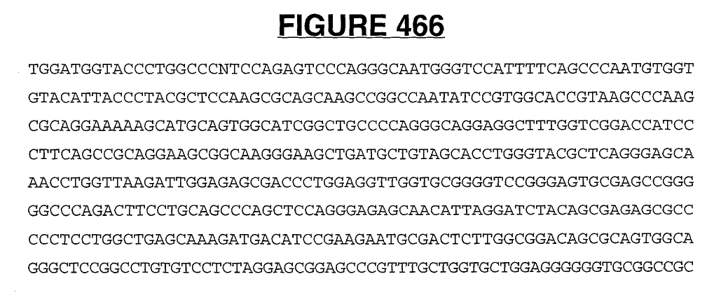

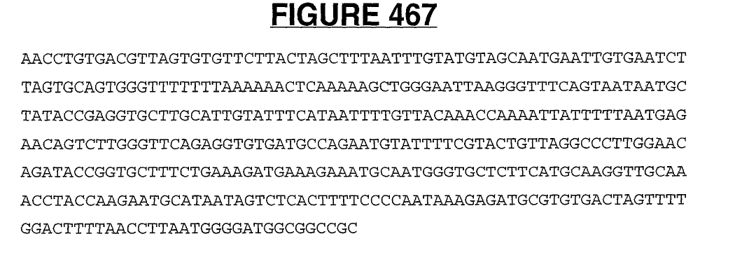

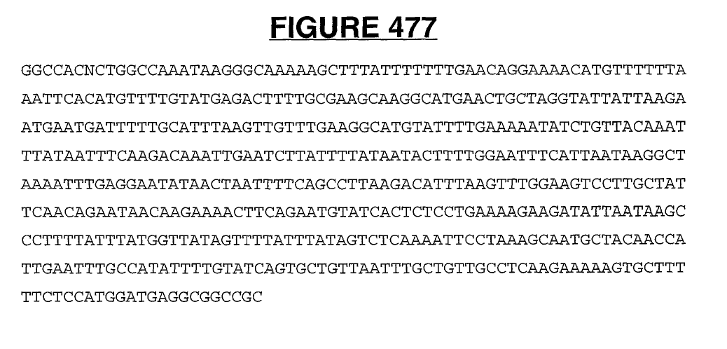

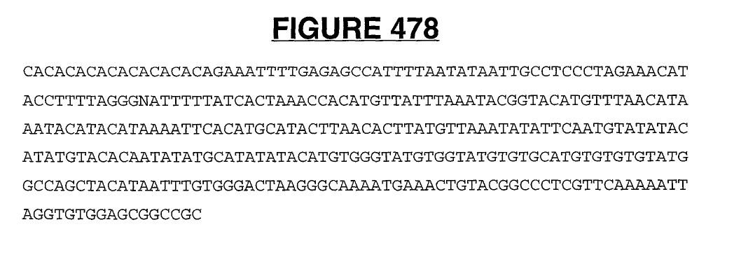

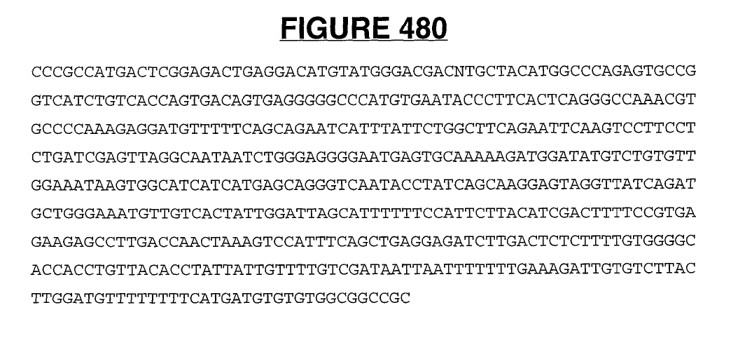

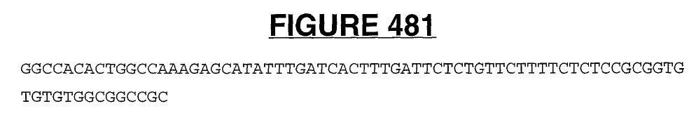

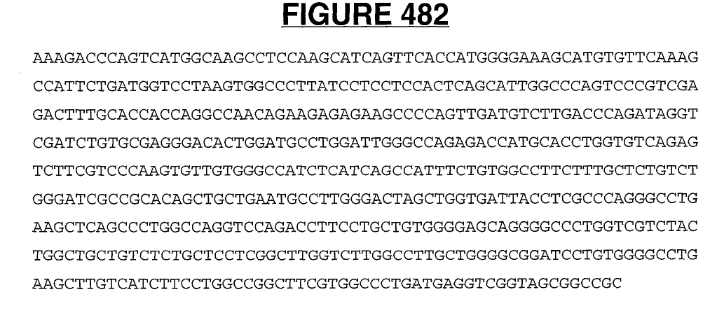

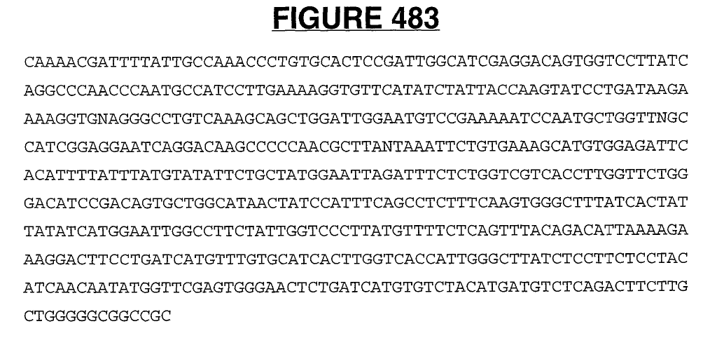

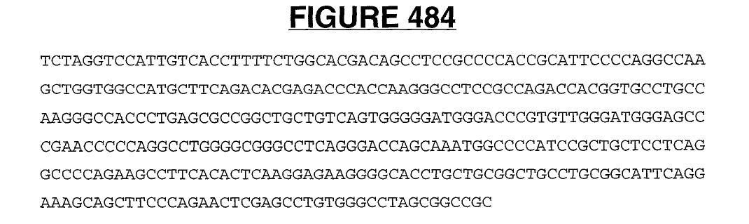

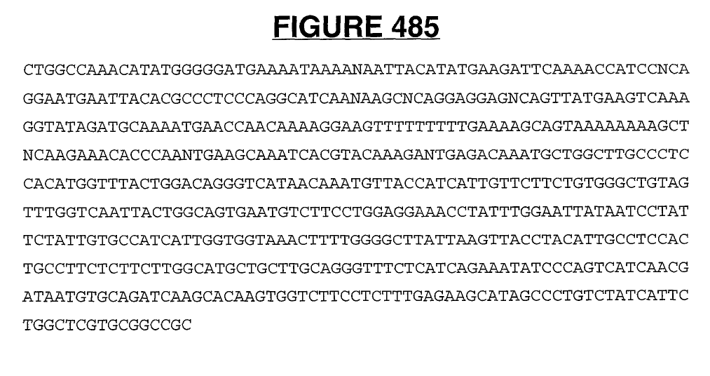

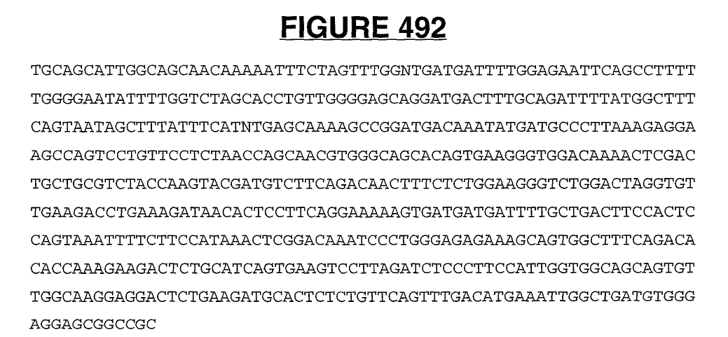

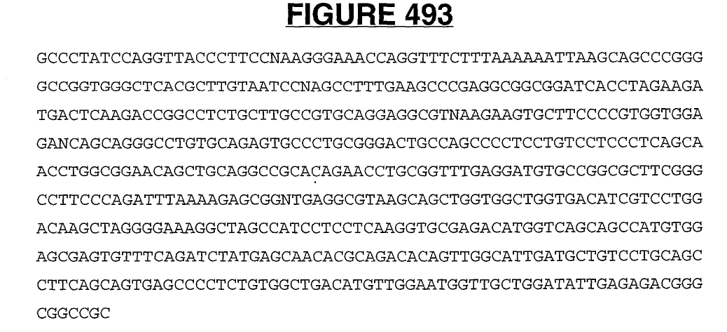

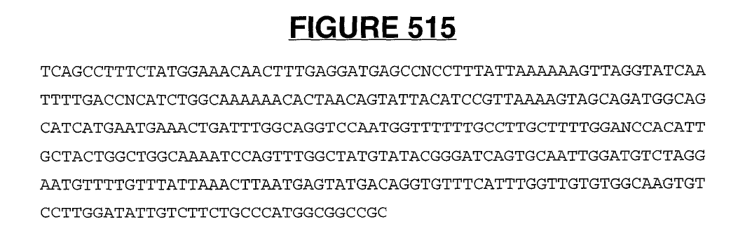

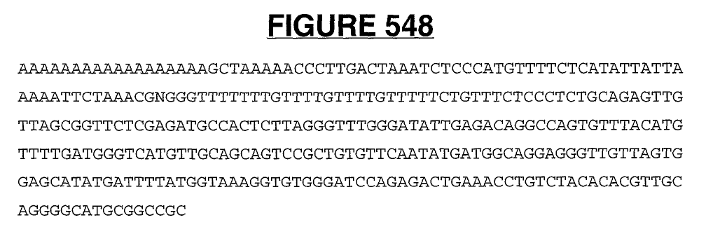

FIGS. 79-80, 97, 110, 245-252, 254-260, 263-265, 413-421, 433-437, 444-449, 454-456, 462-467, 477-478, 480-485, 492-493, 515 and 548.

(f) Human thyroid tissue

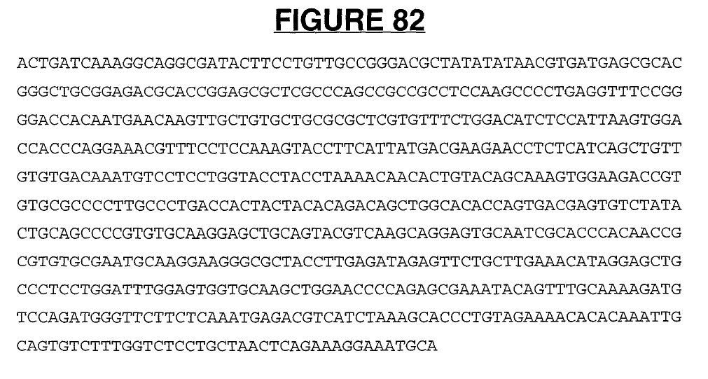

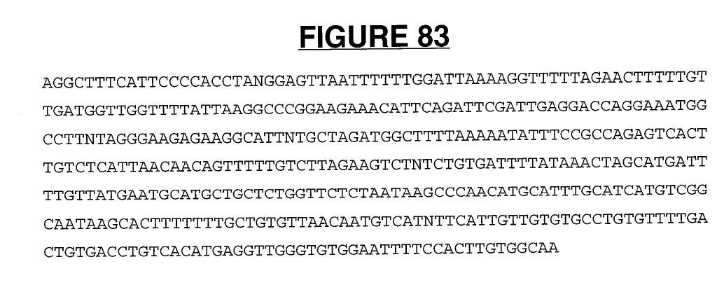

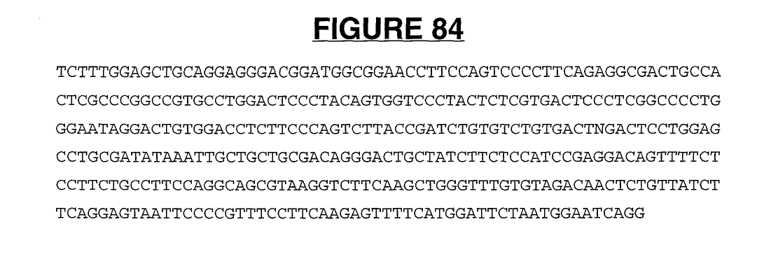

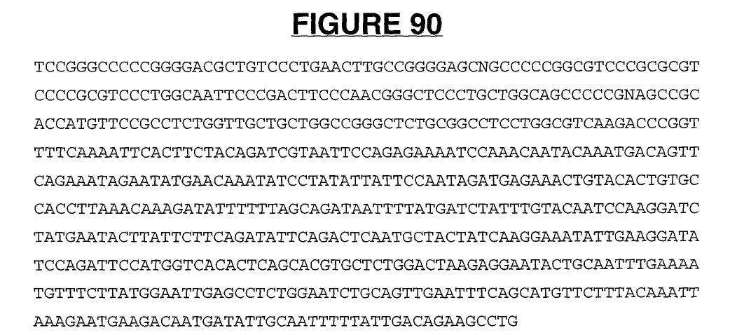

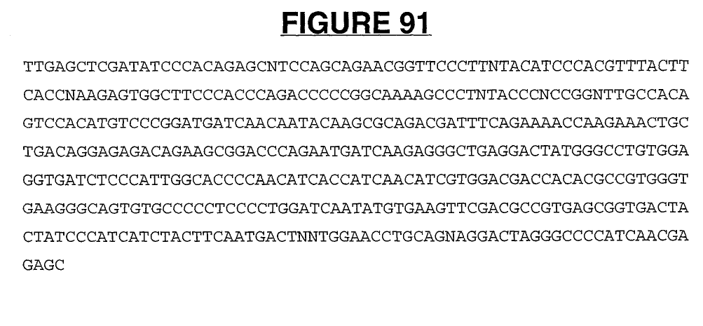

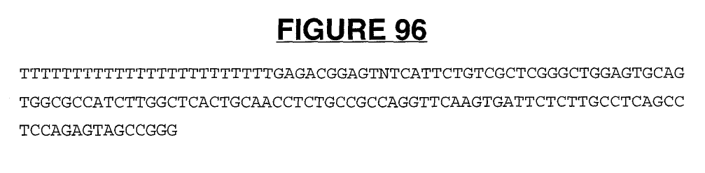

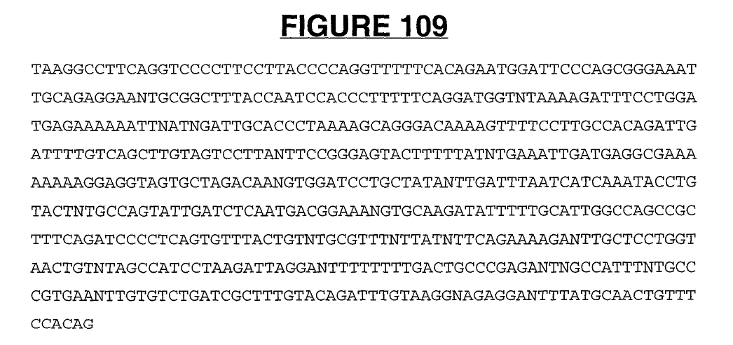

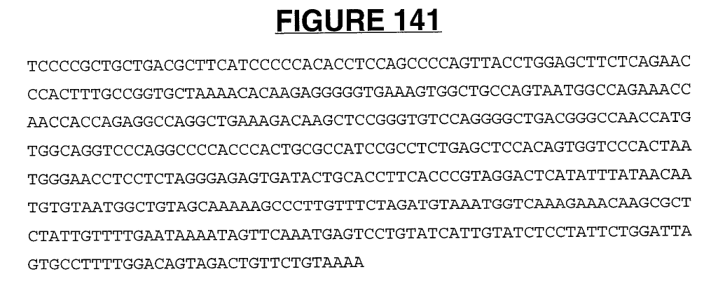

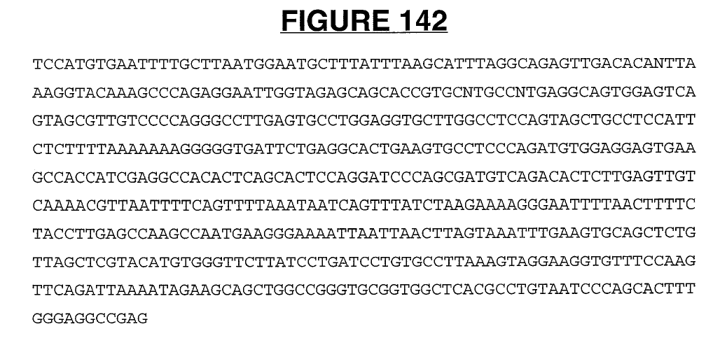

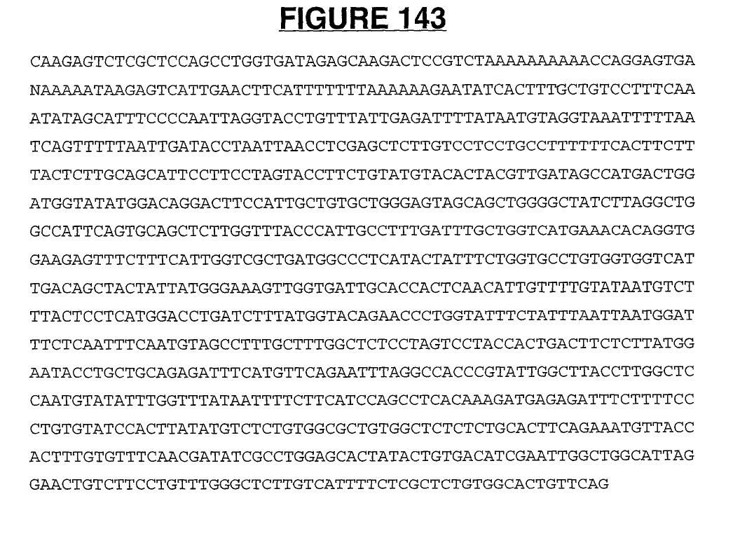

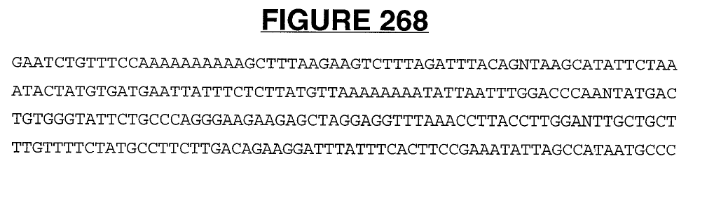

FIGS. 82-84, 90-91, 96, 109, 141-143 and 268.

(g) Human small intestine tissue

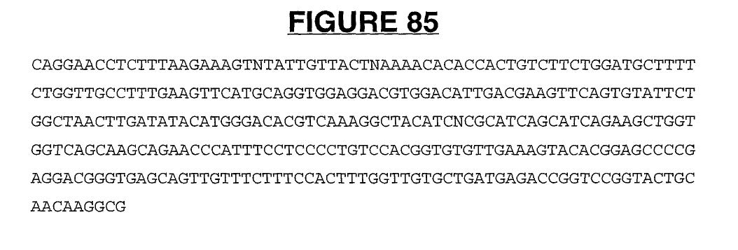

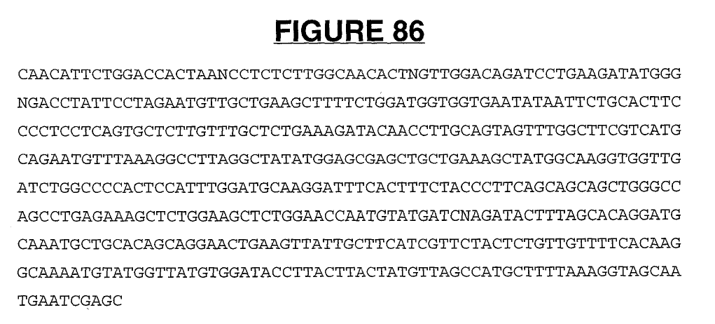

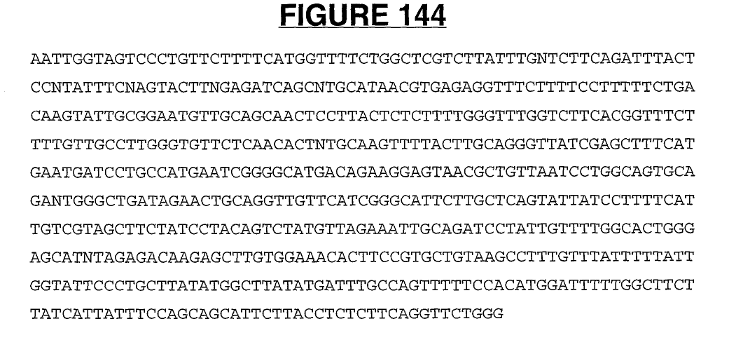

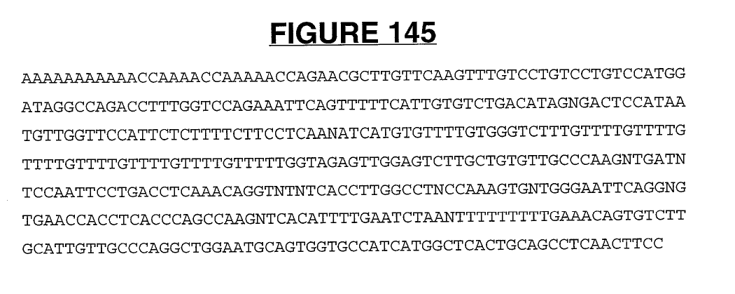

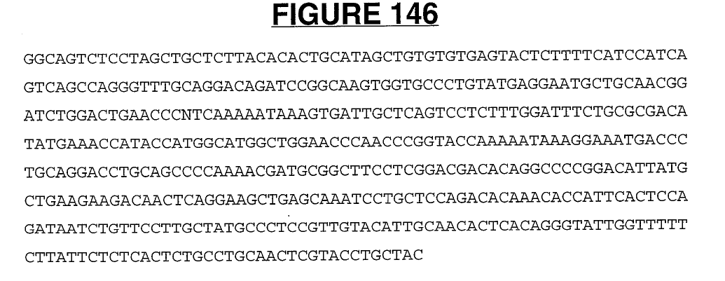

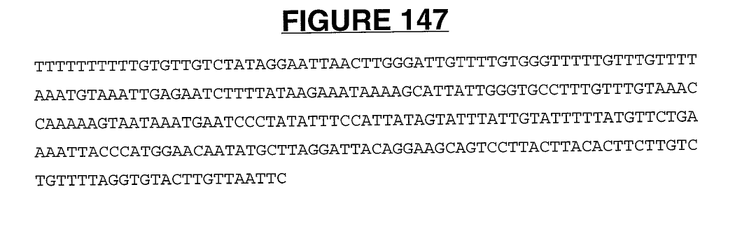

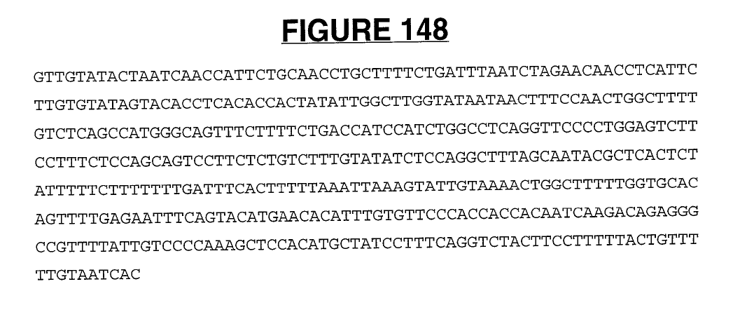

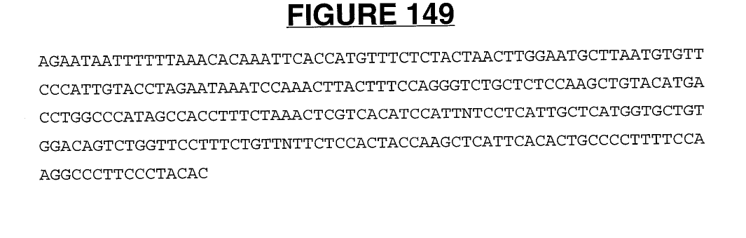

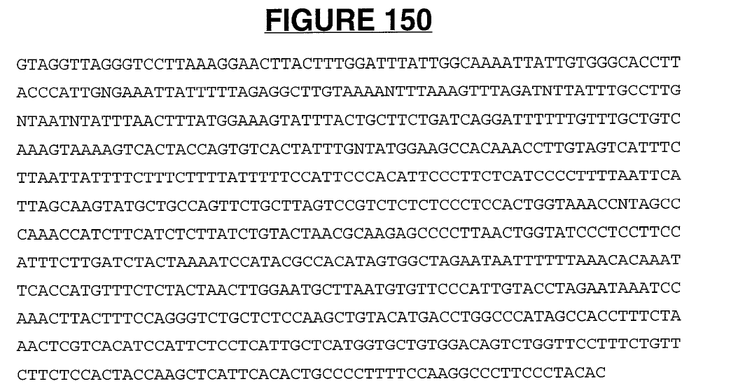

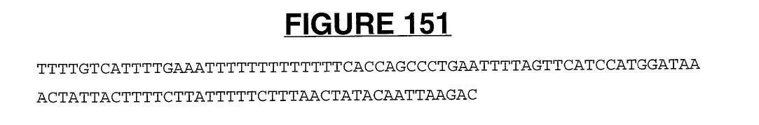

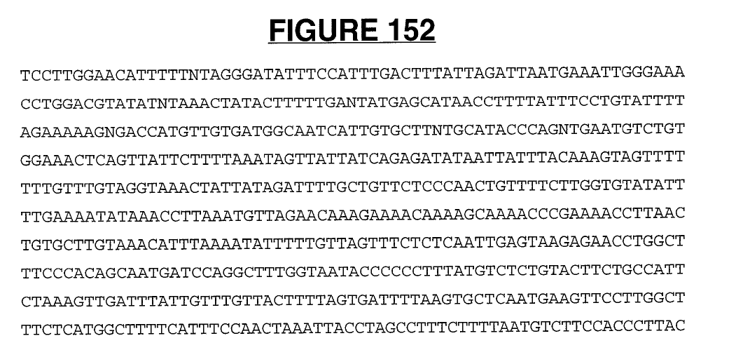

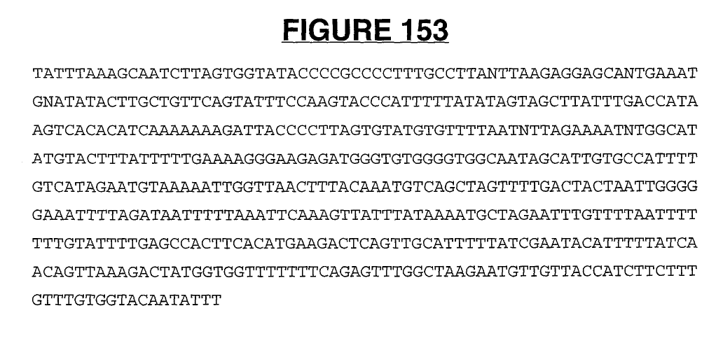

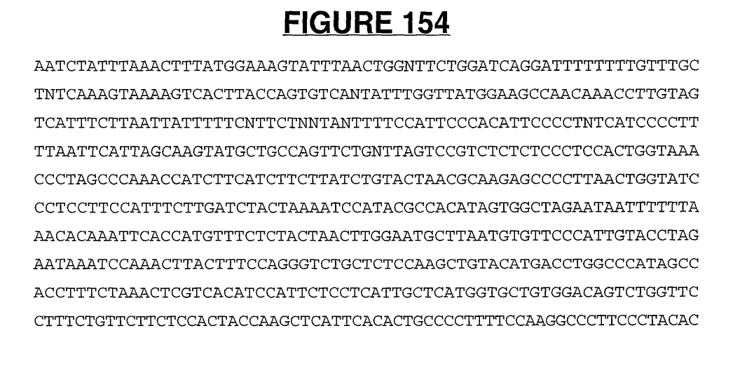

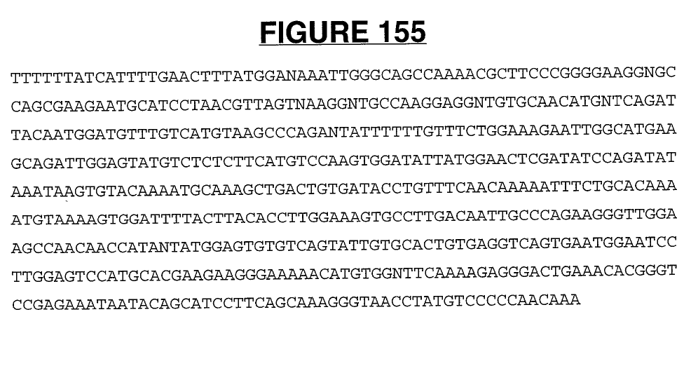

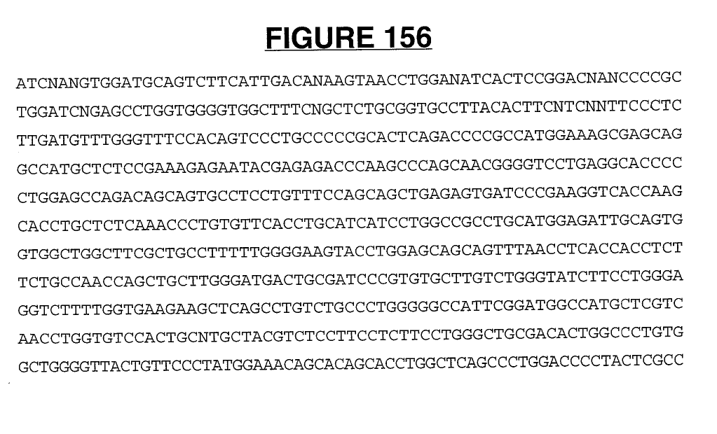

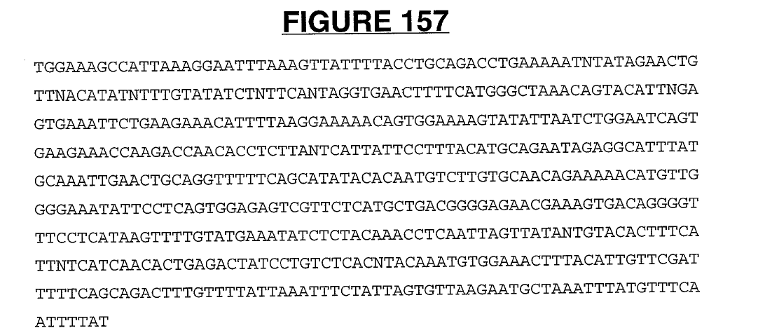

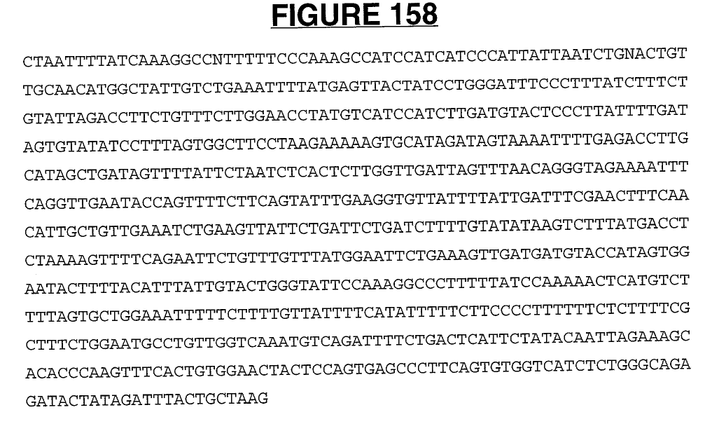

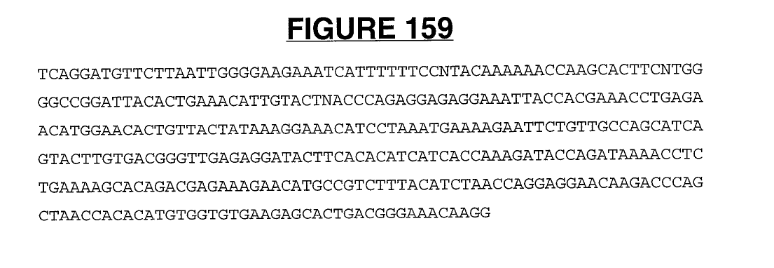

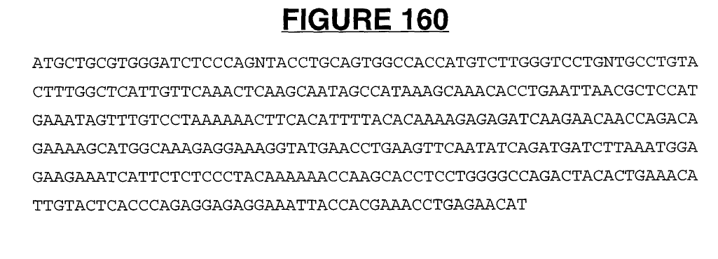

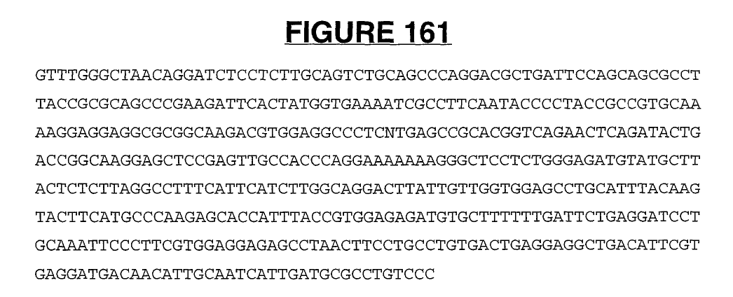

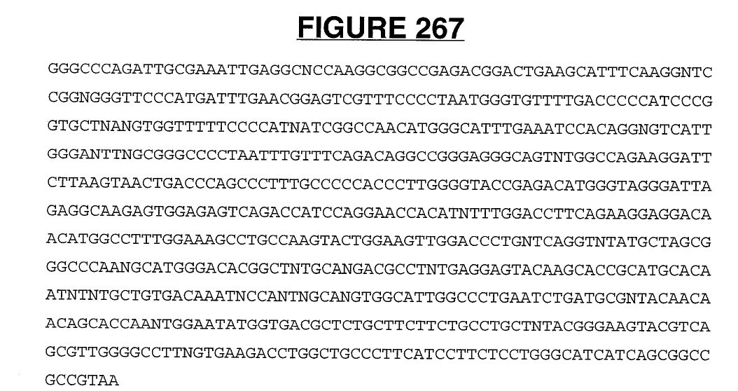

FIGS. 85-86, 144-161 and 267.

(h) Human colon carcinoma tissue

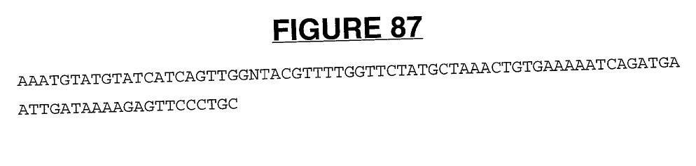

FIG. 87.

(i) Human lung endothelial cells

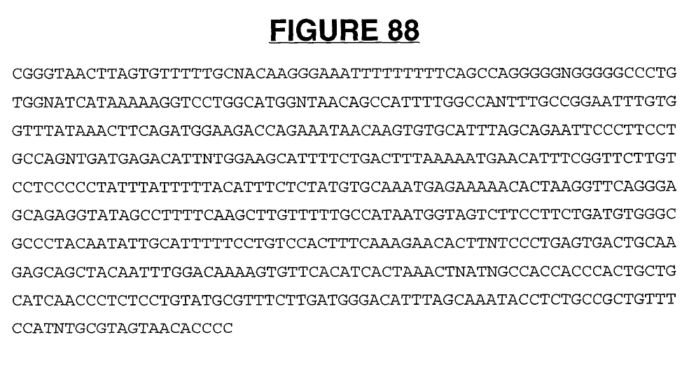

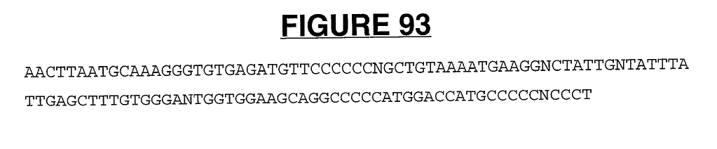

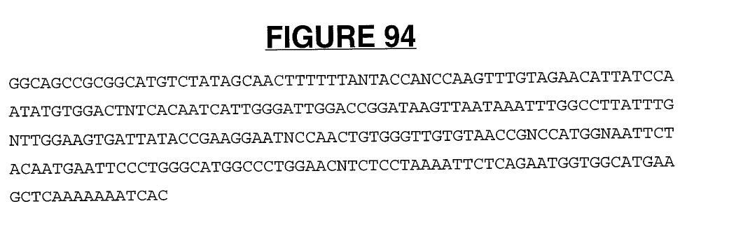

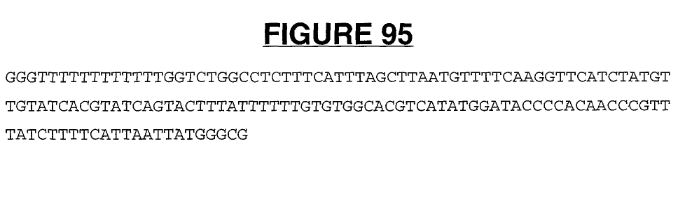

FIGS. 88 and 93-95.

Human hypothalamus tissue

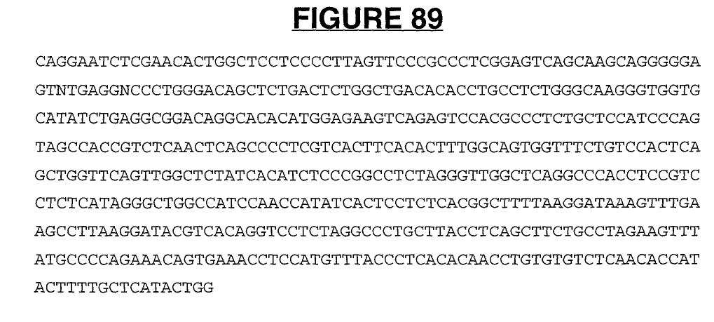

FIG. 89.

(k) Human breast carcinoma tissue

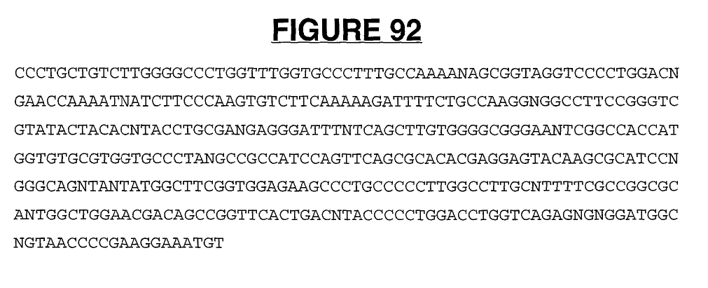

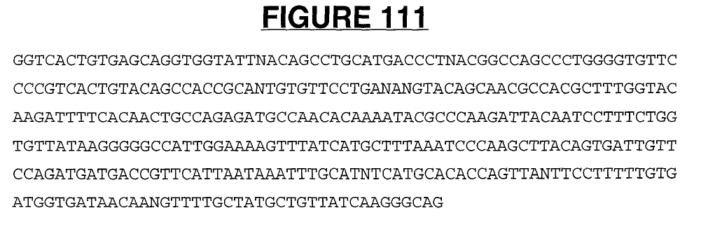

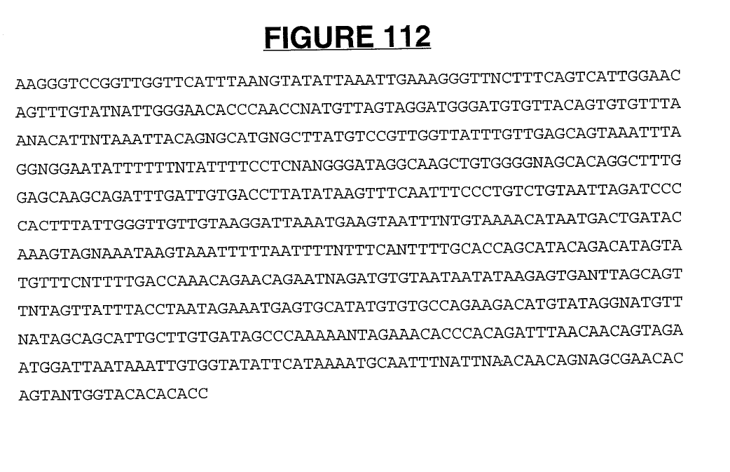

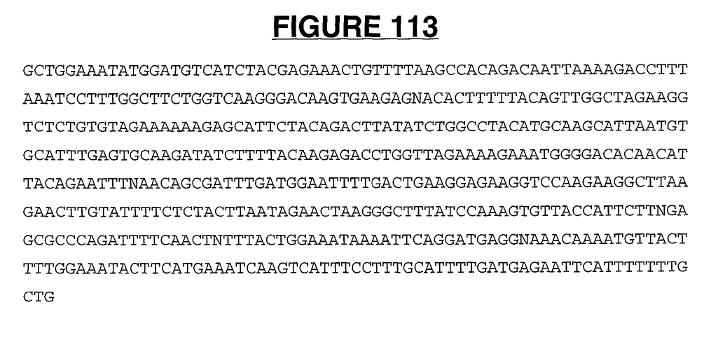

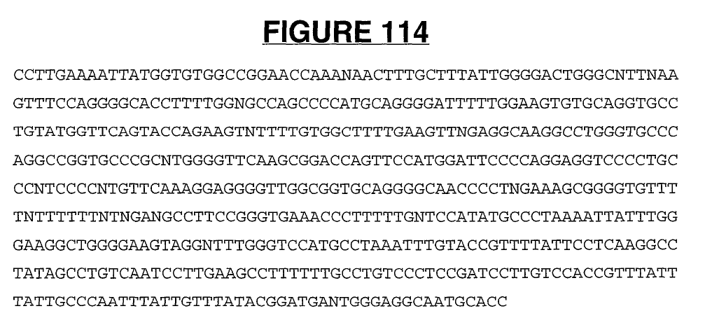

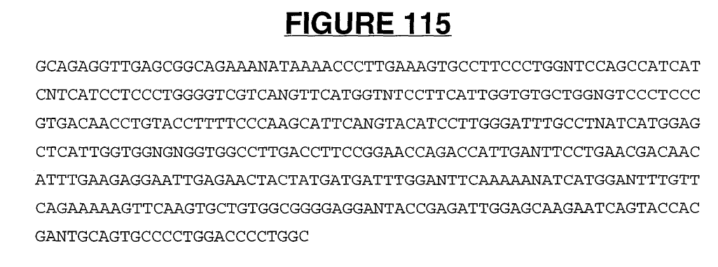

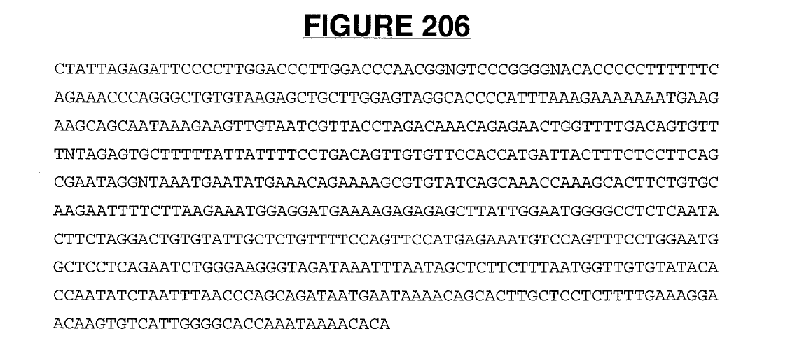

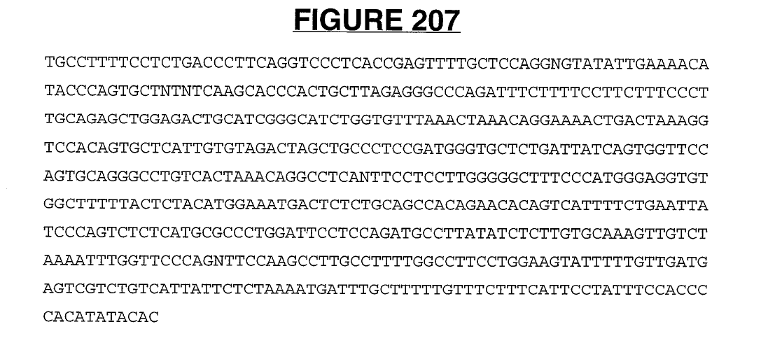

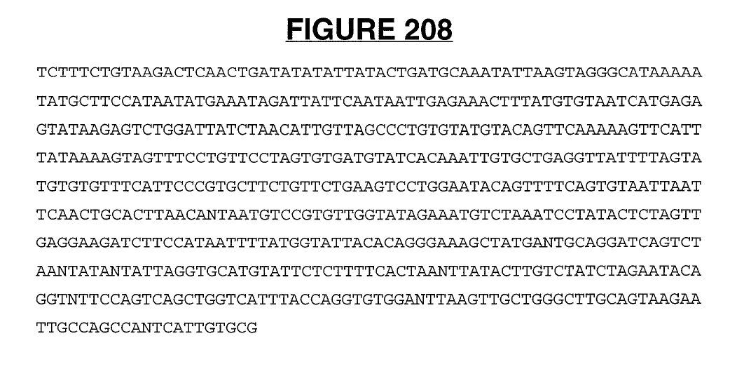

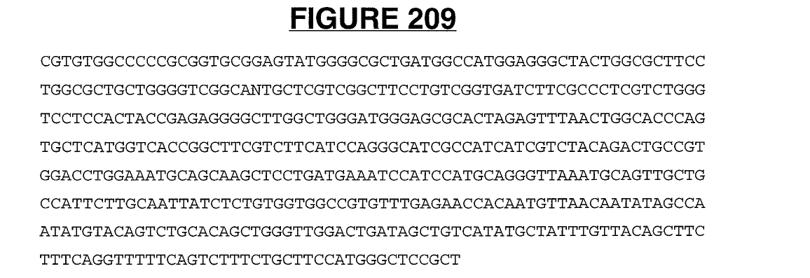

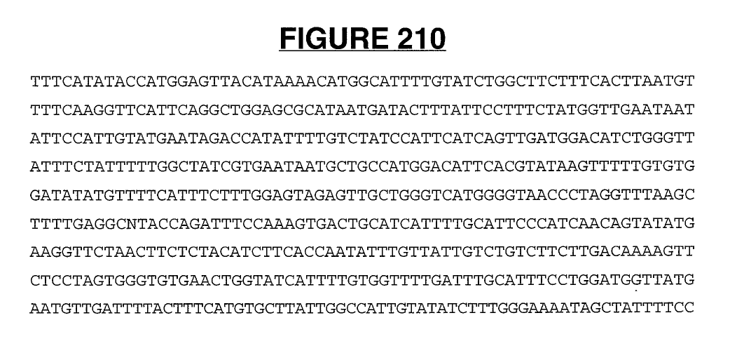

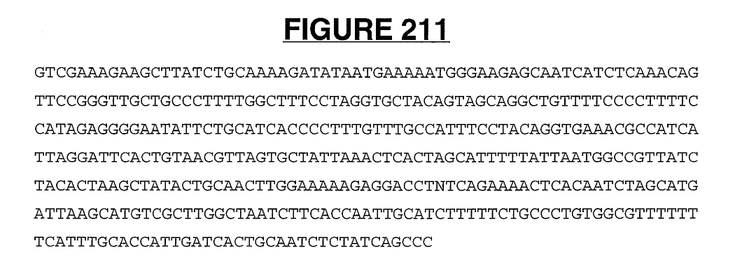

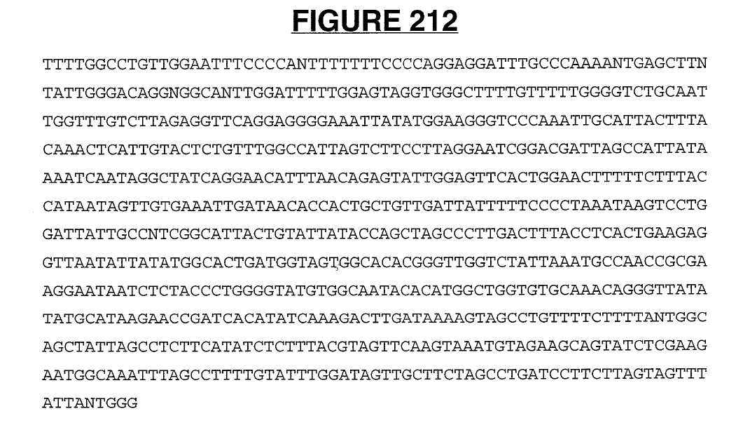

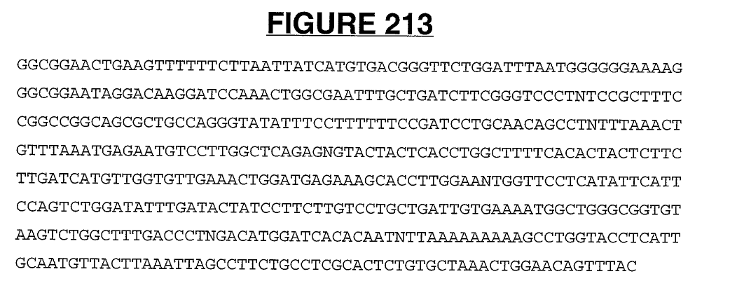

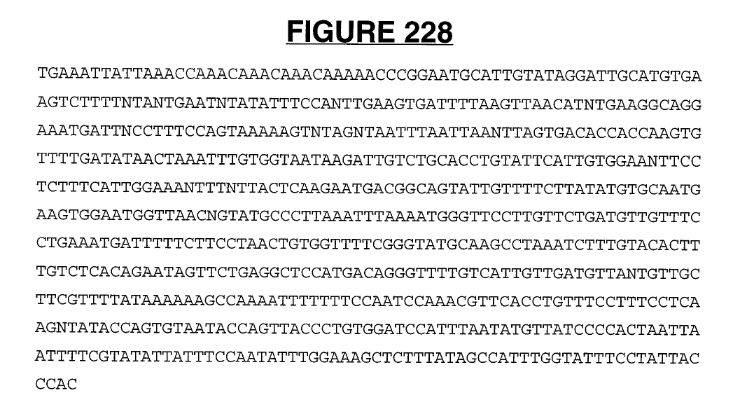

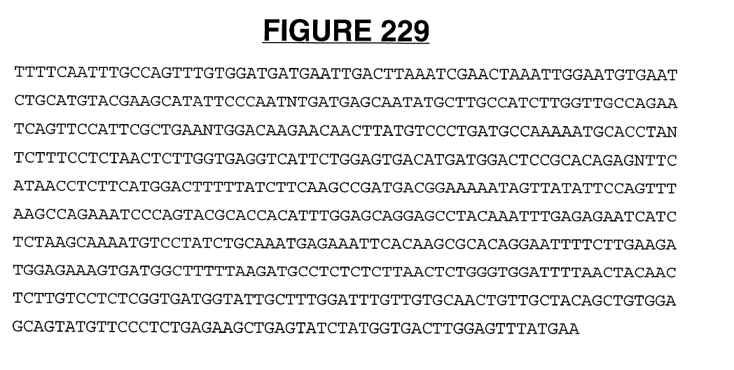

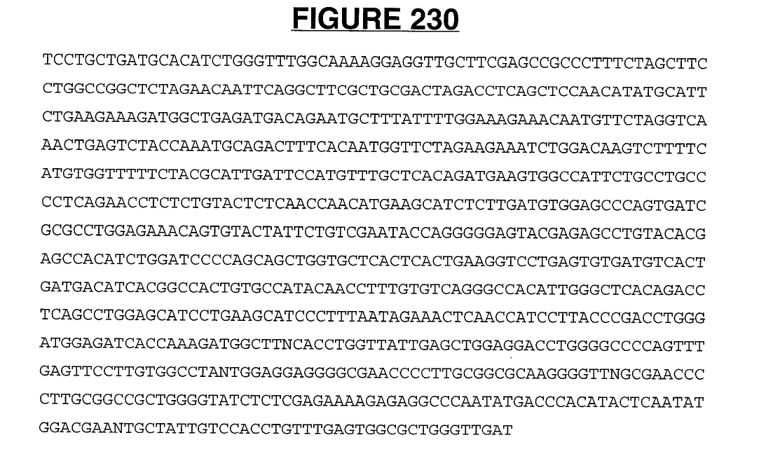

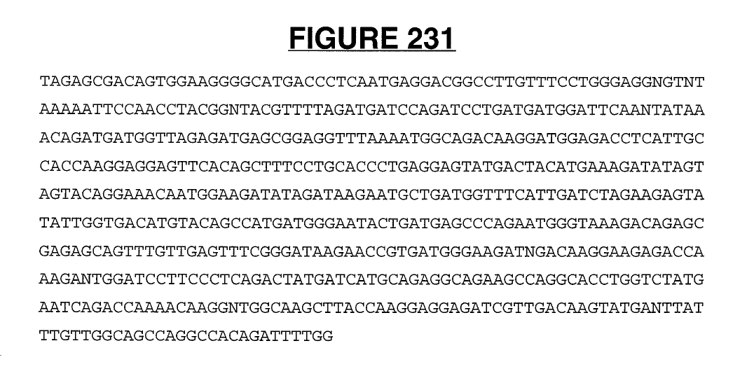

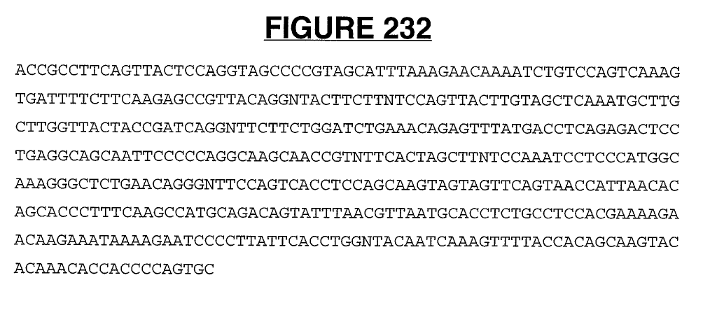

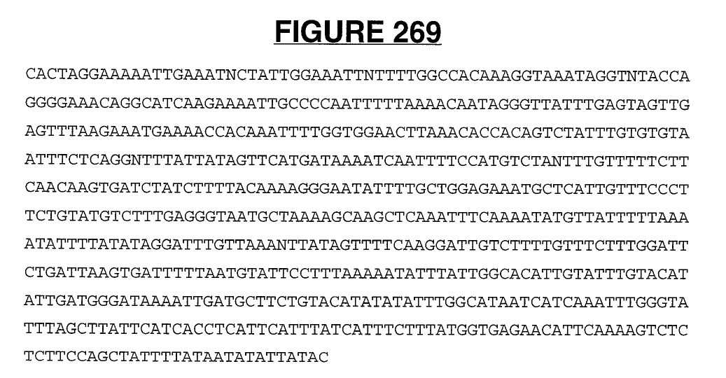

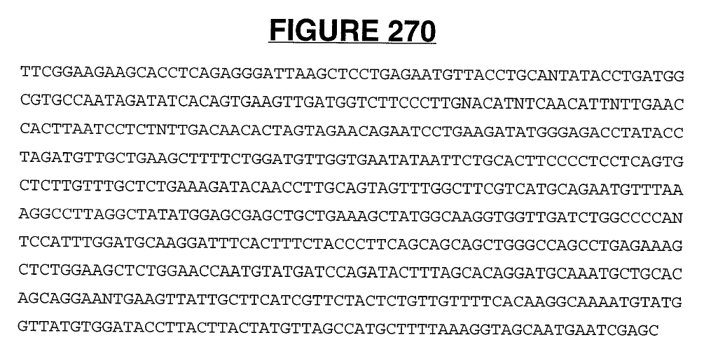

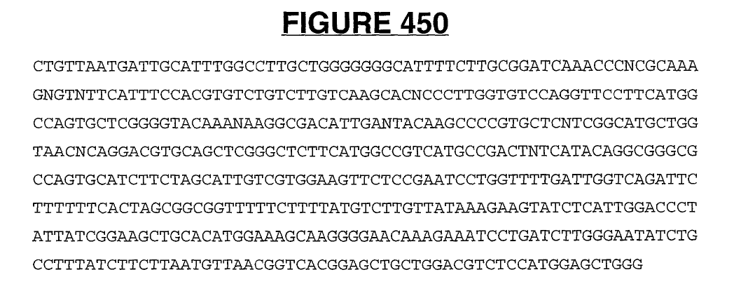

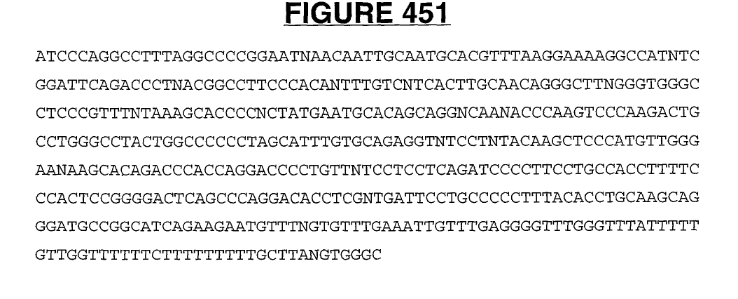

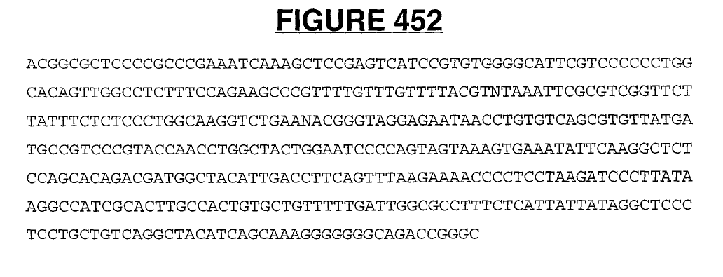

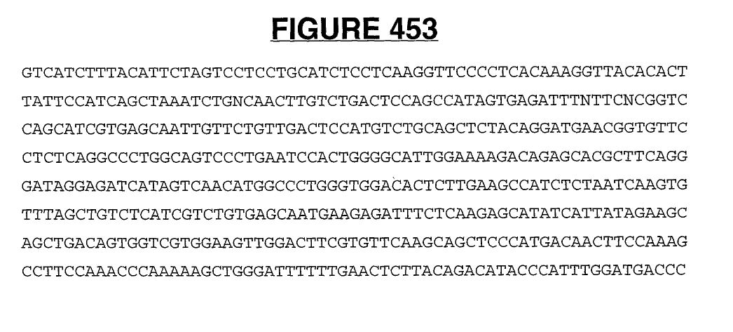

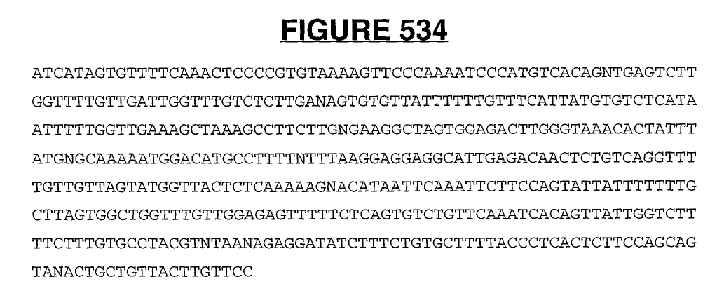

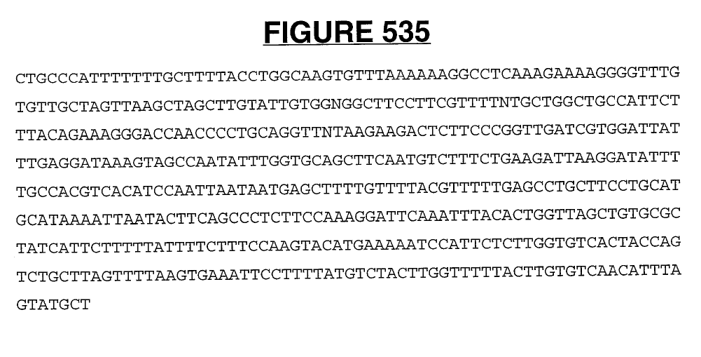

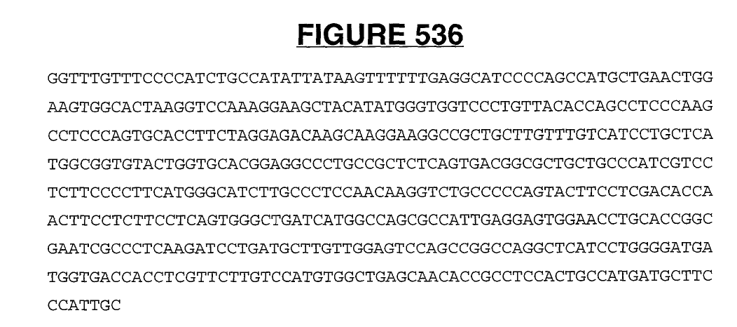

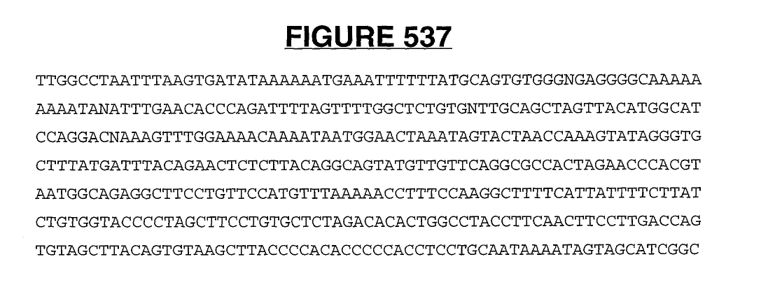

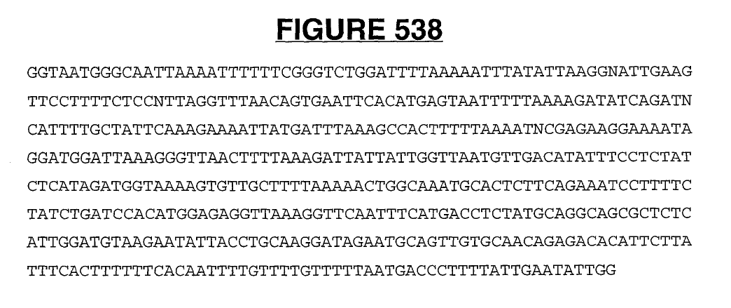

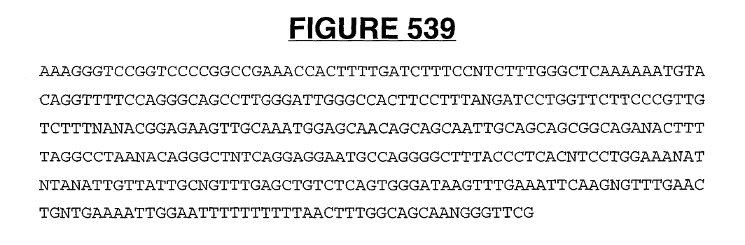

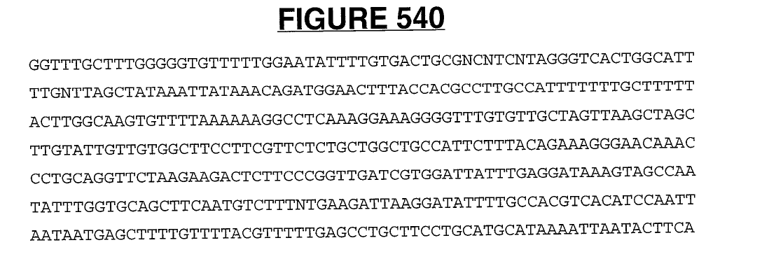

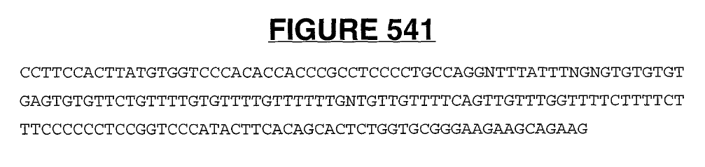

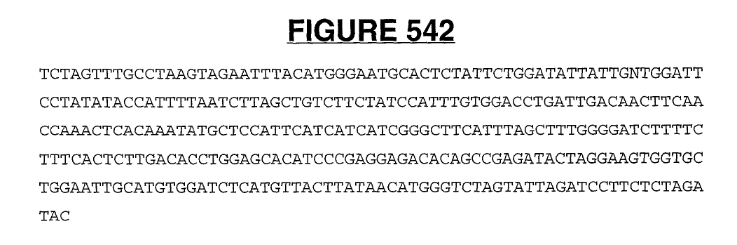

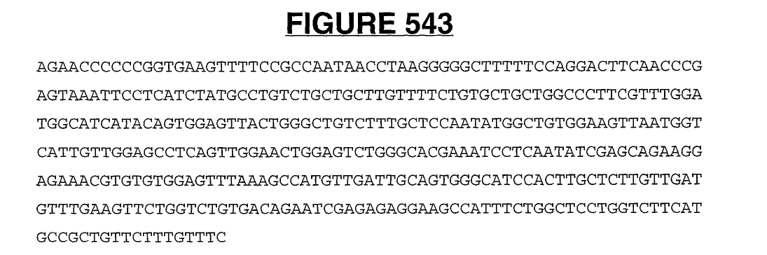

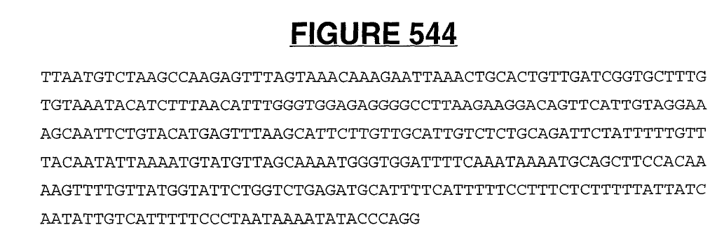

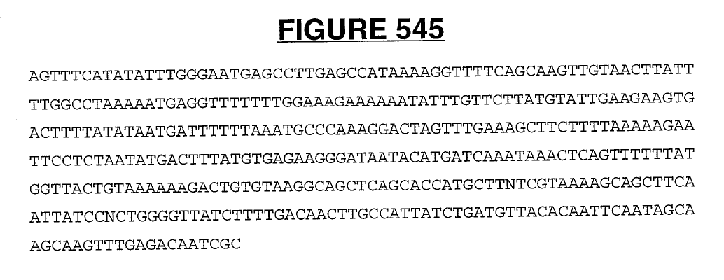

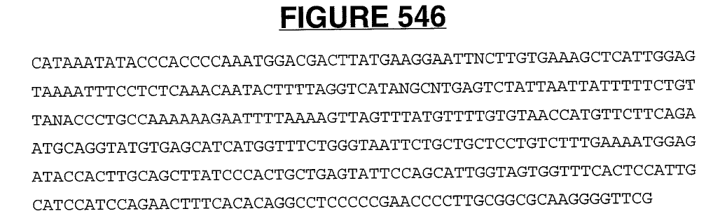

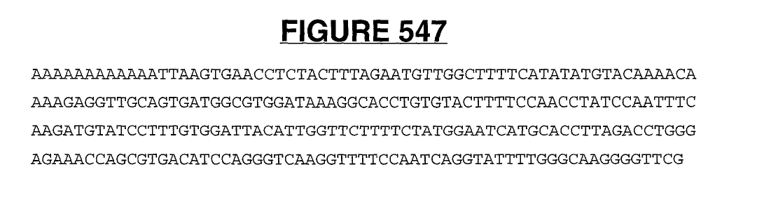

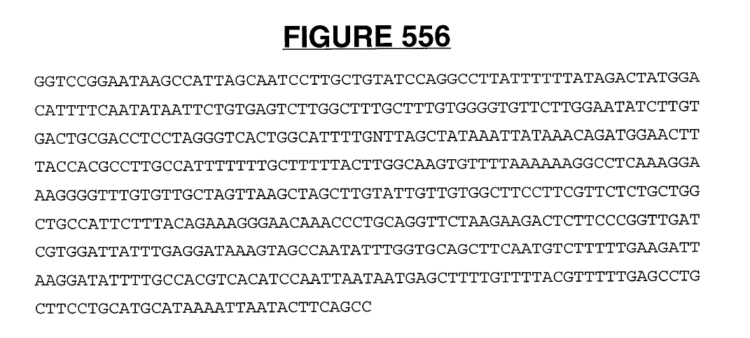

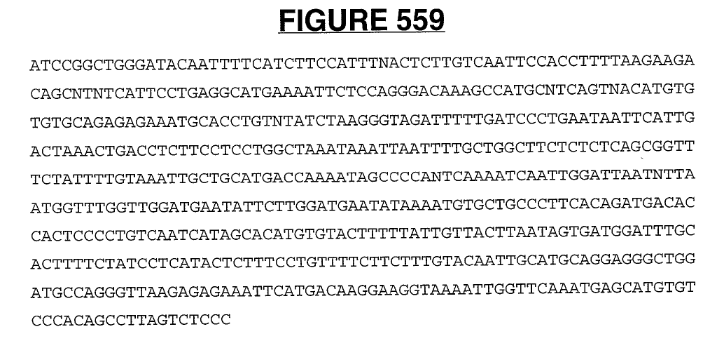

FIGS. 92, 111-115, 206-213, 228-232, 269-270, 450-453, 534-547, 556 and 559.

(l) Human aortic endothelial cells

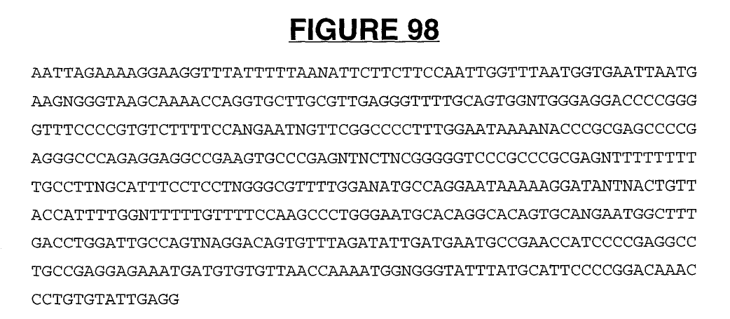

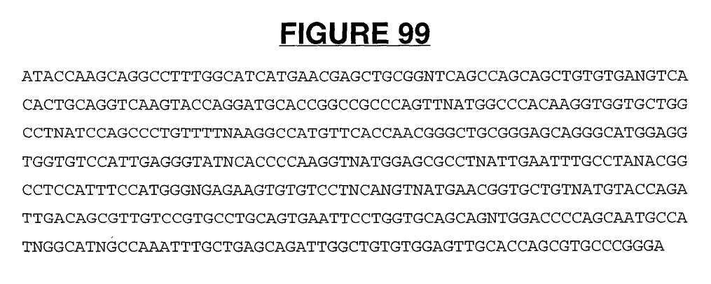

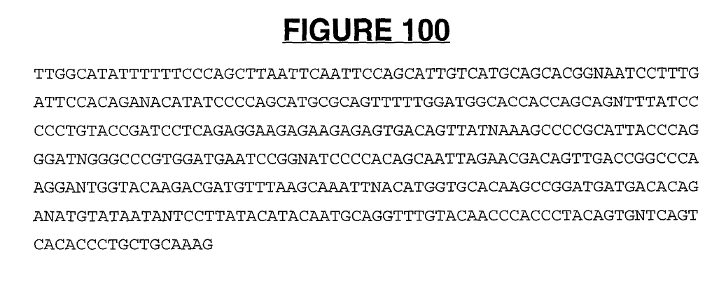

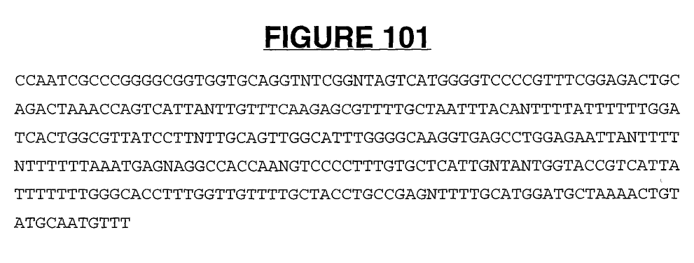

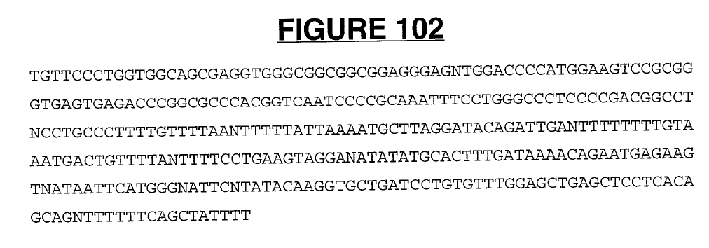

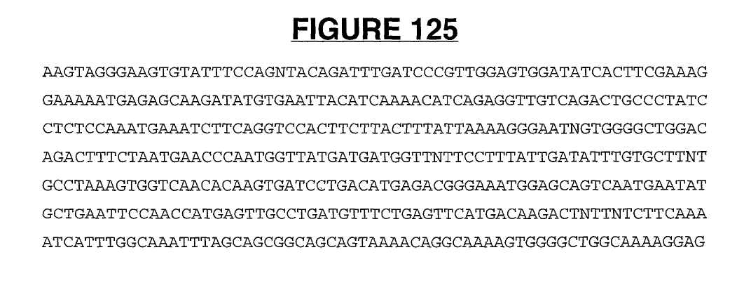

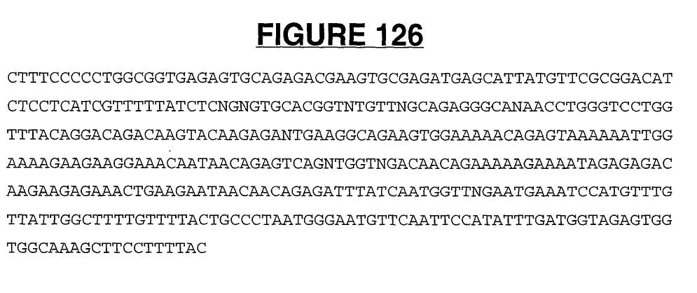

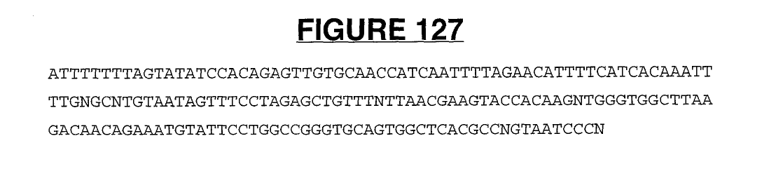

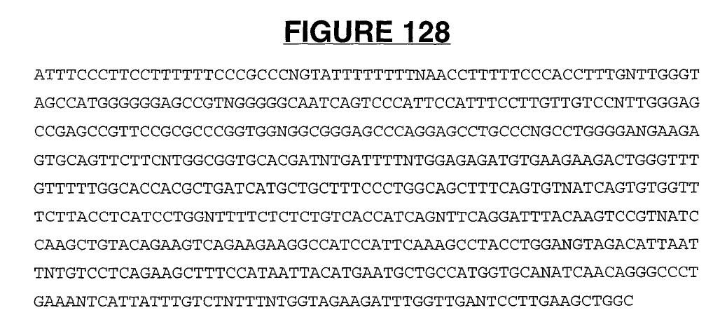

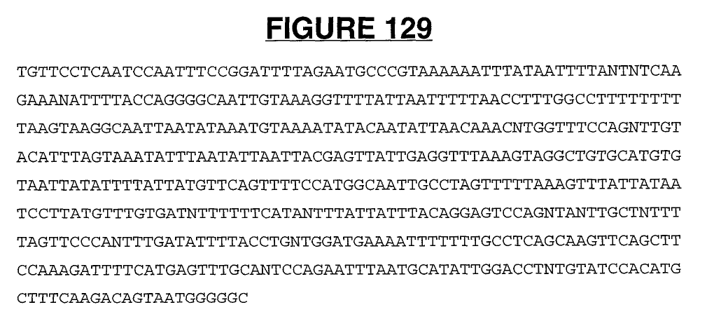

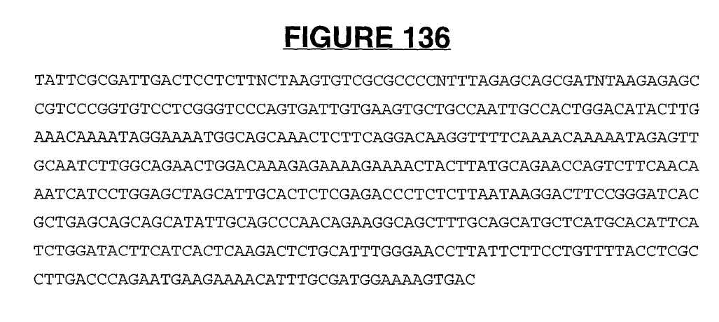

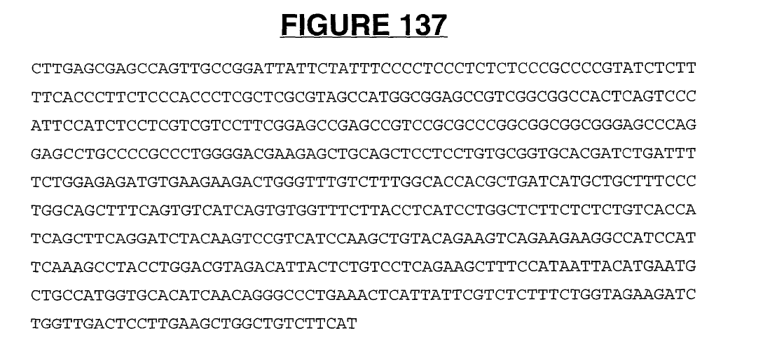

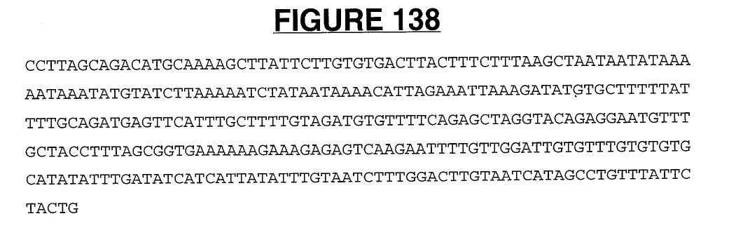

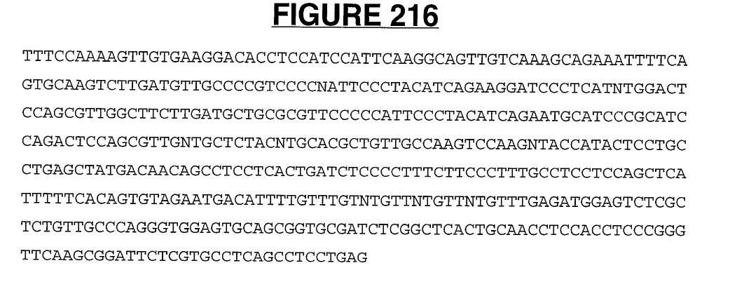

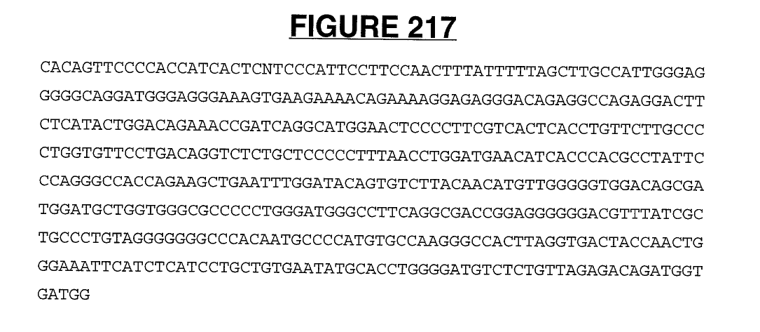

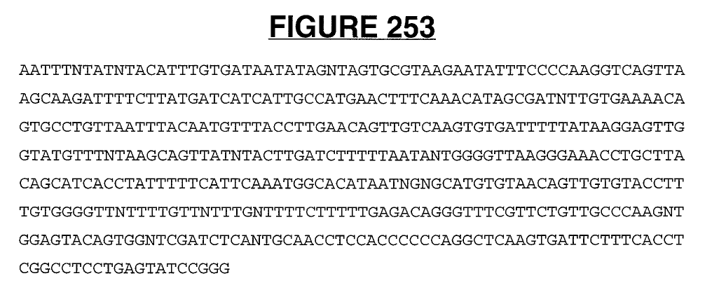

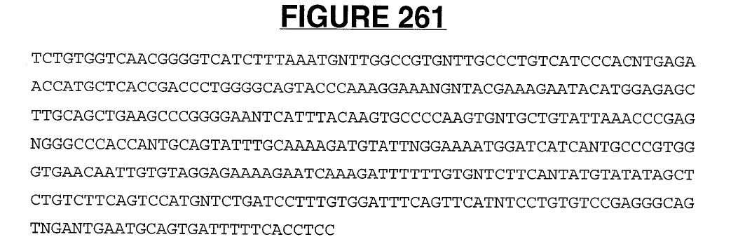

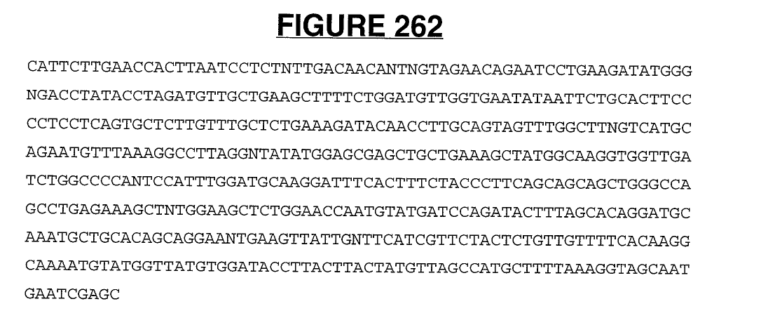

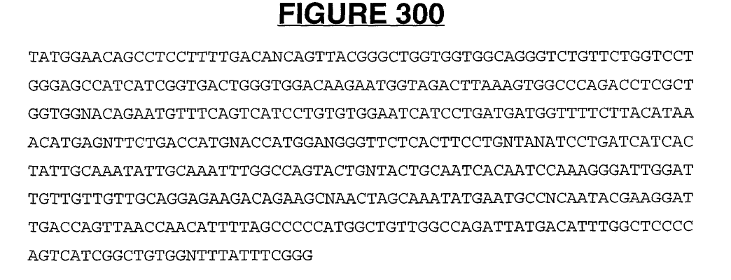

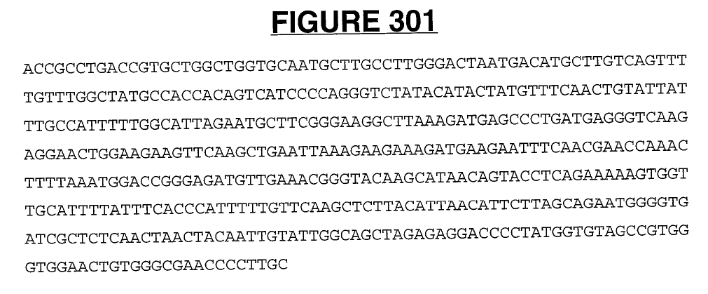

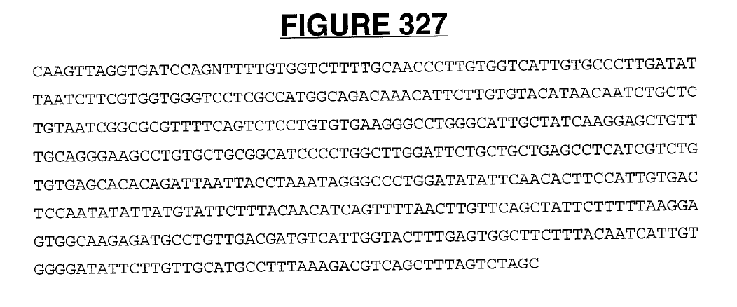

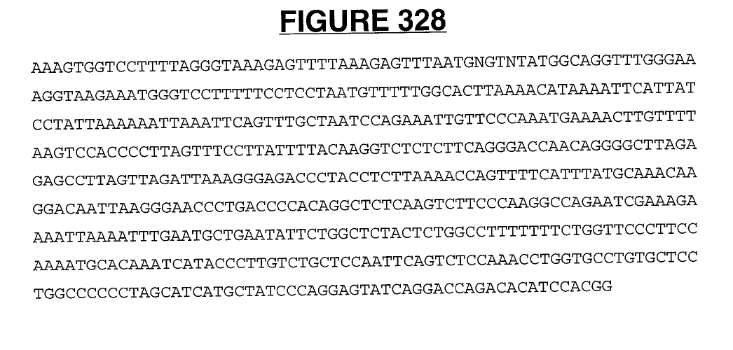

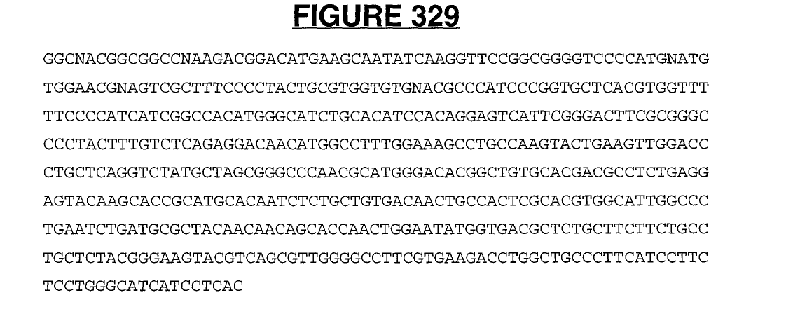

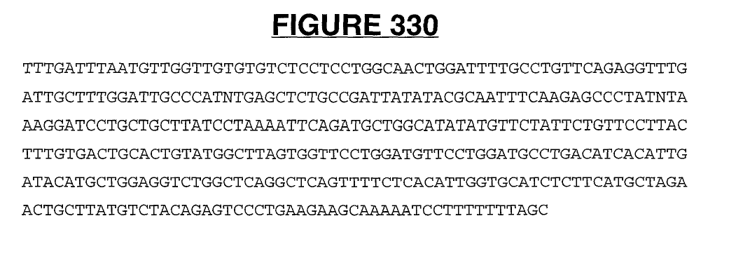

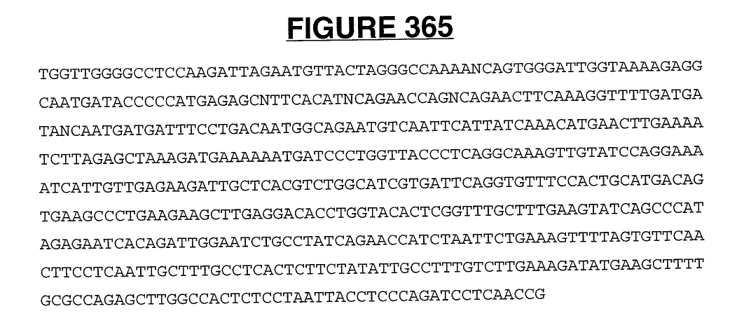

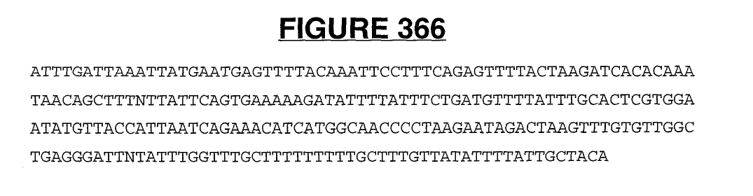

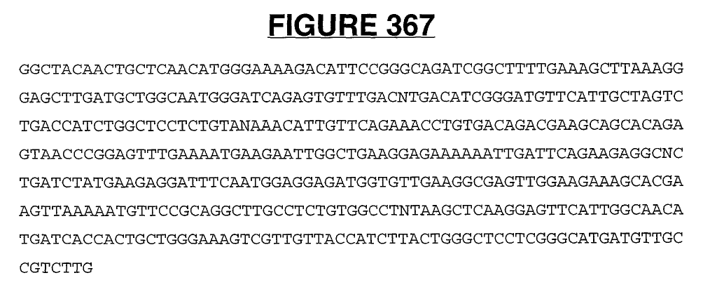

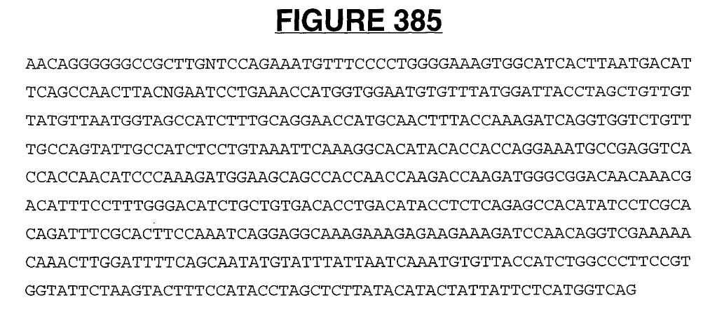

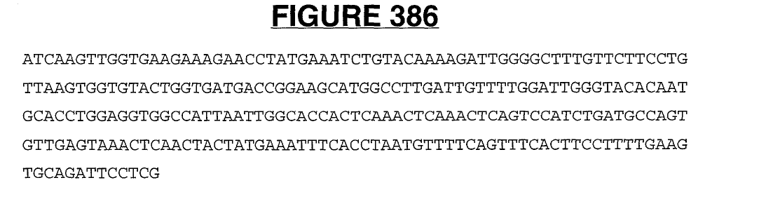

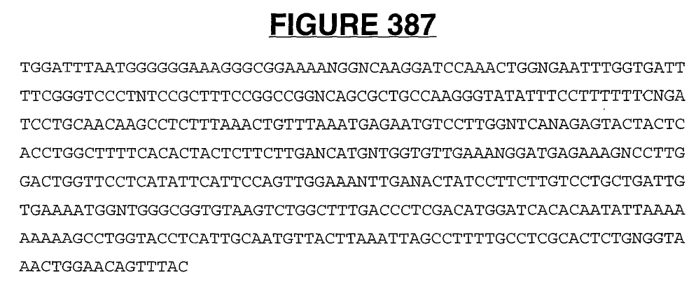

FIGS. 98-102, 125-129, 136-138, 216-217, 253, 261-262, 300-301, 327-330, 365-367 and 385-387.

(m) Human uterus tissue

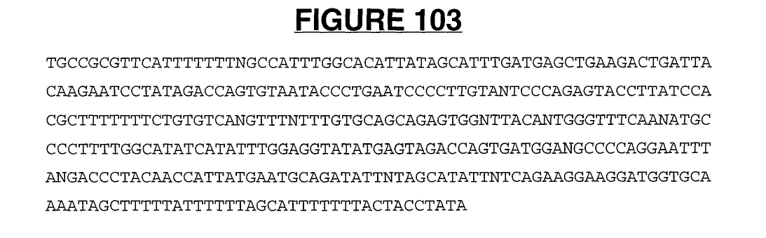

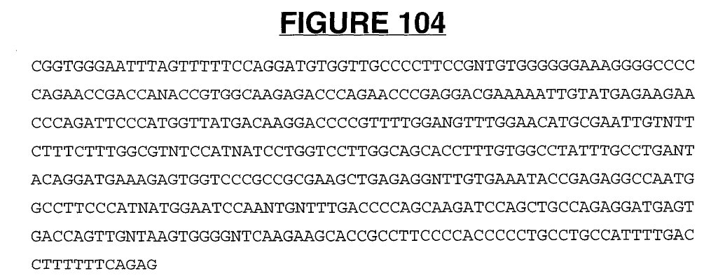

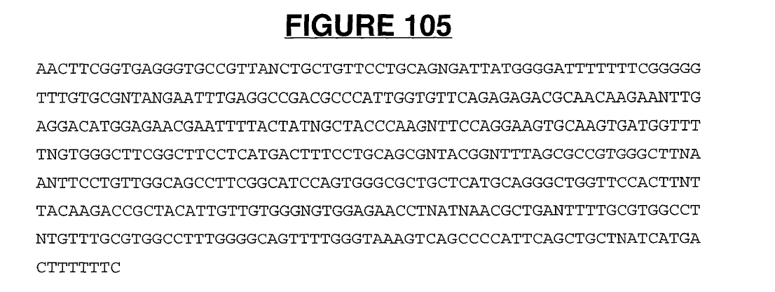

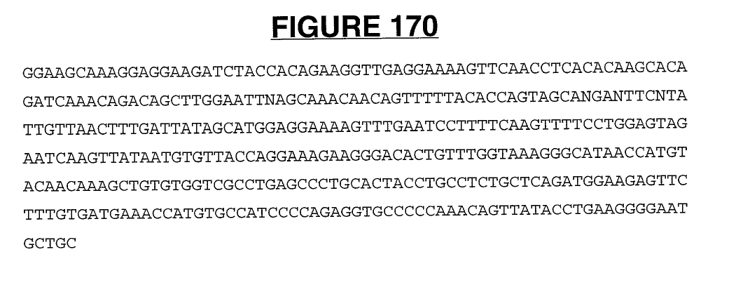

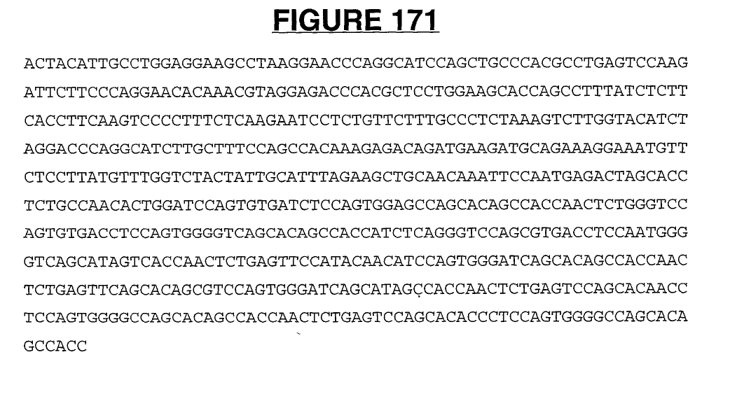

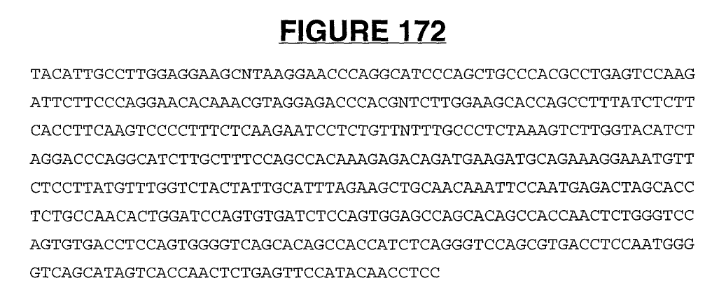

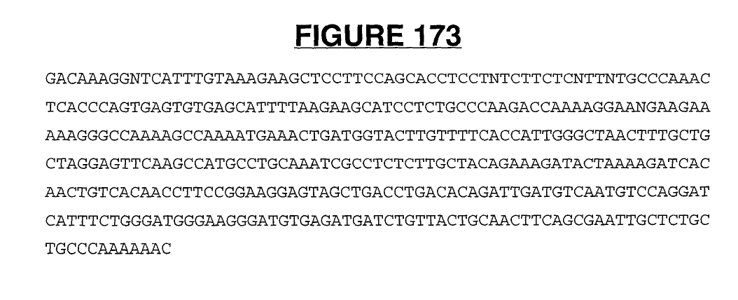

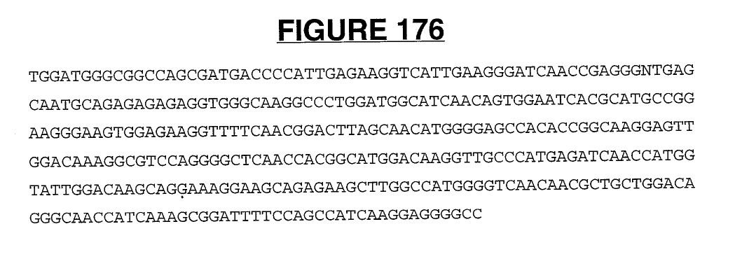

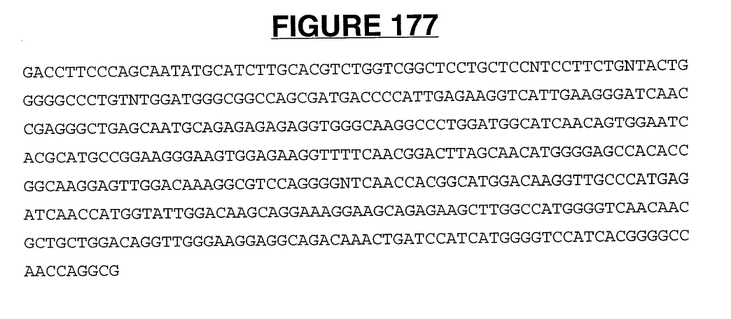

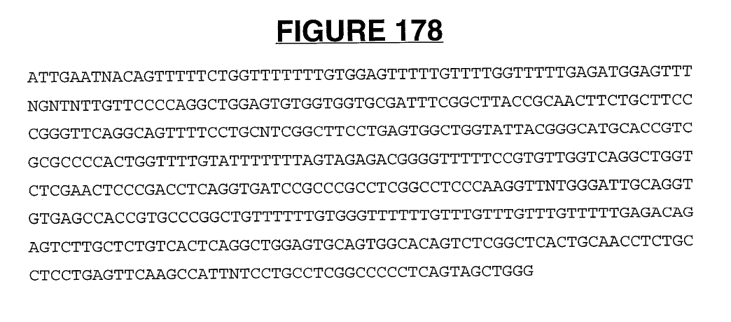

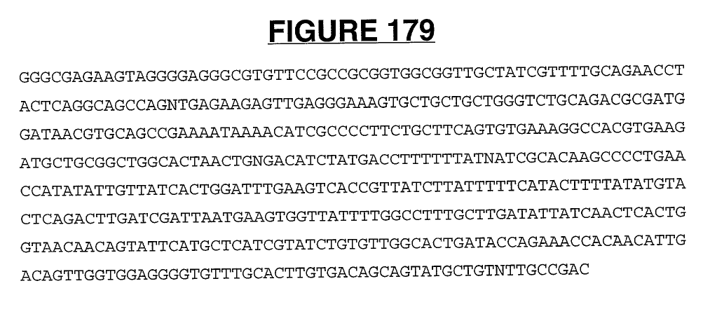

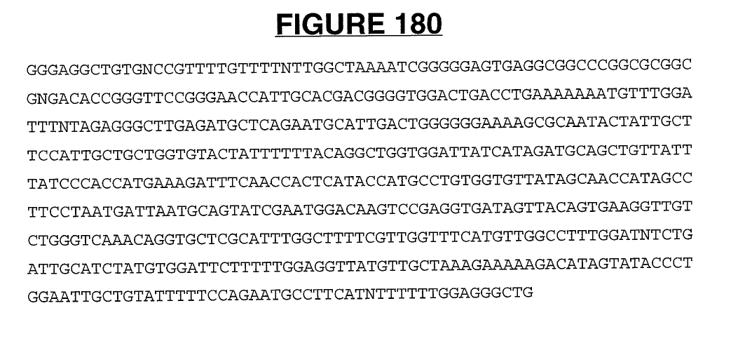

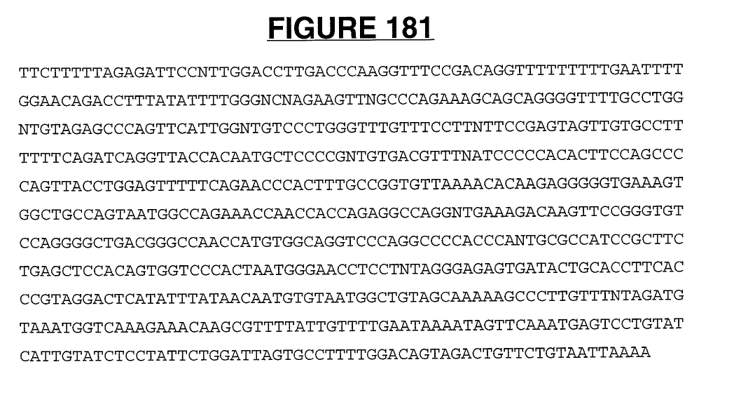

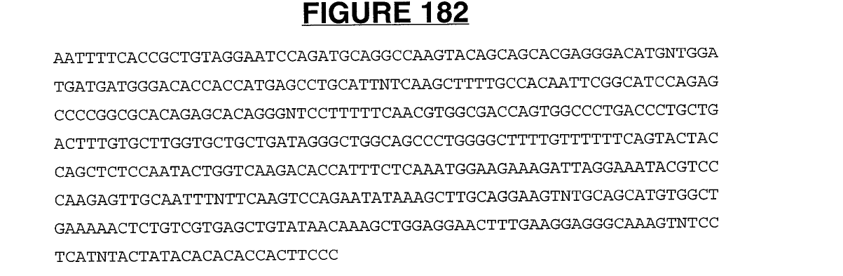

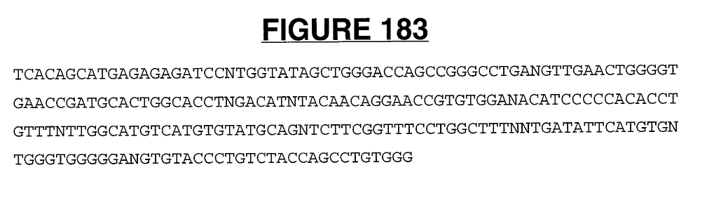

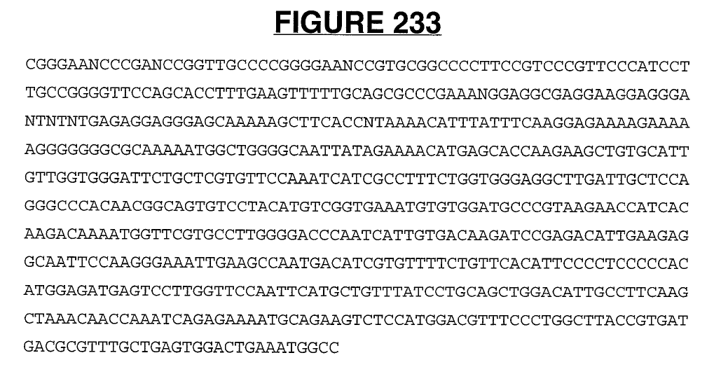

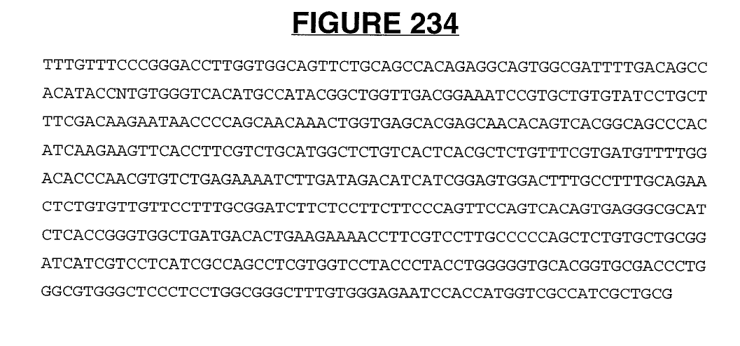

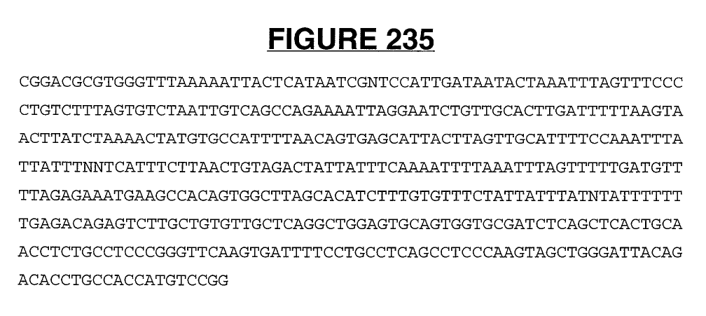

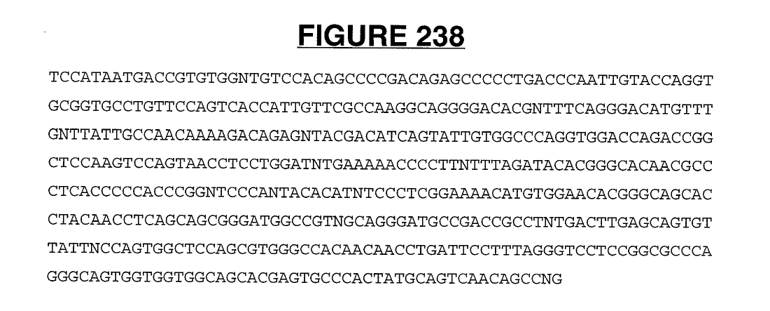

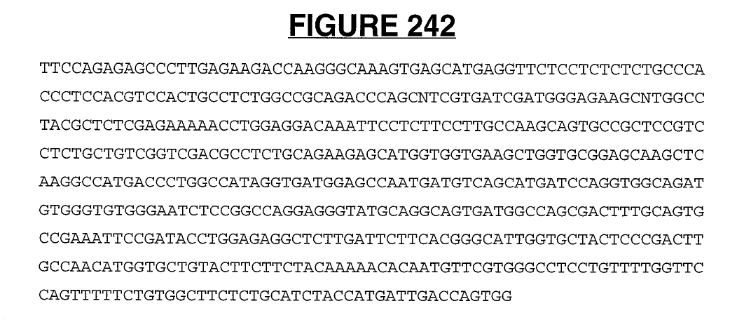

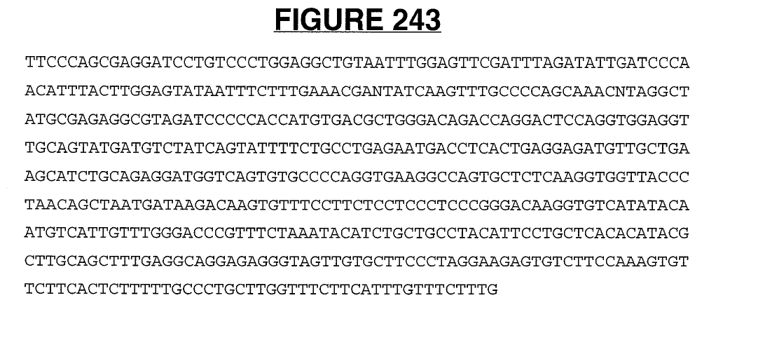

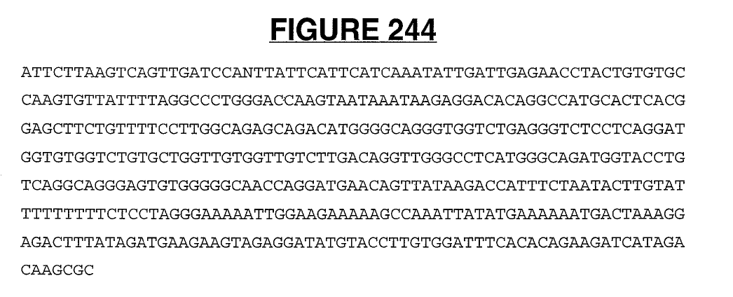

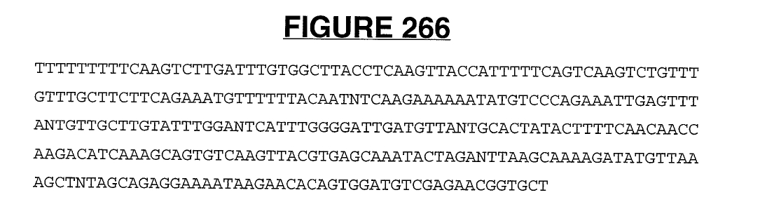

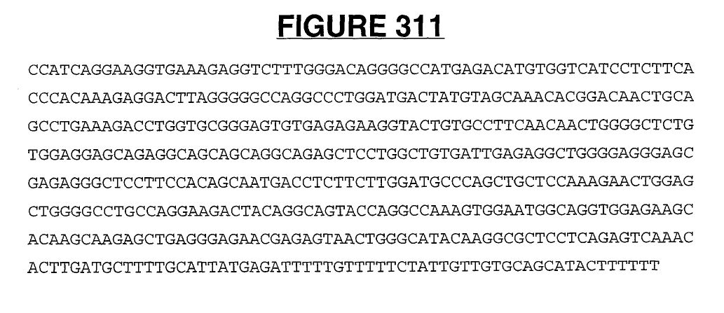

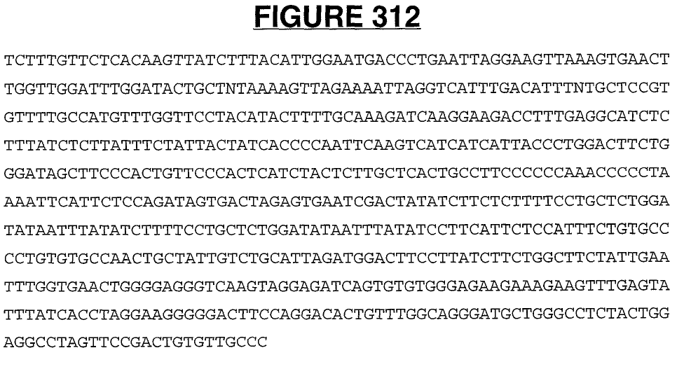

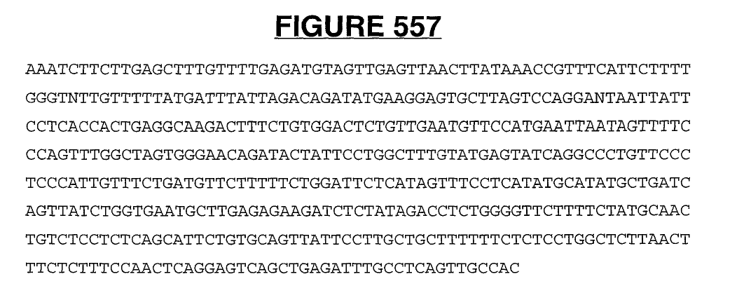

FIGS. 103-106, 170-173, 176-183, 233-235, 238, 242-244, 266, 311-312 and 557.

(n) Human lung carcinoma tissue

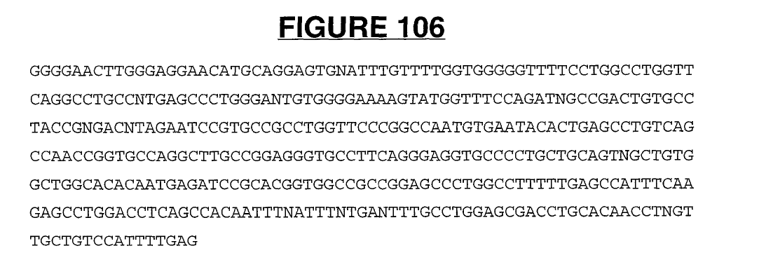

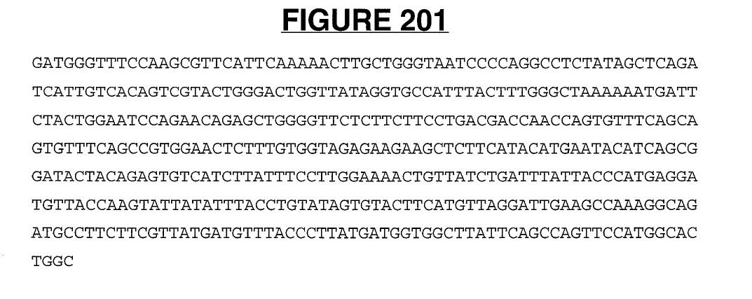

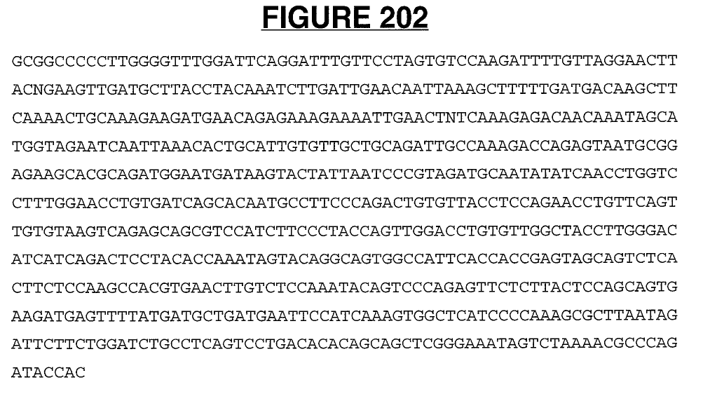

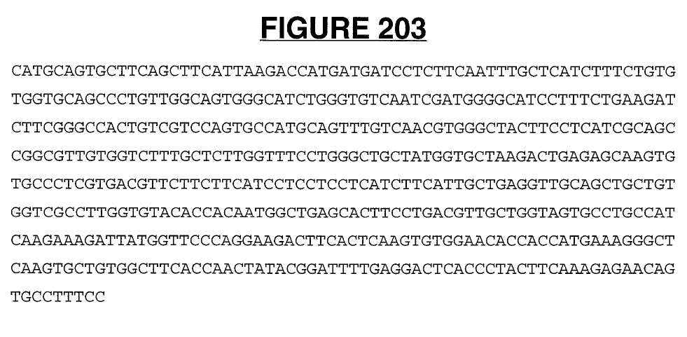

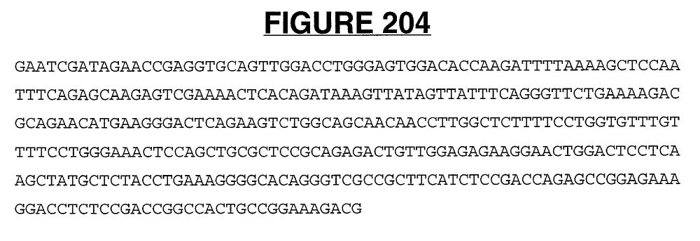

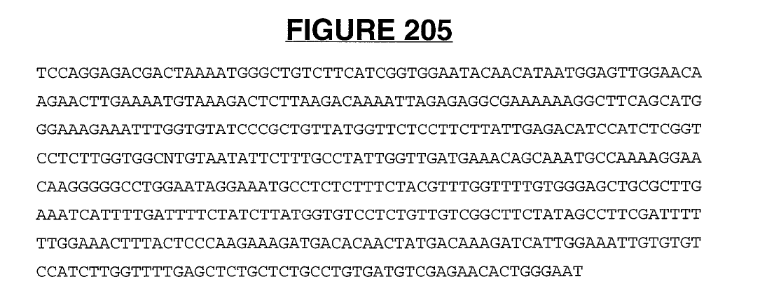

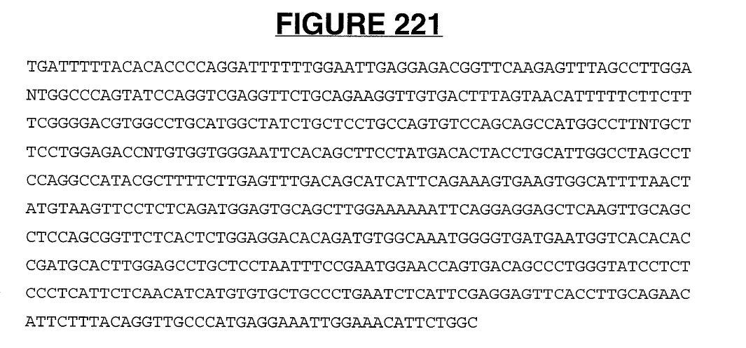

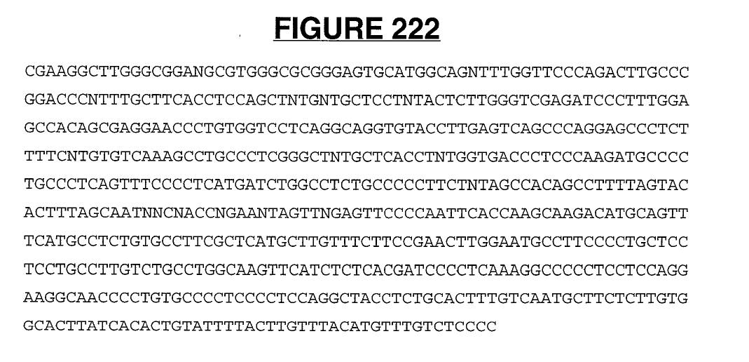

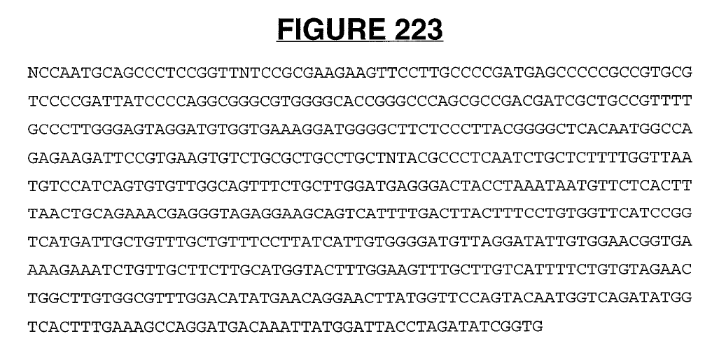

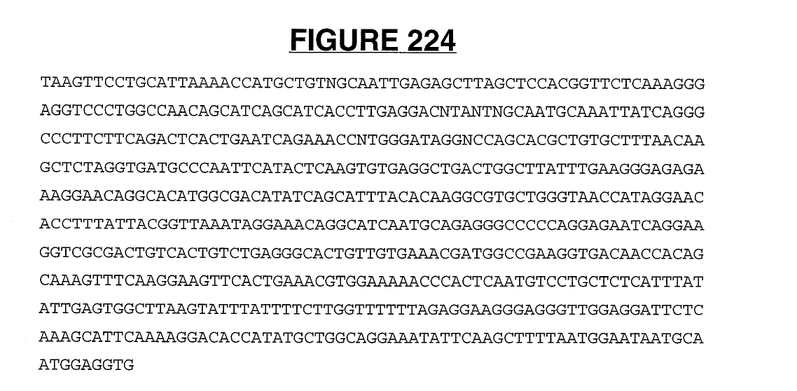

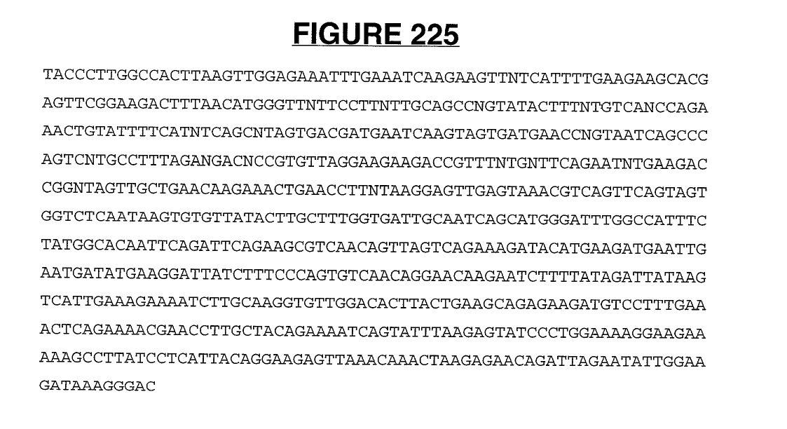

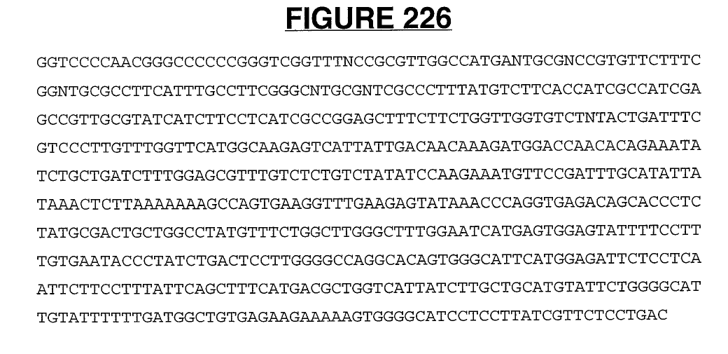

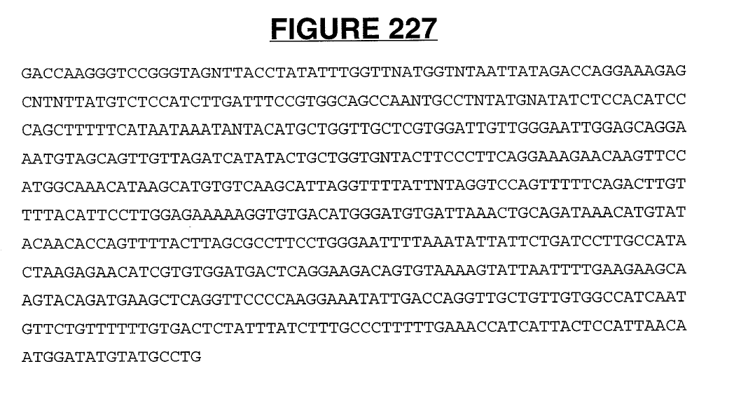

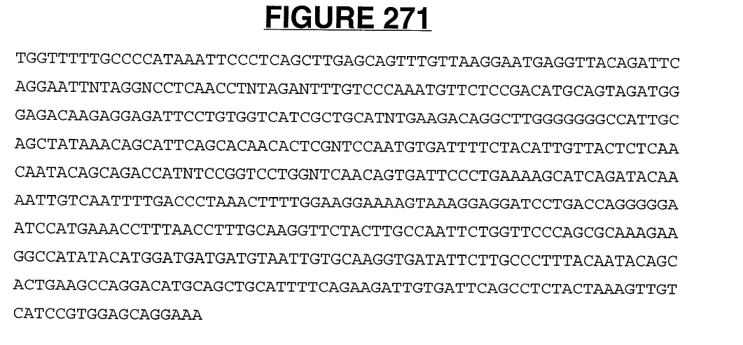

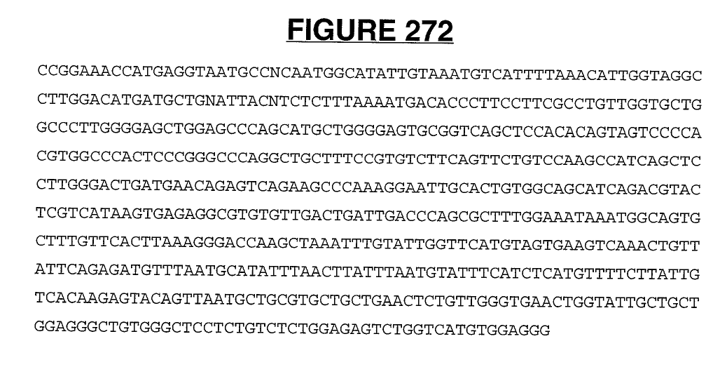

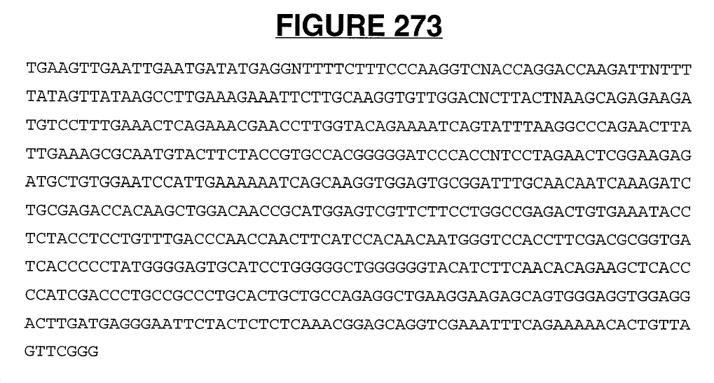

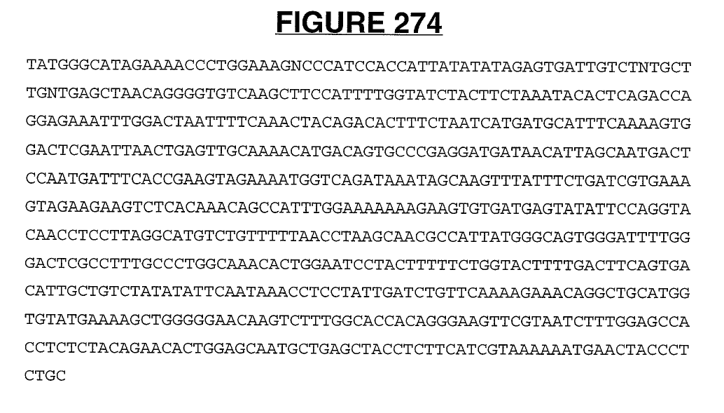

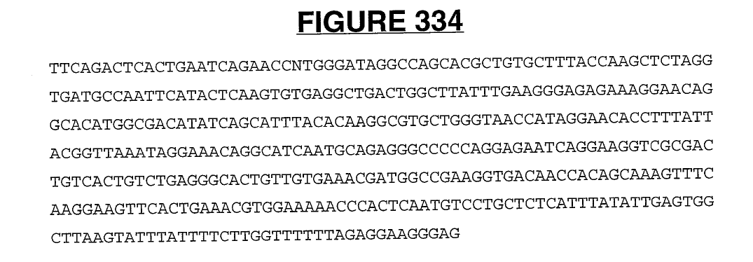

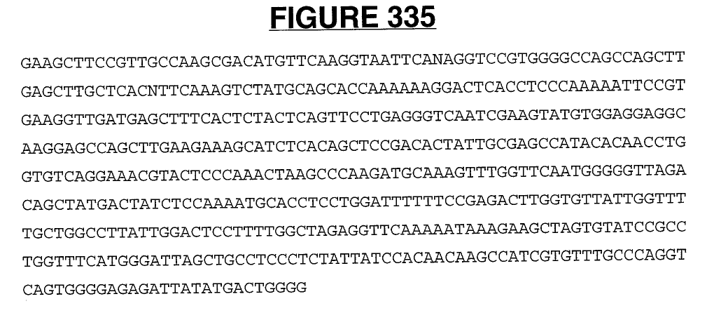

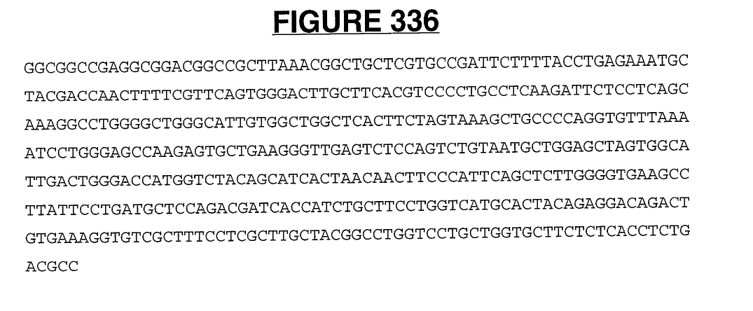

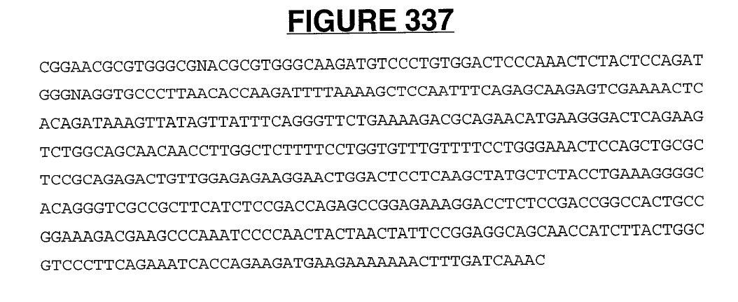

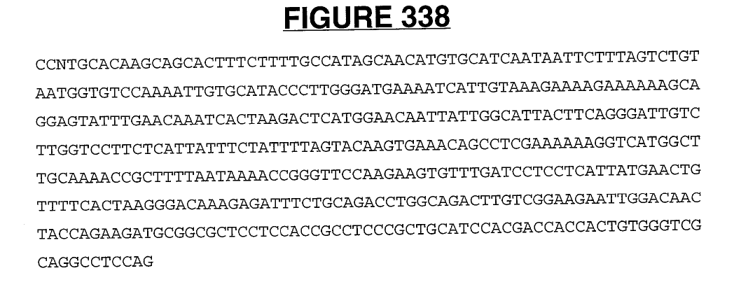

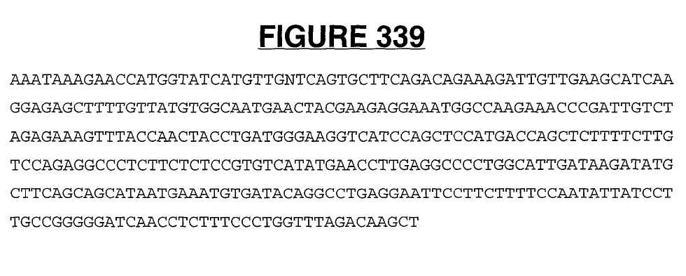

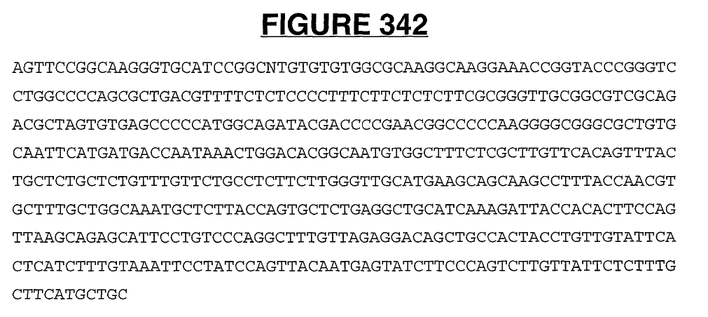

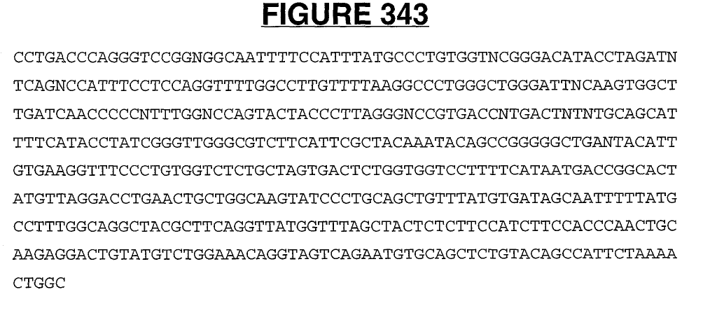

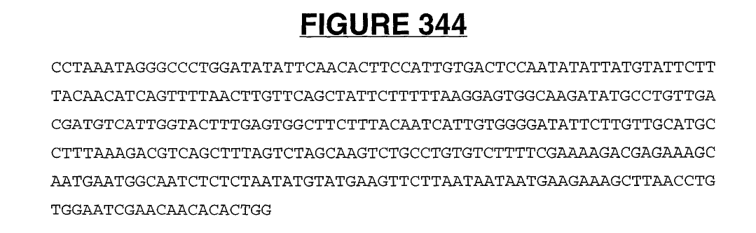

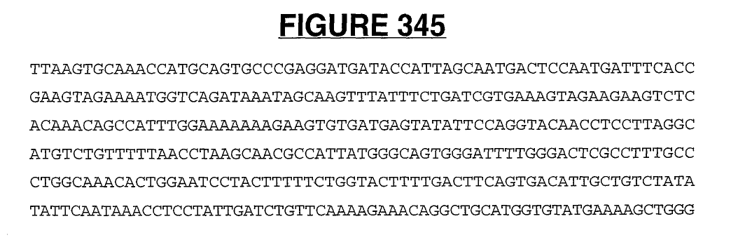

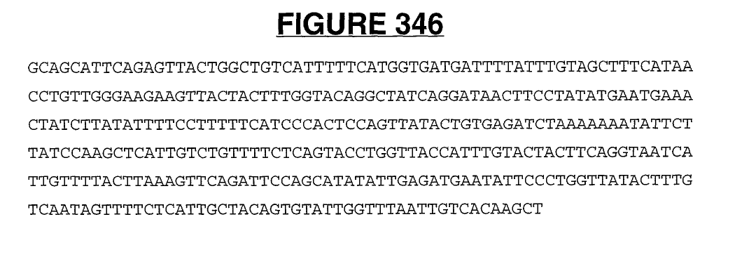

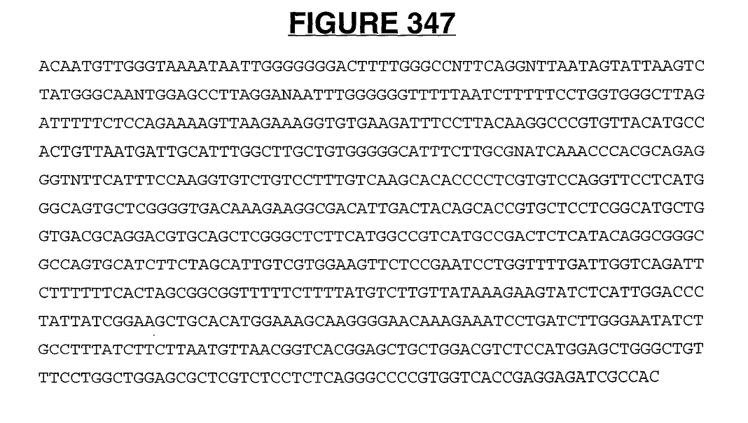

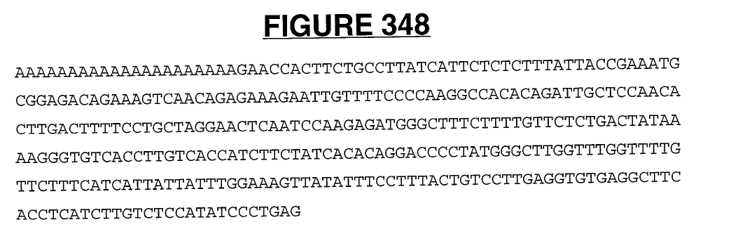

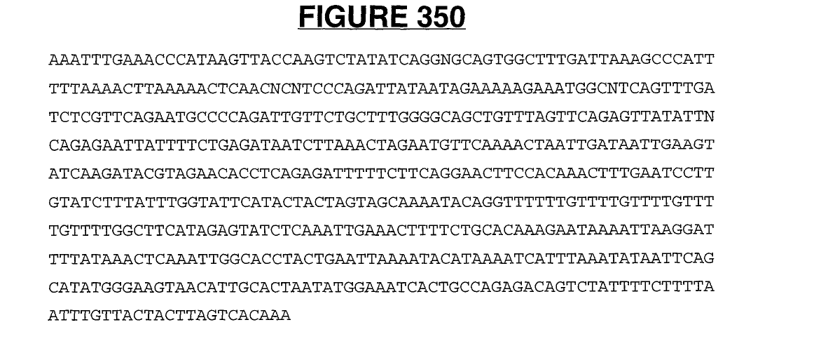

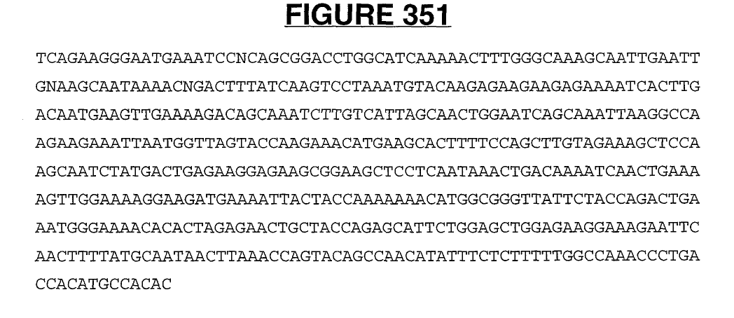

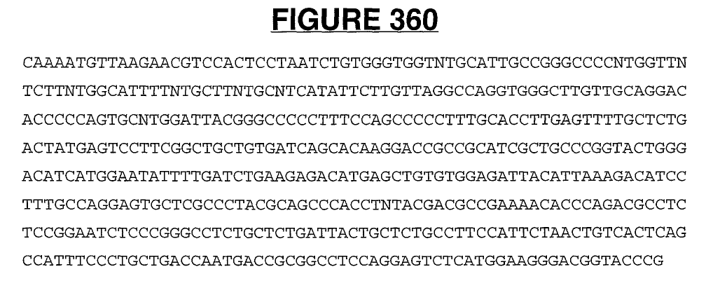

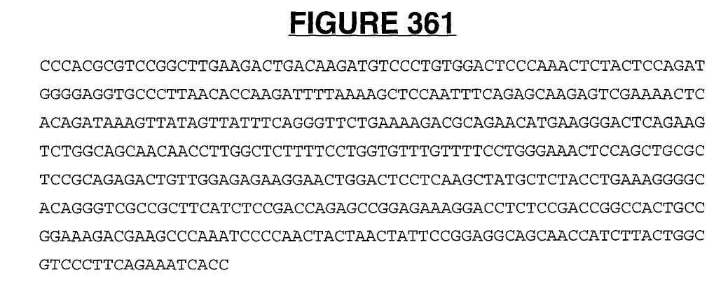

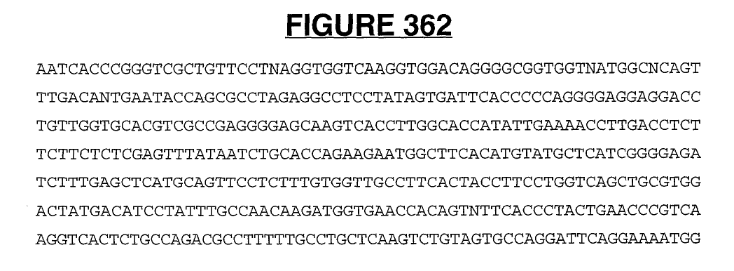

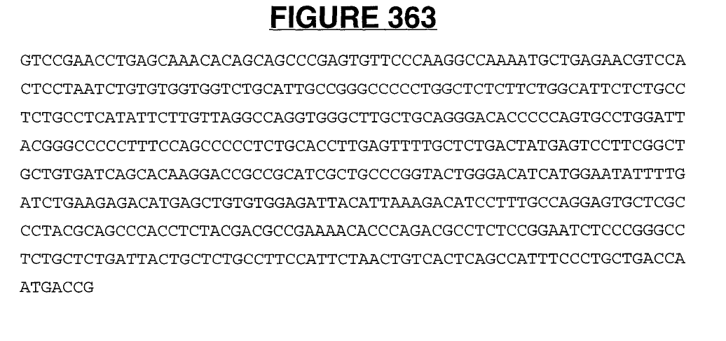

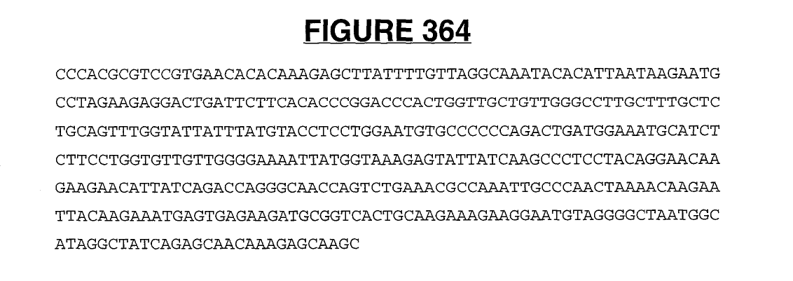

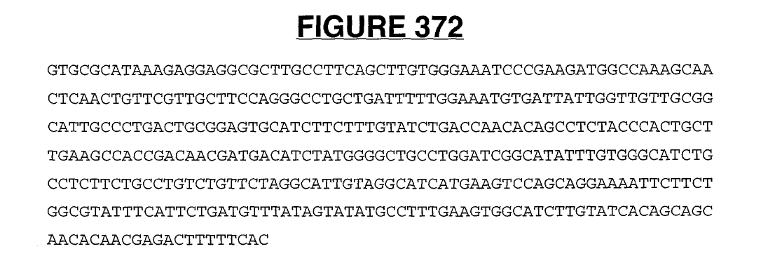

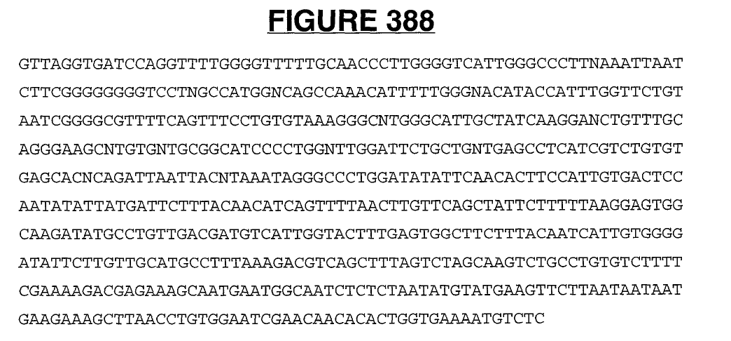

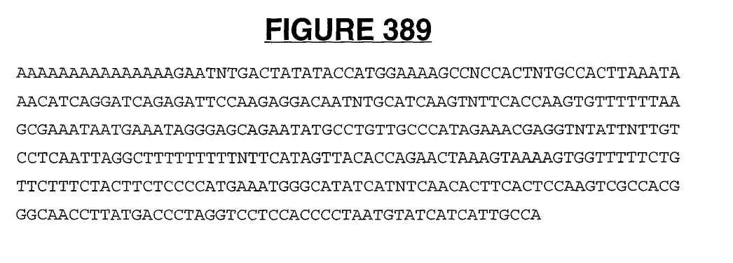

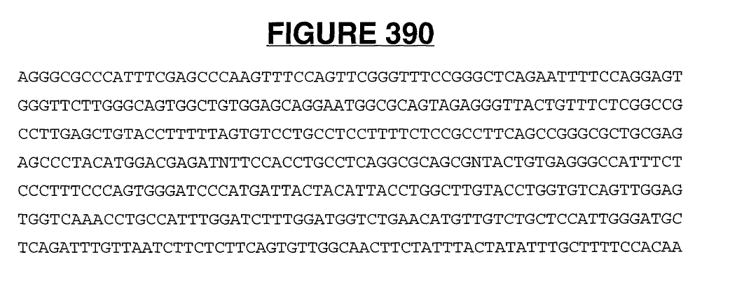

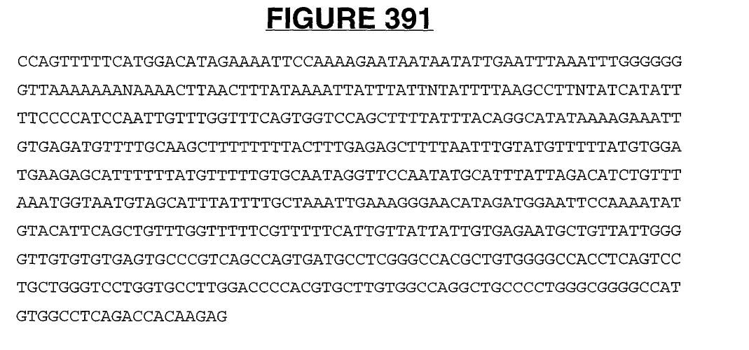

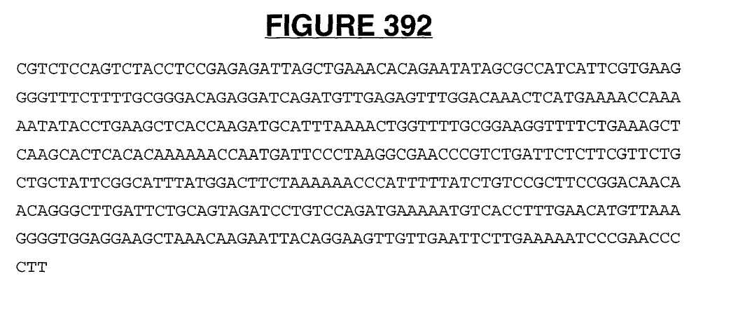

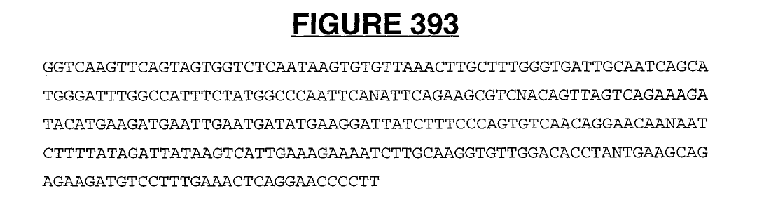

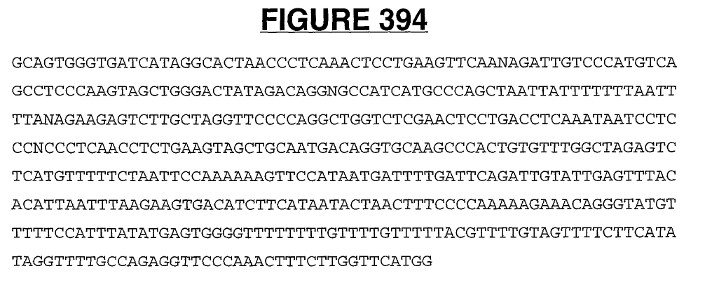

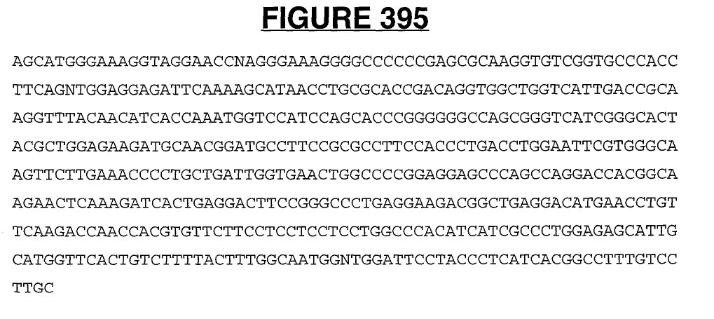

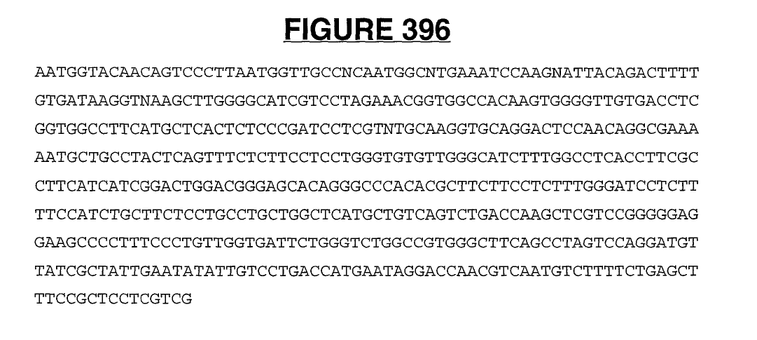

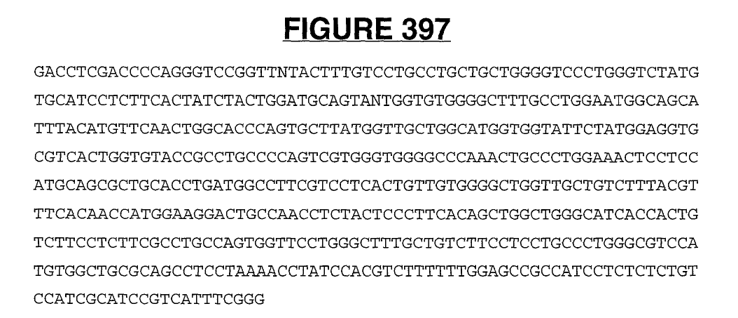

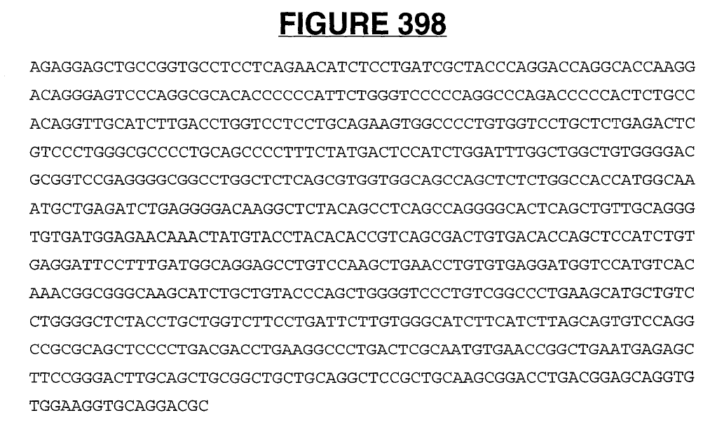

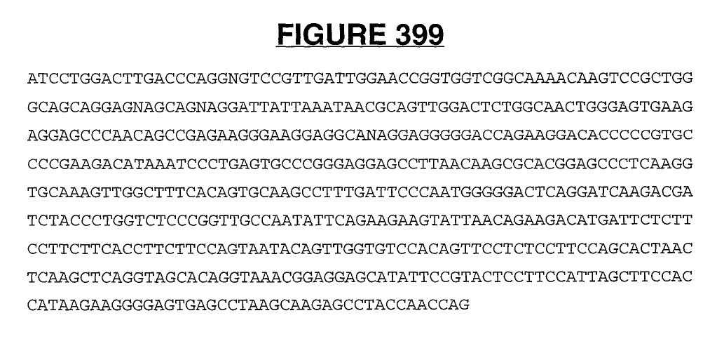

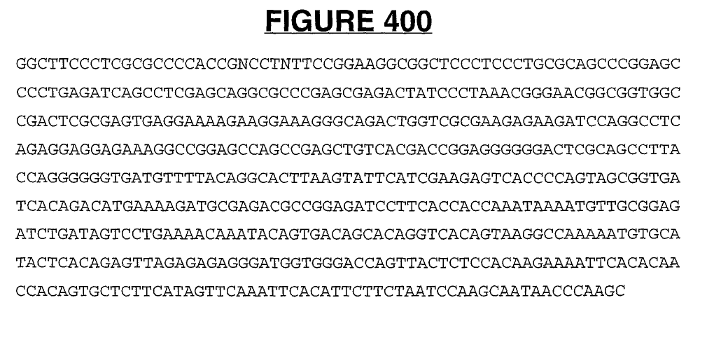

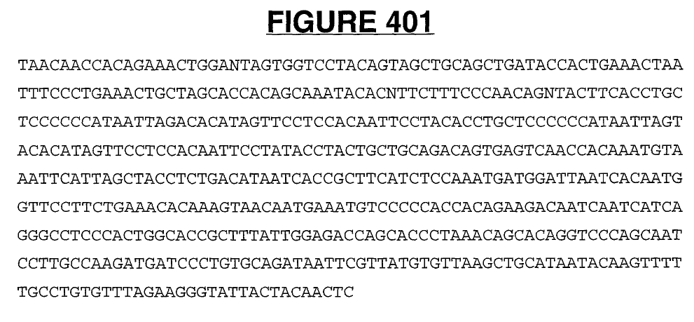

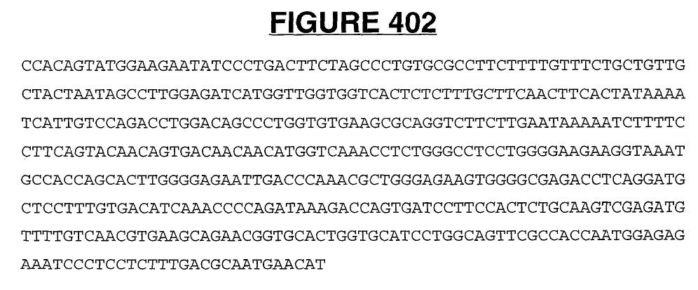

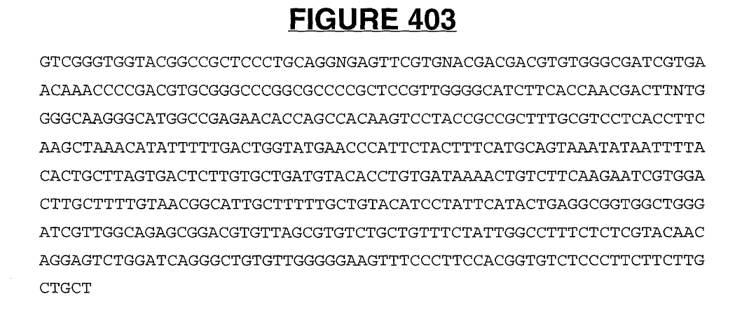

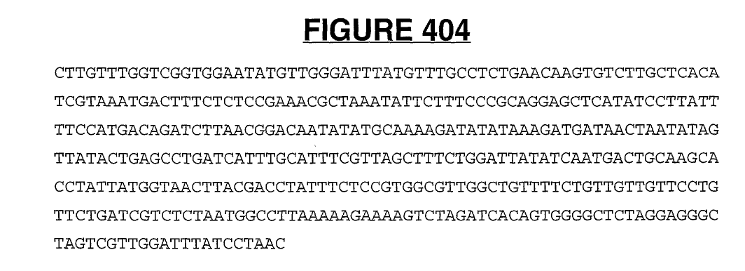

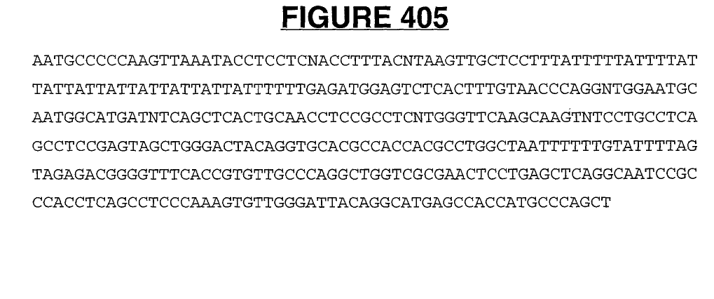

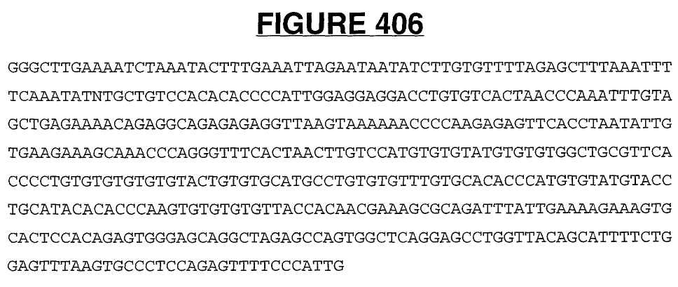

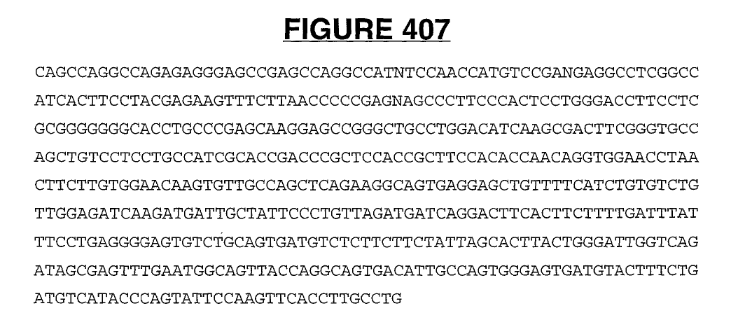

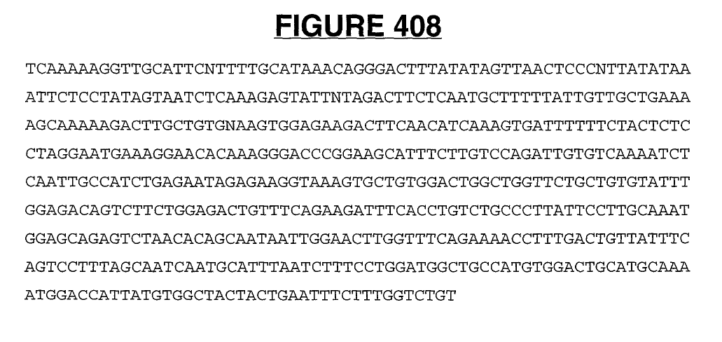

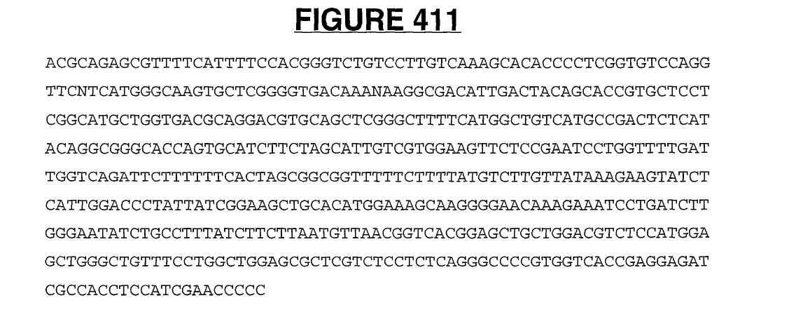

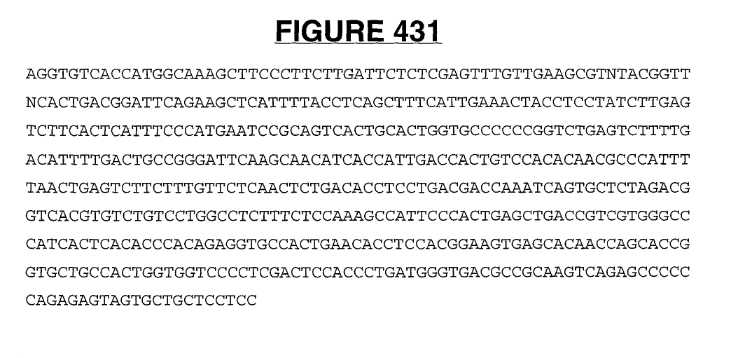

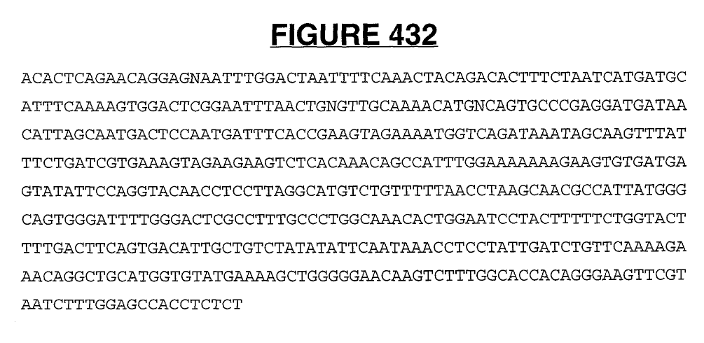

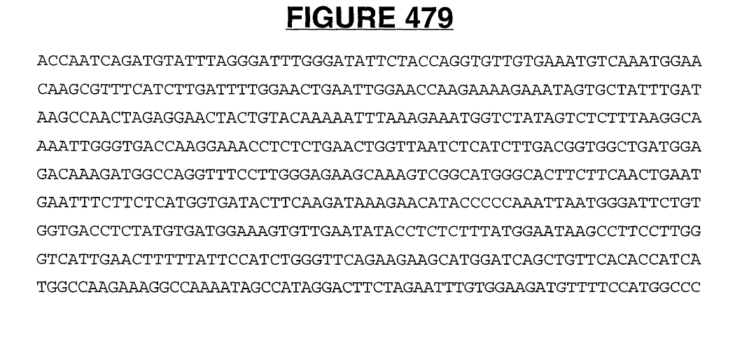

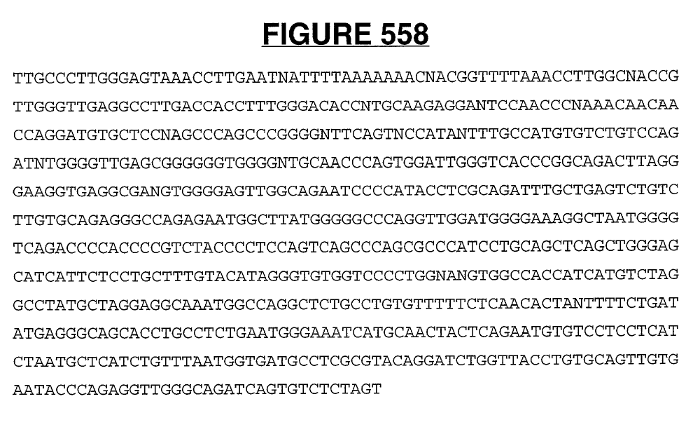

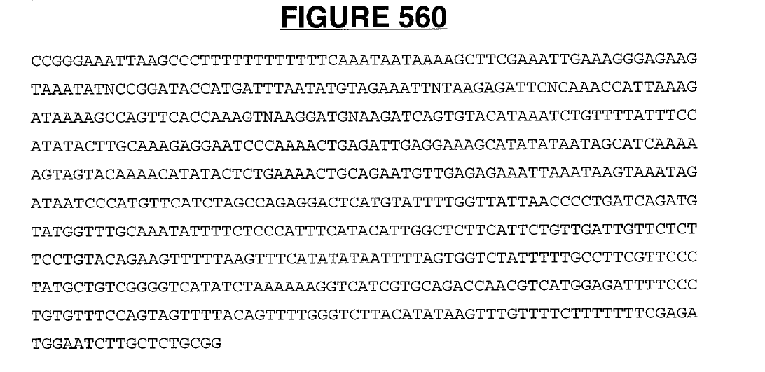

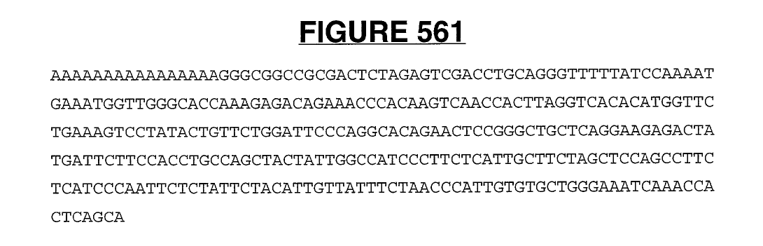

FIGS. 106-108, 201-205, 221-227, 271-274, 334-339, 342-348, 350-351, 360-364, 372, 388-408, 411, 431-432, 479, 558 and 560-561.

(o) Human mammary epithelial cells

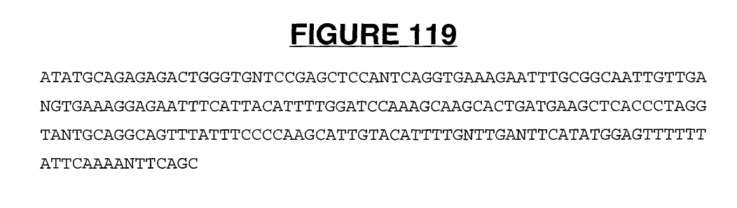

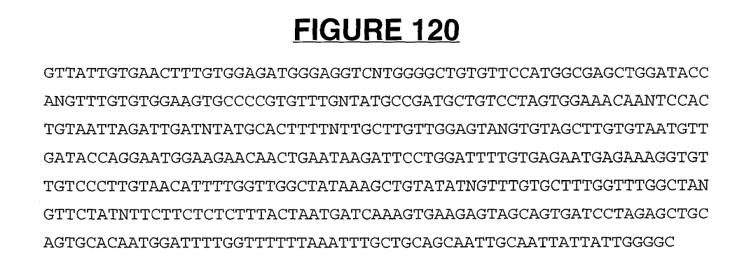

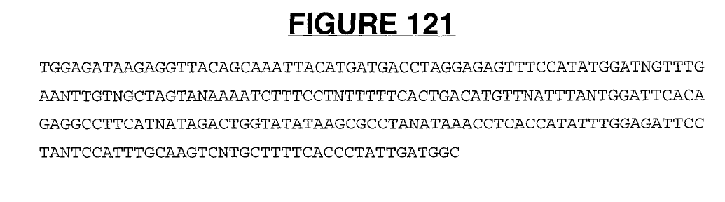

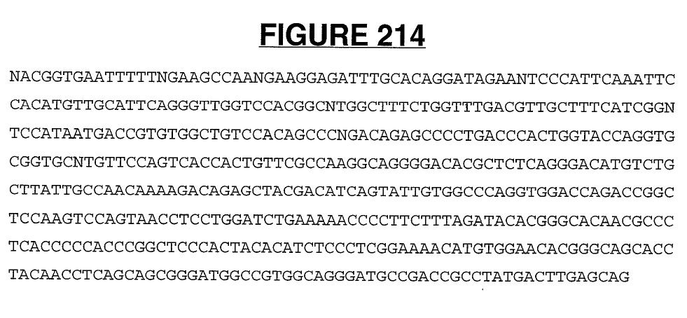

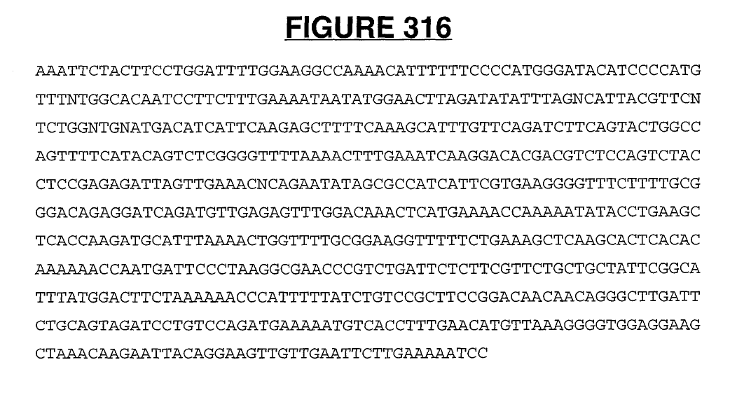

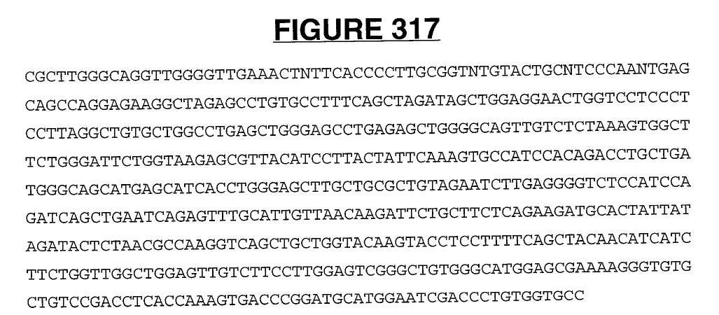

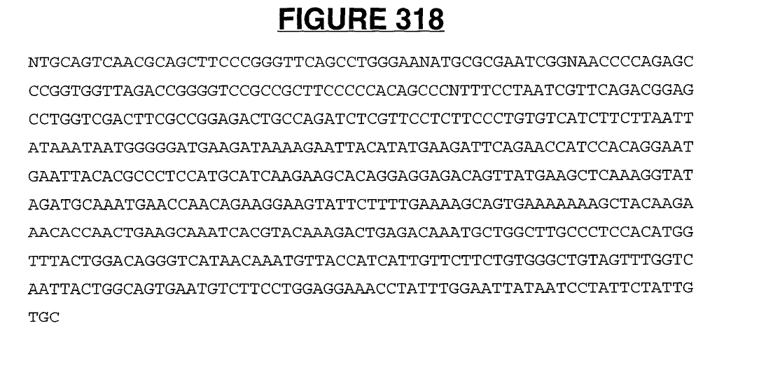

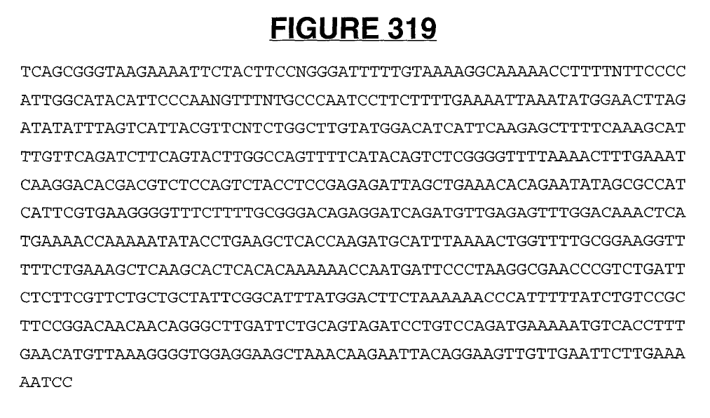

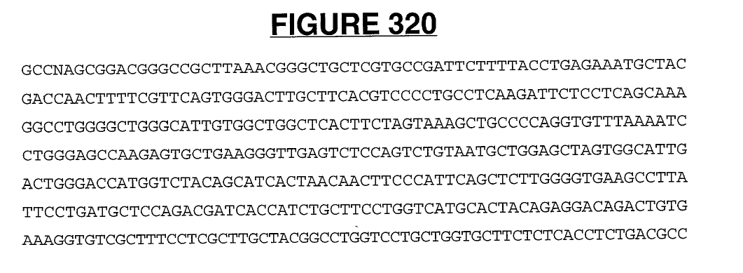

FIGS. 119-121, 214 and 316-320.

(p) Human chronic myelogenous leukemia tissue

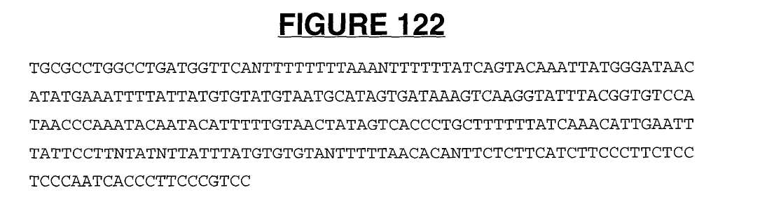

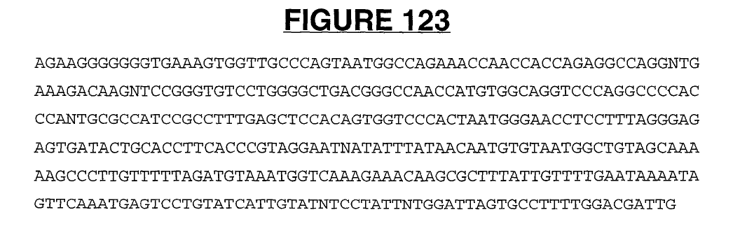

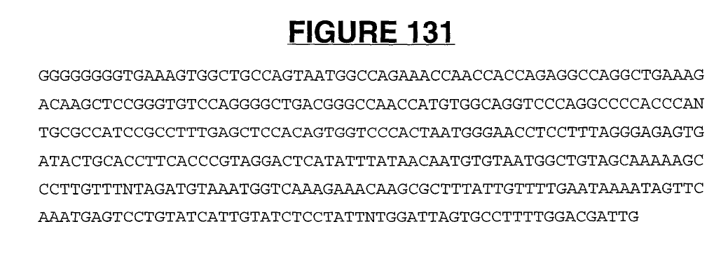

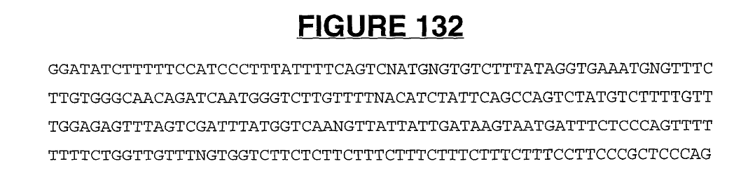

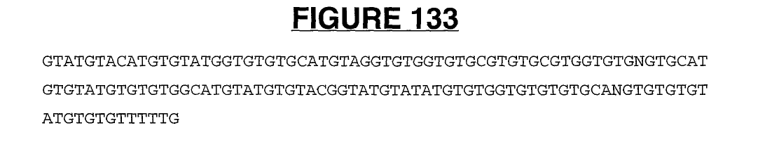

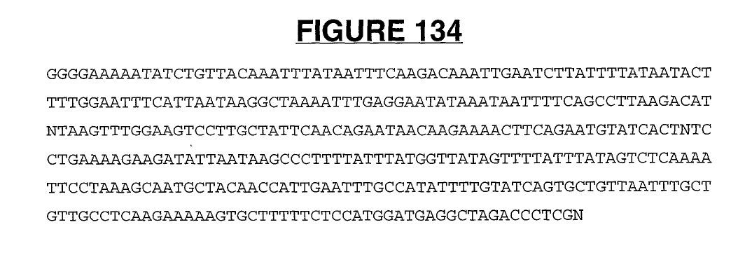

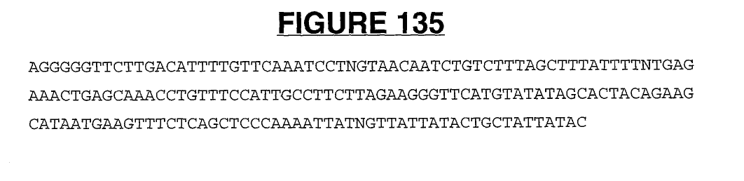

FIGS. 122-123 and 131-135.

(q) Human spinal cord tissue

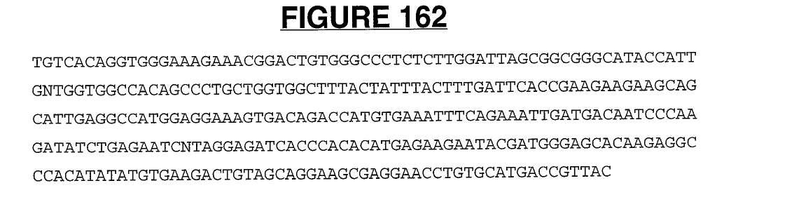

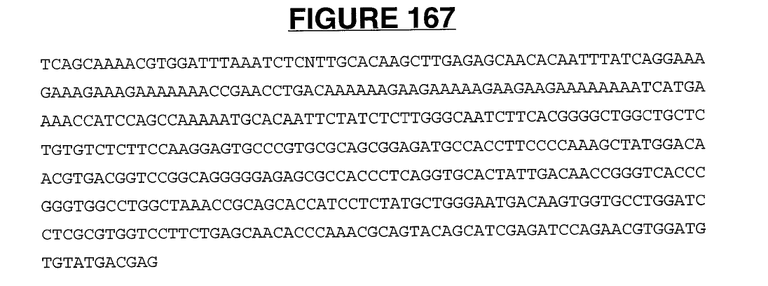

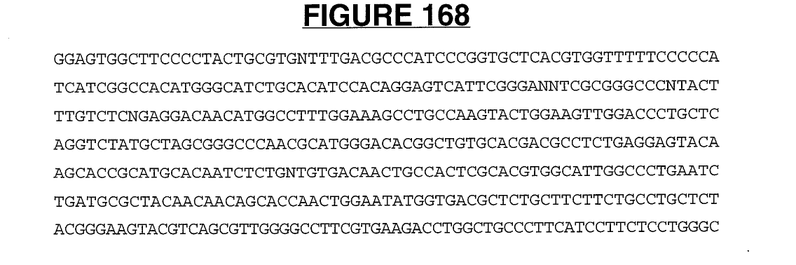

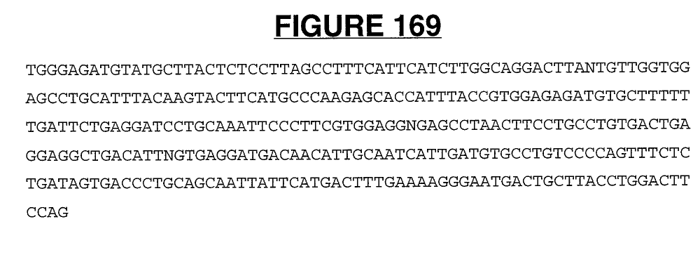

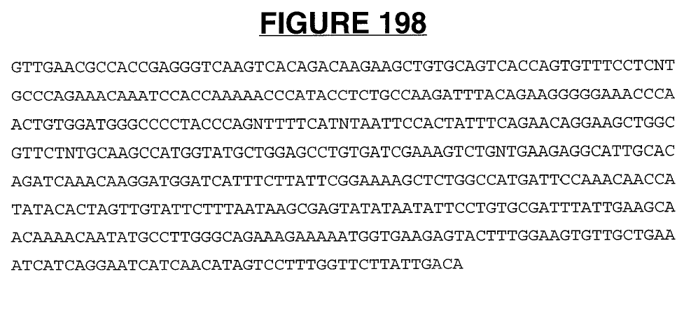

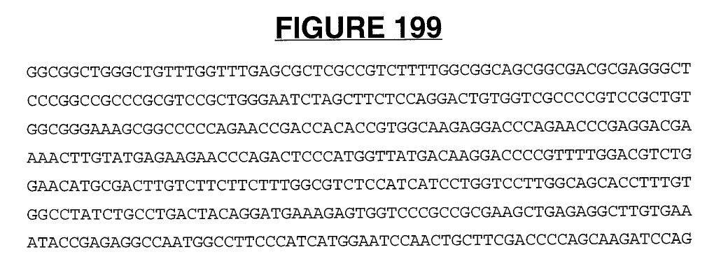

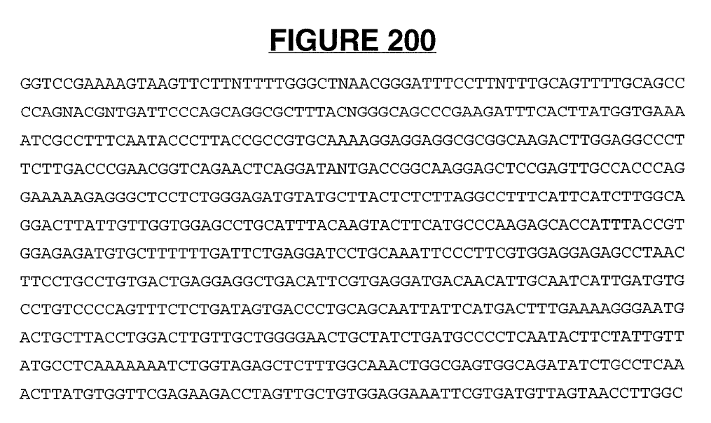

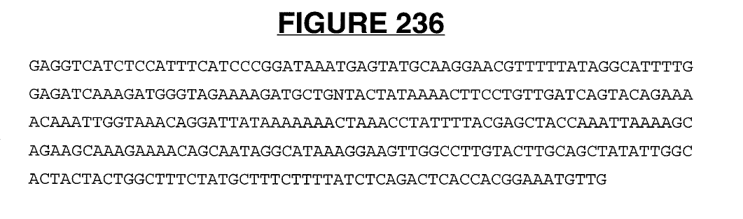

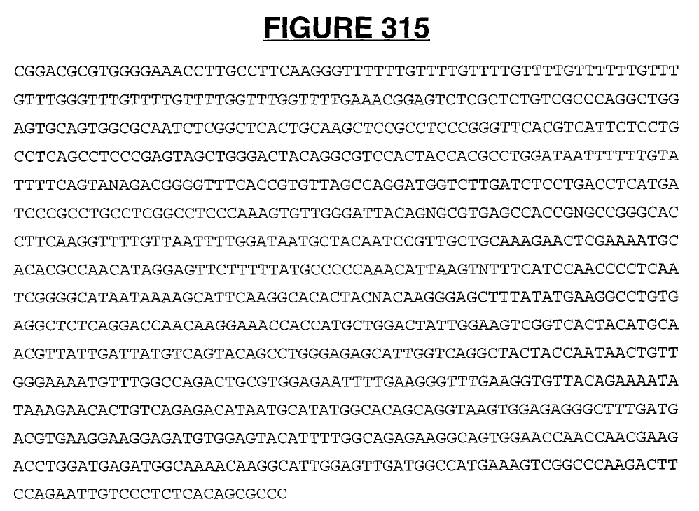

FIGS. 162, 167-169, 198-200, 236 and 315.

(r) Human fetal brain tissue

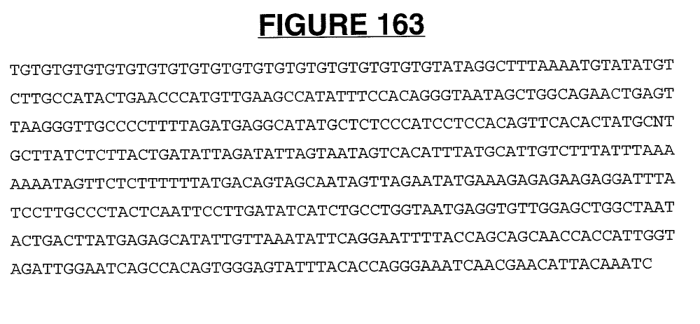

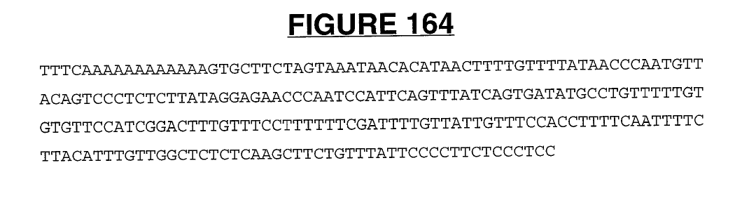

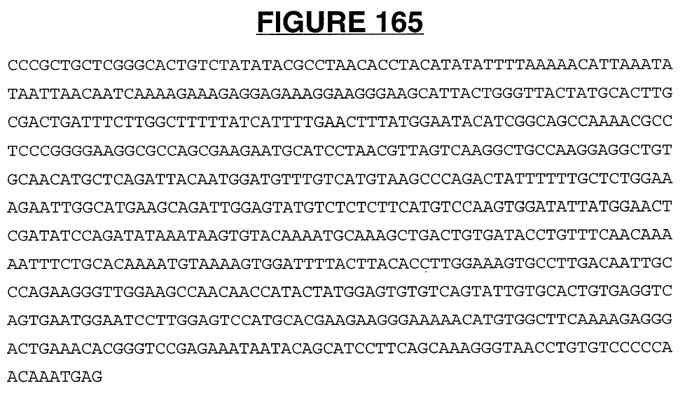

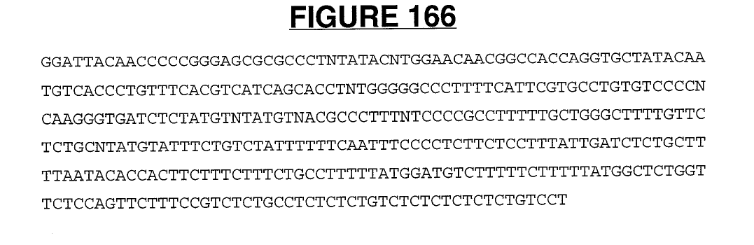

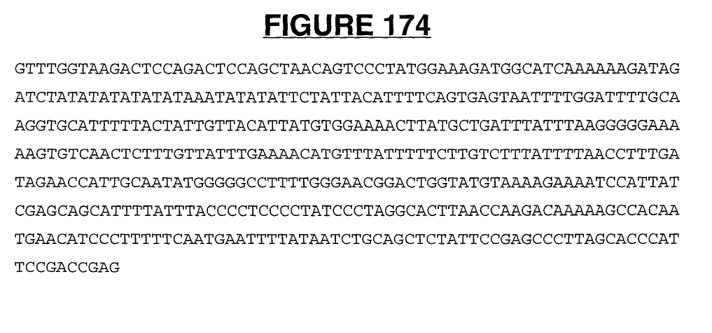

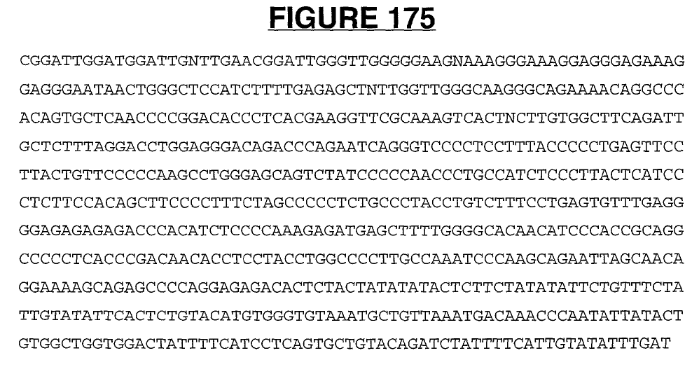

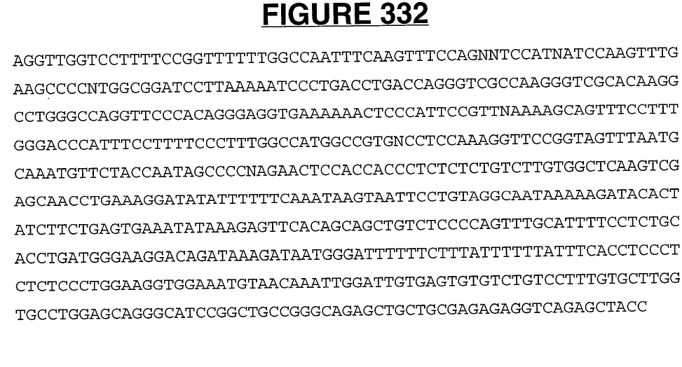

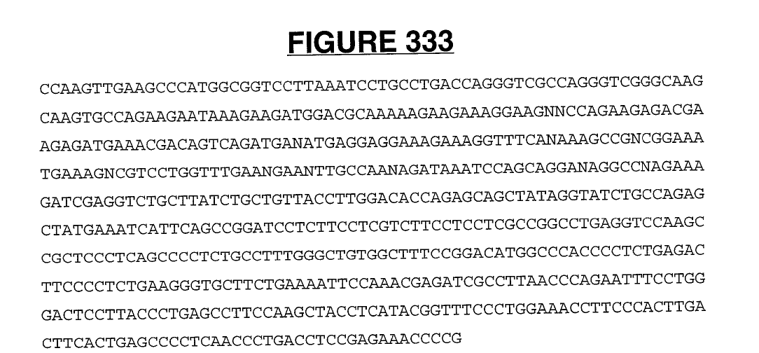

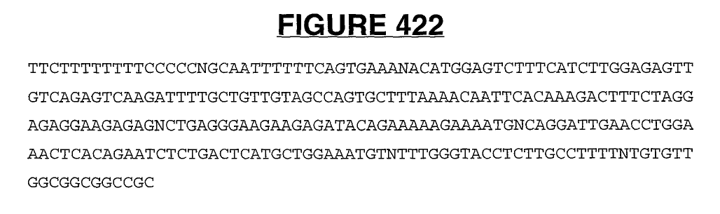

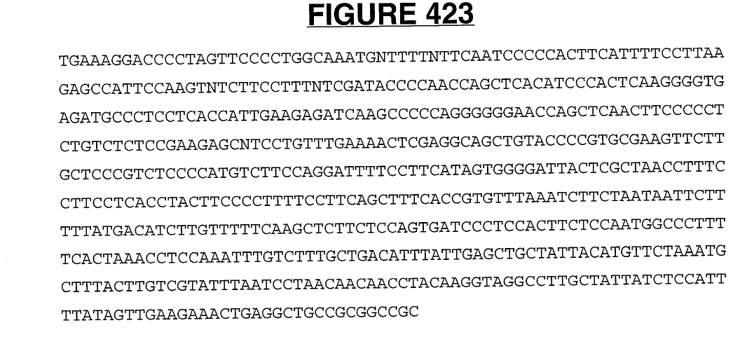

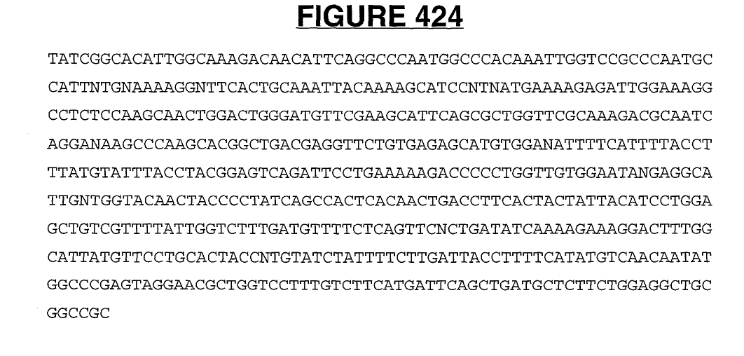

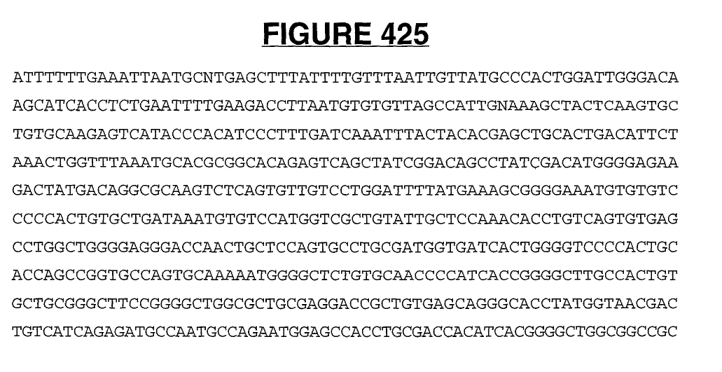

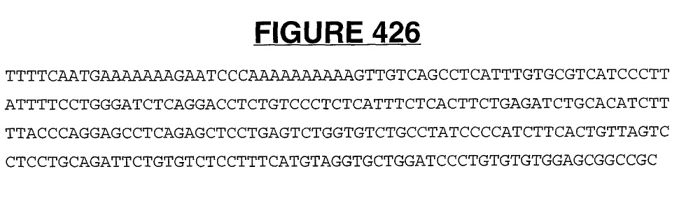

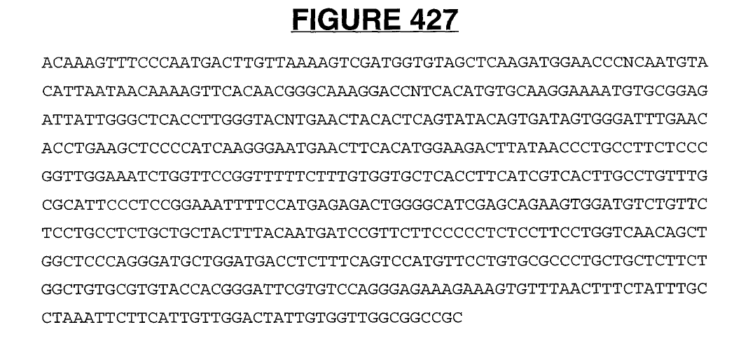

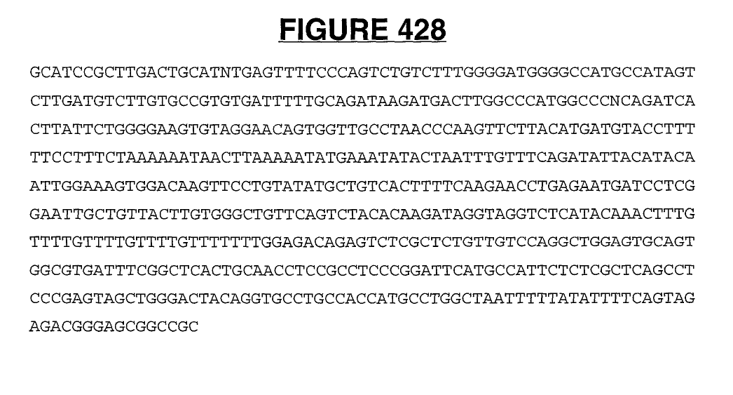

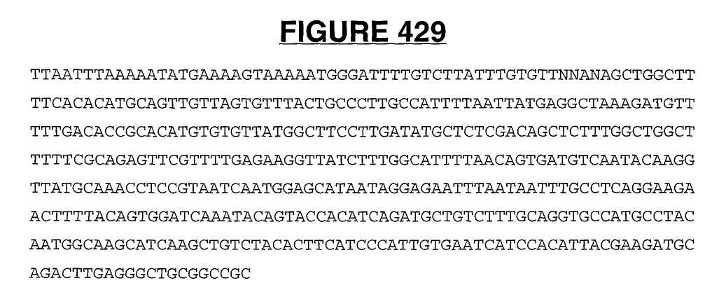

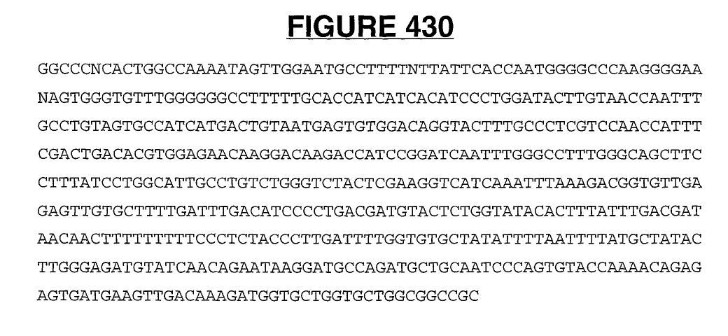

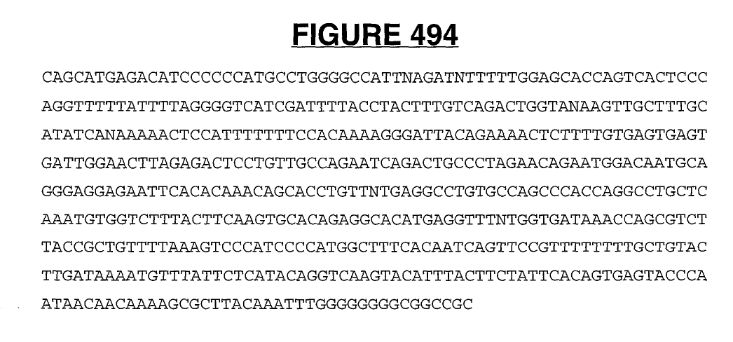

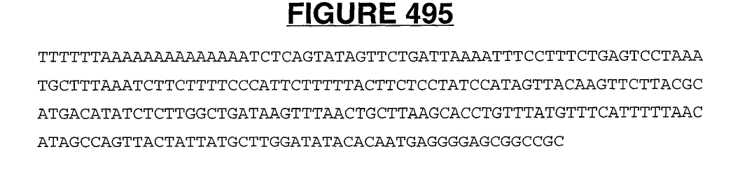

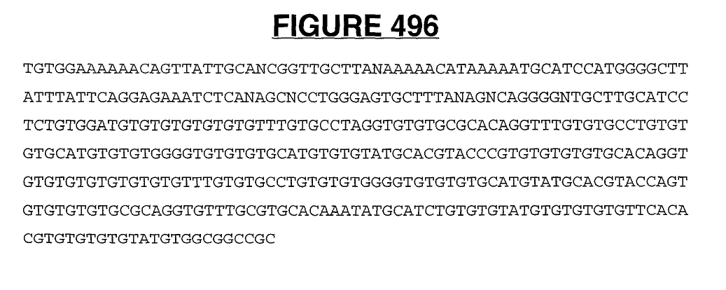

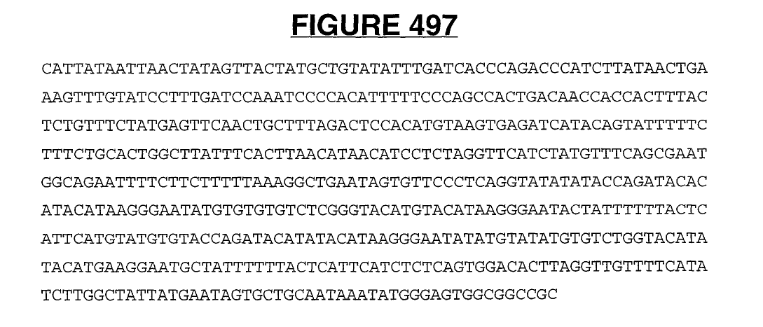

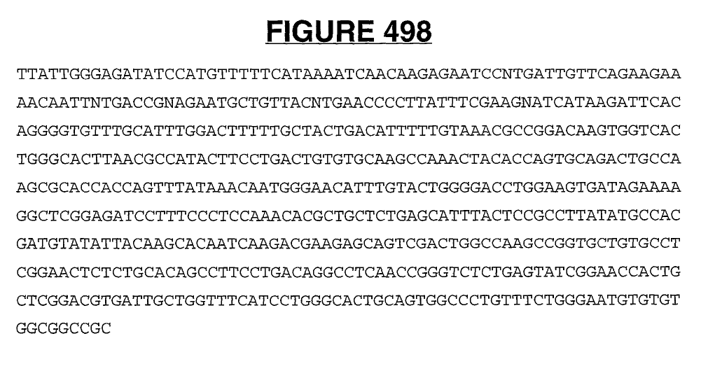

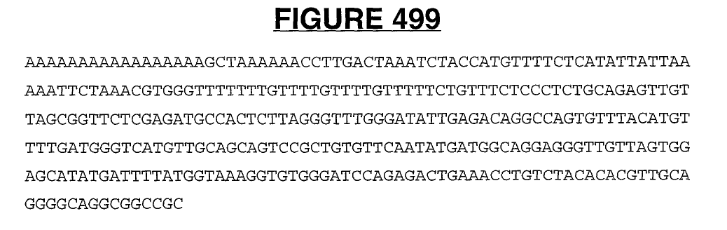

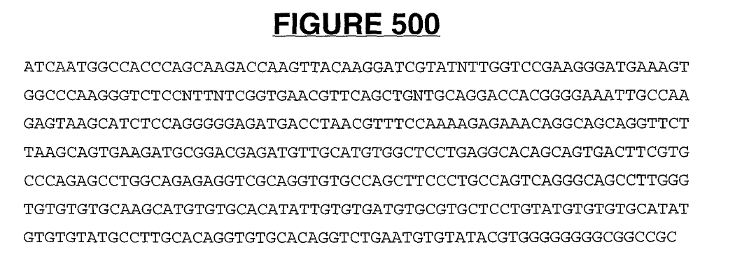

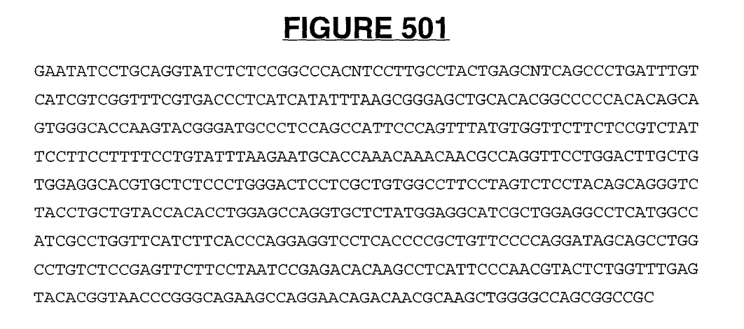

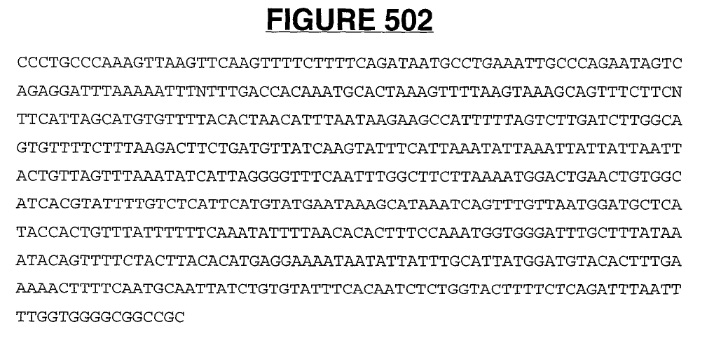

FIGS. 163-166, 174-175, 332-333, 422-430 and 494-502.

(s) Human fetal kidney tissue

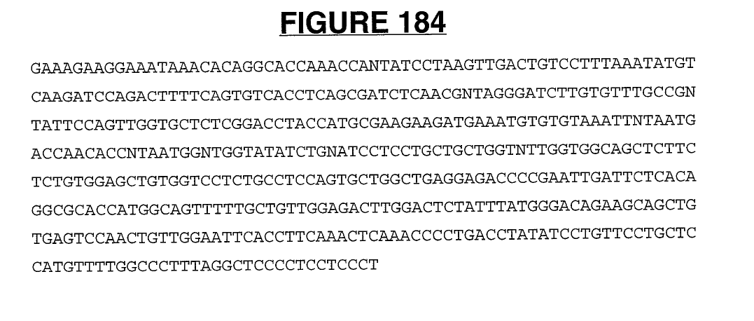

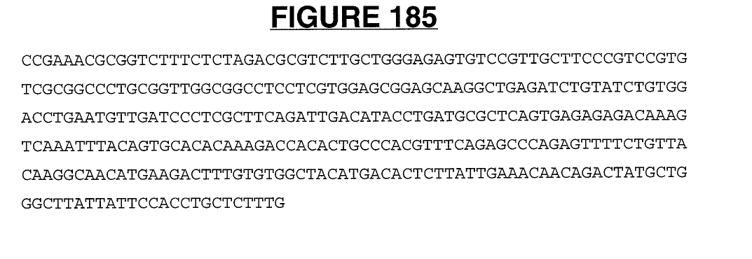

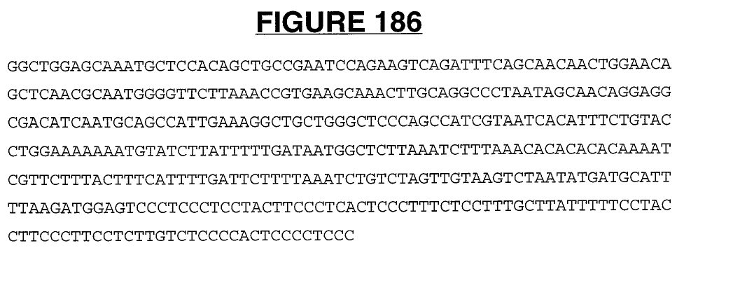

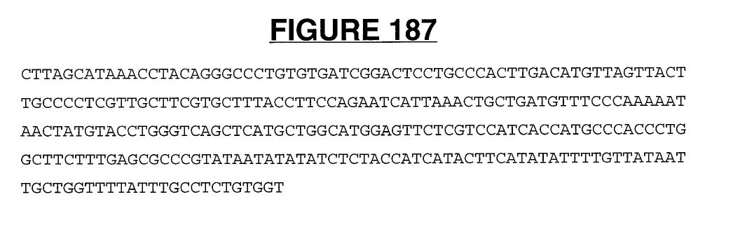

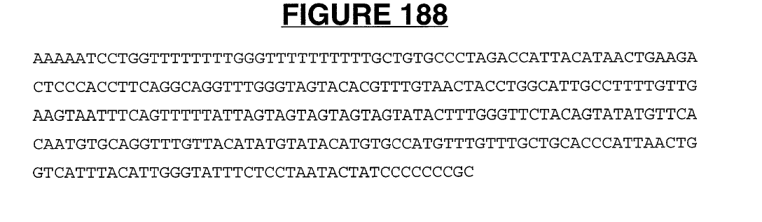

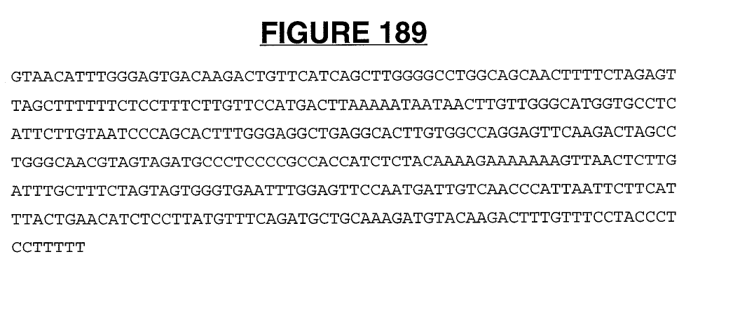

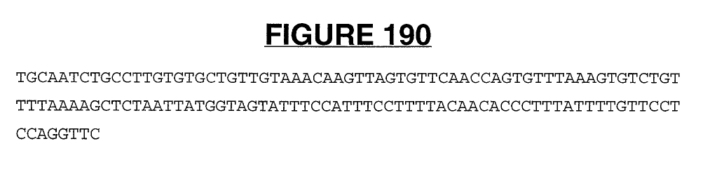

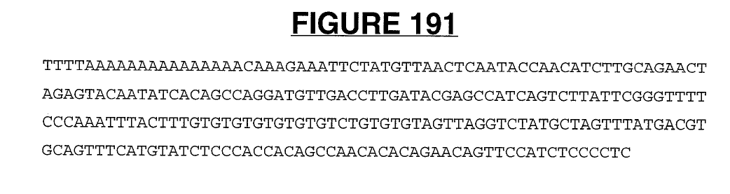

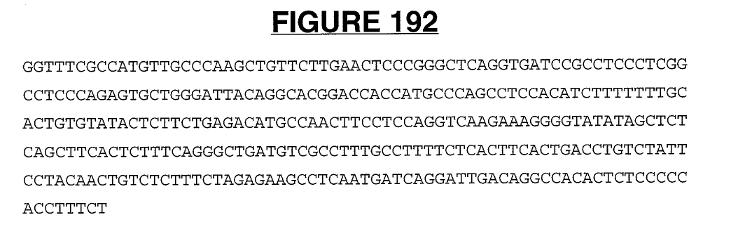

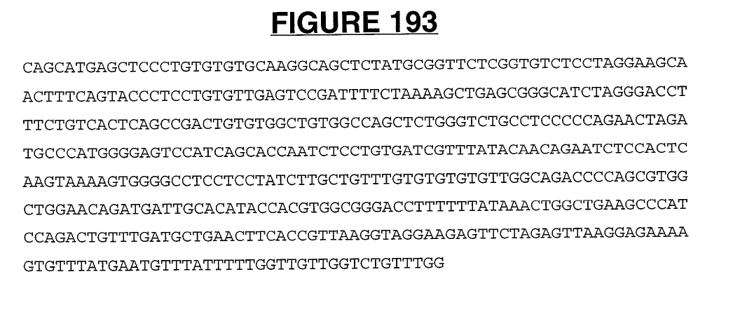

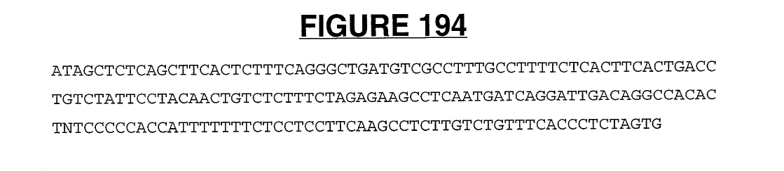

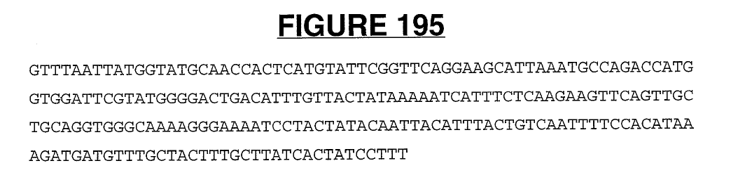

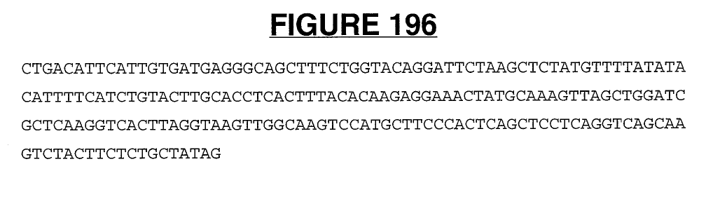

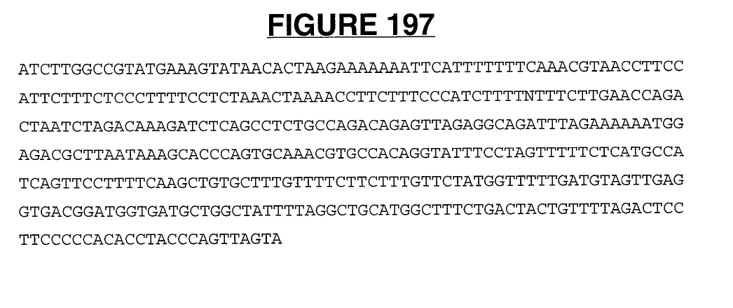

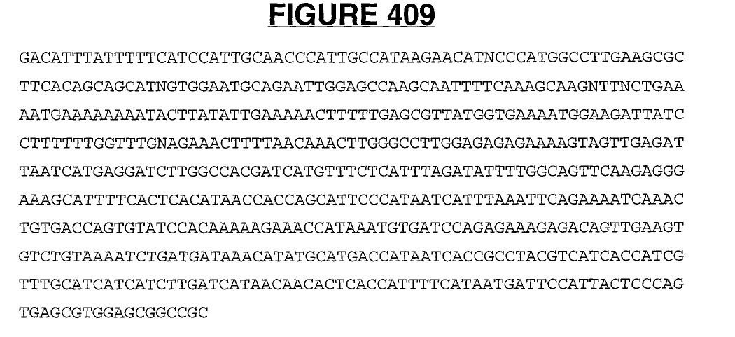

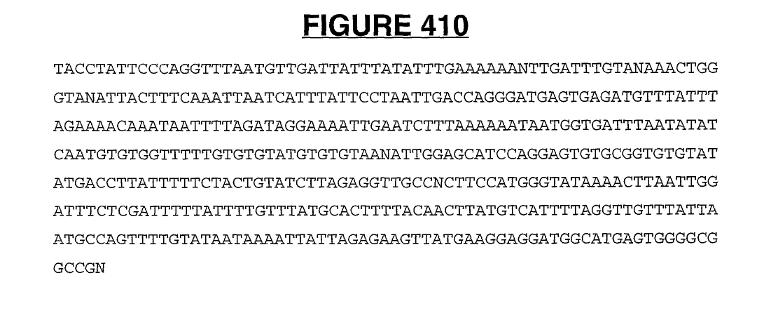

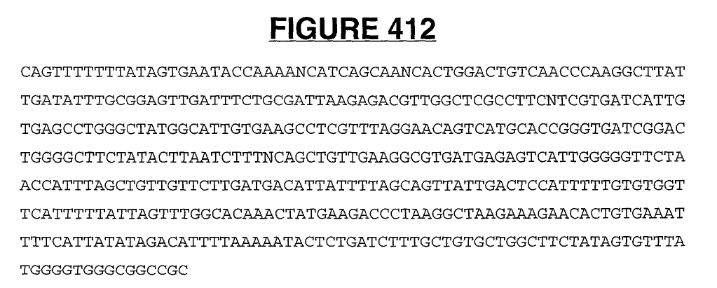

FIGS. 184-197, 409-410 and 412.

(t) Human prostate tissue

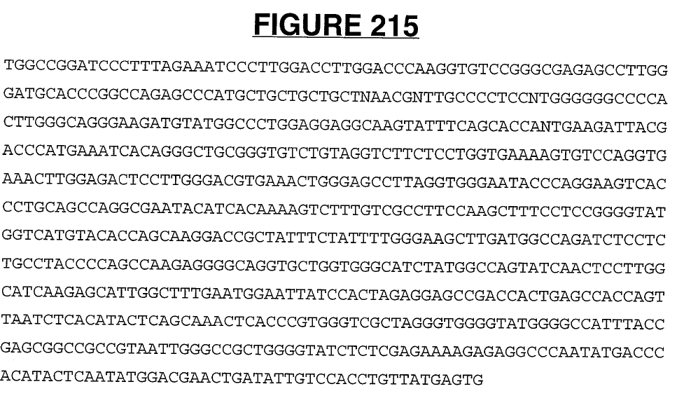

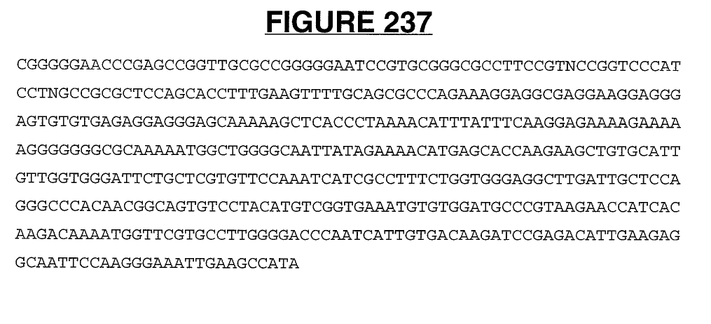

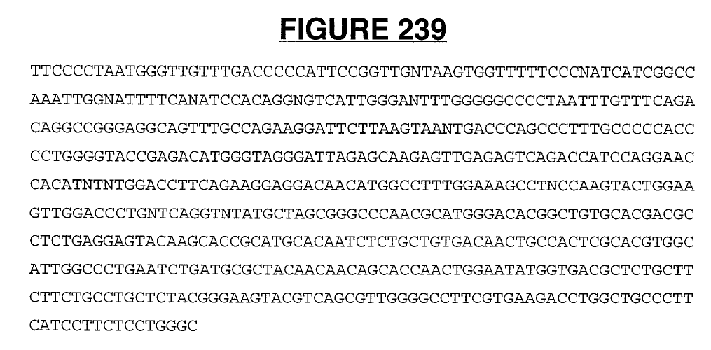

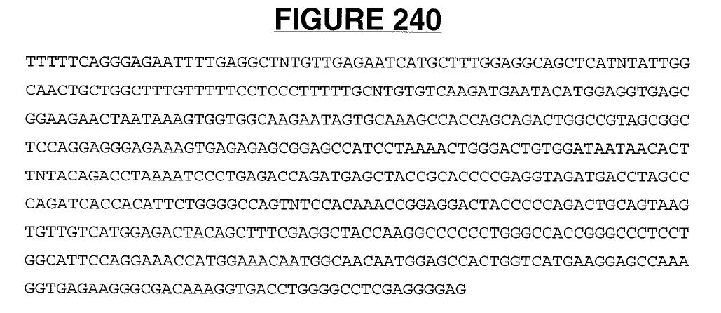

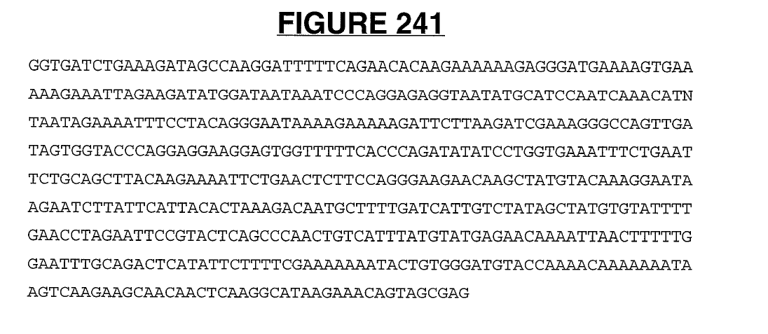

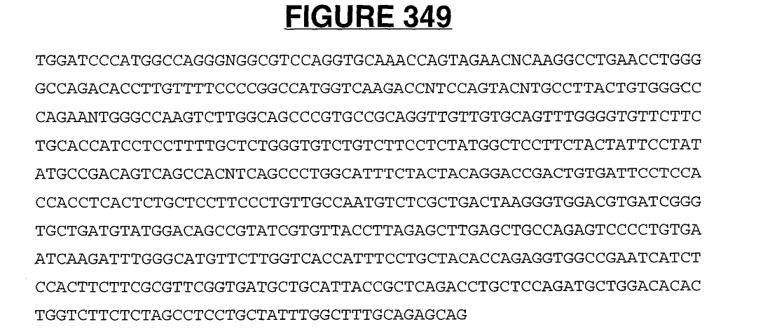

FIGS. 215, 237, 239-241 and 349.

(u) Human mammary gland tissue

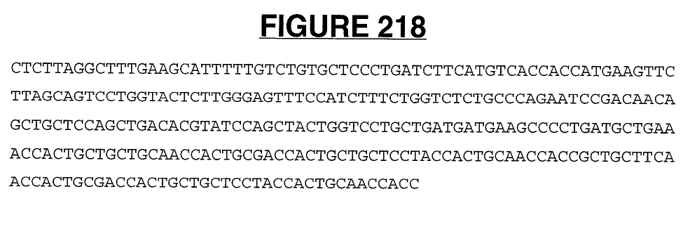

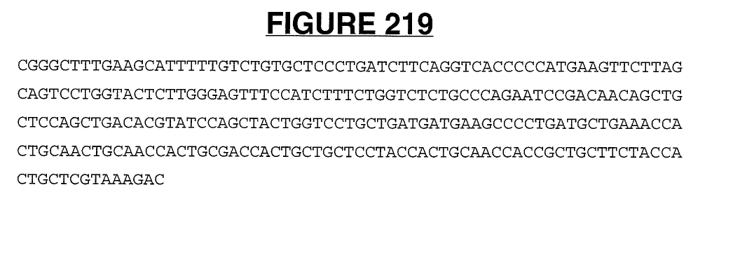

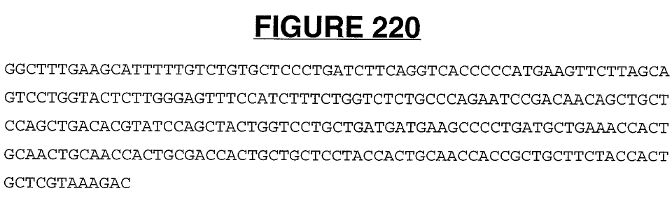

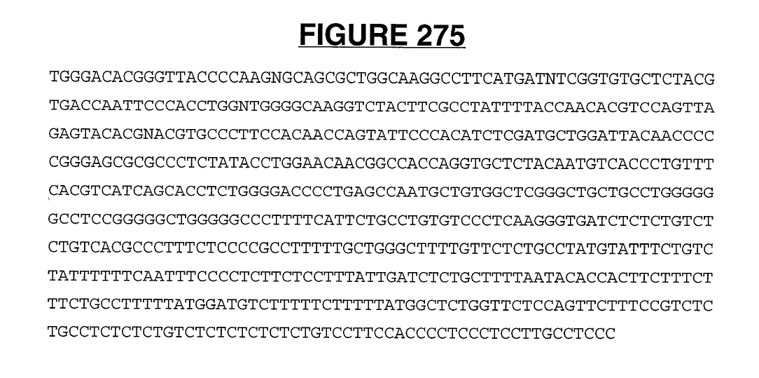

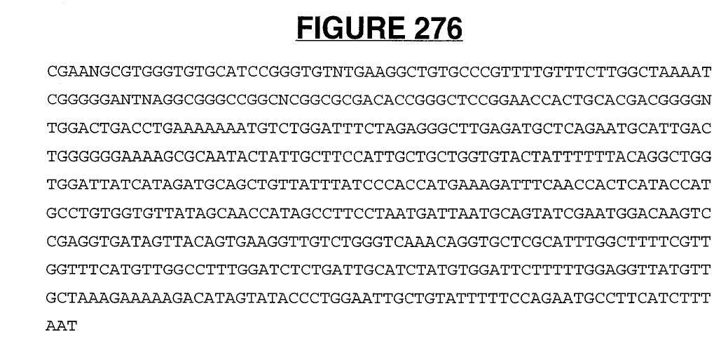

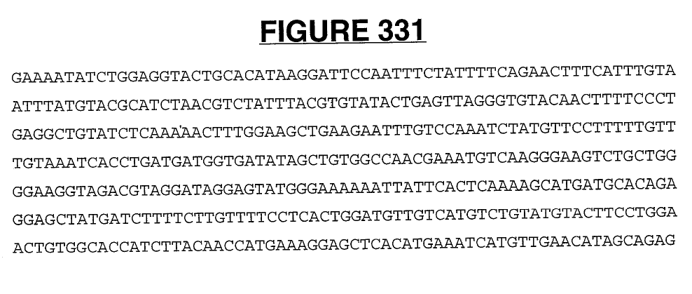

FIGS. 218-220, 275-276 and 331.

(v) Human adenocarcinoma tissue

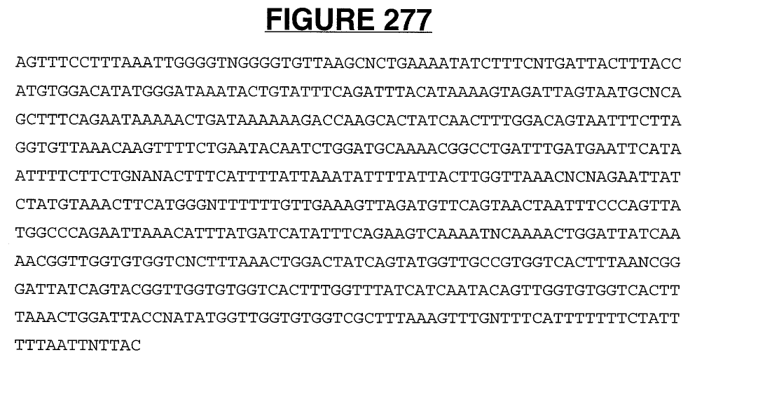

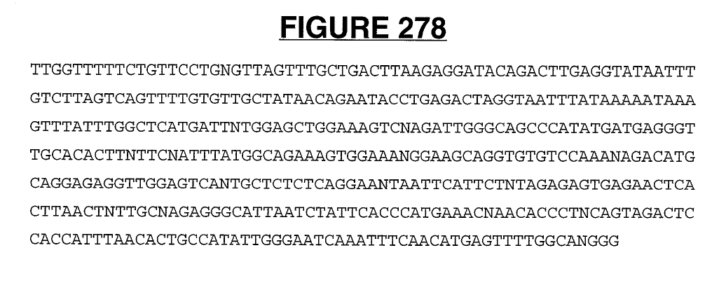

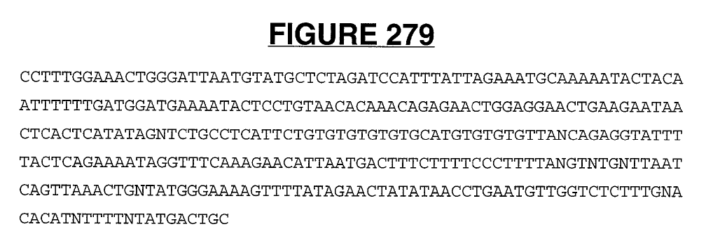

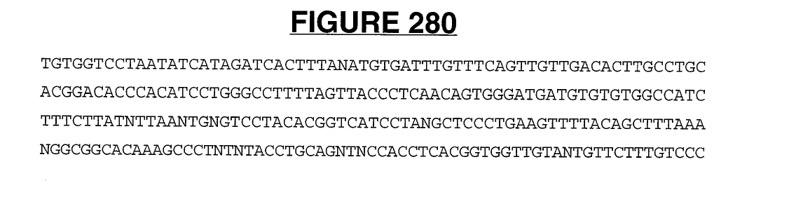

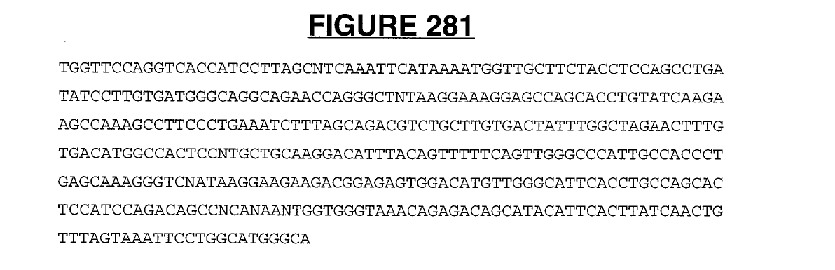

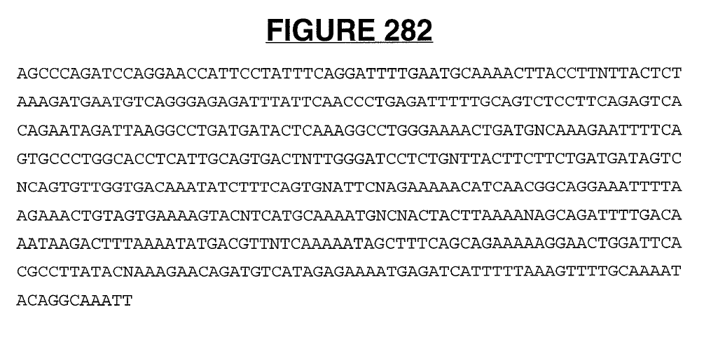

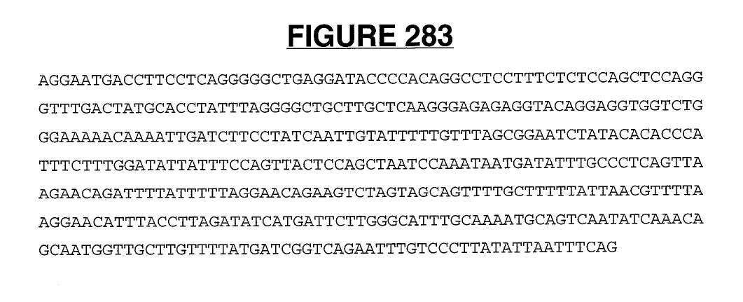

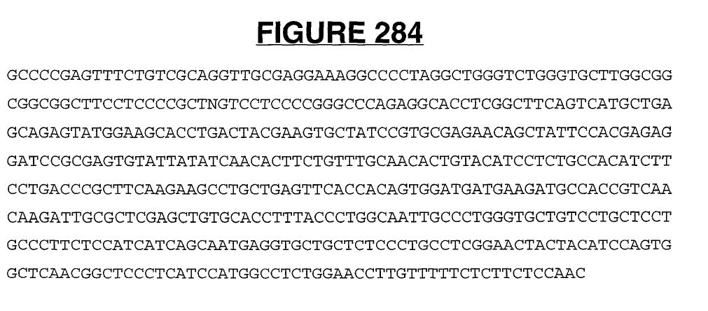

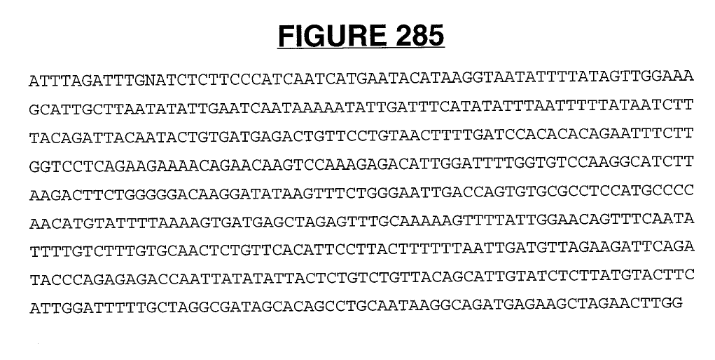

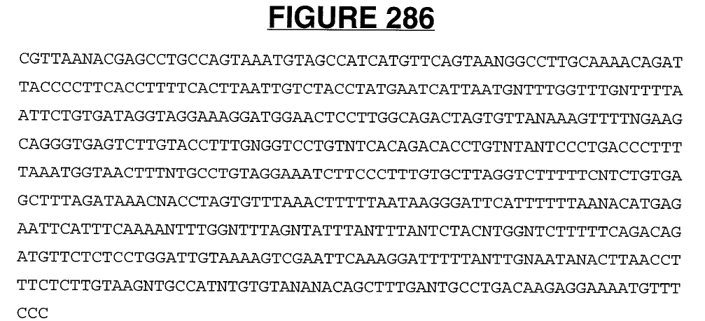

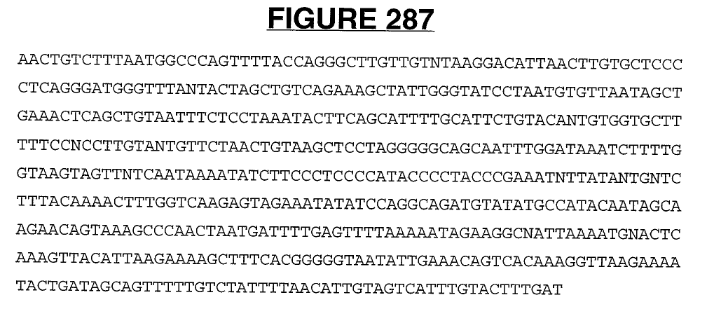

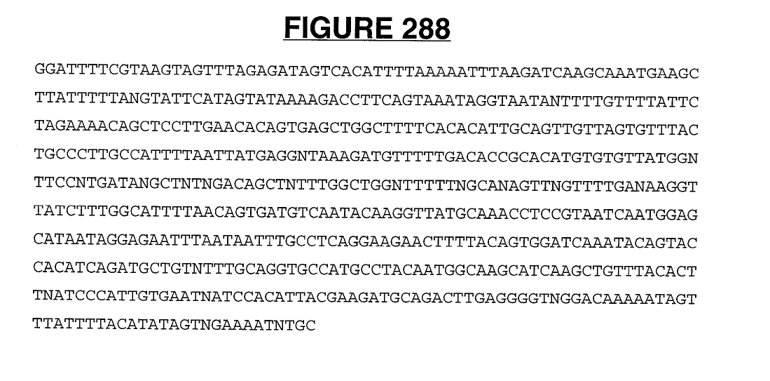

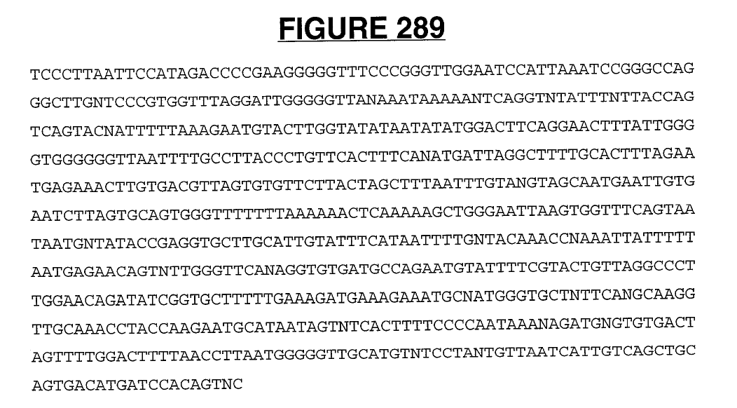

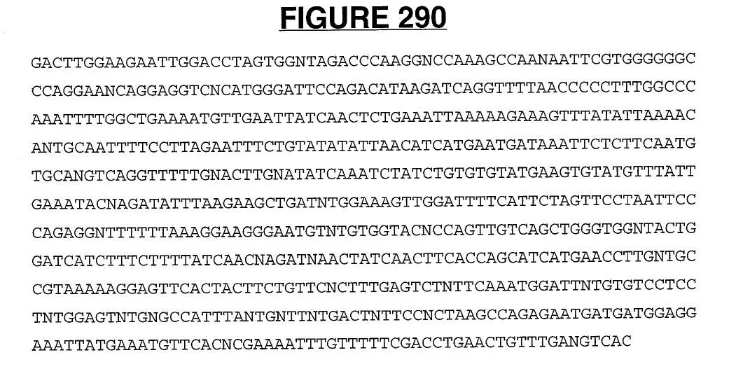

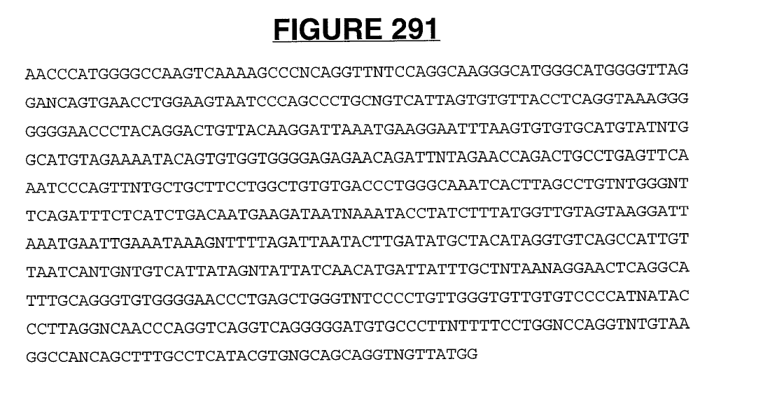

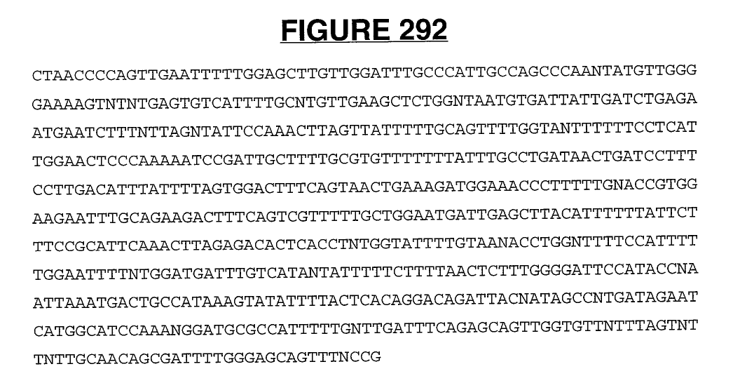

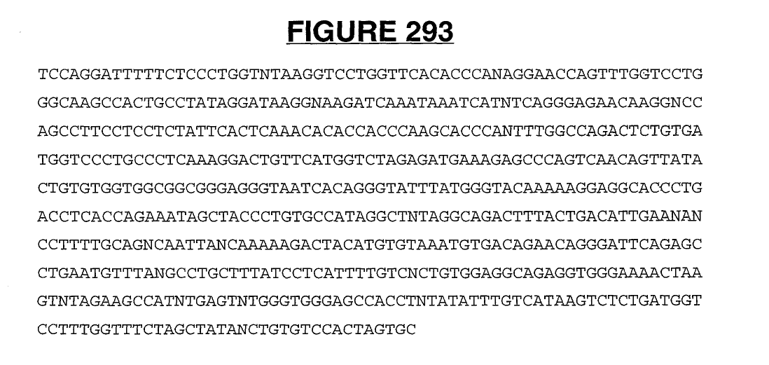

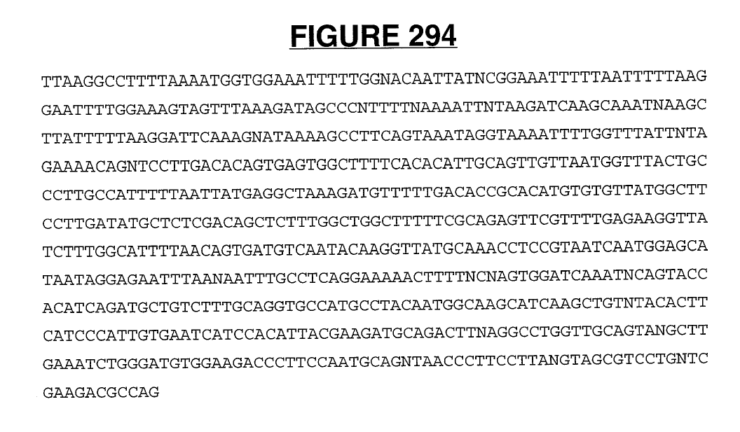

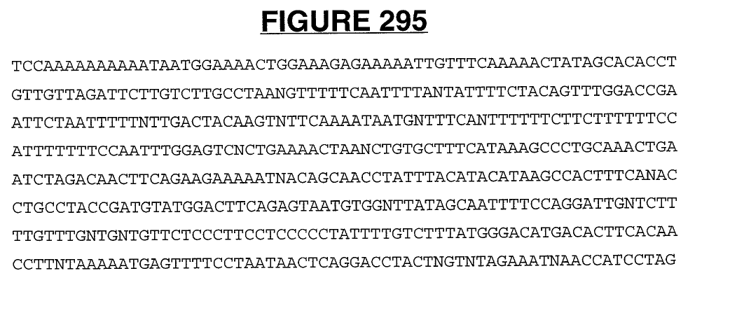

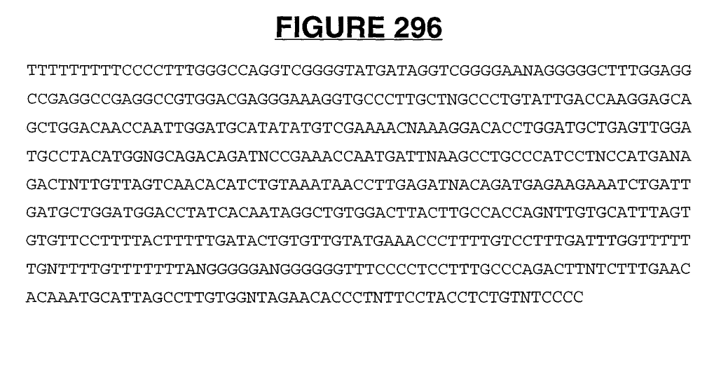

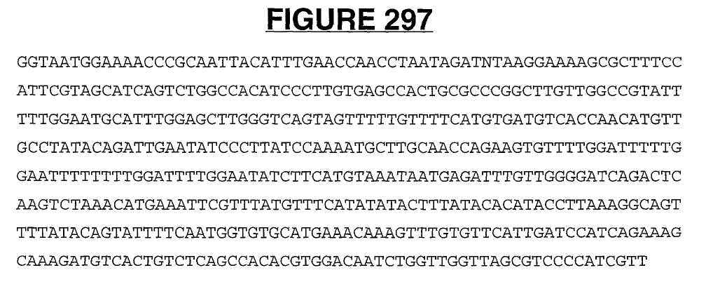

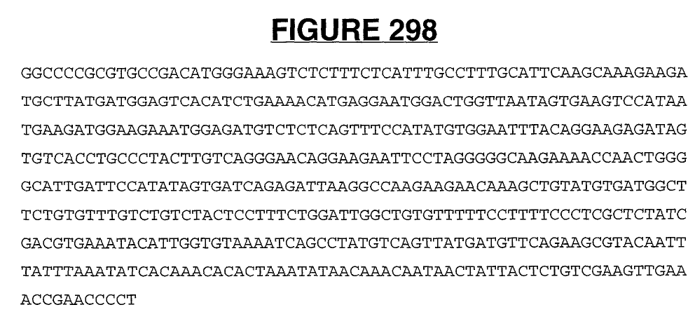

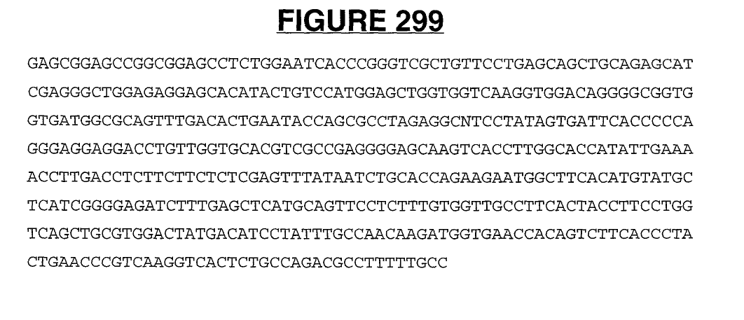

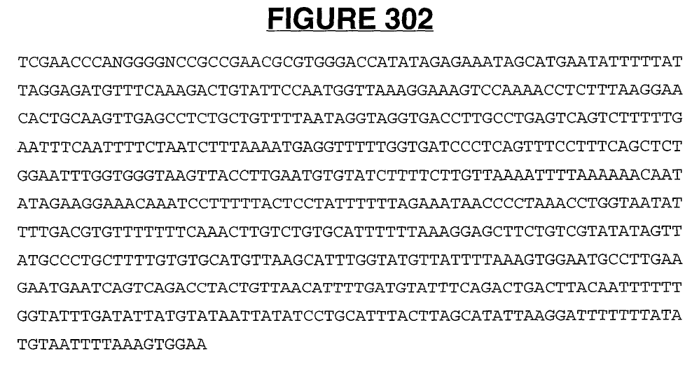

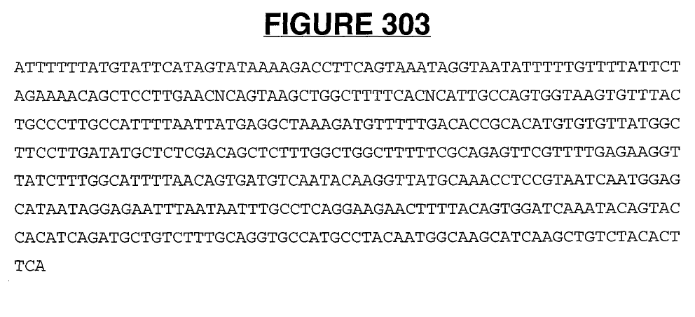

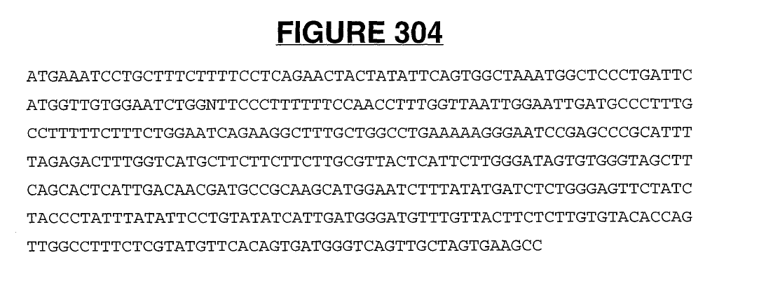

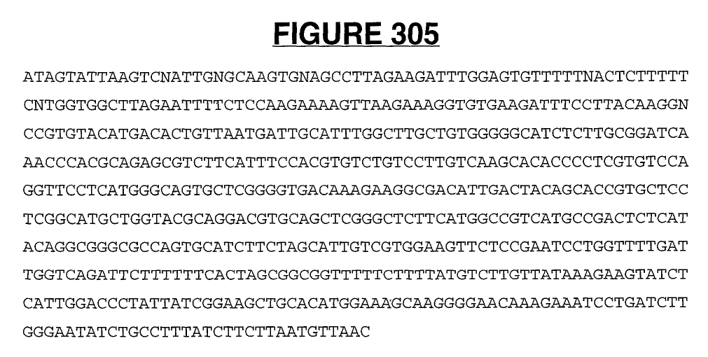

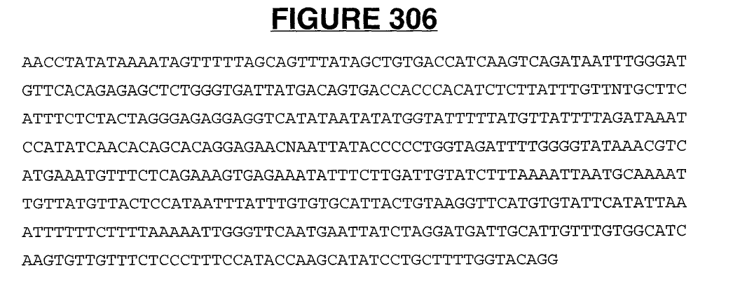

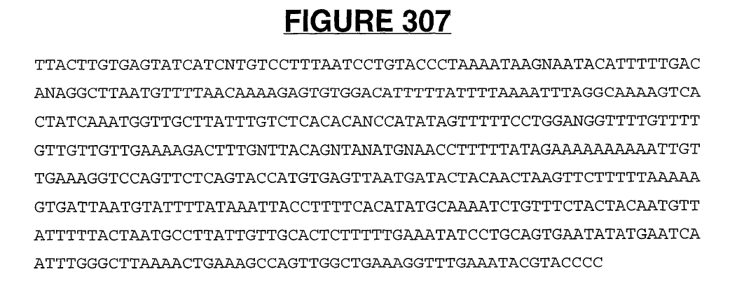

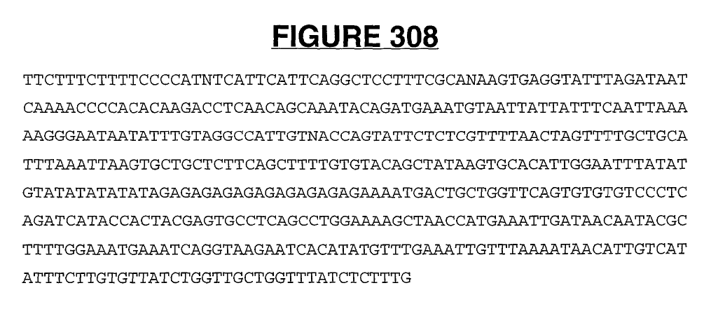

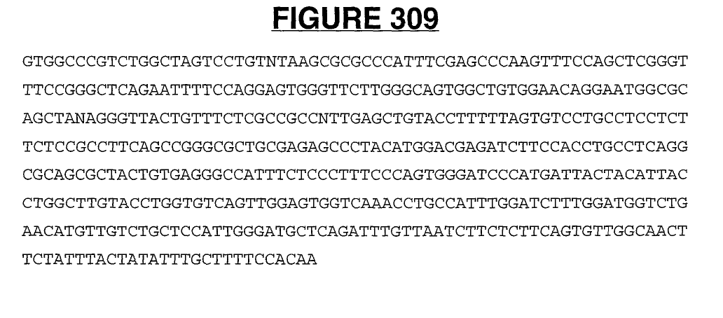

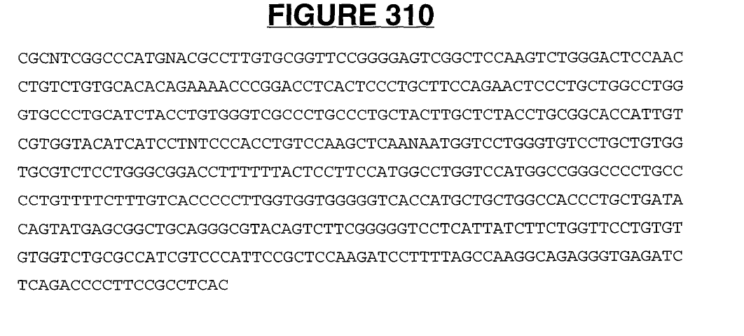

FIGS. 277-299 and 302-310.

(w) Human fetal small intestine tissue

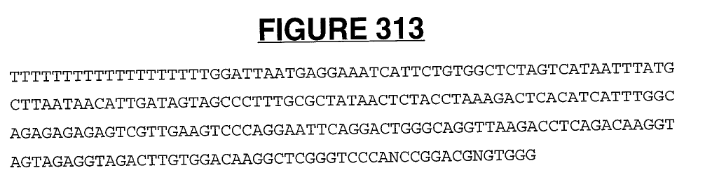

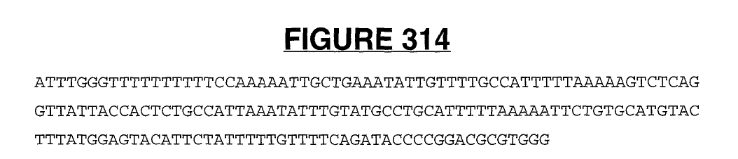

FIGS. 313-314.

(x) Human fetal lung tissue

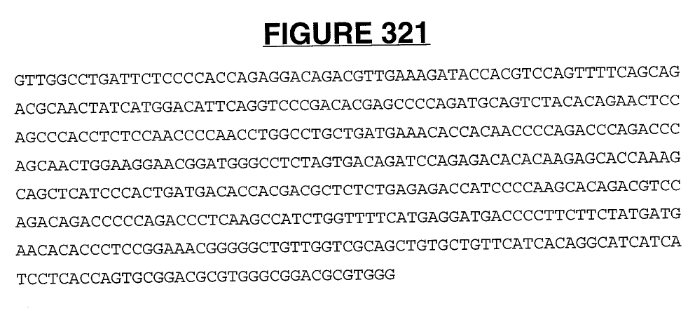

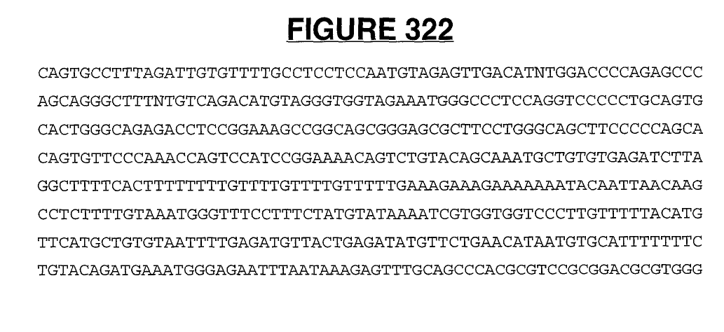

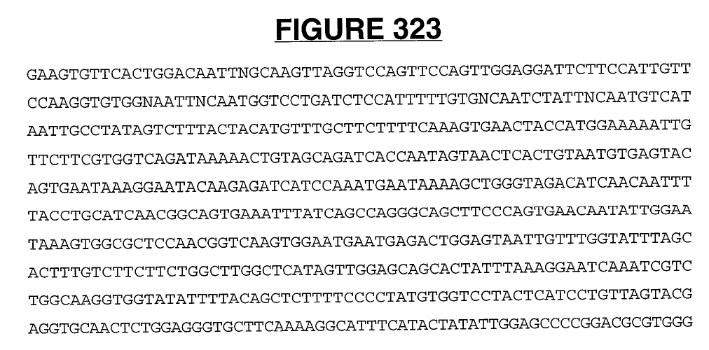

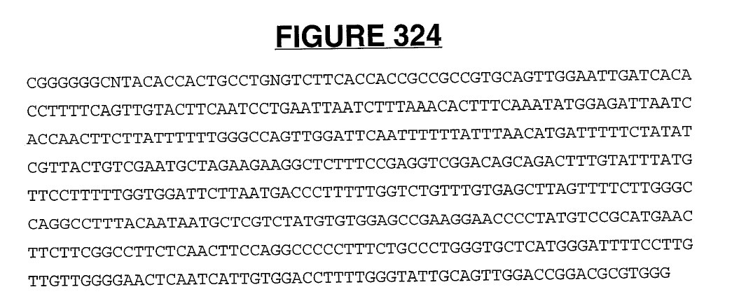

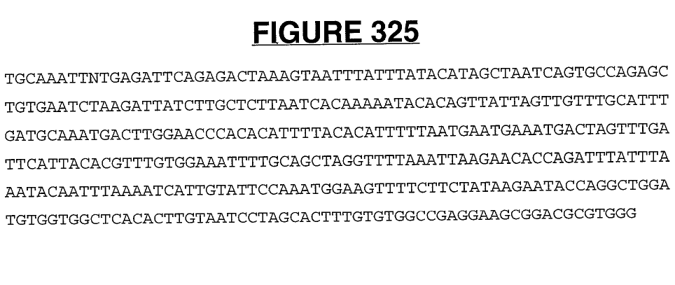

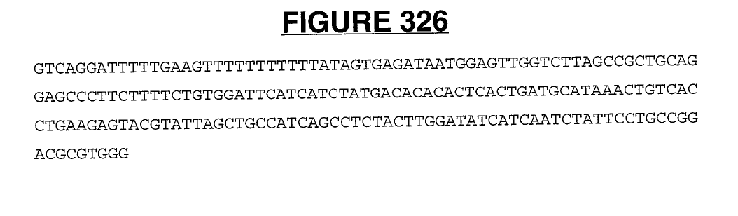

FIGS. 321-326.

(y) Human testis tissue

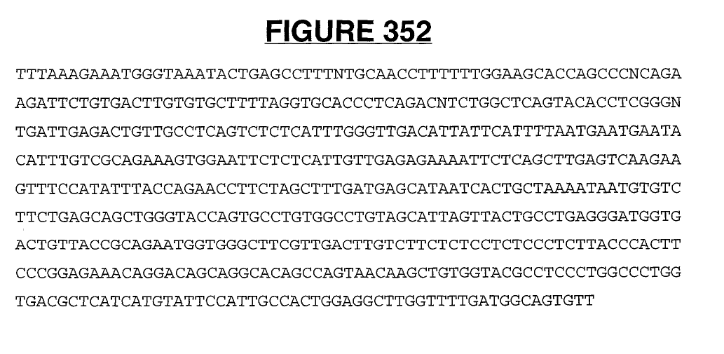

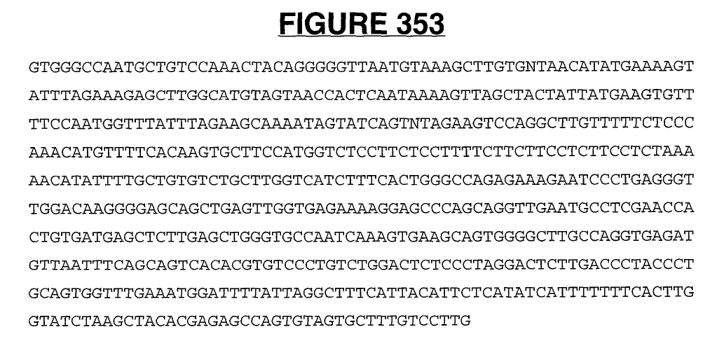

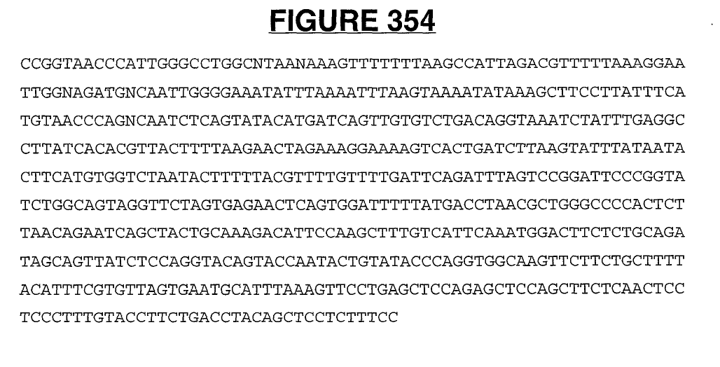

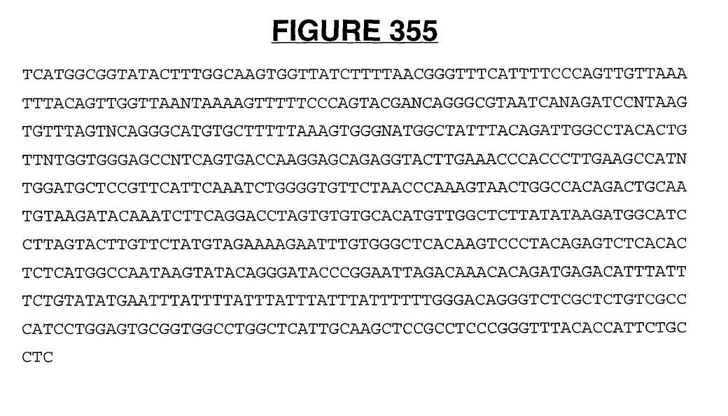

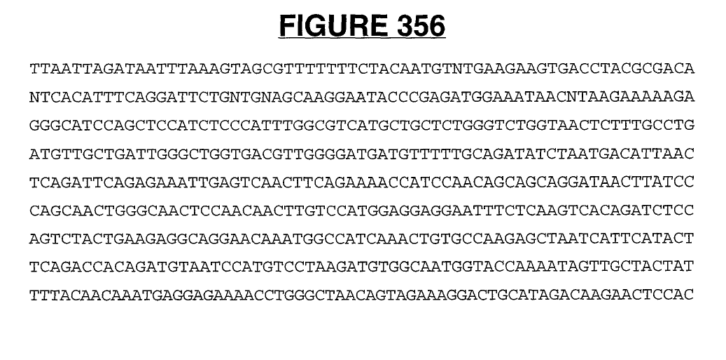

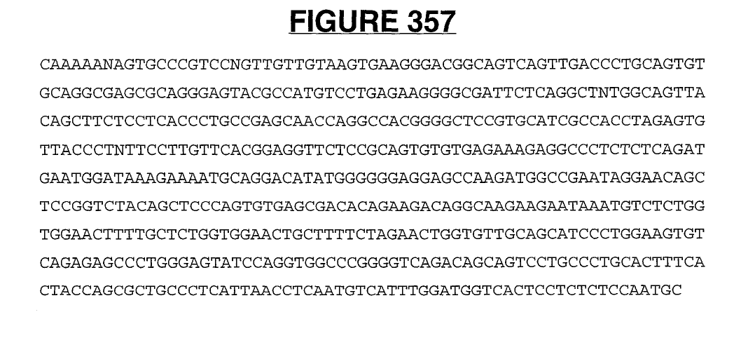

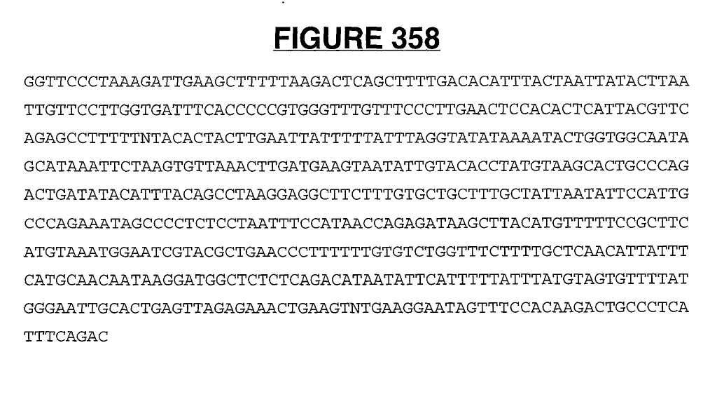

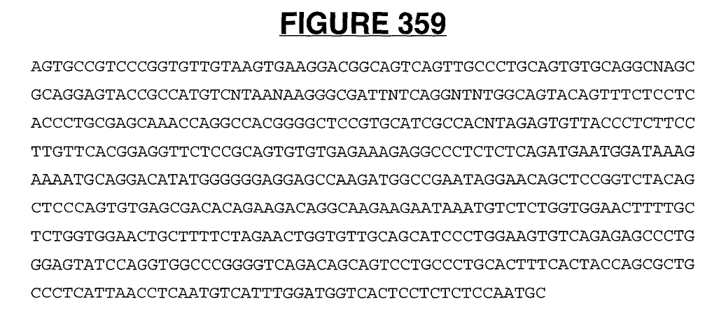

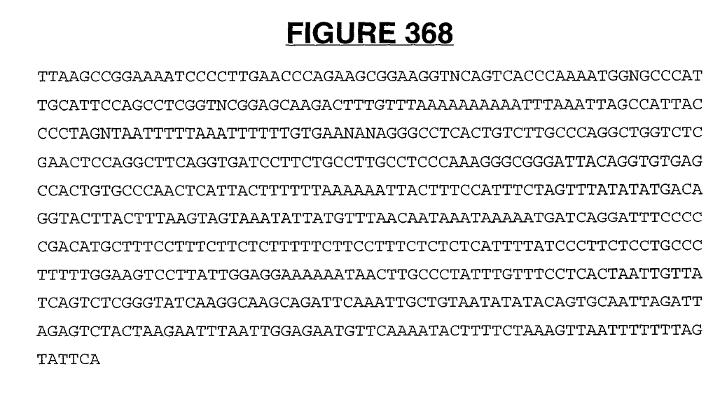

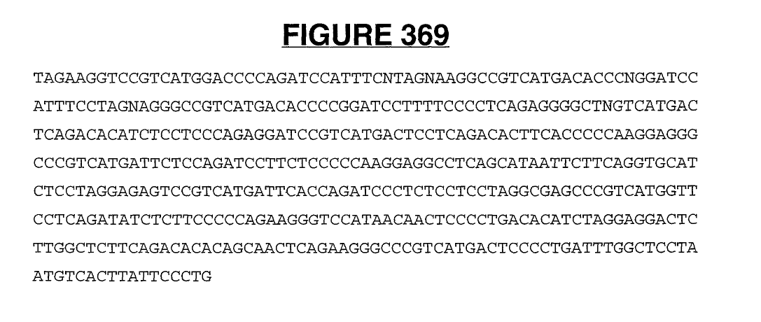

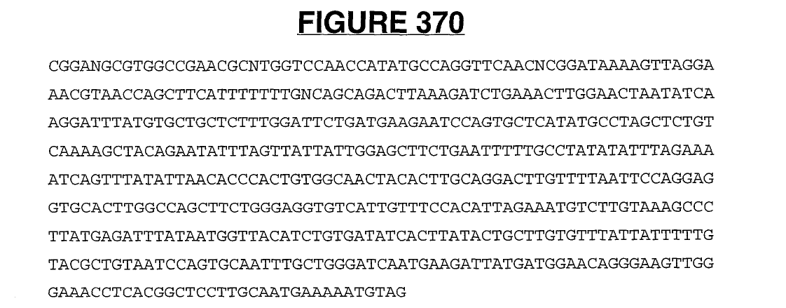

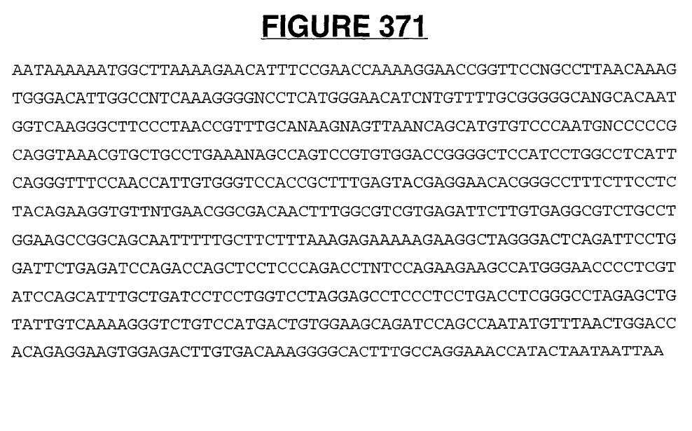

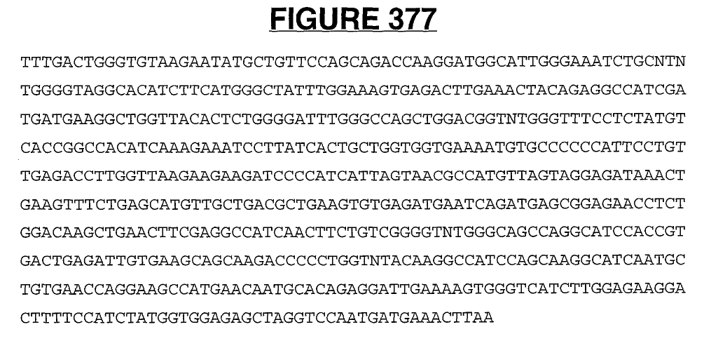

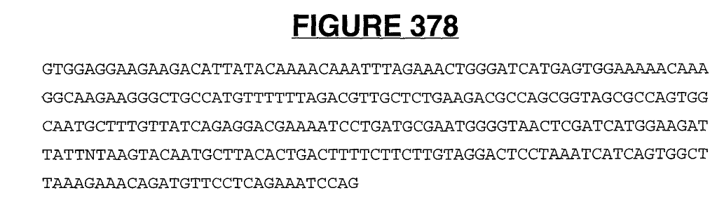

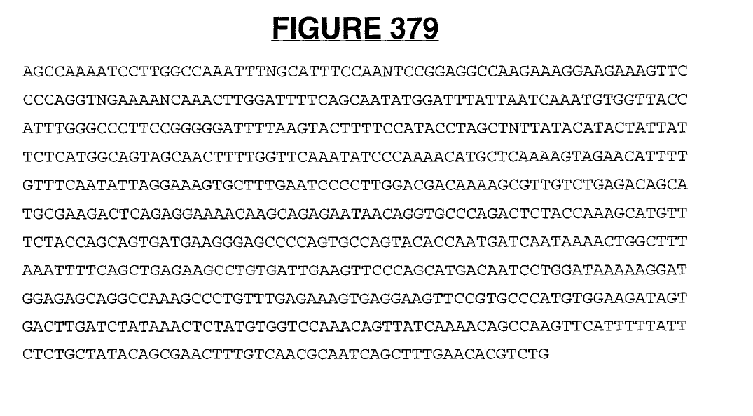

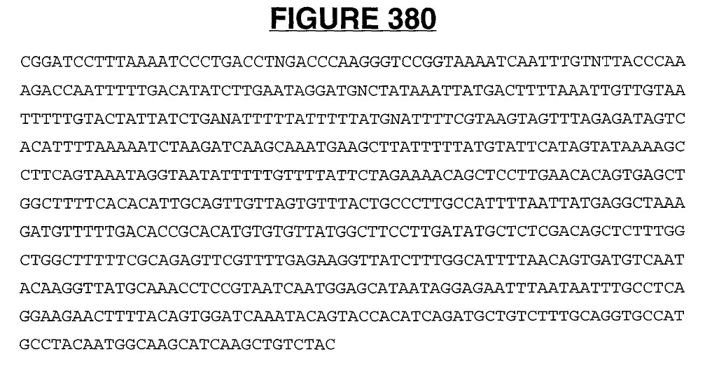

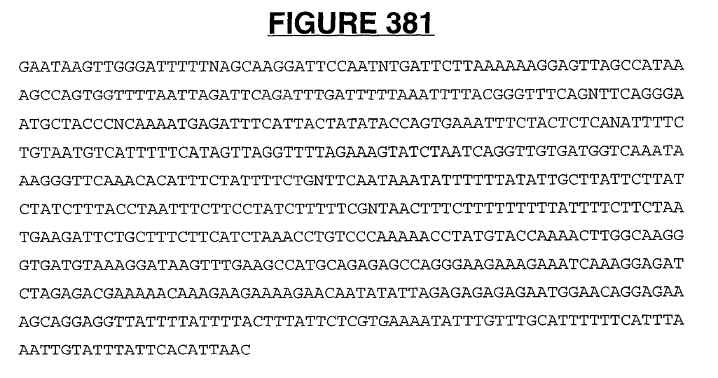

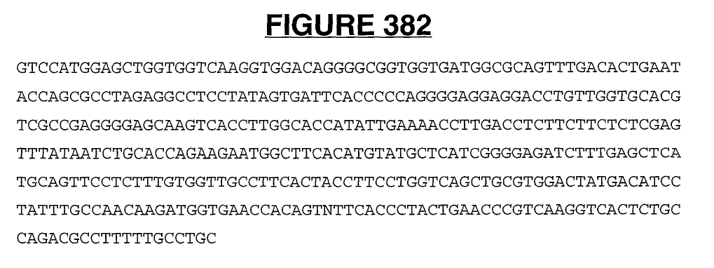

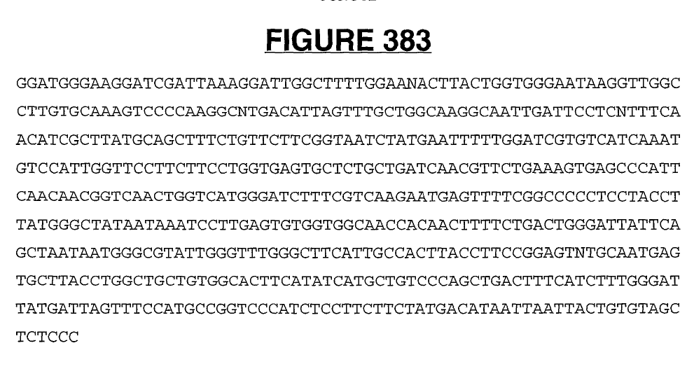

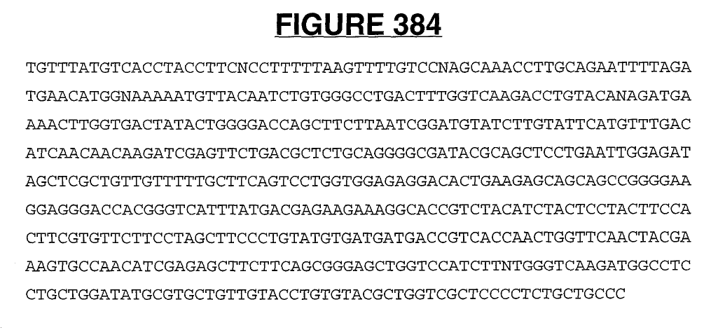

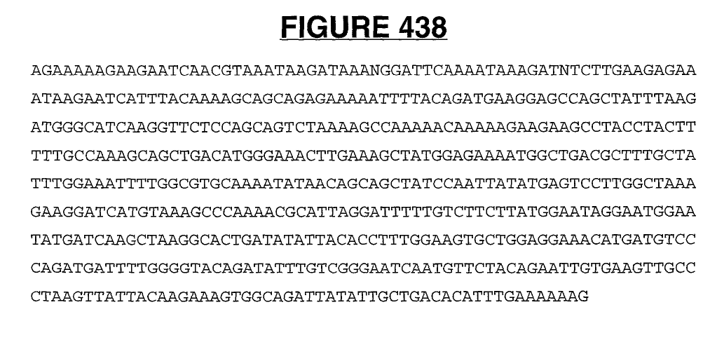

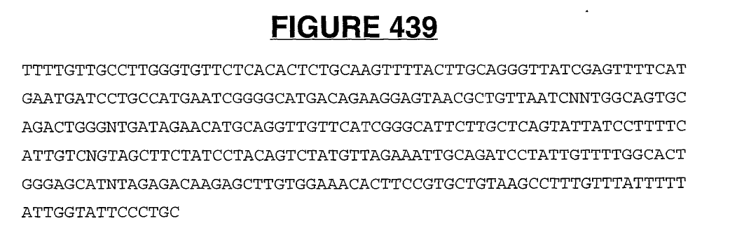

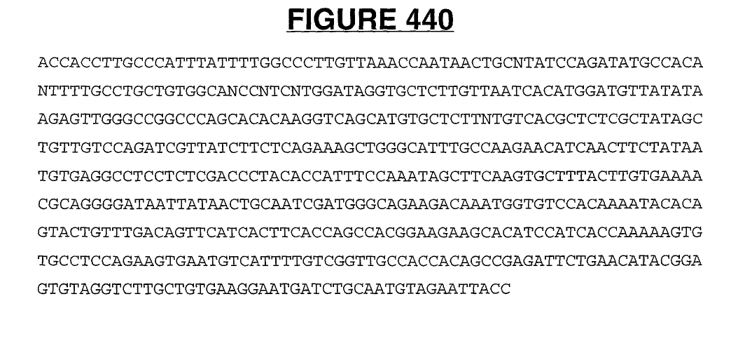

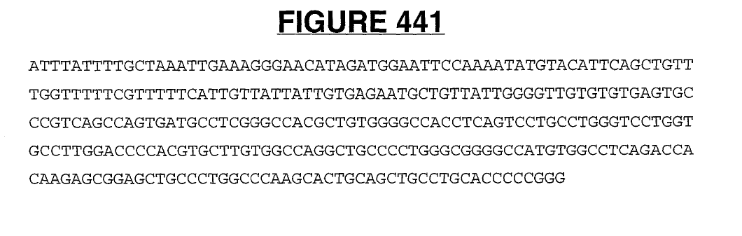

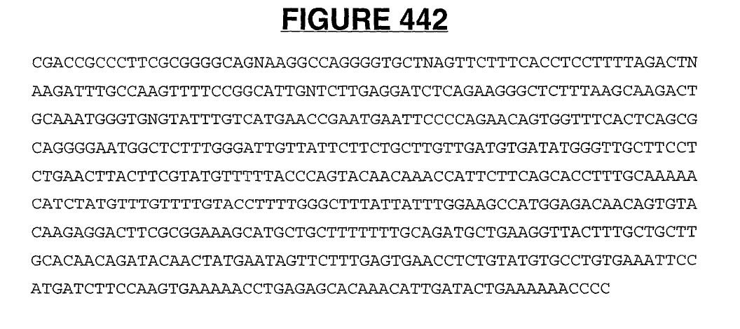

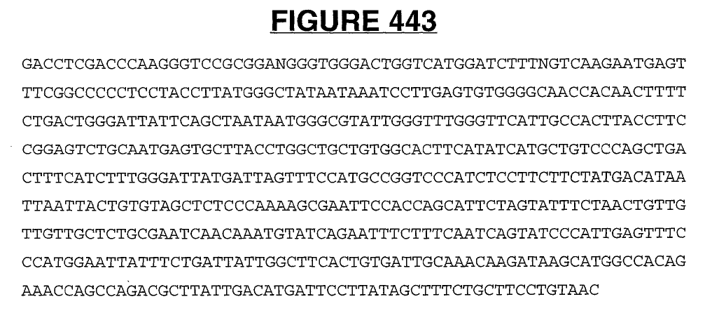

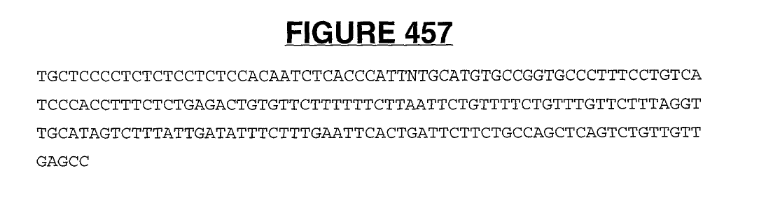

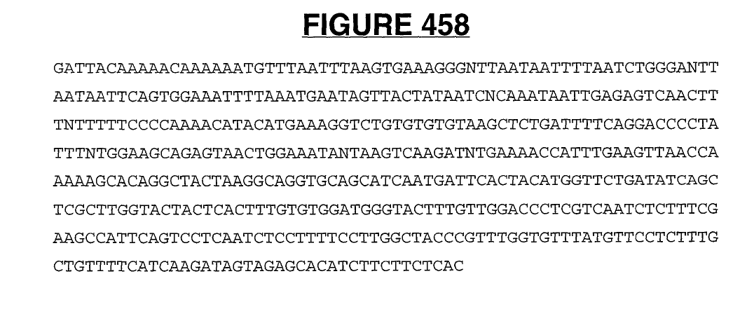

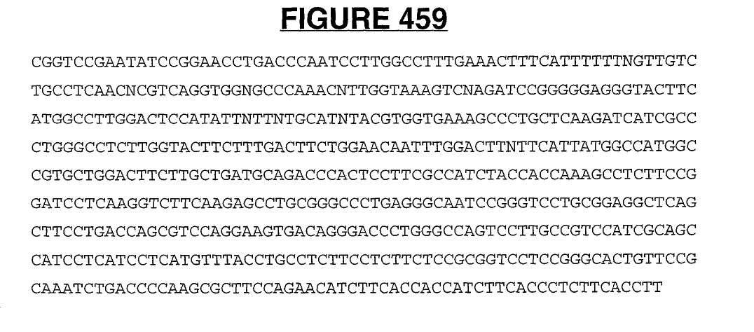

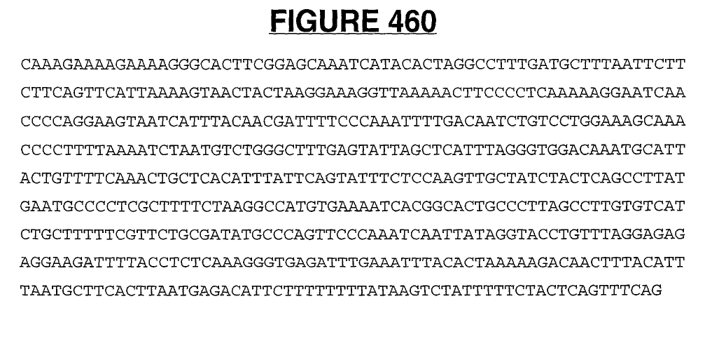

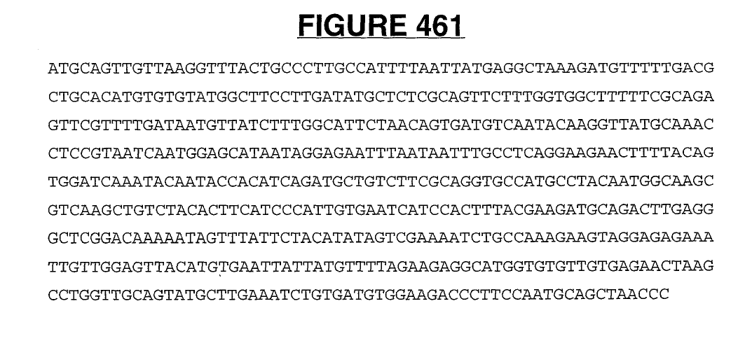

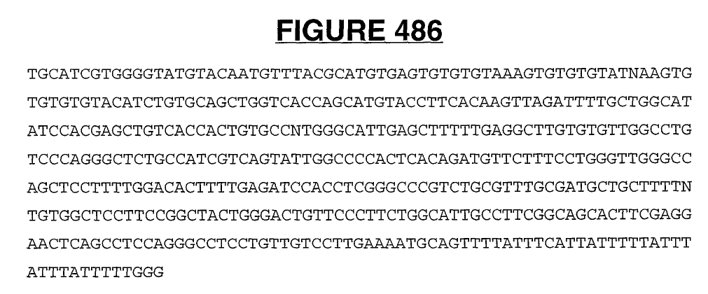

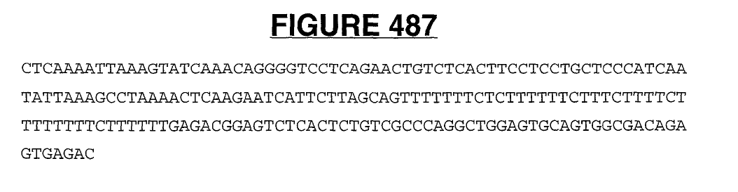

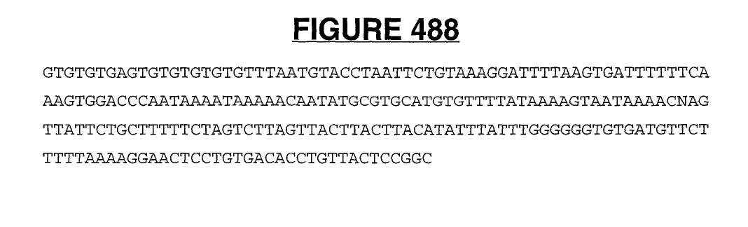

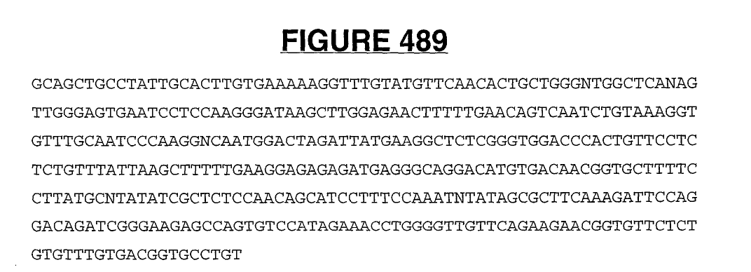

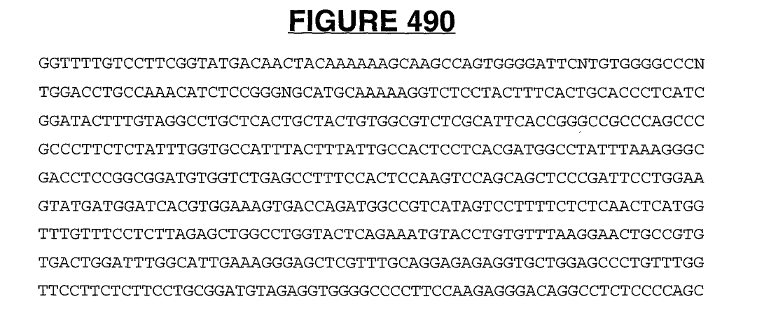

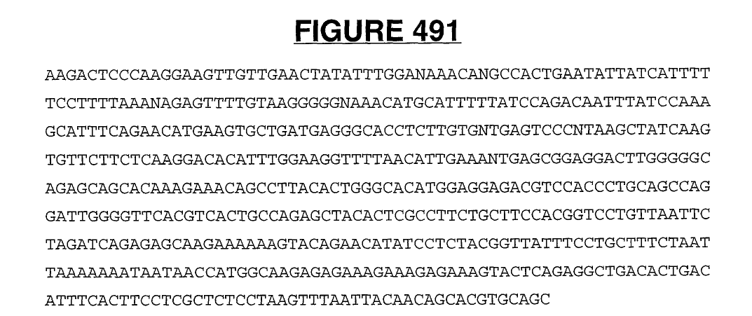

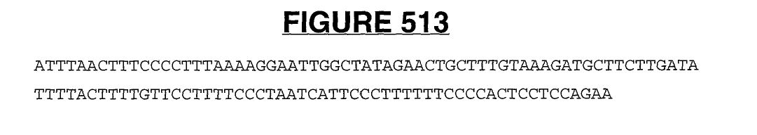

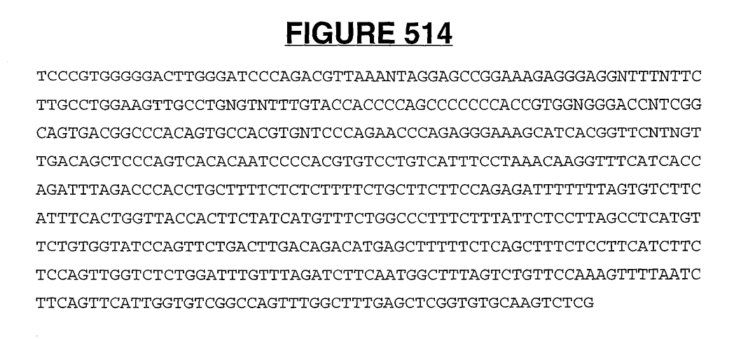

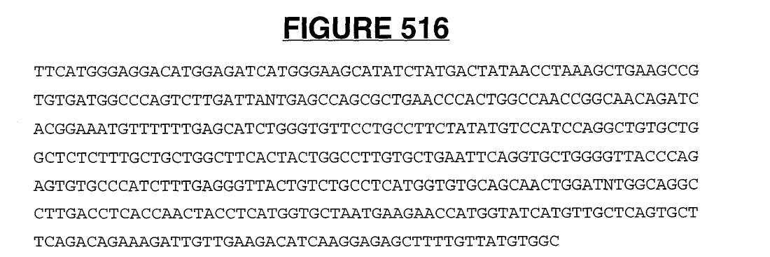

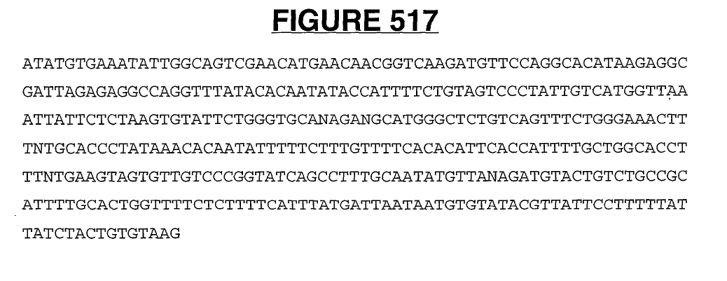

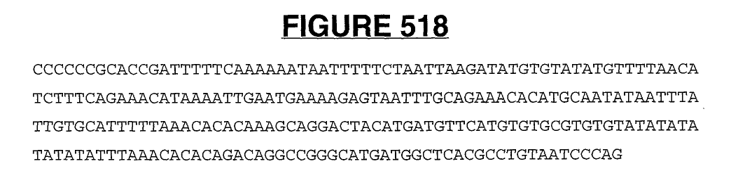

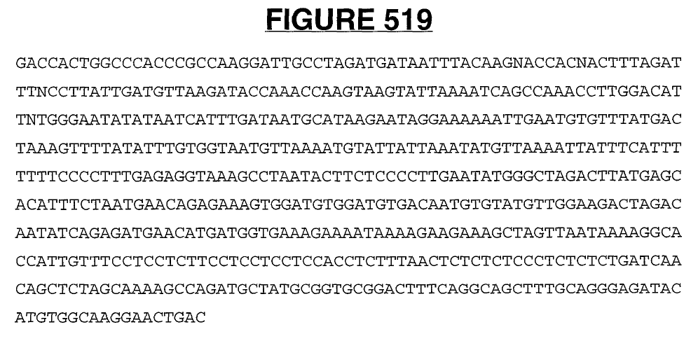

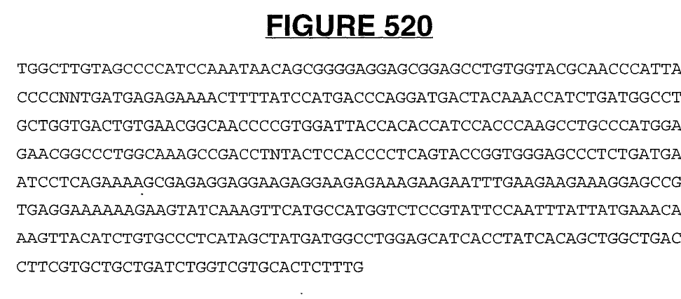

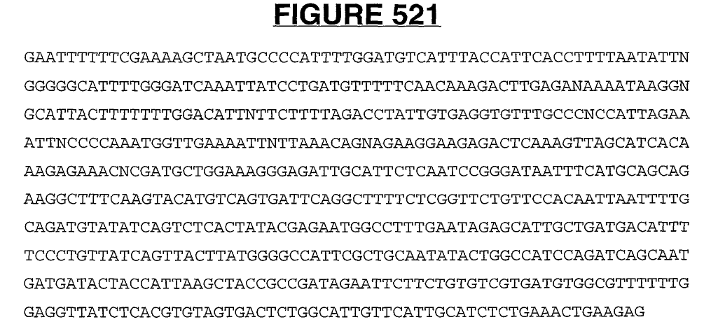

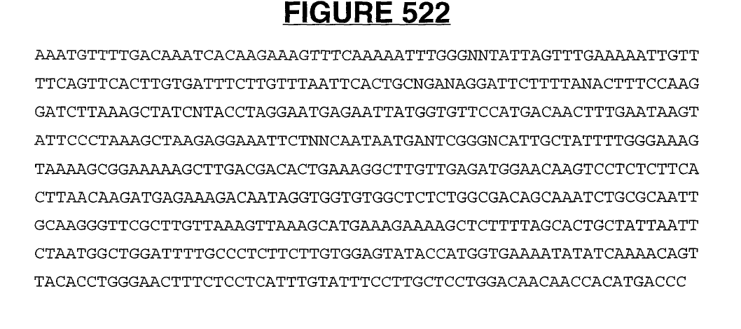

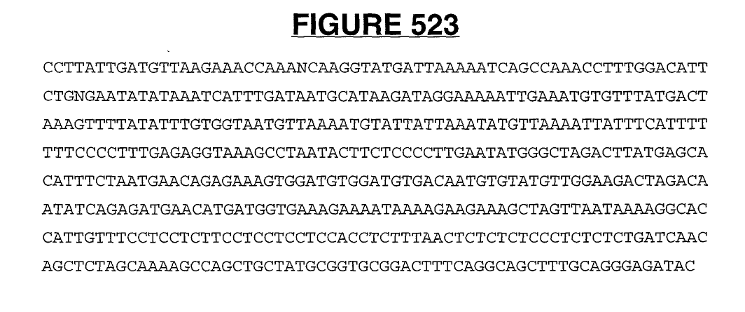

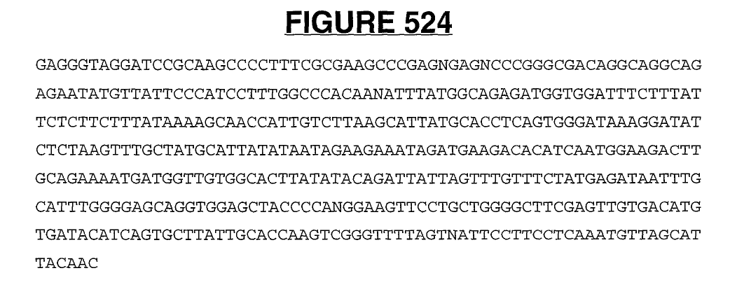

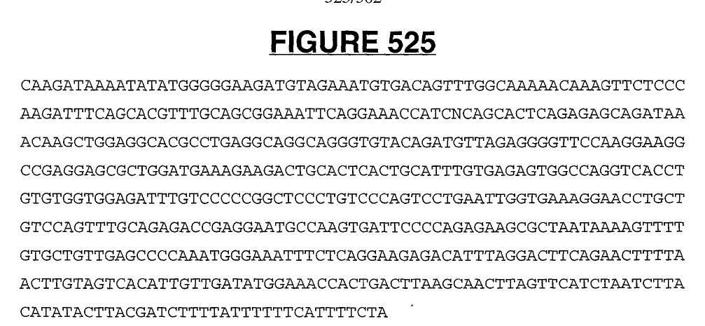

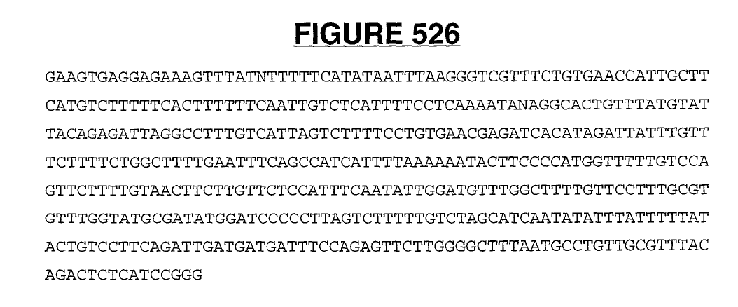

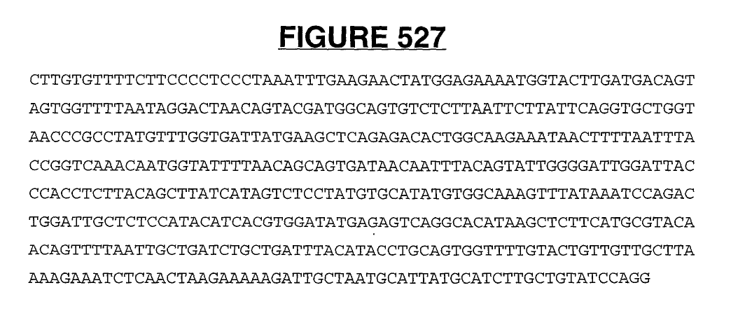

FIGS. 352-359, 368-371, 377-384, 438-443, 457-461, 486-491, 513-514, 516-527 and 562.

(z) Human MCF-7 cells

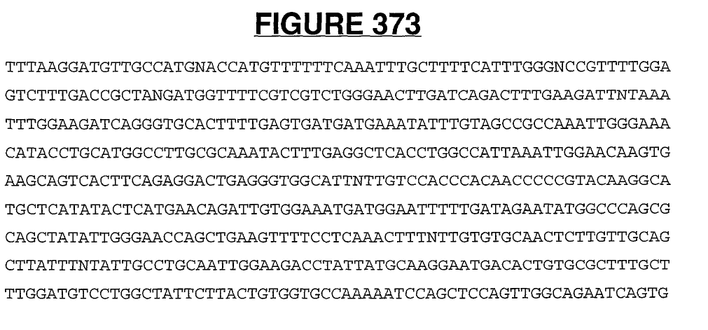

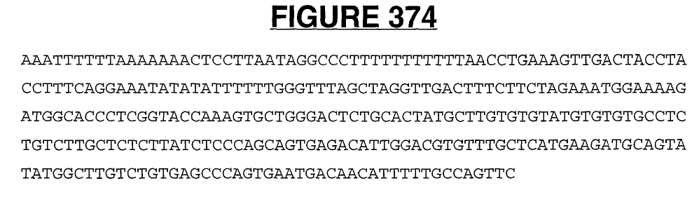

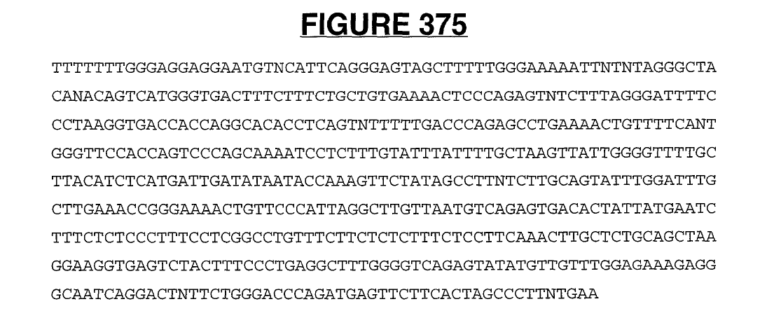

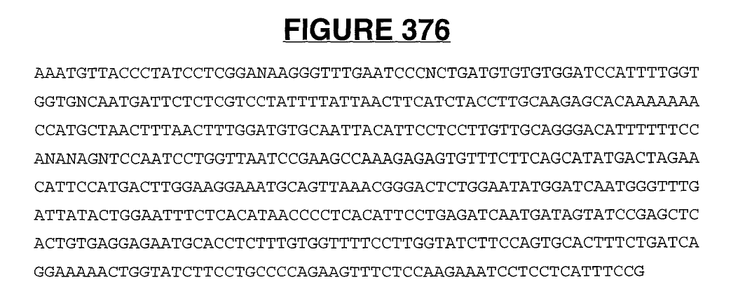

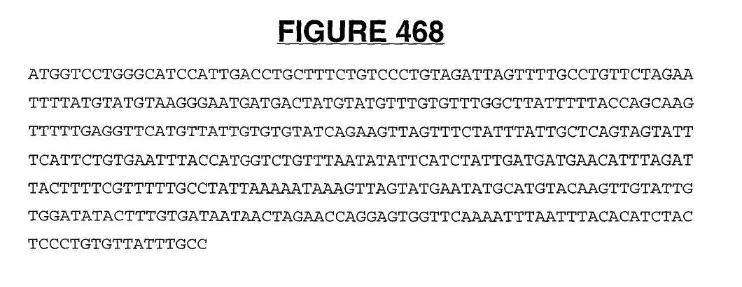

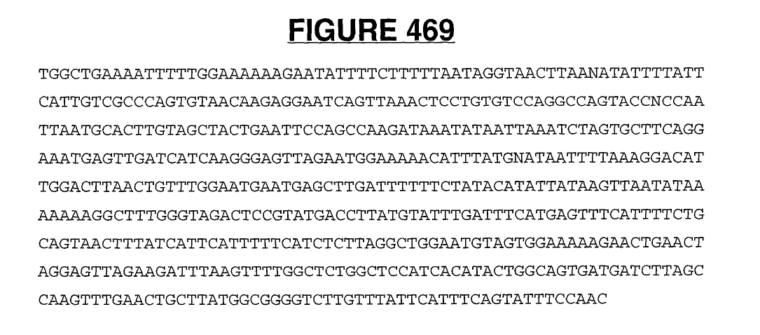

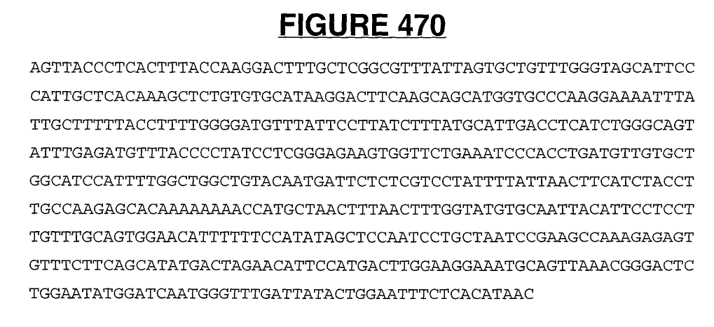

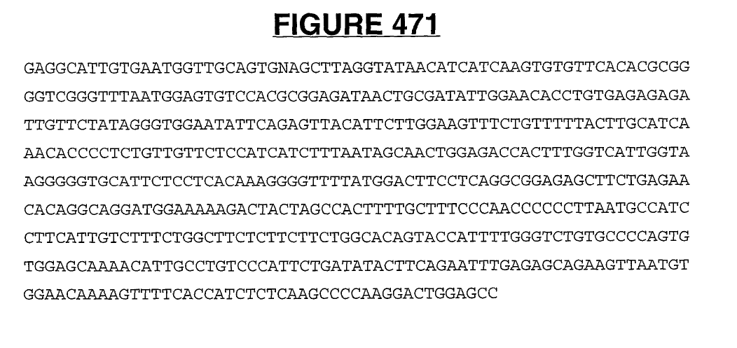

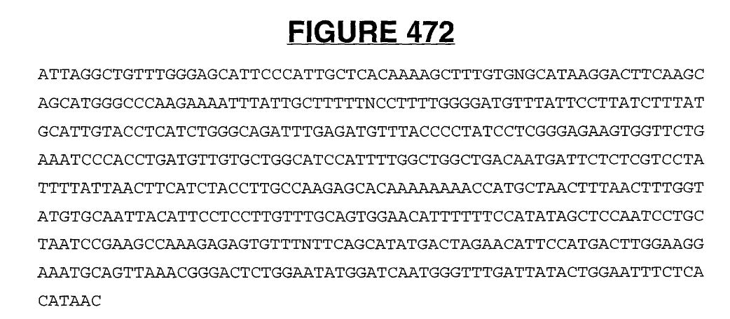

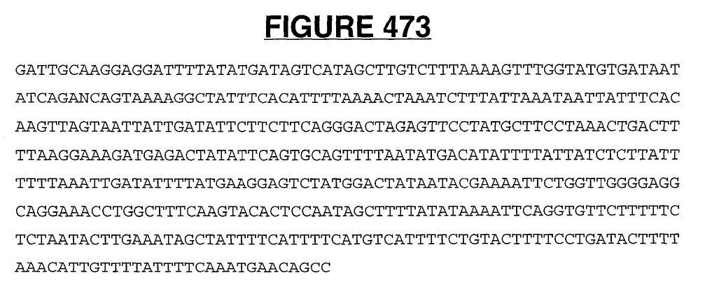

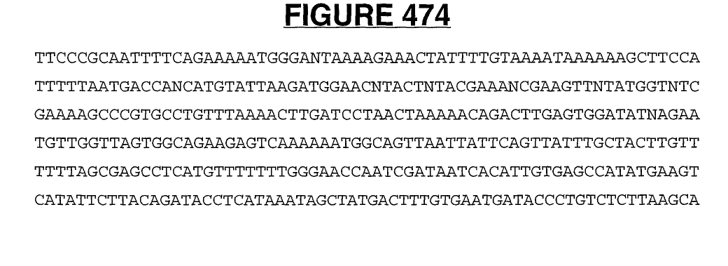

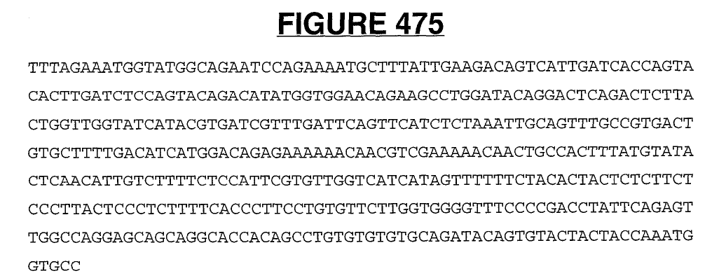

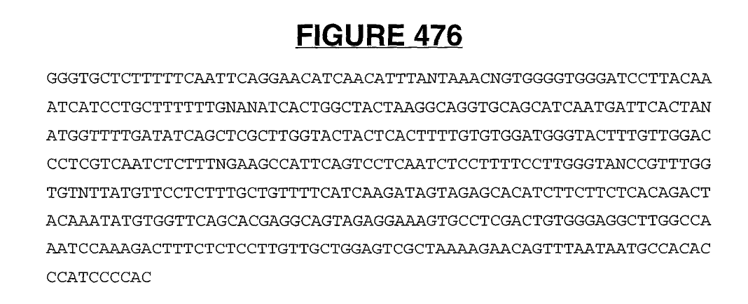

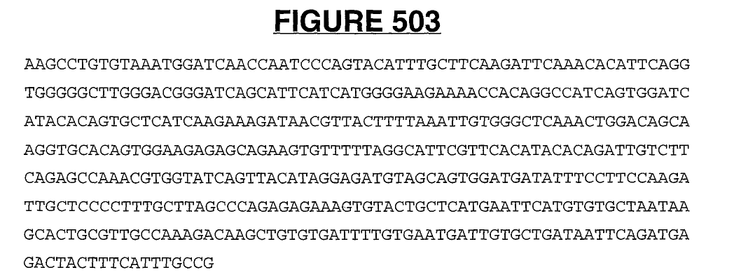

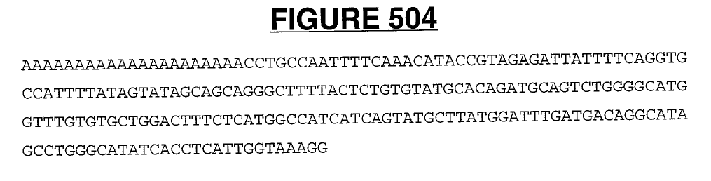

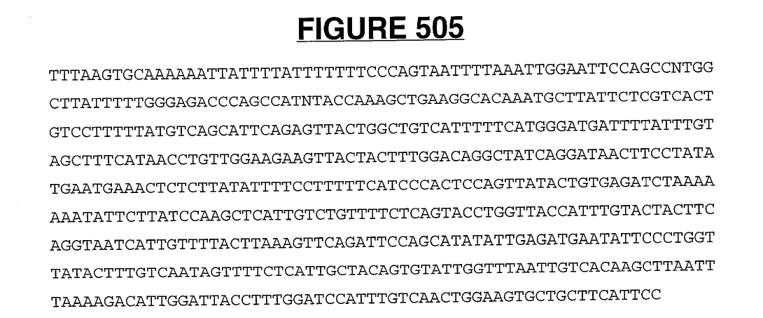

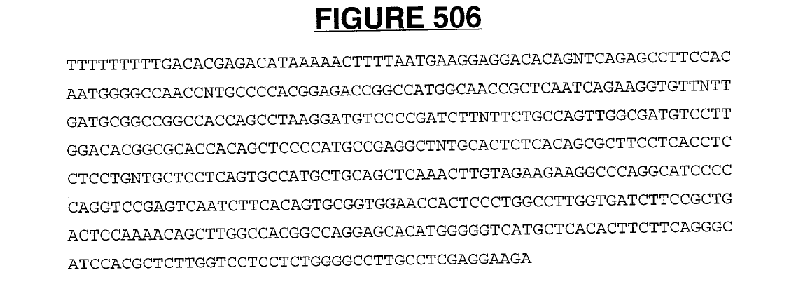

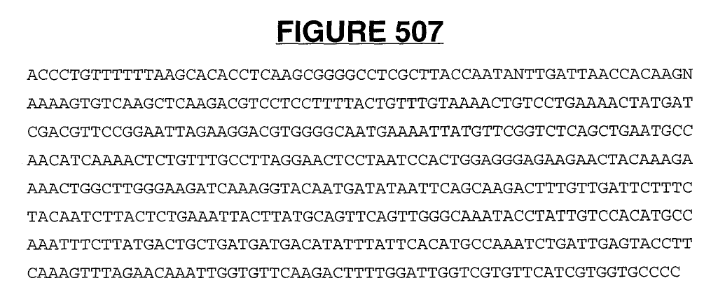

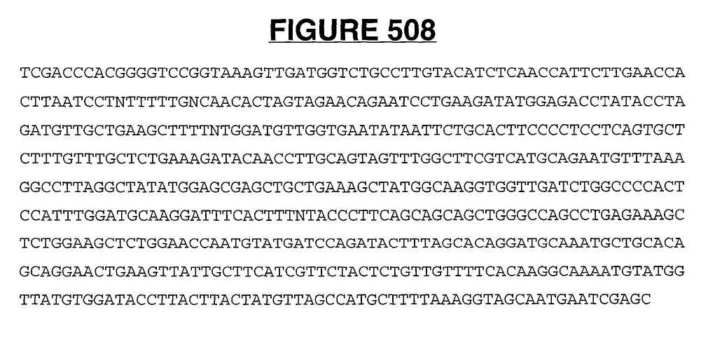

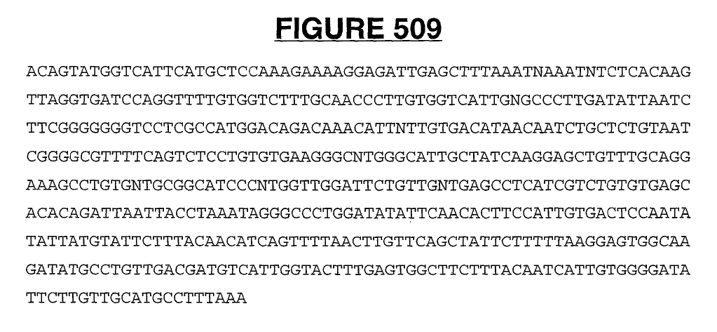

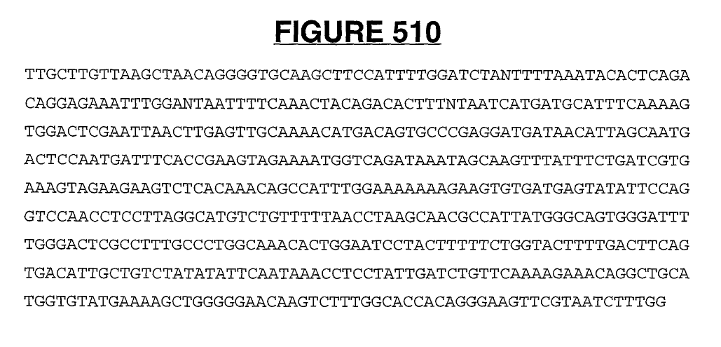

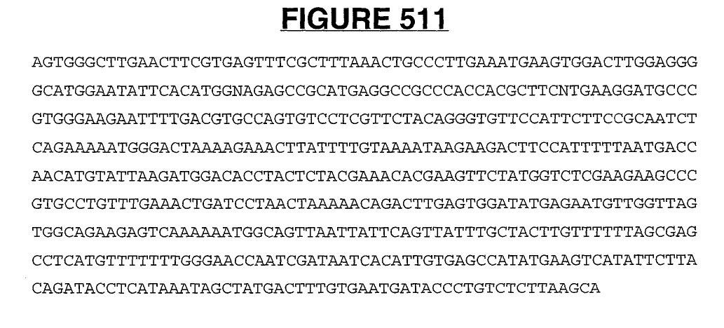

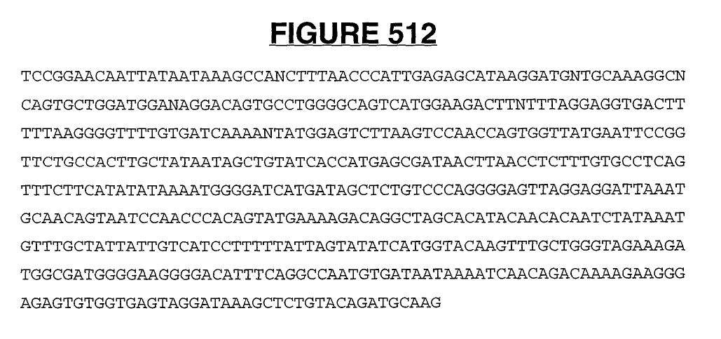

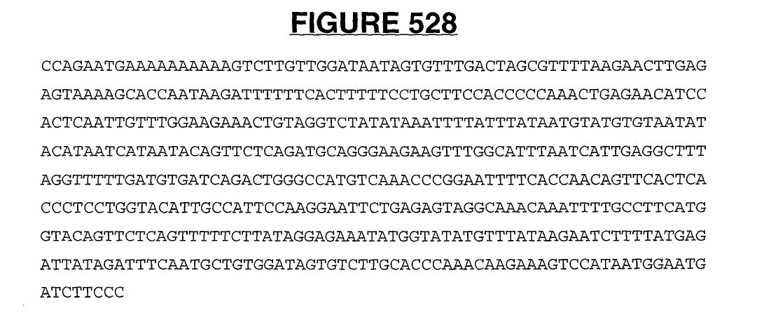

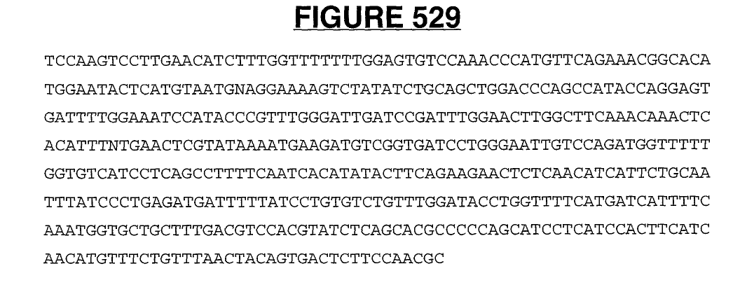

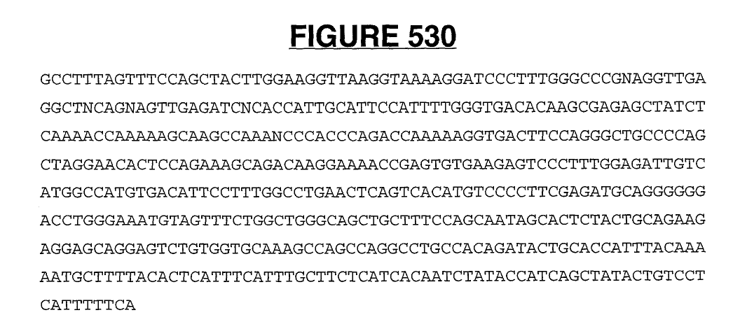

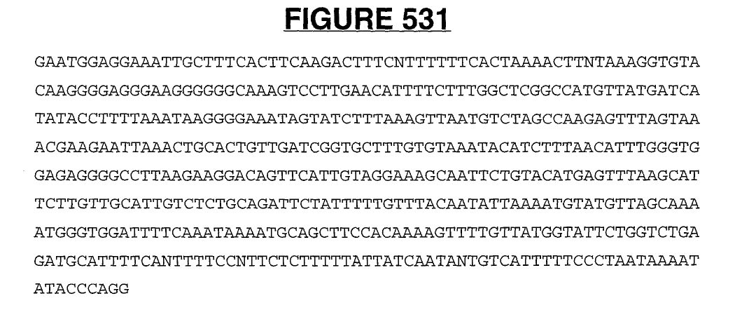

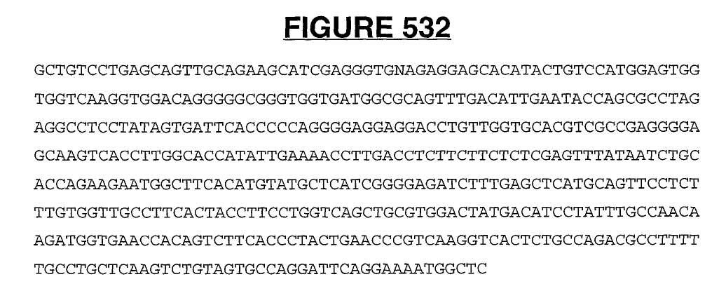

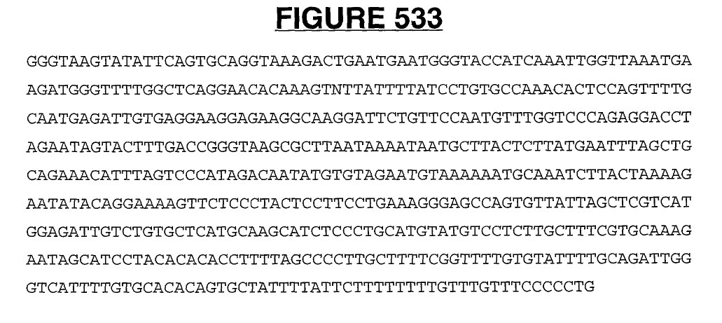

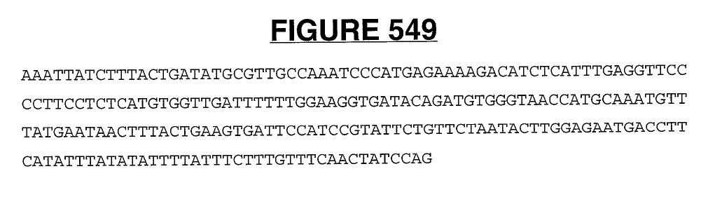

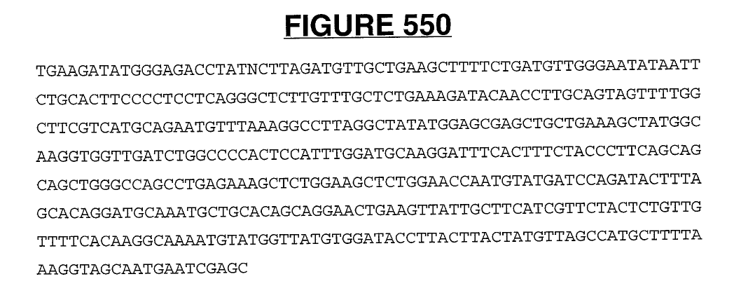

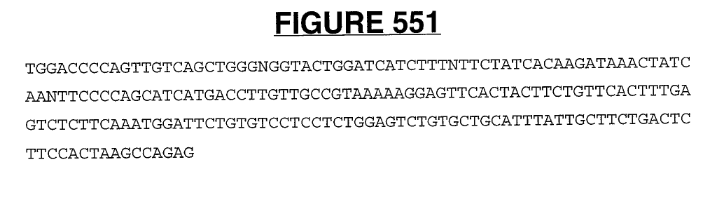

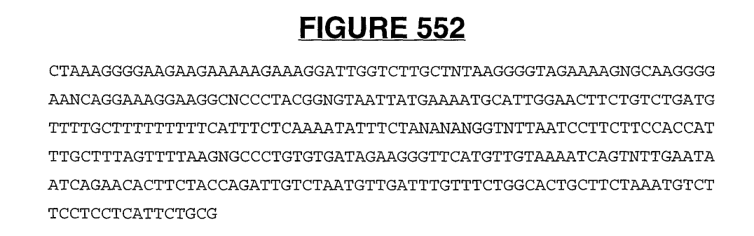

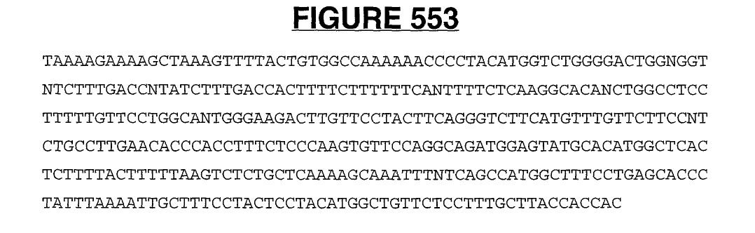

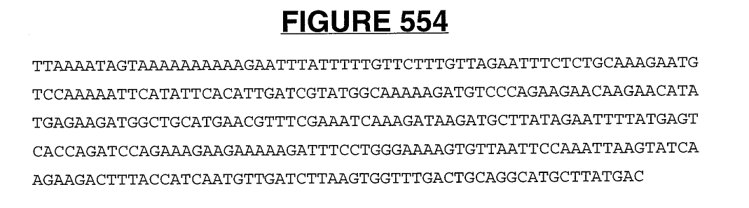

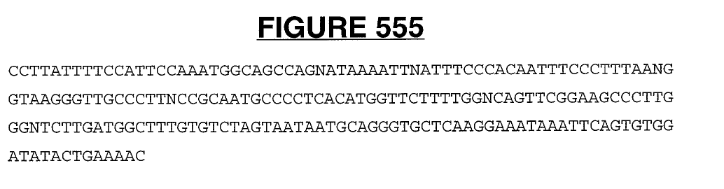

FIGS. 373-376, 468-476, 503-512, 528-533 and 549-555.

### EXAMPLE 2

### Identification of Full-Length cDNA Molecules

Oligonucleotide probes may be generated from the sequence of any of the SRT polynucleotide sequences disclosed herein, including those shown in FIGS. 1 to 562 and used to screen human cDNA libraries prepared as described in paragraph 1 of Example 1 above. The cloning vector may be pRK5B (pRKSB is a precursor of pRK5D that does not contain the SfiI site; see, Holmes et al., Science 253:1278-1280 (1991)), and the cDNA size cut may be less than 2800 bp. The oligonucleotides probes may be synthesized: 1) to identify by PCR a cDNA library that contained the sequence of interest, and 2) for use as probes to isolate a clone of the full-length coding sequence for SRT. Forward and reverse PCR primers generally range from 20 to 30 nucleotides and are often designed to give a PCR product of about 100-1000 bp in length. The probe sequences are typically 40-55 bp in length. In order to screen several libraries for a full-length clone, DNA from the libraries may be screened by PCR amplification, as per Ausubel et al., Current Protocols in Molecular Biology, supra, with the PCR primer pair. A positive library may then be used to isolate clones encoding the gene of interest using the probe oligonucleotide and one of the primer pairs.

### EXAMPLE 3

### Use of SRT Polynucleotides as Hybridization Probes

The following method describes use of a nucleotide sequence encoding SRT as a hybridization probe.

DNA comprising the coding sequence of full-length or mature SRT is employed as a probe to screen for homologous DNAs (such as those encoding naturally-occurring variants of SRT) in human tissue cDNA libraries or human tissue genomic libraries.

Hybridization and washing of filters containing either library DNAs is performed under the following high stringency conditions. Hybridization of radiolabeled SRT-derived probe to the filters is performed in a solution of 50% formamide, 5×SSC, 0.1% SDS, 0.1% sodium pyrophosphate, 50 mM sodium phosphate, pH 6.8, 2×Denhardt's solution, and 10% dextran sulfate at 42° C. for 20 hours. Washing of the filters is performed in an aqueous solution of 0.1x SSC and 0.1% SDS at 42° C.

DNAs having a desired sequence identity with the DNA encoding full-length native sequence SRT can then be identified using standard techniques known in the art.

### EXAMPLE 4

### Expression of SRT in E. coli

This example illustrates preparation of an unglycosylated form of SRT by recombinant expression in E. coli.

The DNA sequence encoding SRT is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the SRT coding region, lambda transcriptional terminator, and an argu gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized SRT protein can then be purified using a metal chelating column under conditions that allow tight binding of the protein.

SRT may be expressed in E. coli in a poly-His tagged form, using the following procedure. The DNA encoding SRT is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galErpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate•2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded SRT polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### EXAMPLE 5

### Expression of SRT in Mammalian Cells

This example illustrates preparation of a potentially glycosylated form of SRT by recombinant expression in mammalian cells.

The vector, pRK5 (see EP 307,247, published Mar. 15, 1989), is employed as the expression vector. Optionally, the SRT DNA is ligated into pRK5 with selected restriction enzymes to allow insertion of the SRT DNA using ligation methods such as described in Sambrook et al., supra. The resulting vector is called pRK5-SRT.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-SRT DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of SRT polypeptide. The cultures containing transfected cells may undergo further incubation (in serum free medium) and the medium is tested in selected bioassays.

In an alternative technique, SRT may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-SRT DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed SRT can then be concentrated and purified by any selected method, such as dialysis and/or column chromatography.

In another embodiment, SRT can be expressed in CHO cells. The pRK5-SRT can be transfected into CHO cells using known reagents such as CaPo₄ or DEAE-dextran. As described above, the cell cultures can be incubated, and the medium replaced with culture medium (alone) or medium containing a radiolabel such as ³⁵S-methionine. After determining the presence of SRT polypeptide, the culture medium may be replaced with serum free medium. Preferably, the cultures are incubated for about 6 days, and then the conditioned medium is harvested. The medium containing the expressed SRT can then be concentrated and purified by any selected method.

Epitope-tagged SRT may also be expressed in host CHO cells. The SRT may be subcloned out of the pRK5 vector. The subclone insert can undergo PCR to fuse in frame with a selected epitope tag such as a poly-his tag into a Baculovirus expression vector. The poly-his tagged SRT insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged SRT can then be concentrated and purified by any selected method, such as by Ni²⁺-chelate affinity chromatography.

SRT may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### EXAMPLE 6

### Expression of SRT in Yeast

The following method describes recombinant expression of SRT in yeast.

First, yeast expression vectors are constructed for intracellular production or secretion of SRT from the ADH2/GAPDH promoter. DNA encoding SRT and the promoter is inserted into suitable restriction enzyme sites in the selected plasmid to direct intracellular expression of SRT. For secretion, DNA encoding SRT can be cloned into the selected plasmid, together with DNA encoding the ADH2/GAPDH promoter, a native SRT signal peptide or other mammalian signal peptide, or, for example, a yeast alpha-factor or invertase secretory signal/leader sequence, and linker sequences (if needed) for expression of SRT.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant SRT can subsequently be isolated and purified by removing the yeast cells from the fermentation medium by centrifugation and then concentrating the medium using selected cartridge filters. The concentrate containing SRT may further be purified using selected column chromatography resins.

### EXAMPLE 7

### Expression of SRT in Baculovirus-Infected Insect Cells

The following method describes recombinant expression of SRT in Baculovirus-infected insect cells.

The sequence coding for SRT is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding SRT or the desired portion of the coding sequence of SRT such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by OReilley et al., Baculovirus expression vectors: A Laboratory Manual, Oxford: Oxford University Press (1994).

Expressed poly-his tagged SRT can then be purified, for example, by Ni²+-chelate affinity chromatography as follows. Extracts are prepared from recombinant virus-infected Sf9 cells as described by Rupert et al. Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged SRT are pooled and dialyzed against loading buffer.

Alternatively, purification of the IgG tagged (or Fc tagged) SRT can be performed using known chromatography techniques, including for instance, Protein A or protein G column chromatography.

### EXAMPLE 8

### Preparation of Antibodies That Bind SRT

This example illustrates preparation of monoclonal antibodies which can specifically bind SRT.

Techniques for producing the monoclonal antibodies are known in the art and are described, for instance, in Goding, supra. Immunogens that may be employed include purified SRT, fusion proteins containing SRT, and cells expressing recombinant SRT on the cell surface. Selection of the immunogen can be made by the skilled artisan without undue experimentation.

Mice, such as Balb/c, are immunized with the SRT immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-SRT antibodies.

After a suitable antibody titer has been detected, the animals “positive” for antibodies can be injected with a final intravenous injection of SRT. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against SRT. Determination of “positive” hybridoma cells secreting the desired monoclonal antibodies against SRT is within the skill in the art.

The positive hybridoma cells can be injected intraperitoneally into syngeneic Balb/c mice to produce ascites containing the anti-SRT monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### EXAMPLE 9

### Purification of SRT Polypeptides Using Specific Antibodies

Native or recombinant SRT polypeptides may be purified by a variety of standard techniques in the art of protein purification. For example, pro-SRT polypeptide, mature SRT polypeptide, or pre-SRT polypeptide is purified by immunoaffinity chromatography using antibodies specific for the SRT polypeptide of interest. In general, an immunoaffinity column is constructed by covalently coupling the anti-SRT polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of SRT polypeptide by preparing a fraction from cells containing SRT polypeptide in a soluble form. This preparation is derived by solubilization of the whole cell or of a subcellular fraction obtained via differential centrifugation by the addition of detergent or by other methods well known in the art. Alternatively, soluble SRT polypeptide containing a signal sequence may be secreted in useful quantity into the medium in which the cells are grown.

A soluble SRT polypeptide-containing preparation is passed over the immunoaffinity column, and the column is washed under conditions that allow the preferential absorbance of SRT polypeptide (e.g., high ionic strength buffers in the presence of detergent). Then, the column is eluted under conditions that disrupt antibody/SRT polypeptide binding (e.g., a low pH buffer such as approximately pH 2-3, or a high concentration of a chaotrope such as urea or thiocyanate ion), and SRT polypeptide is collected.

### EXAMPLE 10

### Drug Screening

This invention is particularly useful for screening compounds by using SRT polypeptides or binding fragment thereof in any of a variety of drug screening techniques. The SRT polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the SRT polypeptide or fragment. Drugs are screened against such transformed cells in competitive binding assays. Such cells, either in viable or fixed form, can be used for standard binding assays. One may measure, for example, the formation of complexes between SRT polypeptide or a fragment and the agent being tested. Alternatively, one can examine the diminution in complex formation between the SRT polypeptide and its target cell or target receptors caused by the agent being tested.

Thus, the present invention provides methods of screening for drugs or any other agents which can affect a SRT polypeptide-associated disease or disorder. These methods comprise contacting such an agent with an SRT polypeptide or fragment thereof and assaying (I) for the presence of a complex between the agent and the SRT polypeptide or fragment, or (ii) for the presence of a complex between the SRT polypeptide or fragment and the cell, by methods well known in the art. In such competitive binding assays, the SRT polypeptide or fragment is typically labeled. After suitable incubation, free SRT polypeptide or fragment is separated from that present in bound form, and the amount of free or uncomplexed label is a measure of the ability of the particular agent to bind to SRT polypeptide or to interfere with the SRT polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a SRT polypeptide, the peptide test compounds are reacted with SRT polypeptide and washed. Bound SRT polypeptide is detected by methods well known in the art. Purified SRT polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding SRT polypeptide specifically compete with a test compound for binding to SRT polypeptide or fragments thereof. In this manner, the antibodies can be used to detect the presence of any peptide which shares one or more antigenic determinants with SRT polypeptide.

### EXAMPLE 11

### Rational Drug Design

The goal of rational drug design is to produce structural analogs of biologically active polypeptide of interest (i.e., a SRT polypeptide) or of small molecules with which they interact, e.g., agonists, antagonists, or inhibitors. Any of these examples can be used to fashion drugs which are more active or stable forms of the SRT polypeptide or which enhance or interfere with the function of the SRT polypeptide in vivo (c.f., Hodgson, Bio/Technology, 9: 19-21 (1991)).

In one approach, the three-dimensional structure of the SRT polypeptide, or of an SRT polypeptide-inhibitor complex, is determined by x-ray crystallography, by computer modeling or, most typically, by a combination of the two approaches. Both the shape and charges of the SRT polypeptide must be ascertained to elucidate the structure and to determine active site(s) of the molecule. Less often, useful information regarding the structure of the SRT polypeptide may be gained by modeling based on the structure of homologous proteins. In both cases, relevant structural information is used to design analogous SRT polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the SRT polypeptide may be made available to perform such analytical studies as X-ray crystallography. In addition, knowledge of the SRT polypeptide amino acid sequence provided herein will provide guidance to those employing computer modeling techniques in place of or in addition to x-ray crystallography.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed is:

1. An isolated nucleic acid molecule comprising a nucleotide sequence having at least about 80% nucleic acid sequence identity to (a) the DNA molecule of any one of FIG. 1 to 562, or (b) the complement of the DNA molecule of (a).

2. The isolated nucleic acid molecule of claim 1 comprising the nucleotide sequence shown in any one of FIG. 1 to 562, or the complement thereof.

3. The isolated nucleic acid molecule of claim 1 consisting essentially of a nucleotide sequence having at least about 80% nucleic acid sequence identity to (a) the DNA molecule of any one of FIG. 1 to 562, or (b) the complement of the DNA molecule of (a).

4. The isolated nucleic acid molecule of claim 1 consisting essentially of the nucleotide sequence shown in any one of FIG. 1 to 562, or the complement thereof.

5. The isolated nucleic acid molecule of claim 1 consisting of a nucleotide sequence having at least about 80% nucleic acid sequence identity to (a) the DNA molecule of any one of FIG. 1 to 562, or (b) the complement of the DNA molecule of (a).

6. The isolated nucleic acid molecule of claim 1 consisting of the nucleotide sequence shown in any one of FIG. 1 to 562, or the complement thereof.

7. An isolated nucleic acid molecule which hybridizes to (a) the DNA molecule of any one of FIG. 1 to 562, or (b) the complement of the DNA molecule of (a).

8. The isolated nucleic acid molecule of claim 7 which hybridizes to the complement of the DNA molecule of any one of FIG. 1 to 562.

9. The isolated nucleic acid molecule of claim 7, wherein said hybridization occurs under stringent hybridization conditions.

10. An isolated nucleic acid molecule comprising at least about 10 consecutive nucleotides contained within (a) the DNA molecule of any one of FIG. 1 to 562, or (b) the complement of the DNA molecule of (a).

11. The isolated nucleic acid molecule of claim 10 comprising at least about 10 consecutive nucleotides contained within the complement of the DNA molecule of any one of FIG. 1 to 562.

12. The isolated nucleic acid molecule of claim 10 which is from about 10 to about 1000 nucleotides in length.

13. The isolated nucleic acid molecule of claim 10 which is from about 10 to about 500 nucleotides in length.

14. The isolated nucleic acid molecule of claim 10 which is from about 10 to about 100 nucleotides in length.

15. The isolated nucleic acid molecule of claim 10 which is from about 10 to about 50 nucleotides in length.

16. The isolated nucleic acid molecule of claim 11 which is fully complementary to the DNA molecule of any one of FIG. 1 to 562.

17. The isolated nucleic acid molecule of claim 10 which is detectably labeled.

18. A method of detecting the presence of a cDNA molecule which encodes a mammalian polypeptide in a mammalian cDNA library, said method comprising:
contacting said cDNA library with an oligonucleotide probe that hybridizes to the DNA molecule of any one of FIG. 1 to 562, wherein said contacting is performed under conditions suitable for hybridization of said probe to a cDNA molecule in said library and wherein hybridization of said probe to a cDNA molecule in said library is indicative of the presence of cDNA molecule which encodes a mammalian polypeptide in said cDNA library.

19. The method of claim 18, wherein said hybridization is performed under stringent hybridization conditions.

20. The method of claim 18, wherein said oligonucleotide probe comprises at least about consecutive nucleotides contained within the complement of the DNA molecule of any one of FIG. 1 to 562.

21. The method of claim 18, wherein said mammalian polypeptide is a human polypeptide.

22. A vector comprising the nucleic acid molecule of claim 1.

23. The vector of claim 22, wherein said nucleic acid molecule is operably linked to control sequences recognized by a host cell transformed with the vector.

24. A host cell comprising the vector of claim 22.

25. The host cell of claim 24, wherein said cell is a CHO cell.

26. The host cell of claim 24, wherein said cell is an E. coli.

27. The host cell of claim 24, wherein said cell is a yeast cell.

28. An isolated SRT polypeptide encoded by the nucleic acid molecule of claim 1.

29. An antibody which binds to the isolated SRT polypeptide of claim 28.

30. The antibody of claim 29 which is a monoclonal antibody.

31. The antibody of claim 29 which is a humanized antibody.
